SEQUENCE LISTING (629 sequences)

SEQ ID NO: 1 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 2 - protein, 310 aa, synthetic construct
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 3 - protein, 310 aa, synthetic construct [sequence omitted]

SEQ ID NO: 4 - DNA, 933 bp, Homo sapiens
  source 1..933: mol_type = genomic DNA
atgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcctga

SEQ ID NO: 5 - DNA, 933 bp, Homo sapiens; identical to SEQ ID NO: 4
  source 1..933: mol_type = genomic DNA

SEQ ID NO: 6 - protein, 311 aa, Mus sp.
MRRAALWLWLCALALRLQPALPQIVAVNVPPEDQDGSGDDSDNFSGSGTGALPDTLSRQTPSTWKDVWLLTATPTAPEPTSSNTETAFTSVLPAGEKPEEGEPVLHVEAEPGFTARDKEKEVTTRPRETVQLPITQRASTVRVTTAQAAVTSHPHGGMQPGLHETSAPTAPGQPDHQPPRVEGGGTSVIKEVVEDGTANQLPAGEGSGEQDFTFETSGENTAVAAVEPGLRNQPPVDEGATGASQSLLDRKEVLGGVIAGGLVGLIFAVCLVAFMLYRMKKKDEGSYSLEEPKQANGGAYQKPTKQEEFYA

SEQ ID NO: 7 - DNA, 936 bp, Mus sp.
  source 1..936: mol_type = genomic DNA
atgagacgcgcggcgctctggctctggctctgcgcgctggcgctgcgcctgcagcctgccctcccgcaaattgtggctgtaaatgttcctcctgaagatcaggatggctctggggatgactctgacaacttctctggctctggcacaggtgctttgccagatactttgtcacggcagacaccttccacttggaaggacgtgtggctgttgacagccacgcccacagctccagagcccaccagcagcaacaccgagactgcttttacctctgtcctgccagccggagagaagcccgaggagggagagcctgtgctccatgtagaagcagagcctggcttcactgctcgggacaaggaaaaggaggtcaccaccaggcccagggagaccgtgcagctccccatcacccaacgggcctcaacagtcagagtcaccacagcccaggcagctgtcacatctcatccgcacgggggcatgcaacctggcctccatgagacctcggctcccacagcacctggtcaacctgaccatcagcctccacgtgtggagggtggcggcacttctgtcatcaaagaggttgtcgaggatggaactgccaatcagcttcccgcaggagagggctctggagaacaagacttcacctttgaaacatctggggagaacacagctgtggctgccgtagagcccggcctgcggaatcagcccccggtggacgaaggagccacaggtgcttctcagagccttttggacaggaaggaagtgctgggaggtgtcattgccggaggcctagtgggcctcatctttgctgtgtgcctggtggctttcatgctgtaccggatgaagaagaaggacgaaggcagctactccttggaggagcccaaacaagccaatggcggtgcctaccagaaacccaccaagcaggaggagttctacgcctga

SEQ ID NO: 8 - protein, 39 aa, synthetic construct
QIVATNLPPEDQDGSGDDSDNFSGSGAGALQDITLSQQT

SEQ ID NO: 9 - protein, 45 aa, synthetic construct
ALQDITLSQQTPSTWKDTQLLTAIPTSPEPTGLEATAASTSTLPA

SEQ ID NO: 10 - protein, 34 aa, synthetic construct
ASTSTLPAGEGPKEGEAVVLPEVEPGLTAREQEA

SEQ ID NO: 11 - protein, 15 aa, synthetic construct
ASTSTLPAGEGPKEG

SEQ ID NO: 12 - protein, 40 aa, synthetic construct
EPGLTAREQEATPRPRETTQLPTTHQASTTTATTAQEPAT

SEQ ID NO: 13 - protein, 35 aa, synthetic construct
QEPATSHPHRDMQPGHHETSTPAGPSQADLHTPHT

SEQ ID NO: 14 - protein, 75 aa, synthetic construct
HTPHTEDGGPSATERAAEDGASSQLPAAEGSGEQDFTFETSGENTAVVAVEPDRRNQSPVDQGATGASQGLLDRK

SEQ ID NO: 15 - protein, 39 aa, synthetic construct
HTPHTEDGGPSATERAAEDGASSQLPAAEGSGEQDFTFE

SEQ ID NO: 16 - protein, 41 aa, synthetic construct
DFTFETSGENTAVVAVEPDRRNQSPVDQGATGASQGLLDRK

SEQ ID NO: 17 - protein, 26 aa, synthetic construct
TAVVAVEPDRRNQSPVDQGATGASQG

SEQ ID NO: 18 - protein, 4 aa, synthetic construct
DFTF

SEQ ID NO: 19 - protein, 4 aa, synthetic construct
FTFE

SEQ ID NO: 20 - protein, 4 aa, synthetic construct
TFET

SEQ ID NO: 21 - protein, 4 aa, synthetic construct
FETS

SEQ ID NO: 22 - protein, 4 aa, synthetic construct
ETSG

SEQ ID NO: 23 - protein, 4 aa, synthetic construct
TSGE

SEQ ID NO: 24 - protein, 4 aa, synthetic construct
SGEN

SEQ ID NO: 25 - protein, 4 aa, synthetic construct
GENT

SEQ ID NO: 26 - protein, 4 aa, synthetic construct
ENTA

SEQ ID NO: 27 - protein, 4 aa, synthetic construct
NTAV

SEQ ID NO: 28 - protein, 4 aa, synthetic construct
TAVV

SEQ ID NO: 29 - protein, 4 aa, synthetic construct
AVVA

SEQ ID NO: 30 - protein, 4 aa, synthetic construct
VVAV

SEQ ID NO: 31 - protein, 4 aa, synthetic construct
VAVE

SEQ ID NO: 32 - protein, 4 aa, synthetic construct
AVEP

SEQ ID NO: 33 - protein, 4 aa, synthetic construct
VEPD

SEQ ID NO: 34 - protein, 4 aa, synthetic construct
EPDR

SEQ ID NO: 35 - protein, 4 aa, synthetic construct
PDRR

SEQ ID NO: 36 - protein, 4 aa, synthetic construct
DRRN

SEQ ID NO: 37 - protein, 4 aa, synthetic construct
RRNQ

SEQ ID NO: 38 - protein, 4 aa, synthetic construct
RNQS

SEQ ID NO: 39 - protein, 4 aa, synthetic construct
NQSP

SEQ ID NO: 40 - protein, 4 aa, synthetic construct
QSPV

SEQ ID NO: 41 - protein, 4 aa, synthetic construct
SPVD

SEQ ID NO: 42 - protein, 4 aa, synthetic construct
PVDQ

SEQ ID NO: 43 - protein, 4 aa, synthetic construct
VDQG

SEQ ID NO: 44 - protein, 4 aa, synthetic construct
DQGA

SEQ ID NO: 45 - protein, 4 aa, synthetic construct
QGAT

SEQ ID NO: 46 - protein, 4 aa, synthetic construct
GATG

SEQ ID NO: 47 - protein, 4 aa, synthetic construct
ATGA

SEQ ID NO: 48 - protein, 4 aa, synthetic construct
TGAS

SEQ ID NO: 49 - protein, 4 aa, synthetic construct
GASQ

SEQ ID NO: 50 - protein, 4 aa, synthetic construct
ASQG

SEQ ID NO: 51 - protein, 4 aa, synthetic construct
SQGL

SEQ ID NO: 52 - protein, 4 aa, synthetic construct
QGLL

SEQ ID NO: 53 - protein, 4 aa, synthetic construct
GLLD

SEQ ID NO: 54 - protein, 4 aa, synthetic construct
LLDR

SEQ ID NO: 55 - protein, 4 aa, synthetic construct
LDRK

SEQ ID NO: 56 - protein, 5 aa, synthetic construct
DFTFE

SEQ ID NO: 57 - protein, 5 aa, synthetic construct
FTFET

SEQ ID NO: 58 - protein, 5 aa, synthetic construct
TFETS

SEQ ID NO: 59 - protein, 5 aa, synthetic construct
FETSG

SEQ ID NO: 60 - protein, 5 aa, synthetic construct
ETSGE

SEQ ID NO: 61 - protein, 5 aa, synthetic construct
TSGEN

SEQ ID NO: 62 - protein, 5 aa, synthetic construct
SGENT

SEQ ID NO: 63 - protein, 5 aa, synthetic construct
GENTA

SEQ ID NO: 64 - protein, 5 aa, synthetic construct
ENTAV

SEQ ID NO: 65 - protein, 5 aa, synthetic construct
NTAVV

SEQ ID NO: 66 - protein, 5 aa, synthetic construct
TAVVA

SEQ ID NO: 67 - protein, 5 aa, synthetic construct
AVVAV

SEQ ID NO: 68 - protein, 5 aa, synthetic construct
VVAVE

SEQ ID NO: 69 - protein, 5 aa, synthetic construct
VAVEP

SEQ ID NO: 70 - protein, 5 aa, synthetic construct
AVEPD

SEQ ID NO: 71 - protein, 5 aa, synthetic construct
VEPDR

SEQ ID NO: 72 - protein, 5 aa, synthetic construct
EPDRR

SEQ ID NO: 73 - protein, 5 aa, synthetic construct
PDRRN

SEQ ID NO: 74 - protein, 5 aa, synthetic construct
DRRNQ

SEQ ID NO: 75 - protein, 5 aa, synthetic construct
RRNQS

SEQ ID NO: 76 - protein, 5 aa, synthetic construct
RNQSP

SEQ ID NO: 77 - protein, 5 aa, synthetic construct
NQSPV

SEQ ID NO: 78 - protein, 5 aa, synthetic construct
QSPVD

SEQ ID NO: 79 - protein, 5 aa, synthetic construct
SPVDQ

SEQ ID NO: 80 - protein, 5 aa, synthetic construct
PVDQG

SEQ ID NO: 81 - protein, 5 aa, synthetic construct
VDQGA

SEQ ID NO: 82 - protein, 5 aa, synthetic construct
DQGAT

SEQ ID NO: 83 - protein, 5 aa, synthetic construct
QGATG

SEQ ID NO: 84 - protein, 5 aa, synthetic construct
GATGA

SEQ ID NO: 85 - protein, 5 aa, synthetic construct
ATGAS

SEQ ID NO: 86 - protein, 5 aa, synthetic construct
TGASQ

SEQ ID NO: 87 - protein, 5 aa, synthetic construct
GASQG

SEQ ID NO: 88 - protein, 5 aa, synthetic construct
ASQGL

SEQ ID NO: 89 - protein, 5 aa, synthetic construct
SQGLL

SEQ ID NO: 90 - protein, 5 aa, synthetic construct
QGLLD

SEQ ID NO: 91 - protein, 5 aa, synthetic construct
GLLDR

SEQ ID NO: 92 - protein, 5 aa, synthetic construct
LLDRK

SEQ ID NO: 93 - protein, 6 aa, synthetic construct
DFTFET

SEQ ID NO: 94 - protein, 6 aa, synthetic construct
FTFETS

SEQ ID NO: 95 - protein, 6 aa, synthetic construct
TFETSG

SEQ ID NO: 96 - protein, 6 aa, synthetic construct
FETSGE

SEQ ID NO: 97 - protein, 6 aa, synthetic construct
ETSGEN

SEQ ID NO: 98 - protein, 6 aa, synthetic construct
TSGENT

SEQ ID NO: 99 - protein, 6 aa, synthetic construct
SGENTA

SEQ ID NO: 100 - protein, 6 aa, synthetic construct
GENTAV

SEQ ID NO: 101 - protein, 6 aa, synthetic construct
ENTAVV

SEQ ID NO: 102 - protein, 6 aa, synthetic construct
NTAVVA

SEQ ID NO: 103 - protein, 6 aa, synthetic construct
TAVVAV

SEQ ID NO: 104 - protein, 6 aa, synthetic construct
AVVAVE

SEQ ID NO: 105 - protein, 6 aa, synthetic construct
VVAVEP

SEQ ID NO: 106 - protein, 6 aa, synthetic construct
VAVEPD

SEQ ID NO: 107 - protein, 6 aa, synthetic construct
AVEPDR

SEQ ID NO: 108 - protein, 6 aa, synthetic construct
VEPDRR

SEQ ID NO: 109 - protein, 6 aa, synthetic construct
EPDRRN

SEQ ID NO: 110 - protein, 6 aa, synthetic construct
PDRRNQ

SEQ ID NO: 111 - protein, 6 aa, synthetic construct
DRRNQS

SEQ ID NO: 112 - protein, 6 aa, synthetic construct
RRNQSP

SEQ ID NO: 113 - protein, 6 aa, synthetic construct
RNQSPV

SEQ ID NO: 114 - protein, 6 aa, synthetic construct
NQSPVD

SEQ ID NO: 115 - protein, 6 aa, synthetic construct
QSPVDQ

SEQ ID NO: 116 - protein, 6 aa, synthetic construct
SPVDQG

SEQ ID NO: 117 - protein, 6 aa, synthetic construct
PVDQGA

SEQ ID NO: 118 - protein, 6 aa, synthetic construct
VDQGAT

SEQ ID NO: 119 - protein, 6 aa, synthetic construct
DQGATG

SEQ ID NO: 120 - protein, 6 aa, synthetic construct
QGATGA

SEQ ID NO: 121 - protein, 6 aa, synthetic construct
GATGAS

SEQ ID NO: 122 - protein, 6 aa, synthetic construct
ATGASQ

SEQ ID NO: 123 - protein, 6 aa, synthetic construct
TGASQG

SEQ ID NO: 124 - protein, 6 aa, synthetic construct
GASQGL

SEQ ID NO: 125 - protein, 6 aa, synthetic construct
ASQGLL

SEQ ID NO: 126 - protein, 6 aa, synthetic construct
SQGLLD

SEQ ID NO: 127 - protein, 6 aa, synthetic construct
QGLLDR

SEQ ID NO: 128 - protein, 6 aa, synthetic construct
GLLDRK

SEQ ID NO: 129 - protein, 4 aa, synthetic construct
HTPH

SEQ ID NO: 130 - protein, 4 aa, synthetic construct
TPHT

SEQ ID NO: 131 - protein, 4 aa, synthetic construct
PHTE

SEQ ID NO: 132 - protein, 4 aa, synthetic construct
HTED

SEQ ID NO: 133 - protein, 4 aa, synthetic construct
TEDG

SEQ ID NO: 134 - protein, 4 aa, synthetic construct
EDGG

SEQ ID NO: 135 - protein, 4 aa, synthetic construct
DGGP

SEQ ID NO: 136 - protein, 4 aa, synthetic construct
GGPS

SEQ ID NO: 137 - protein, 4 aa, synthetic construct
GPSA

SEQ ID NO: 138 - protein, 4 aa, synthetic construct
PSAT

SEQ ID NO: 139 - protein, 4 aa, synthetic construct
SATE

SEQ ID NO: 140 - protein, 4 aa, synthetic construct
ATER

SEQ ID NO: 141 - protein, 4 aa, synthetic construct
TERA

SEQ ID NO: 142 - protein, 4 aa, synthetic construct
ERAA

SEQ ID NO: 143 - protein, 4 aa, synthetic construct
RAAE

SEQ ID NO: 144 - protein, 4 aa, synthetic construct
AAED

SEQ ID NO: 145 - protein, 4 aa, synthetic construct
AEDG

SEQ ID NO: 146 - protein, 4 aa, synthetic construct
EDGA

SEQ ID NO: 147 - protein, 4 aa, synthetic construct
DGAS

SEQ ID NO: 148 - protein, 4 aa, synthetic construct
GASS

SEQ ID NO: 149 - protein, 4 aa, synthetic construct
ASSQ

SEQ ID NO: 150 - protein, 4 aa, synthetic construct
SSQL

SEQ ID NO: 151 - protein, 4 aa, synthetic construct
SQLP

SEQ ID NO: 152 - protein, 4 aa, synthetic construct
QLPA

SEQ ID NO: 153 - protein, 4 aa, synthetic construct
LPAA

SEQ ID NO: 154 - protein, 4 aa, synthetic construct
PAAE

SEQ ID NO: 155 - protein, 4 aa, synthetic construct
AAEG

SEQ ID NO: 156 - protein, 4 aa, synthetic construct
AEGS

SEQ ID NO: 157 - protein, 4 aa, synthetic construct
EGSG

SEQ ID NO: 158 - protein, 4 aa, synthetic construct
GSGE

SEQ ID NO: 159 - protein, 4 aa, synthetic construct
SGEQ

SEQ ID NO: 160 - protein, 4 aa, synthetic construct
GEQD

SEQ ID NO: 161 - protein, 4 aa, synthetic construct
EQDF

SEQ ID NO: 162 - protein, 4 aa, synthetic construct
QDFT

SEQ ID NO: 163 - protein, 5 aa, synthetic construct
HTPHT

SEQ ID NO: 164 - protein, 5 aa, synthetic construct
TPHTE

SEQ ID NO: 165 - protein, 5 aa, synthetic construct
PHTED

SEQ ID NO: 166 - protein, 5 aa, synthetic construct
HTEDG

SEQ ID NO: 167 - protein, 5 aa, synthetic construct
TEDGG

SEQ ID NO: 168 - protein, 5 aa, synthetic construct
EDGGP

SEQ ID NO: 169 - protein, 5 aa, synthetic construct
DGGPS

SEQ ID NO: 170 - protein, 5 aa, synthetic construct
GGPSA

SEQ ID NO: 171 - protein, 5 aa, synthetic construct
GPSAT

SEQ ID NO: 172 - protein, 5 aa, synthetic construct
PSATE

SEQ ID NO: 173 - protein, 5 aa, synthetic construct
SATER

SEQ ID NO: 174 - protein, 5 aa, synthetic construct
ATERA

SEQ ID NO: 175 - protein, 5 aa, synthetic construct
TERAA

SEQ ID NO: 176 - protein, 5 aa, synthetic construct
ERAAE

SEQ ID NO: 177 - protein, 5 aa, synthetic construct
RAAED

SEQ ID NO: 178 - protein, 5 aa, synthetic construct
AAEDG

SEQ ID NO: 179 - protein, 5 aa, synthetic construct
AEDGA

SEQ ID NO: 180 - protein, 5 aa, synthetic construct
EDGAS

SEQ ID NO: 181 - protein, 5 aa, synthetic construct
DGASS

SEQ ID NO: 182 - protein, 5 aa, synthetic construct
GASSQ

SEQ ID NO: 183 - protein, 5 aa, synthetic construct
ASSQL

SEQ ID NO: 184 - protein, 5 aa, synthetic construct
SSQLP

SEQ ID NO: 185 - protein, 5 aa, synthetic construct
SQLPA

SEQ ID NO: 186 - protein, 5 aa, synthetic construct
QLPAA

SEQ ID NO: 187 - protein, 5 aa, synthetic construct
LPAAE

SEQ ID NO: 188 - protein, 5 aa, synthetic construct
PAAEG

SEQ ID NO: 189 - protein, 5 aa, synthetic construct
AAEGS

SEQ ID NO: 190 - protein, 5 aa, synthetic construct
AEGSG

SEQ ID NO: 191 - protein, 5 aa, synthetic construct
EGSGE

SEQ ID NO: 192 - protein, 5 aa, synthetic construct
GSGEQ

SEQ ID NO: 193 - protein, 5 aa, synthetic construct
SGEQD

SEQ ID NO: 194 - protein, 5 aa, synthetic construct
GEQDF

SEQ ID NO: 195 - protein, 5 aa, synthetic construct
EQDFT

SEQ ID NO: 196 - protein, 5 aa, synthetic construct
QDFTF

SEQ ID NO: 197 - protein, 6 aa, synthetic construct
HTPHTE

SEQ ID NO: 198 - protein, 6 aa, synthetic construct
TPHTED

SEQ ID NO: 199 - protein, 6 aa, synthetic construct
PHTEDG

SEQ ID NO: 200 - protein, 6 aa, synthetic construct
HTEDGG

SEQ ID NO: 201 - protein, 6 aa, synthetic construct
TEDGGP

SEQ ID NO: 202 - protein, 6 aa, synthetic construct
EDGGPS

SEQ ID NO: 203 - protein, 6 aa, synthetic construct
DGGPSA

SEQ ID NO: 204 - protein, 6 aa, synthetic construct
GGPSAT

SEQ ID NO: 205 - protein, 6 aa, synthetic construct
GPSATE

SEQ ID NO: 206 - protein, 6 aa, synthetic construct
PSATER

SEQ ID NO: 207 - protein, 6 aa, synthetic construct
SATERA

SEQ ID NO: 208 - protein, 6 aa, synthetic construct
ATERAA

SEQ ID NO: 209 - protein, 6 aa, synthetic construct
TERAAE

SEQ ID NO: 210 - protein, 6 aa, synthetic construct
ERAAED

SEQ ID NO: 211 - protein, 6 aa, synthetic construct
RAAEDG

SEQ ID NO: 212 - protein, 6 aa, synthetic construct
AAEDGA

SEQ ID NO: 213 - protein, 6 aa, synthetic construct
AEDGAS

SEQ ID NO: 214 - protein, 6 aa, synthetic construct
EDGASS

SEQ ID NO: 215 - protein, 6 aa, synthetic construct
DGASSQ

SEQ ID NO: 216 - protein, 6 aa, synthetic construct
GASSQL

SEQ ID NO: 217 - protein, 6 aa, synthetic construct
ASSQLP

SEQ ID NO: 218 - protein, 6 aa, synthetic construct
SSQLPA

SEQ ID NO: 219 - protein, 6 aa, synthetic construct
SQLPAA

SEQ ID NO: 220 - protein, 6 aa, synthetic construct
QLPAAE

SEQ ID NO: 221 - protein, 6 aa, synthetic construct
LPAAEG

SEQ ID NO: 222 - protein, 6 aa, synthetic construct
PAAEGS

SEQ ID NO: 223 - protein, 6 aa, synthetic construct
AAEGSG

SEQ ID NO: 224 - protein, 6 aa, synthetic construct
AEGSGE

SEQ ID NO: 225 - protein, 6 aa, synthetic construct
EGSGEQ

SEQ ID NO: 226 - protein, 6 aa, synthetic construct
GSGEQD

SEQ ID NO: 227 - protein, 6 aa, synthetic construct
SGEQDF

SEQ ID NO: 228 - protein, 6 aa, synthetic construct
GEQDFT

SEQ ID NO: 229 - protein, 6 aa, synthetic construct
EQDFTF

SEQ ID NO: 230 - protein, 6 aa, synthetic construct
QDFTFE

SEQ ID NO: 231 - protein, 4 aa, synthetic construct
ASTS

SEQ ID NO: 232 - protein, 4 aa, synthetic construct
STST

SEQ ID NO: 233 - protein, 4 aa, synthetic construct
TSTL

SEQ ID NO: 234 - protein, 4 aa, synthetic construct
STLP

SEQ ID NO: 235 - protein, 4 aa, synthetic construct
TLPA

SEQ ID NO: 236 - protein, 4 aa, synthetic construct
LPAG

SEQ ID NO: 237 - protein, 4 aa, synthetic construct
PAGE

SEQ ID NO: 238 - protein, 4 aa, synthetic construct
AGEG

SEQ ID NO: 239 - protein, 4 aa, synthetic construct
GEGP

SEQ ID NO: 240 - protein, 4 aa, synthetic construct
EGPK

SEQ ID NO: 241 - protein, 4 aa, synthetic construct
GPKE

SEQ ID NO: 242 - protein, 4 aa, synthetic construct
PKEG

SEQ ID NO: 243 - protein, 4 aa, synthetic construct
KEGE

SEQ ID NO: 244 - protein, 4 aa, synthetic construct
EGEA

SEQ ID NO: 245 - protein, 4 aa, synthetic construct
GEAV

SEQ ID NO: 246 - protein, 4 aa, synthetic construct
EAVV

SEQ ID NO: 247 - protein, 4 aa, synthetic construct
AVVL

SEQ ID NO: 248 - protein, 4 aa, synthetic construct
VVLP

SEQ ID NO: 249 - protein, 4 aa, synthetic construct
VLPE

SEQ ID NO: 250 - protein, 4 aa, synthetic construct
LPEV

SEQ ID NO: 251 - protein, 4 aa, synthetic construct
PEVE

SEQ ID NO: 252 - protein, 4 aa, synthetic construct
EVEP

SEQ ID NO: 253 - protein, 4 aa, synthetic construct
VEPG

SEQ ID NO: 254 - protein, 4 aa, synthetic construct
EPGL

SEQ ID NO: 255 - protein, 4 aa, synthetic construct
PGLT

SEQ ID NO: 256 - protein, 4 aa, synthetic construct
GLTA

SEQ ID NO: 257 - protein, 4 aa, synthetic construct
LTAR

SEQ ID NO: 258 - protein, 4 aa, synthetic construct
TARE

SEQ ID NO: 259 - protein, 4 aa, synthetic construct
AREQ

SEQ ID NO: 260 - protein, 4 aa, synthetic construct
REQE

SEQ ID NO: 261 - protein, 4 aa, synthetic construct
EQEA

SEQ ID NO: 262-300 - skipped (39 entries)

SEQ ID NO: 301 - protein, 7 aa, synthetic construct
GYSFSSY

SEQ ID NO: 302 - protein, 6 aa, synthetic construct
HPSDST

SEQ ID NO: 303 - skipped

SEQ ID NO: 304 - protein, 16 aa, synthetic construct
KSSKSLLYKDGKTYLN

SEQ ID NO: 305 - protein, 7 aa, synthetic construct
VVSTRAS

SEQ ID NO: 306 - protein, 9 aa, synthetic construct
QQLVEYPYT

SEQ ID NO: 307 - protein, 5 aa, synthetic construct
SYYMH

SEQ ID NO: 308 - protein, 17 aa, synthetic construct
TIHPSDSTTNYNQKFQG

SEQ ID NO: 309 - protein, 6 aa, synthetic construct
YPGAST

SEQ ID NO: 310 - protein, 17 aa, synthetic construct
TIYPGASTTNYNQKFQG

SEQ ID NO: 311 - protein, 16 aa, synthetic construct
KSSKSLLYKDGHTYLN

SEQ ID NO: 312 - protein, 9 aa, synthetic construct
EQLVEYPYT

SEQ ID NO: 313 - protein, 6 aa, synthetic construct
YPEDST

SEQ ID NO: 314 - protein, 17 aa, synthetic construct
TIYPEDSTTNYNQKFQG

SEQ ID NO: 315 - protein, 6 aa, synthetic construct
HPEDST

SEQ ID NO: 316 - protein, 17 aa, synthetic construct
TIHPEDSTTNYNQKFQG

SEQ ID NO: 317 - skipped

SEQ ID NO: 318 - protein, 16 aa, synthetic construct
KSSKSLLYKDGKTYLL

SEQ ID NO: 319 - protein, 17 aa, synthetic construct
DIHPSDSTTNYNQKFQG

SEQ ID NO: 320 - skipped

SEQ ID NO: 321 - protein, 6 aa, synthetic construct
YPGDST

SEQ ID NO: 322 - protein, 17 aa, synthetic construct
TIYPGDSTTNYNQKFQG

SEQ ID NO: 323-439 - skipped (117 entries)

SEQ ID NO: 440 - protein, 37 aa, synthetic construct
ENTAVVAVEPDRRNQSPVDQGATGASQGLLDRKEVLG

SEQ ID NO: 441 - protein, 25 aa, synthetic construct
TAVVAVEPDRRNQSPVDQGATGASQ

SEQ ID NO: 442 - protein, 24 aa, synthetic construct
ENTAVVAVEPDRRNQSPVDQGATG

SEQ ID NO: 443 - protein, 15 aa, synthetic construct
ENTAVVAVEPDRRNQ

SEQ ID NO: 444 - protein, 25 aa, synthetic construct
RNQSPVDQGATGASQGLLDRKEVLG

SEQ ID NO: 445 - protein, 15 aa, synthetic construct
GEAVVLPEVEPGLTA

SEQ ID NO: 446 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 447 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 448 - protein, 9 aa, synthetic construct
GLVGLIFAV

SEQ ID NO: 449-457 - skipped (9 entries)

SEQ ID NO: 458 - protein, 112 aa, synthetic construct
EVQLVQSGAEVKKPGASVKVSCKASGYSFSSYYMHWVRQAPGQGLEWMGTIHPSDSTTNYNQKFQGRVTMTVDTSTRTAYMELSSLRSEDTAVYYCADFVYWGQGTTVTVSS

SEQ ID NO: 459 - protein, 112 aa, synthetic construct
EVQLVQSGAEVKKPGASVKVSCKASGYSFSSYYMHWVRQAPGQGLEWMGTIYPGASTTNYNQKFQGRVTMTVDTSTRTAYMELSSLRSEDTAVYYCADFVYWGQGTTVTVSS

SEQ ID NO: 460 - protein, 112 aa, synthetic construct; identical to SEQ ID NO: 458

SEQ ID NO: 461 - protein, 112 aa, synthetic construct; identical to SEQ ID NO: 458

SEQ ID NO: 462 - protein, 112 aa, synthetic construct
EVQLVQSGAEVKKPGASVKVSCKASGYSFSSYYMHWVRQAPGQGLEWMGTIYPEDSTTNYNQKFQGRVTMTVDTSTRTAYMELSSLRSEDTAVYYCADFVYWGQGTTVTVSS

SEQ ID NO: 463 - protein, 112 aa, synthetic construct
EVQLVQSGAEVKKPGASVKVSCKASGYSFSSYYMHWVRQAPGQGLEWMGTIYPGDSTTNYNQKFQGRVTMTVDTSTRTAYMELSSLRSEDTAVYYCADFVYWGQGTTVTVSS

SEQ ID NO: 464 - protein, 112 aa, synthetic construct
EVQLVQSGAEVKKPGASVKVSCKASGYSFSSYYMHWVRQAPGQGLEWMGTIHPEDSTTNYNQKFQGRVTMTVDTSTRTAYMELSSLRSEDTAVYYCADFVYWGQGTTVTVSS

SEQ ID NO: 465 - protein, 112 aa, synthetic construct
EVQLVQSGAEVKKPGASVKVSCKASGYSFSSYYMHWVRQAPGQGLEWMGTIHPSDSTTNYNQKFQGRVTMTVDTSTRTAYMELSSLRSEDTAVYYCADFLYWGQGTTVTVSS

SEQ ID NO: 466 - protein, 112 aa, synthetic construct
EVQLVQSGAEVKKPGASVKVSCKASGYSFSSYYMHWVRQAPGQGLEWMGTIHPSDSTTNYNQKFQGRVTMTIDTSTRTAYMELSSLRSEDTAVYYCADFVYWGQGTTVTVSS

SEQ ID NO: 467 - protein, 112 aa, synthetic construct; identical to SEQ ID NO: 458

SEQ ID NO: 468 - protein, 112 aa, synthetic construct
QVQLVQSGAEVKKPGASVKVSCKASGYSFSSYYMHWVRQAPGQGLEWMGDIHPSDSTTNYNQKFQGRVTMTVDTSTRTAYMELSSLRSEDTAVYYCANFVYWGQGTTVTVSS

SEQ ID NO: 469 - protein, 112 aa, synthetic construct
QVQLVQSGAEVKKPGASVKVSCKASGYSFSSYYMHWVRQAPGQGLEWMGTIHPSDSTTNYNQKFQGRVTMTVDTSTRTAYMELSSLRSEDTAVYYCANFVYWGQGTTVTVSS

SEQ ID NO: 470 - protein, 112 aa, synthetic construct
DIVMTQTPLSLSVTPGQPASISCKSSKSLLYKDGKTYLNWFLQKPGQSPQLLIYVVSTRASGVPDRFSGSGSGTDFTLKISRVEAEDVGVYYCQQLVEYPYTFGQGTKLEIK

SEQ ID NO: 471 - protein, 112 aa, synthetic construct; identical to SEQ ID NO: 470

SEQ ID NO: 472 - protein, 112 aa, synthetic construct
DIVMTQTPLSLSVTPGQPASISCKSSKSLLYKDGHTYLNWFLQKPGQSPQLLIYVVSTRASGVPDRFSGSGSGTDFTLKISRVEAEDVGVYYCQQLVEYPYTFGQGTKLEIK

SEQ ID NO: 473 - protein, 112 aa, synthetic construct
DIVMTQTPLSLSVTPGQPASISCKSSKSLLYKDGKTYLNWFLQKPGQSPQLLIYVVSTRASGVPDRFSGSGSGTDFTLKISRVEAEDVGVYYCEQLVEYPYTFGQGTKLEIK

SEQ ID NO: 474 - protein, 112 aa, synthetic construct; identical to SEQ ID NO: 470

SEQ ID NO: 475 - protein, 112 aa, synthetic construct; identical to SEQ ID NO: 470

SEQ ID NO: 476 - protein, 112 aa, synthetic construct; identical to SEQ ID NO: 470

SEQ ID NO: 477 - protein, 112 aa, synthetic construct; identical to SEQ ID NO: 470

SEQ ID NO: 478 - protein, 112 aa, synthetic construct; identical to SEQ ID NO: 470

SEQ ID NO: 479 - protein, 112 aa, synthetic construct
DIVMTQTPLSLSVTPGQPASISCKSSKSLLYKDGKTYLLWFLQKPGQSPQLLIYVVSTRASGVPDRFSGSGSGTDFTLKISRVEAEDVGVYYCQQLVEYPYTFGQGTKLEIK

SEQ ID NO: 480 - protein, 112 aa, synthetic construct; identical to SEQ ID NO: 470

SEQ ID NO: 481 - protein, 112 aa, synthetic construct
DIVMTQTPLSLSVTPGQPASISCKSSKSLLYKDGKTYLNWFLQKPGQSPQLLIDVVSTRASGVPDRFSGSGSGTDFTLKISRVEAEDVGVYYCQQLVEYPYTFGQGTKLEIK

SEQ ID NO: 482 - skipped

SEQ ID NO: 483 - DNA, 347 bp, synthetic construct
  source 1..347: mol_type = other DNA
gaagtgcaacttgtccagtctggagcagaagtaaagaagccaggcgcatcagtgaaagtaagctgtaaagcctctggctacagtttttcctcatactacatgcactgggttaggcaggctcctgggcaaggcctggaatggatgggtactatccatccctcagattcaaccacaaactacaaccaaaagtttcaaggaagagtgacaatgaccgttgatacctctacacgcacagcctatatggaacttagcagcctgcgaagcgaggatacagccgtatactactgcgccgacttcgtttattggggtcaaggtactactgtcaccgtcagcagtgcctcaactaa

SEQ ID NO: 484 - DNA, 347 bp, synthetic construct
  source 1..347: mol_type = other DNA
gaagttcagttggtgcagtctggcgcagaagttaagaagcccggggcatcagttaaagtcagttgcaaagcatcaggttattcctttagtagctattatatgcactgggtgcgacaagcccctggtcagggtctggagtggatggggactatttatcctggggcttctactacaaattacaatcagaaattccaggggcgagttaccatgactgtagatactagcactcgaaccgcatatatggaattgtcatccctccgctcagaagatacagcagtgtactattgtgccgactttgtctactggggtcagggtacaactgttacagtctctagtgcctcaactaa

SEQ ID NO: 485 - DNA, 347 bp, synthetic construct; identical to SEQ ID NO: 483
  source 1..347: mol_type = other DNA

SEQ ID NO: 486 - DNA, 347 bp, synthetic construct; identical to SEQ ID NO: 483
  source 1..347: mol_type = other DNA

SEQ ID NO: 487 - DNA, 347 bp, synthetic construct
  source 1..347: mol_type = other DNA
gaggtacagctggtacaaagtggggcggaagttaaaaagccgggggcttcagtgaaggtcagctgtaaggcgagtgggtactctttctcctcctattatatgcactgggtcaggcaggccccaggacaaggtctcgaatggatgggcacgatctacccagaggactccacgaccaactacaatcagaagtttcagggtcgagtgactatgaccgttgatacctcaacgagaaccgcatacatggaactctccagcttgagaagtgaagatacagccgtatattattgcgcggatttcgtctattgggggcaaggtacgactgtcacagttagctcagcctcaactaa

SEQ ID NO: 488 - DNA, 347 bp, synthetic construct
  source 1..347: mol_type = other DNA
gaagtccaattggtccagagcggagcagaggtgaagaagccaggggcctcagttaaagtgtcctgtaaagcatctgggtattccttcagcagctattatatgcactgggtccgccaggctccaggtcaggggcttgaatggatgggcaccatctaccccggagactctacgacgaactataaccaaaaatttcaggggcgcgttaccatgacggtagacacttctacacggacggcgtatatggagctcagcagtcttaggtctgaggatacggctgtttattattgcgcggatttcgtctattggggacagggaacaacggttacggttagcagtgcctcaactaa

SEQ ID NO: 489 - DNA, 347 bp, synthetic construct
  source 1..347: mol_type = other DNA
gaggtacaacttgttcagtctggcgccgaagtcaaaaaacctggggcaagcgtaaaagtgtcttgcaaagcgagcgggtattcttttagtagttactatatgcactgggttaggcaagcgcctggtcaaggtctggagtggatgggaacaattcatcccgaagacagtaccaccaattacaaccagaaattccaaggcagagttaccatgaccgtagatacgtcaacgcggacggcatatatggaactgtcctctctcagatcagaagacacggctgtttattactgcgccgatttcgtgtactggggccaaggcactactgtcaccgtttccagcgcctcaactaa

SEQ ID NO: 490 - DNA, 347 bp, synthetic construct
  source 1..347: mol_type = other DNA
gaagtacagctggtacagtctggtgctgaggttaagaaaccgggagcgtccgttaaggtaagctgtaaggctagtggctactccttttcctcatactatatgcactgggtcaggcaggcaccgggacaaggtctggaatggatgggtacaatacatccgtcagacagcactacgaactataatcagaagtttcaaggacgggtcactatgactgtggacacgagtacgagaacagcctacatggaactctcttctcttcggagcgaggacacggcagtgtactattgtgctgatttcctttactggggccagggtacgactgtgacggtcagttccgcctcaactaa

SEQ ID NO: 491 - DNA, 347 bp, synthetic construct
  source 1..347: mol_type = other DNA
gaagttcaacttgttcagagcggcgcagaggtaaaaaagccgggcgcaagtgttaaggttagctgcaaagcatctgggtatagcttctctagctattatatgcactgggtgcgacaagctcctggtcagggtctggaatggatgggaaccatccacccgagcgactctacgacgaactataatcaaaaattccagggcagagtcacaatgacaatagacacgtccacccggaccgcttatatggaactttcctccctgcgatccgaggatacggccgtttattattgcgccgactttgtgtactgggggcaagggaccacggtaacggtgagttcagcctcaactaa

SEQ ID NO: 492 - DNA, 347 bp, synthetic construct; identical to SEQ ID NO: 483
  source 1..347: mol_type = other DNA

SEQ ID NO: 493 - DNA, 400 bp, synthetic construct
  source 1..400: mol_type = other DNA
ttctgctctgcctggccgggcgcgccttggcccaagtacagcttgtccagtcaggtgcagaggtaaaaaagcccggcgcatcagtgaaggtatcttgtaaagcgtccggttattcattttcatcttactacatgcattgggttcggcaggcaccgggacagggcctggaatggatgggggacatccatccatctgacagcacaacaaattacaatcagaaatttcaaggtcgggtcacaatgaccgtggatacaagcacaagaacagcatatatggaactgagctcacttcggagtgaagatactgccgtgtattattgtgctaatttcgtctattgggggcaggggacgacggtgacagtaagtagtgcctcaactaagggcccctccgtgttcccgtt

SEQ ID NO: 494 - DNA, 335 bp, synthetic construct
  source 1..335: mol_type = other DNA
aagtacagcttgtccagtcaggtgcagaggtaaaaaagcccggcgcatcagtgaaggtatcttgtaaagcgtccggttattcattttcatcttactacatgcattgggttcggcaggcaccgggacagggcctggaatggatggggacgatccatccatctgacagcacaacaaattacaatcagaaatttcaaggtcgggtcacaatgaccgtggatacaagcacaagaacagcatatatggaactgagctcacttcggagtgaagatactgccgtgtattattgtgctaatttcgtctattgggggcaggggacgacggtgacagtaagtagt

SEQ ID NO: 495 - DNA, 336 bp, synthetic construct
  source 1..336: mol_type = other DNA
gatattgtcatgacacaaacccccctgagtcttagcgtcacccctgggcagcccgcttcaataagttgtaagtcctctaaatcattgctgtataaagatgggaagacctatcttaactggtttctccaaaagccagggcagtcaccacaactgttgatctacgttgtaagcaccagggcgagtggagtccccgacagattcagtggtagcggctctggtacagattttaccttgaaaatatctagggtggaagcggaggatgtcggtgtctactactgtcagcagctcgtagaatatccatacacatttggacaaggtacgaaactggagataaaa

SEQ ID NO: 496 - DNA, 336 bp, synthetic construct; identical to SEQ ID NO: 495
  source 1..336: mol_type = other DNA

SEQ ID NO: 497 - DNA, 336 bp, synthetic construct
  source 1..336: mol_type = other DNA
gacatcgtaatgactcaaacccctctgtctttgagtgtcacccctggtcaacctgcatctattagctgcaaatccagtaagtctttgctttacaaagatggacatacatacttgaactggtttttgcagaaacccggtcagagtcctcagcttctgatctatgttgtcagtacccgcgcgtctggggtacctgaccgctttagcggaagtggaagcggtacggactttacgctcaaaatttcaagagtggaggcggaggatgtaggggtctactactgtcaacagcttgtagaatatccttataccttcggacagggtacaaaacttgaaattaaa

SEQ ID NO: 498 - DNA, 336 bp, synthetic construct
  source 1..336: mol_type = other DNA
gacatcgttatgacccaaactcccctctcccttagtgtgactcctgggcagcctgcatctataagttgtaagagttcaaagtcactgttgtataaagatggaaaaacttacctcaactggttcttgcagaagcctggccagtcaccccaacttctgatatacgtcgtctcaacccgagcatctggtgtaccggacaggttttctggtagtggcagtggaacggactttacgttgaaaatatcccgagttgaagcggaagatgtaggcgtctattactgcgagcagctcgtagagtatccctacacgtttggacagggtacgaaacttgagattaag

SEQ ID NO: 499 - DNA, 336 bp, synthetic construct; identical to SEQ ID NO: 495
  source 1..336: mol_type = other DNA

SEQ ID NO: 500 - DNA, 336 bp, synthetic construct; identical to SEQ ID NO: 495
  source 1..336: mol_type = other DNA

SEQ ID NO: 501 - DNA, 336 bp, synthetic construct; identical to SEQ ID NO: 495
  source 1..336: mol_type = other DNA

SEQ ID NO: 502 - DNA, 336 bp, synthetic construct; identical to SEQ ID NO: 495
  source 1..336: mol_type = other DNA

SEQ ID NO: 503 - DNA, 336 bp, synthetic construct; identical to SEQ ID NO: 495
  source 1..336: mol_type = other DNA

SEQ ID NO: 504 - DNA, 336 bp, synthetic construct
  source 1..336: mol_type = other DNA
gatatagtaatgactcagaccccgctcagcctcagcgtaacaccgggacaacctgcctcaatatcatgcaaatcctccaaaagcctcctctacaaagacggaaagacgtatctgctgtggtttcttcagaaaccggggcaatccccgcaacttcttatctatgtcgtatcaaccagagcttccggtgttcccgatcgcttctctggtagcgggagtggaactgacttcacactcaaaatatctagggtcgaggctgaggacgtgggtgtttactattgtcaacagttggtggagtatccctacacattcgggcagggtactaaacttgagattaag

SEQ ID NO: 505 - DNA, 336 bp, synthetic construct; identical to SEQ ID NO: 495
  source 1..336: mol_type = other DNA

SEQ ID NO: 506 - DNA, 393 bp, synthetic construct
  source 1..393: mol_type = other DNA
ttctgctctgcctggccgggcgcgccttggccgatattgtcatgacacaaacccccctgagtcttagcgtcacccctgggcagcccgcttcaataagttgtaagtcctctaaatcattgctgtataaagatgggaagacctatcttaactggtttctccaaaagccagggcagtcaccacaactgttgatcgatgttgtaagcaccagggcgagtggagtccccgacagattcagtggtagcggctctggtacagattttaccttgaaaatatctagggtggaagcggaggatgtcggtgtctactactgtcagcagctcgtagaatatccatacacatttggacaaggtacgaaactggagataaaacgtacggtggcagcgccttccgtgt

SEQ ID NO: 507 - protein, 112 aa, synthetic construct; identical to SEQ ID NO: 463

SEQ ID NO: 508 - protein, 112 aa, synthetic construct; identical to SEQ ID NO: 472

SEQ ID NO: 509 - protein, 112 aa, synthetic construct; identical to SEQ ID NO: 464

SEQ ID NO: 510 - protein, 112 aa, synthetic construct; identical to SEQ ID NO: 472

SEQ ID NO: 511 - protein, 112 aa, synthetic construct; identical to SEQ ID NO: 465

SEQ ID NO: 512 - protein, 112 aa, synthetic construct; identical to SEQ ID NO: 472

SEQ ID NO: 513-526 - skipped (14 entries)

SEQ ID NO: 527 - protein, 442 aa, synthetic construct
QVQLVQSGAEVKKPGASVKVSCKASGYSFSSYYMHWVRQAPGQGLEWMGTIHPSDSTTNYNQKFQGRVTMTVDTSTRTAYMELSSLRSEDTAVYYCANFVYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 528 - protein, 219 aa, synthetic construct
DIVMTQTPLSLSVTPGQPASISCKSSKSLLYKDGKTYLNWFLQKPGQSPQLLIYVVSTRASGVPDRFSGSGSGTDFTLKISRVEAEDVGVYYCQQLVEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 529 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 530 - protein, 15 aa, synthetic construct
EPKSCDKTHTCPPCP

SEQ ID NO: 531 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 532 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 533 - protein, 6 aa, synthetic construct
RTVAAP

SEQ ID NO: 534 - protein, 101 aa, synthetic construct
SVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 535-600 - skipped (66 entries)

SEQ ID NO: 601 - protein, 310 aa, Homo sapiens; identical to SEQ ID NO: 2

SEQ ID NO: 602 - protein, 34 aa, Homo sapiens; identical to SEQ ID NO: 10
  SITE 1: note = N-term biotin-discrete polyethylene glycol-8 linker

SEQ ID NO: 603 - protein, 20 aa, Homo sapiens
  SITE 1: note = N-term biotin-discrete polyethylene glycol-8 linker
GEAVVLPEVEPGLTAREQEA

SEQ ID NO: 604 - protein, 15 aa, Homo sapiens
  SITE 1: note = N-term biotin-discrete polyethylene glycol-8 linker
GEAVVLPEVEPGLTA

SEQ ID NO: 605 - protein, 8 aa, Homo sapiens
  SITE 1: note = N-term biotin-discrete polyethylene glycol-8 linker
VLPEVEPG

SEQ ID NO: 606 - protein, 7 aa, Homo sapiens
  SITE 1: note = N-term biotin-discrete polyethylene glycol-8 linker
LPEVEPG

SEQ ID NO: 607 - protein, 8 aa, Homo sapiens
  SITE 1: note = N-term biotin-discrete polyethylene glycol-8 linker
PEVEPGLT

SEQ ID NO: 608 - protein, 41 aa, Homo sapiens; identical to SEQ ID NO: 16
  SITE 1: note = N-term biotin-discrete polyethylene glycol-8 linker

SEQ ID NO: 609 - protein, 33 aa, Homo sapiens
  SITE 1: note = N-term biotin-discrete polyethylene glycol-8 linker
ENTAVVAVEPDRRNQSPVDQGATGASQGLLDRK

SEQ ID NO: 610 - protein, 15 aa, Homo sapiens
  SITE 1: note = N-term biotin-discrete polyethylene glycol-8 linker
ENTAVVAVEPDRRNQ

SEQ ID NO: 611 - protein, 8 aa, Homo sapiens
  SITE 1: note = N-term biotin-discrete polyethylene glycol-8 linker
VVAVEPDR

SEQ ID NO: 612 - protein, 8 aa, Homo sapiens
  SITE 1: note = N-term biotin-discrete polyethylene glycol-8 linker
VAVEPDRR

SEQ ID NO: 613 - protein, 7 aa, Homo sapiens
  SITE 1: note = N-term biotin-discrete polyethylene glycol-8 linker
AVEPDRR

SEQ ID NO: 614 - protein, 17 aa, synthetic construct
GEAVVLPEVEPGGLTAR

SEQ ID NO: 615 - protein, 15 aa, synthetic construct
ENTAVVAVEPDRRNQ

SEQ ID NO: 616 - protein, 115 aa, synthetic construct
EVQLVQSGAEVKKPGASVKVSCKASGYSFSSYYMHWVRQAPGQGLEWMGTIHPSDSTTNYNQKFQGRVTMTVDTSTRTAYMELSSLRSEDTAVYYCADFVYWGQGTTVTVSSAST

SEQ ID NO: 617 - protein, 112 aa, synthetic construct; identical to SEQ ID NO: 469

SEQ ID NO: 618 - protein, 112 aa, synthetic construct; identical to SEQ ID NO: 465

SEQ ID NO: 619 - protein, 16 aa, synthetic construct
GEAVVLPEVEPGLTAR

SEQ ID NO: 620 - protein, 6 aa, synthetic construct
LPEVEP

SEQ ID NO: 621 - protein, 20 aa, synthetic construct
GEAVVLPEVEPGLTAREQEA

SEQ ID NO: 622 - protein, 6 aa, synthetic construct
HHHHHH

SEQ ID NO: 623 - protein, 6 aa, synthetic construct
LVEPLR

SEQ ID NO: 624 - protein, 11 aa, synthetic construct
  VARIANT 7: note = Any amino acid
  VARIANT 8: note = Any amino acid
  VARIANT 9: note = Any amino acid
  VARIANT 10: note = Any amino acid
LPEVEPXXXXR

SEQ ID NO: 625 - protein, 8 aa, synthetic construct
  VARIANT 7: note = Any amino acid
VVAVEPXR

SEQ ID NO: 626 - DNA, 336 bp, synthetic construct
  source 1..336: mol_type = other DNA
gaagtccaattggtccagagcggagcagaggtgaagaagccaggggcctcagttaaagtgtcctgtaaagcatctgggtattccttcagcagctattatatgcactgggtccgccaggctccaggtcaggggcttgaatggatgggcaccatctaccccggagactctacgacgaactataaccaaaaatttcaggggcgcgttaccatgacggtagacacttctacacggacggcgtatatggagctcagcagtcttaggtctgaggatacggctgtttattattgcgcggatttcgtctattggggacagggaacaacggttacggttagcagt

SEQ ID NO: 627 - DNA, 336 bp, synthetic construct
  source 1..336: mol_type = other DNA
gaggtacaacttgttcagtctggcgccgaagtcaaaaaacctggggcaagcgtaaaagtgtcttgcaaagcgagcgggtattcttttagtagttactatatgcactgggttaggcaagcgcctggtcaaggtctggagtggatgggaacaattcatcccgaagacagtaccaccaattacaaccagaaattccaaggcagagttaccatgaccgtagatacgtcaacgcggacggcatatatggaactgtcctctctcagatcagaagacacggctgtttattactgcgccgatttcgtgtactggggccaaggcactactgtcaccgtttccagc

SEQ ID NO: 628 - DNA, 336 bp, synthetic construct
  source 1..336: mol_type = other DNA
gaagtacagctggtacagtctggtgctgaggttaagaaaccgggagcgtccgttaaggtaagctgtaaggctagtggctactccttttcctcatactatatgcactgggtcaggcaggcaccgggacaaggtctggaatggatgggtacaatacatccgtcagacagcactacgaactataatcagaagtttcaaggacgggtcactatgactgtggacacgagtacgagaacagcctacatggaactctcttctcttcggagcgaggacacggcagtgtactattgtgctgatttcctttactggggccagggtacgactgtgacggtcagttcc

SEQ ID NO: 629 - protein, 16 aa, synthetic construct
GENTAVVAVEPDRRNQ

(19) United States
(12) Patent Application Publication
(10) Pub. No.: US 2023/0348614 A1
(43) Pub. Date: Nov. 2, 2023

(54) ENGINEERED ANTIBODY MOLECULES TO CD138 AND USES THEREOF

(71) Applicant: VISTERRA, INC., Waltham, MA (US)

(72) Inventors: James R. Myette, Waltham, MA (US); Boopathy Ramakrishnan, Braintree, MA (US); Bharat Chaganty, Concord, MA (US); Brahm Yachnin, Waltham, MA (US)

(21) Appl. No.: 18/058,334

(22) Filed: Nov. 23, 2022

Related U.S. Application Data
(60) Provisional application No. 63/283,033, filed on Nov. 24, 2021.

(51) Int. Cl. C07K 16/28; A61K 47/68

(52) U.S. Cl. CPC C07K 16/2896; A61K 47/6849; C07K 2317/52; C07K 2317/55; C07K 2317/732; C07K 2317/734; C07K 2317/92; C07K 2317/94

(57) ABSTRACT

Antibody molecules that specifically bind to CD138 in a tumor microenvironment (e.g., at acidic pH) are disclosed. The antibody molecules can be used to treat, prevent, and/or diagnose disorders, such as multiple myeloma.

### CROSS REFERENCE TO RELATED APPLICATIONS

This application claims the benefit of U.S. Provisional Application No. 63/283,033, filed Nov. 24, 2021. The contents of the aforementioned application are hereby incorporated by reference in their entirety.

### SEQUENCE LISTING

The instant application contains a Sequence Listing which has been submitted electronically in XML format and is hereby incorporated by reference in its entirety. Said XML copy, created on Jan. 26, 2023, is named P2029-704510_SL.xml and is 404,090 bytes in size.

### BACKGROUND

CD138 is a transmembrane heparan sulfate proteoglycan that is expressed on differentiated plasma cells and is a diagnostic biomarker of multiple myeloma (MM). Multiple myeloma tumors generally develop in bone, but occasionally are found in other tissues. Disease with a single plasma cell tumor is known as an isolated (or solitary) plasmacytoma. When more than one plasmacytoma is present, it is known as multiple myeloma. In the United States, the estimated new cases are about 30,000 in 2017 and more than 10,000 deaths are expected to occur.

There is a need for developing new approaches for treating, preventing and diagnosing diseases and disorders associated with CD138.

### SUMMARY

This disclosure provides, at least in part, antibody molecules that bind to CD138, e.g., human CD138, and that comprise one or more properties, e.g., one or more functional, biophysical, and structural properties disclosed herein. Without wising to be bound by theory, it is believed that in some embodiments, the anti-CD138 antibody molecules described herein can bind to CD138 in a pH-selective manner and specifically target CD138-expressing tumor cells.

In an embodiment, the antibody molecule exhibits improved binding affinity for CD138 (e.g., human CD138) at an acidic pH (e.g., a pH less than 7.4, e.g., a pH of about 6.0-6.5, 6.5-7.0, or 7.0-7.3, e.g., about 6.0, 6.1, 6.2, 6.3, 6.4, 6.5, 6.6, 6.7, 6.8, 6.9, 7.0, 7.1, 7.2, or 7.3). In an embodiment, the antibody molecule binds to CD138 in a tumor (e.g., CD138 on the surface of tumor cells, e.g., tumor cells in a tumor microenvironment, e.g., a tumor microenvironment having a lower pH than physiological pH) more strongly than to CD138 under physiological conditions (e.g., CD138 on the surface of a non-tumor cell or a cell not located in a tumor microenvironment, or CD138 on the surface of a cell in an environment of physiological pH).

In an embodiment, the antibody molecule can have reduced toxicity, decreased immunogenicity, greater therapeutic efficacy (e.g., lower tumor burden and/or increased overall survival), improved target binding (e.g., affinity), improved in vitro or in vivo stability, and higher mammalian recombinant expression levels. In an embodiment, the antibody molecule is capable of causing an effector function (e.g., an antibody-dependent cellular cytotoxicity (ADCC) activity) on a cell expressing CD138. In an embodiment, the antibody molecule preferentially binds to a membrane-bound CD138 versus a soluble CD138. In an embodiment, the antibody molecule binds to an epitope in an extracellular region of CD138 that is proximal to the transmembrane domain. In an embodiment, the antibody molecule binds to an epitope in an integrin binding domain (IBD) of CD138. In an embodiment, the antibody molecule does not bind exclusively to the IBD of CD138. In an embodiment, the antibody molecule binds to a plurality of epitopes on CD138 (e.g., at least two epitopes on CD138). In an embodiment, the antibody molecule binds to a first epitope in an extracellular region of CD138 proximal to the transmembrane domain, e.g., a first epitope comprising the amino acid sequence ENTAVVAVEPDRRNQ, or an amino acid sequence differing by no more than 1, 2, 3, 4, or 5 amino acids (e.g., substitution, deletion, or insertion) therefrom. In an embodiment, the antibody molecule binds to a second epitope in an integrin-binding domain (IDB) of CD138, e.g., a second epitope comprising the amino acid sequence GEAVVLPEVEPGLTAR, or an amino acid sequence differing by no more than 1, 2, 3, 4, or 5 amino acids (e.g., substitution, deletion, or insertion) therefrom. While not wishing to be bound by theory, it is believed that in an embodiment, improved or optimal cytotoxicity can be achieved, by targeting certain extracellular region(s) on membrane-bound CD138 that is proximal to the cell membrane.

In an embodiment, the antibody molecule is selected from Table 1, or competes for binding to CD138 with an anti-CD138 monoclonal antibody selected from Table 1. In an embodiment, the antibody molecule binds to the same or overlapping epitope as the epitope recognized by an anti-CD138 monoclonal antibody selected from Table 1. In an embodiment, the antibody molecule comprises one or more heavy chain variable regions (VHs) and/or one or more light chain variable regions (VLs) described in Table 1. In an embodiment, the antibody molecule comprises the heavy chain (HC) and the light chain (LC) described in Tables 6-8. In an embodiment, the antibody molecule comprises one or more heavy chain CDRs and/or one or more light chain CDRs described in Tables 1, 7, or 8.

In an embodiment, the antibody molecule comprises a heavy chain variable region (VH) comprising an amino acid sequence selected from Table 2, or competes for binding to CD138 with an anti-CD138 monoclonal antibody comprising a VH comprising an amino acid sequence selected from Table 2. In an embodiment, the antibody molecule comprises a light chain variable region (VL) comprising an amino acid sequence selected from Table 2, or competes for binding to CD138 with an anti-CD138 monoclonal antibody comprising a VL comprising an amino acid sequence selected from Table 2. In an embodiment, the antibody molecule comprises a VH and a VL, each comprising an amino acid sequence selected from Table 2, or competes for binding to CD138 with an anti-CD138 monoclonal antibody comprising a VH and a VL, each comprising an amino acid sequence selected from Table 2. In an embodiment, the antibody molecule binds to the same or overlapping epitope as the epitope recognized by an anti-CD138 monoclonal antibody comprising a VH and/or a VL, each comprising an amino acid sequence selected from Table 2. In an embodiment, the antibody molecule comprises one or more (e.g., 1, 2, or 3) heavy chain CDRs and/or one or more (e.g., 1, 2, or 3) light chain CDRs described in Table 2.

In an embodiment, the antibody molecule comprises a heavy chain (HC) comprising an amino acid sequence selected from Table 6 or 7, or competes for binding to CD138 with an anti-CD 138 monoclonal antibody comprising an HC comprising an amino acid sequence selected from Table 6 or 7. In an embodiment, the antibody molecule comprises a light chain (LC) comprising an amino acid sequence selected from Table 6 or 8, or competes for binding to CD138 with an anti-CD138 monoclonal antibody comprising a LC comprising an amino acid sequence selected from Table 6 or 8. In an embodiment, the antibody molecule comprises an HC and an LC, each comprising an amino acid sequence selected from Table 6 or 7 or Table 6 or 8, or competes for binding to CD138 with an anti-CD138 monoclonal antibody comprising an HC and an LC, each comprising an amino acid sequence selected from Table 6 or 7 or Table 6 or 8. In an embodiment, the antibody molecule binds to the same or overlapping epitope as the epitope recognized by an anti-CD138 monoclonal antibody comprising an HC and/or an LC, each comprising an amino acid sequence selected from Table 6 or 7 or Table 6 or 8. In an embodiment, the antibody molecule comprises one or more (e.g., 1, 2, or 3) heavy chain CDRs and/or one or more (e.g., 1, 2, or 3) light chain CDRs described in Table 7 or 8.

In an embodiment, antibody molecule-drug conjugates (ADCs), nucleic acid molecules encoding the antibody molecules, expression vectors, host cells, compositions (e.g., pharmaceutical compositions), kits, containers, and methods for making the antibody molecules, are also provided. The antibody molecules disclosed herein can be used (alone or in combination with other agents or therapeutic modalities) to treat, prevent and/or diagnose disorders associated with CD138, e.g., cancer or precancerous conditions (e.g., multiple myeloma or smoldering myeloma).

Accordingly, in certain aspects, this disclosure provides an antibody molecule (e.g., a pH-selective antibody molecule), e.g., an antibody molecule described herein (e.g., a pH-selective antibody molecule described herein), having one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or all) of the following properties a)-dd):
a) Binds to CD138 at a higher affinity at a pH of about 5.5-7.0 (e.g., a pH of 5.5-6.0, 6.0-6.5, or 6.5-7.0) than at physiological pH (e.g., a pH of about 7.4),
b) Binds to CD138 at a lower KD at a pH of about 5.5-7.0 (e.g., a pH of 5.5-6.0, 6.0-6.5, or 6.5-7.0) than at physiological pH (e.g., a pH of about 7.4),
c) Binds to CD138 (e.g., human CD138) with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM,
d) Binds to a membrane-bound CD138 with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM,
e) Binds to a soluble CD138 i) with high affinity, e.g., with a dissociation constant (K_{D}) of less than about 100 nM, typically about 10 nM, and more typically, about 10-0.001 nM, about 10-0.01 nM, about 5-0.01 nM, about 3-0.05 nM, about 1-0.1 nM, or stronger, e.g., less than about 80, 70, 60, 50, 40, 30, 20, 10, 8, 6, 4, 3, 2, 1, 0.5, 0.2, 0.1, 0.05, 0.01, 0.005, or 0.001 nM; or ii) with low affinity, e.g., with a dissociation constant (K_{D}) of greater than about 100 nM, e.g., greater than about 200, 300, 400, or 500 nM,
f) Binds to a membrane-bound CD138, or an intact ectodomain of CD138, i) preferably over a soluble CD138, e.g., the binding affinity to a membrane-bound CD138, or an intact ectodomain of CD138, is at least 2, 3, 4, 5, 6, 7, 8, 9, or 10-fold higher than the binding affinity to a soluble CD138; or ii) with a binding affinity similar to the binding affinity to a soluble CD138, e.g., the binding affinity to a membrane-bound CD138, or an intact ectodomain of CD138, is less than about 10%, 20%, 30%, 40%, 50%, 60%, 70%, 80%, 90%, or 100% higher than the binding affinity to a soluble CD138,
g) Binds to one or more (e.g., 2, 3, 4, 5, 6, 7, 8, 9, 10, 11, 12, 13, 14, 15, 20, 25, 30, 35, 40, 45, 50, 55, 60, 65, 70, 75, or more) amino acid residues of CD138 in an extracellular region proximal to the transmembrane domain of CD138, e.g., within 75, 70, 65, 60, 55, 50, 45, 40, 35, 30, 25, 20, 15, 10, or 5 amino acids from the N-terminus of the transmembrane domain,
h) i) Binds to an extracellular region of CD138 distant from the transmembrane domain, e.g., the C-terminus of the region is at least 100, 110, 120, 130, 140, 150, 160, 170, 180, 190, or 200 amino acids from the N-terminus of the transmembrane domain; or ii) does not bind, or binds with low affinity, to an extracellular region of CD138 distant from the transmembrane domain, e.g., the C-terminus of the region is at least 100, 110, 120, 130, 140, 150, 160, 170, 180, 190, or 200 amino acids from the N-terminus of the transmembrane domain,
i) Binds to the integrin binding domain (IBD) of CD138 or a region N-terminal to the IDB; or ii) does not bind, or binds with low affinity, to the IBD of CD138 or a region N-terminal to the IDB,
j) Binds to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in an extracellular region proximal to the transmembrane domain, e.g., a region comprising amino acids 176-250 (e.g., 176-214 or 210-250) of any of SEQ ID NOS: 1-3 or 450, optionally, wherein the epitope further comprises four or more (e.g., 5, 6, 7, 8, 9, 10, 11, 12, 13, 14, 15, 20, 25, 30, 35, or more) consecutive amino acid residues in an extracellular region distant from the transmembrane domain, e.g., a region comprising amino acids 23-50, 51-95, 88-121, 88-102, or 111-150 of any of SEQ ID NOS: 1-3 or 450,
k) Binds to two or more different regions in CD 138, e.g., a multivalent (e.g., bivalent, trivalent, or tetravalent) antibody molecule comprising two sets of identical, or substantially identical, VH-VL pairs that each bind to the same two or more regions, or comprising different sets of VH-VL pairs that each independently bind to different regions,
amino acid sequence differing by no more than 1, 2, 3, 4, or 5 amino acids (e.g., substitution, deletion, or insertion) therefrom, and to a second epitope comprising the amino acid sequence GEAVVLPEVEPGLTAR, or an amino acid sequence differing by no more than 1, 2, 3, 4, or 5 amino acids (e.g., substitution, deletion, or insertion) therefrom,
m) Binds to a first epitope located in a membrane-proximal region of CD138 and to a second epitope located in an integrin-binding domain (IBD) of CD138,
n) Does not bind to an epitope on CD138 comprising four or more (e.g., 5, 6, 7, 8, 9, 10, 11, 12, 13, 14, 15, 20, 25, 30, 35, or more) consecutive amino acid residues in an extracellular region distant from the transmembrane domain, e.g., a region comprising amino acids 23-50, 51-95, 88-121, 88-101, or 111-150 of any of SEQ ID NOS: 1-3 or 450,
o) Binds to a cancer or precancerous cell (e.g., a myeloma cell) expressing CD138 with high affinity,
p) Binds to an Fc receptor (FcR) (e.g., one or more of FcγRI, FcγRIIa, FcγRIIb, FcγRIIc, FcγRIIIa, or FcγRIIIb) on the surface of an immune cell (e.g., a natural killer (NK) cell, a macrophage, a monocyte, or an eosinophil),
q) Causes an effector function (e.g., an ADCC activity) on a target cell expressing CD138,
r) Binds to C1q and causes complement-dependent cytotoxicity (CDC) on a target cell expressing CD138,
s) Mediates homotypic adhesion of one or more CD138-expressing cells,
t) Inhibits the action of a protease on a membrane-bound CD138, e.g., to reduce shedding of CD138;
u) Reduces (e.g., inhibits) one or more biological activities of a cell expressing CD138, in vitro, ex vivo, or in vivo,
v) Reduces (e.g., inhibits) one or more functions of CD138 (e.g., binding of CD138 to a ligand), in vitro, ex vivo, or in vivo,
w) Reduces (e.g., inhibits) proliferation of a cancer or precancerous cell expressing CD138,
x) Binds to the same, similar, or overlapping epitope on CD138 as the epitope recognized by an anti-CD138 monoclonal antibody described herein,
y) Shows the same or similar binding affinity or specificity, or both, as an anti-CD138 monoclonal antibody described herein,
z) Shows the same or similar binding affinity or specificity, or both, as an antibody molecule (e.g., a pH-selective antibody molecule) comprising a heavy chain variable region and/or light chain variable region described herein, e.g., a heavy chain variable region and/or light chain variable region of any of the anti-CD138 monoclonal antibodies described herein,
aa) Shows the same or similar binding affinity or specificity, or both, as an antibody molecule (e.g., a pH-selective antibody molecule) comprising one or more (e.g., two or three) heavy chain CDRs and/or one or more (e.g., two or three) light chain CDRs described herein, e.g., one or more (e.g., two or three) heavy chain CDRs and/or one or more (two or three) light chain CDRs of any of the anti-CD 138 monoclonal antibodies described herein,
bb) Shows the same or similar binding affinity or specificity, or both, as an antibody molecule (e.g., a pH-selective antibody molecule) comprising an amino acid sequence described herein,
cc) Shows the same or similar binding affinity or specificity, or both, as an antibody molecule (e.g., a pH-selective antibody molecule) comprising an amino acid sequence encoded by a nucleotide sequence described herein,
dd) Inhibits, e.g., competitively inhibits, the binding of a second antibody molecule to CD138, wherein the second antibody molecule is an antibody molecule described herein (e.g., a pH-selective antibody molecule described herein),
ee) Competes for binding with a second antibody molecule to CD138,wherein the second antibody molecule is a humanized anti-CD138 monoclonal antibody described herein,
ff) Has one or more biological properties of a humanized anti-CD138 monoclonal antibody described herein,
gg) Has one or more structural properties of a humanized anti-CD 138 monoclonal antibody described herein, or
hh) Has one or more pharmacokinetic properties of a humanized anti-CD138 monoclonal antibody described herein.

In an aspect, the disclosure features an anti-CD138 antibody molecule (e.g., a pH-selective anti-CD138 antibody molecule) comprising one or both of:
(a) a heavy chain variable region (VH), wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of an anti-CD138 monoclonal antibody described herein (e.g., any of antibodies 28-0, 29-0, Ab17, Ab18, Ab29, Ab28, Ab30, Ab32, Ab34, Ab43, Ab71, Ab137, Ab173, Ab174, or Ab175, e.g., as listed in Table 1 or 2; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD 138 antibody; or (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody; or
(b) a light chain variable region (VL), wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD 138 antibody; or (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

In an embodiment, the VH comprises: (i) an HCDR1 comprising an amino acid sequence that differs by no more than 1, 2, or 3 amino acid residues from, or has at least 85, 90, 95, 99 or 100% homology with, the amino acid sequence of the HCDR1 of the anti-CD138 antibody; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; and (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD 138 antibody.

In an embodiment, the VH comprises: (i) an HCDR1 comprising the amino acid sequence of the HCDR1 of the anti-CD 138 antibody; (ii) an HCDR2 comprising the amino acid sequence of the HCDR2 of the anti-CD138 antibody; and (iii) an HCDR3 comprising the amino acid sequence of the HCDR3 of the anti-CD138 antibody.

In an embodiment, the VL comprises: (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; and (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD 138 antibody.

In an embodiment, the VL comprises: (i) an LCDR1 comprising the amino acid sequence of the LCDR1 of the anti-CD 138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

In an embodiment, the antibody molecule comprises:
(a) a VH comprising: (i) an HCDR1 comprising an amino acid sequence that differs by no more than 1, 2, or 3 amino acid residues from, or has at least 85, 90, 95, 99 or 100% homology with, the amino acid sequence of the HCDR1 of the anti-CD138 antibody; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD 138 antibody; and (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody, and
(b) a VL comprising: (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD138 antibody; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD 138 antibody; and (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

In an embodiment, the antibody molecule comprises: (a) a VH comprising: (i) an HCDR1 comprising the amino acid sequence of the HCDR1 of the anti-CD138 antibody; (ii) an HCDR2 comprising the amino acid sequence of the HCDR2 of the anti-CD 138 antibody; and (iii) an HCDR3 comprising the amino acid sequence of the HCDR3 of the anti-CD138 antibody, and (b) a VL comprising: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD 138 antibody.

In an embodiment, the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD 138 antibody. In an embodiment, the antibody molecule the VH comprises the amino acid sequence of the VH of the anti-CD 138 antibody.

In an embodiment, the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VL of the anti-CD 138 antibody. In an embodiment, the VL comprises the amino acid sequence of the VL of the anti-CD 138 antibody.

In an embodiment, (a) the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody; and (b) the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VL of the anti-CD 138 antibody.

In an embodiment, the VH comprises the amino acid sequence of the VH of the anti-CD 13 8 antibody and the VL comprises the amino acid sequence of the VL of the anti-CD 138 antibody.

In an embodiment, the antibody molecule comprises an Fc region (e.g., an Fc region described herein). In an embodiment, the antibody molecule comprises a heavy chain constant region of IgG, e.g., IgG1. In an embodiment, the antibody molecule comprises a light chain constant region of kappa.

In an embodiment, the antibody molecule comprises a heavy chain (HC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the HC of the anti-CD138 antibody. In an embodiment, the HC comprises the amino acid sequence of the HC of the anti-CD138 antibody.

In an embodiment, the antibody molecule comprises a light chain (LC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the LC of the anti-CD138 antibody. In an embodiment, the LC comprises the amino acid sequence of the LC of the anti-CD138 antibody.

In an embodiment, the antibody molecule comprises (a) a heavy chain (HC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the HC of the anti-CD 138 antibody; and (b) a light chain (LC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the LC of the anti-CD 138 antibody. In an embodiment, the HC comprises the amino acid sequence of the HC of the anti-CD 138 antibody and the LC comprises the amino acid sequence of the LC of the anti-CD 138 antibody.

In an embodiment, the VH of an antibody molecule described herein does not comprise the amino acid sequence of SEQ ID NO: 471. In an embodiment, the VL of an antibody molecule described herein does not comprise the amino acid sequence of SEQ ID NO: 475. In an embodiment, the VH of an antibody molecule described herein does not comprise the amino acid sequence of SEQ ID NO: 471, and the VL of an antibody molecule described herein does not comprise the amino acid sequence of SEQ ID NO: 475. In an embodiment, the heavy chain of an antibody molecule described herein does not comprise the amino acid sequence of SEQ ID NO: 527. In an embodiment, the light chain of an antibody molecule described herein does not comprise the amino acid sequence of SEQ ID NO: 528. In an embodiment, the heavy chain of an antibody molecule described herein does not comprise the amino acid sequence of SEQ ID NO: 527, and the light chain of an antibody molecule described herein does not comprise the amino acid sequence of SEQ ID NO: 528.

In an aspect, the disclosure features an antibody molecule, which competes for binding to CD138 with an anti-CD138 monoclonal antibody described herein (e.g., any of antibodies 28-0, 29-0, Ab17, Ab18, Ab29, Ab28, Ab30, Ab32, Ab34, Ab43, Ab71, Ab137, Ab173, Ab174, or Ab175).

In an aspect, the disclosure features an antibody molecule, which binds, or substantially binds, to an epitope that completely or partially overlaps with the epitope of a humanized anti-CD 138 monoclonal antibody described herein (e.g., any of antibodies 28-0, 29-0, Ab17, Ab18, Ab29, Ab28, Ab30, Ab32, Ab34, Ab43, Ab71, Ab137, Ab173, Ab174, or Ab175).

In an aspect, the disclosure features an antibody-molecule drug conjugate (ADC) comprising an antibody molecule described herein (e.g., a pH-selective antibody molecule described herein), optionally comprising a cytotoxic agent, further optionally comprising a linker.

In an aspect, the disclosure features a composition comprising an antibody molecule described herein (e.g., a pH-selective antibody molecule described herein), or an ADC described herein, optionally, wherein the composition is a pharmaceutical composition.

In an embodiment, the composition further comprises a pharmaceutically acceptable carrier.

In an aspect, the disclosure features a nucleic acid molecule encoding a heavy chain variable region (VH), a light chain variable region (VL), or both, of an antibody molecule described herein (e.g., a pH-selective antibody molecule described herein).

In an aspect, the disclosure features a vector comprising a nucleic acid molecule described herein.

In an aspect, the disclosure features a cell comprising a nucleic acid molecule described herein or a vector described herein, optionally, wherein the cell is an isolated cell.

In an aspect, the disclosure features a kit comprising an antibody molecule described herein (e.g., a pH-selective antibody molecule described herein), an ADC described herein, or a composition described herein, and instructions to use of the antibody molecule or composition.

In an aspect, the disclosure features a container comprising an antibody molecule described herein (e.g., a pH-selective antibody molecule described herein), an ADC described herein, or a composition described herein.

In an aspect, the disclosure features a method of producing a humanized anti-CD 138 antibody molecule, the method comprising culturing a cell described herein under conditions that allow production of an antibody molecule (e.g., a pH-selective antibody molecule described herein), thereby producing the antibody molecule.

In an embodiment, the method further comprises isolating or purifying the antibody molecule.

In an aspect, the disclosure features an antibody molecule of described herein (e.g., a pH-selective antibody molecule described herein), an ADC described herein, or a composition described herein, for use in a method of treating a cancer in a subject, or use of an antibody molecule of described herein (e.g., a pH-selective antibody molecule described herein), an ADC described herein, or a composition described herein, in the manufacture of a medicament for treating a cancer in a subject, e.g., in accordance with a method described herein.

In an embodiment, the cancer is a hematological cancer. In an embodiment, the cancer is a multiple myeloma. In an embodiment, the cancer is a solid tumor, e.g., a solid tumor described herein. In an embodiment, the antibody molecule reduces tumor burden in a subject, e.g., a subject having multiple myeloma.

In an embodiment, the antibody molecule, ADC, or composition is administered to the subject intravenously. In an embodiment, the antibody molecule is administered to the subject intraperitoneally.

In an embodiment, the antibody molecule, ADC, or composition is administered once a week, twice a week, once every two weeks, once every three weeks, or once every four weeks.

In an embodiment, the use further comprises determining the level of CD138 in a sample from the subject. In an embodiment, the use further comprises administering to the subject a second therapy for cancer.

In an aspect, the disclosure features an antibody molecule described herein (e.g., a pH-selective antibody molecule described herein), an ADC described herein, or a composition described herein, for use in a method of treating a precancerous condition or preventing a cancer in a subject, or use of an antibody molecule of described herein (e.g., a pH-selective antibody molecule described herein), an ADC described herein, or a composition described herein, in the manufacture of a medicament for treating a precancerous condition or preventing a cancer in a subject, e.g., in accordance with a method described herein.

In an embodiment, the precancerous condition is smoldering myeloma or monoclonal gammopathy of undetermined significance (MGUS). In an embodiment, the cancer is multiple myeloma.

In an aspect, the disclosure features a method of causing an ADCC activity, the method comprising contacting a cell or subject an antibody molecule described herein (e.g., a pH-selective antibody molecule described herein), an ADC described herein, or a composition described herein, thereby causing the ADCC activity.

In an aspect, the disclosure features a method of treating a cancer, the method comprising administering to a subject in need thereof an effective amount of an antibody molecule described herein (e.g., a pH-selective antibody molecule described herein), an ADC described herein, or a composition described herein, thereby treating the cancer.

In an aspect, the disclosure features a method of treating a precancerous condition or preventing a cancer, the method comprising administering to a subject in need thereof an effective amount of an antibody molecule described herein (e.g., a pH-selective antibody molecule described herein), an ADC described herein, or a composition described herein, thereby treating the precancerous condition or preventing the cancer.

In an aspect, the disclosure features, a method of detecting an anti-CD138 molecule, the method comprising contacting a cell or a subject with an antibody molecule described herein (e.g., a pH-selective antibody molecule described herein), thereby detecting the CD138 molecule.

In an embodiment, the antibody molecule is coupled with a detectable label. In an embodiment, the CD138 molecule is detected in vitro, ex vivo, or in vivo.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIG. 1 is a graph showing mAb 4320 binding to CD138-related peptides. The graph shows evaluation of mAb 4320-CD138 peptide binding using biolayer interferometry. Peptide sequences are listed in Table 11. *non-binding peptides include 138-2-1, 138-2, and 138-3; 138-6-2 and 138-6-3.

FIG. 2 is a diagram showing a sequence alignment of overlapping epitope peptides in CD138 for the exemplary anti-CD138 antibody molecules described herein.

FIG. 3 is a table showing the mapping of the epitope on CD138 for anti-CD 138 antibody 4320 by alanine-scanning mutagenesis. Select binding mutants are included based on stringency cut-off of ≤ 60%. Data normalized as percentage wild-type binding and is reported as means as well as range. Binding data include both mAb 4320 and anti-CD138 antibody BB4 for comparative purposes. Amino acid positions corresponding to “peptide 2” region or “peptide 6” region are highlighted in bold.

FIGS. 4A-4C are a series of diagrams showing CD138 peptide 138-2-5 in complex with exemplary anti-CD138 antibody molecule 4320. Refined structure of the complex showing the peptide 138-2-5 bound between the heavy chain (VH, left) and light chain (VL, right) (FIG. 4A). 4320 is represented as a ribbon; CD138 peptide is highlighted in dark gray and includes electron density. Hydrogen bonds between Glu 10 and Arg 16 of the peptide and the Fab3220 (FIG. 4B). Hydrogen bonds between the very N-terminal pyroglutamic acid (PCA) of the heavy chain and the main chain of Glu 2 in the peptide is highlighted (FIG. 4C). Electron densities are shown.

FIGS. 5A-5B is a series of diagrams showing CD138 peptide 1386-5 in complex with exemplary anti-CD138 antibody molecule 4320. Refined structure of the complex showing the peptide 138-6-5 (chain I) bound between the heavy chain A (VH, left) and light chain B (VL, right) (FIG. 5A). Peptide is drawn as sticks with Glu 9 highlighted for orientation. Hydrogen bonds between Glu10 and Arg12 of the peptide and the 4320 Fab. Electron density (2m|Fo|-D|Fc|) contoured at the 1 σ level is included (FIG. 5B). For simplicity only one complex in the asymmetric unit is shown in but is present in all four crystal projections.

FIGS. 6A-6B is a series of diagrams showing the mAb 4320-CD138 binding interface, as generated by molecular modeling. Model was generated using Pymol (Schrodinger, Inc.) using coordinates and atomic positions corresponding to the x-ray crystal structure.. CD138 peptides are represented as sticks while the mAb 4320 Fab (VH and VL) is represented as space filled atoms overlayed with ribbon representation for clarity. Respective peptide sequences are also listed. 4320-peptide 138-2-5 (FIG. 6A). 4320-peptide 138-6-5 (FIG. 6B). N-terminus of peptide 138-6-5, comprised of four amino acids (ENTA), is not resolved but represented as a dotted line. C terminal glutamine likewise is not present. Respective sequences are listed. Unresolved sequence of 138-6-5 is shaded in gray. Core epitopes are underlined. Minimal “VEP” sequence common to both sequences is highlighted in bold.

FIG. 7 is a diagram showing a superimposition of the core epitope of CD138. Model depicts the superimposition of two regions of CD138 referred above as peptide 2 and peptide 6, corresponding to peptides 138-2-5 and 138-6-5, respectively. CD138 peptides are represented as sticks. Peptide 2 is depicted in darker gray; peptide 6 is depicted in lighter gray. Respective amino acid sequences are also included as in FIG. 6. Unresolved sequence of peptide 6 (138-6-5) is shaded in gray. Core epitopes are underlined. Minimal “VEP” sequence common to both sequences is highlighted in bold.

FIG. 8 is a series of graphs showing pH selective binding of mAb 4320 variant anti-CD 138 antibodies to a CD138+ cell line (U266 cells).

FIG. 9 is a diagram showing the epitope interaction between mAb 4320 HCDR1 and CD138. The CDR1:H35 residue interacts with the peptide Glu residue of ‘VEP” motif. H35 residue is held by W47 hydrogen bond with its ND atom as proton acceptor; NE2 interacts as a proton donor with the carboxylate group of the core epitope. H35 exists in is basic form with NE2 protonated as expected from crystallization pH (pH 8.0).

FIG. 10 is a series of diagrams showing the mAb 4320 paratope-CD138 peptide interface. Selected residues corresponding to 4320 variants are highlighted. H35 is indicated by a box with dashes.

FIG. 11 is a series of diagrams showing an extended evaluation of the pH-dependent binding profile of anti-CD138 antibodies to cell surface CD138. Binding to cell surface CD138 of CD138+ myelogenous cell line U266 was measured by flow cytometry under varying cell culture pH ranging from 6 to 7.4 as described. A. Antibody binding profiles as a function of antibody concentrations as measured at different pH. Cell binding is reported as mean fluorescence intensity (MFI). B. Summary of pH dependency of anti-CD138 antibody binding. Binding intensity (MFI) corresponding to a single antibody concentration of 1.8 µg/mL(from data in A) is plotted as a titration of varying pH values.

FIG. 12 is a series of graphs showing pH selective binding of anti-CD 138 antibodies to recombinant CD138 extracellular domain. Recombinant CD138 corresponds to the extracellular domain and is present in soluble form. Binding was evaluated at varying pH ranging from 7.4 to 6.0 and evaluated by biolayer interferometry. Darker sensorgrams correspond to increasing pH (as likewise depicted in key).

FIG. 13 is a series of graphs showing secondary evaluation of pH-dependent antibody binding to CD 138, as measured by Octet. Modifications to the assay are described. The assay was conducted at two different buffer pH (7.4 vs. 6.0). Antibody titration was completed through 2-fold dilutions ranging from 25 nM down to 0.8 nM. Apparent kinetic values derived from this analysis are summarized in Error! Reference source not found..

FIG. 14 is a series of graphs showing an assessment of pH dependent anti-CD 138 antibody binding using surface plasmon resonance (SPR). Select antibodies are included by example. Sensorgrams are depicted from top to bottom based on decreasing CD138 concentrations. Binding parameters of these and other CD138 antibodies are summarized in Table 6.

### DETAILED DESCRIPTION

The disclosure is based, at least in part, on the discovery that the pH-selective anti-CD138 antibody molecules described herein can be used to target cells in a tumor microenvironment. Without wising to be bound by theory, it is believed that in some embodiments, a tumor microenvironment is acidic compared to normal tissues (which generally have a physiological pH of about 7.4), for example, due to high metabolic activity (e.g., metabolic acidosis), insufficient perfusions, and/or hypoxia. In an embodiment, the anti-CD138 antibody molecule exhibits stronger binding affinity for CD138 (e.g., human CD138) at acidic pH (e.g., pH 5.5-7.0, e.g., pH 5.5-6.0, 6.0-6.5, or 6.5-7.0) than at physiological pH (e.g., pH 7.4). In an embodiment, the anti-CD138 antibody molecule exhibits stronger binding affinity for CD138 in a tumor microenvironment compared to an otherwise-similar non-tumor or healthy tissue. In an embodiment, the anti-CD138 molecule has a KD for CD138 at acidic pH (e.g., pH 5.5-7.0, e.g., pH 5.5-6.0, 6.0-6.5, or 6.5-7.0) at least 2, 3, 4, 5, 6, 7, 8, 9, or 10 times lower than the KD for CD138 at physiological pH (e.g., pH 7.4). In an embodiment, the ratio of KD for CD138 at physiological pH to KD for CD138 at acidic pH is at least 4:1 (e.g., about 4:1, 5:1, 6:1, 7:1, 8:1, 9:1, 10: 1, or more). Without wishing to be bound by theory, it is believed that in some embodiment, improved CD138 target selectivity in tumor environments and/or at acidic pH can result in improved delivery of an agent, e.g., a cytotoxic agent (e.g., an anti-CD138 antibody-drug conjugate), to tumor cells and/or reduced delivery of the agent to other cells (e.g., cells not in a tumor environment and/or cells at physiological pH).

Advantageously, at least several of the antibody molecules describe herein have improved ability to inhibit cells expressing CD138, e.g., by eliciting an effector function. Without wishing to be bound by theory, it is believed that in an embodiment, anti-CD138 antibodies that bind to a desired epitope described herein have increased effector functions and preferential binding to the membrane-associated form of CD138. Targeting CD138 effectively can result in broad activity and favorable therapeutic index across myelomas and other cancers.

The antibody molecules described herein can have one or more improved properties, e.g., one or more properties described herein, e.g., compared to a parental antibody molecule. For example, the improved properties can include, but are not limited to, therapeutic efficacy, mitigation of immunogenicity, improvement of biophysical, physicochemical, and pharmaceutical properties, improvement of target binding, biological activity, and higher recombinant expression in mammalian cell lines used for purposes of antibody production. In an embodiment, the antibody molecule has greater therapeutic efficacy (e.g., lower tumor burden and/or increased survival). In an embodiment, the antibody molecule has increased stability (in vitro and/or in vivo). In an embodiment, the antibody molecule has higher expression level (e.g., in cell lines). In an embodiment, the antibody molecule has comparable or improved CD138 binding affinity, effector function (e.g., ADCC activity), or both.

The anti-CD138 antibody molecules (e.g., humanized anti-CD138 antibody molecules) described herein can be used for the treatment of a number of disorders, e.g., multiple myeloma and other oncology indications. Without wishing to be bound by theory, it is believed that in an embodiment, the disorders involve CD138 positive cancer cells and/or CD138 mediated biological activities relevant to disease pathophysiology. For example, CD138 plays an important role in KRAS driven pathways underlying tumorigenesis and resistance in various carcinomas exemplified by pancreatic ductal adenocarcinoma.

The anti-CD138 antibody molecules (e.g., humanized anti-CD138 antibody molecules) described herein, can have biological activities that are particularly suitable for treating human disorders. For example, an exemplary humanized anti-CD138 antibody molecule, mAb 4320, exhibits potent in vitro activity relevant to its immune mediated therapeutic mechanism of action. These attributes include, e.g., both sub-nanomolar binding to CD138+ myeloma cells and antibody-dependent cell-mediated cytotoxicity (ADCC) against several MM cell lines, e.g., as assessed in biologically relevant natural killer (NK) cell-based ADCC assays using human-derived NK cells. This potent cell killing activity is both dose-dependent and target-dependent and has been shown to be highly effective against a number of variably expressing CD138 multiple myeloma cell lines including drug resistant MM cell lines, e.g., stable cell lines that are propagated to be resistant to either bortezomib or lenalidomide, two front-line therapies commonly used in combination as standard of care in patients for purposes of induction, consolidation, or maintenance therapy. mAb 4320 effectively kills autologously-derived myeloma cells from relapsed/refractory patients who do not respond to such treatments. Other relevant mechanisms of action, such as antibody-dependent cellular phagocytosis (ADCP), direct inhibition of myeloma cell survival, and blocking of integrin binding, can also exist. mAb 4320 efficacy in vivo also has been demonstrated, as a single agent or in combination with a proteasome inhibitor (e.g., bortezomib) to achieve a synergistic effect, in a murine xenograft model of disease involving the use of disseminated MM1.S tumors in CB.17 mice.

Antibody-drug conjugates (ADCs), nucleic acid molecules encoding the antibody molecules, expression vectors, host cells, compositions (e.g., pharmaceutical compositions), kits, and methods for making the antibody molecules, are also provided. The antibody molecules and pharmaceutical compositions disclosed herein can be used (alone or in combination with other agents or therapeutic modalities) to treat, prevent and/or diagnose disorders and conditions, e.g., disorders and conditions associated with CD138, e.g., cancer or precancerous conditions.

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When “about” or “approximately” is present before a series of numbers or a range, it is understood that “about” or “approximately” can modify each of the numbers in the series or range. Similarly, when “at least,” “more than,” “no more than,” “less than,” “no less than,” or “within” is present before a series of numbers or a range, it is understood that “at least,” “more than,” “no more than,” “less than,” “no less than,” or “within” can modify each of the numbers in the series or range. As used herein, ranges include both the upper and lower limit.

The compositions and methods disclosed herein encompass polypeptides and nucleic acids having the sequences specified, or sequences substantially identical or similar thereto, e.g., sequences at least 85%, 90%, 95% identical or higher to the sequence specified.

In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, e.g., at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch ((1970) J. Mol. Biol. 48:444-453) algorithm which has been incorporated into the GAP program in the GCG software package (available at gcg.com), using either a Blossum 62 matrix or a PAM250 matrix, and a gap weight of 16, 14, 12, 10, 8, 6, or 4 and a length weight of 1, 2, 3, 4, 5, or 6. In certain embodiments, the percent identity between two nucleotide sequences is determined using the GAP program in the GCG software package (available at gcg.com), using a NWSgapdna.CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score = 100, wordlength = 12 to obtain nucleotide sequences homologous to a nucleic acid as described herein. BLAST protein searches can be performed with the XBLAST program, score = 50, wordlength = 3 to obtain amino acid sequences homologous to protein molecules described herein. To obtain gapped alignments for comparison purposes, Gapped BLAST can be utilized as described in Altschul et al., (1997) Nucleic Acids Res. 25:3389-3402. When utilizing BLAST and gapped BLAST programs, the default parameters of the respective programs (e.g., XBLAST and NBLAST) can be used. See ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6X sodium chloride/sodium citrate (SSC) at about 45° C., followed by two washes in 0.2X SSC, 0.1% SDS at least at 50° C. (the temperature of the washes can be increased to 55° C. for low stringency conditions); 2) medium stringency hybridization conditions in 6X SSC at about 45° C., followed by one or more washes in 0.2X SSC, 0.1% SDS at 60° C.; 3) high stringency hybridization conditions in 6X SSC at about 45° C., followed by one or more washes in 0.2X SSC, 0.1% SDS at 65° C.; and preferably 4) very high stringency hybridization conditions are 0.5 M sodium phosphate, 7% SDS at 65° C., followed by one or more washes at 0.2X SSC, 1% SDS at 65° C. Very high stringency conditions 4) are suitable conditions and the ones that should be used unless otherwise specified.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D-or L- optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or noncoding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat,” a disorder, e.g., a myeloma, means that a subject (e.g., a human) who has a disorder, e.g., a myeloma, and/or experiences a symptom of a disorder, e.g., a myeloma, will, in an embodiment, suffer less a severe symptom and/or recover faster when an antibody molecule is administered than if the antibody molecule were never administered. In an embodiment, when a myeloma is treated, a bone marrow biopsy will show fewer clonal plasma cells, after effective treatment for myeloma. For example, a diagnostic assay will detect fewer clonal plasma cells in a biological sample of a subject after administration of an antibody molecule described herein for the effective treatment of a myeloma. Other assays, urine tests, or blood tests, can also be used to monitor treatment in a patient, or to detect the presence, e.g., decreased presence (or absence), of a symptom of a myeloma, after treatment of a myeloma in the subject. In an embodiment, when a myeloma is treated, the level of β2 microglobulin (β2M) in serum or urine will be decreased, after effective treatment for myeloma. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., a myeloma. In an embodiment, treatment is of a subject who does not exhibit certain signs of a disorder, e.g., a myeloma, and/or of a subject who exhibits only early signs of a disorder, e.g., nephropathy. In an embodiment, treatment is of a subject who exhibits one or more established signs of a disorder, e.g., a myeloma. In an embodiment, treatment is of a subject diagnosed as suffering from a disorder, e.g., a myeloma.

As used herein, the term “prevent,” a disorder, e.g., a myeloma, means that a subject (e.g., a human) is less likely to have the disorder, e.g., a myeloma, if the subject receives the antibody molecule.

Various aspects of the compositions and methods herein are described in further detail below. Additional definitions are set out throughout the specification.

### CD138

CD138 is a protein which in human is encoded by the SDC1 gene. CD138 is also known as Syndecan 1, Syndecan Proteoglycan 1, CD138 Antigen, SYND1, SDC, Syndecan-1, or Syndecan.

CD138 is a transmembrane (type I) heparan sulfate proteoglycan (HSPG) and is a member of the syndecan proteoglycan family. CD138 is highly expressed on differentiated plasma cells (PCs) and is both a primary diagnostic biomarker of multiple myeloma (MM) as well as an indicator of clinically poor prognosis. CD138 is also stably and significantly overexpressed on multiple myeloma cells derived from patients during multiple stages of disease progression with greater than 70% of these patients exhibiting increased CD138 cell surface expression on MM cells autologously derived from fresh BM aspirates. CD138 gene expression likewise is increased by several-fold in patient derived MM cells relative to “normal” plasma cells derived from healthy patient controls. Without wishing to be bound by theory, it is believed that in an embodiment, CD138 is targeted for immunotherapy for MM, including, but not limited to, smoldering myeloma, a relatively early and largely asymptomatic phase of disease amenable to early treatment intervention. Without wishing to be bound by theory, it is believed that in an embodiment, targeting CD138 can provide additional therapeutic benefit based on its key functions as a promoter of myeloma cell growth, adhesion and survival and other key aspects of myeloma cancer biology. The syndecans mediate cell binding, cell signaling, and cytoskeletal organization, and syndecan receptors are required for internalization of the HIV-1 tat protein. CD138 functions as an integral membrane protein and participates in cell proliferation, cell migration and cell-matrix interactions via its receptor for extracellular matrix proteins. Altered CD138 expression has been detected in several different tumor types.

The core of CD138 includes three major domains: 1) short cytoplasmic domain; 2) plasma membrane-spanning hydrophobic domain; and 3) long extracellular domain. The functions of CD138 domains are described, e.g., in Stepp et al. Adv Wound Care (New Rochelle). 2015; 4(4):235-249). The cytoplasmic domains can transmit signals and also bind to anchoring molecules including PDZ family members. The heparan sulfate chains of CD138 also serve important biological functions. In mammals, CD138 is a major heparan sulfate proteoglycan (HSPG) on epithelial cells with high levels of expression (Fuki et al. J Clin Invest. 1997; 100(6):1611-1622). Without wishing to be bound by theory, it is believed that the HSPGs of CD138 allow the proteoglycan to bind to the heparin-binding sites present on a number of ECM proteins, growth factors, cytokines, and other proteins (Stepp et al. Adv Wound Care (New Rochelle). 2015; 4(4):235-249).

For example, the signal peptide comprises residues 1-22; the extracellular domain comprises residues 23-254; the transmembrane domain comprises residues 255-275; the cytoplasmic domain comprises residues 276-310; or the integrin binding domain (IBD) comprises residues 88-122, of a human CD138 protein, e.g., any of SEQ ID NOS: 1-3 or 450.

In an embodiment, an anti-CD138 antibody molecule described herein can modulate (e.g., inhibit) the binding of CD138 to one or more proteins that interact (e.g., bind directly or indirectly) with the extracellular domain of CD138. In an embodiment, an anti-CD138 antibody molecule described herein can modulate (e.g., inhibit) a function associated with a protein that interacts (e.g., bind directly or indirectly) with the extracellular domain of CD138. In an embodiment, a CD138-interacting protein binds to the extracellular domain of CD138 directly. In an embodiment, a CD138-interacting protein binds to the extracellular domain of CD138 through a glycosaminoglycan (GAG) chain.

Exemplary of CD138-interacting proteins and their functions are described, e.g., in Stepp et al. Adv Wound Care (New Rochelle). 2015; 4(4):235-249, the content of which is incorporated by reference in its entirety.

For example, proteins that are capable of interacting with the extracellular domain of CD138 directly or indirectly include, but are not limited to, a matrix protein (e.g., a laminin, a fibronectin, thrombospondin, collagen, fibrin, HB-GAM, tenascin, vitronectin, fibrillin, or tropoelastin), a protease (e.g., MMP7, MMP9, ADAMTS4, MT1-PPT, neutrophil elastase, cathepsin G, or carboxypeptidase), a receptor (e.g., an integrin, αᵥβ₃, a_{vP}5, a₆β4, a₂β₁ (a₃β₁, or a_{M}β₂), a cytokine or growth factor (e.g., a morphogen (e.g., activin, BMP-2, BMP-4, chordin, Sonic Hedgehog, a Frizzled related protein, a Sprouty peptide, any of Wnt1 to Wnt13, an antiangiogenic factor (e.g., angistatin or endostatin), a growth factor (e.g., amphiregulin, batacellulin, HB-EGF, neuregulin, any of FGF1 to FGF23, PDGF, GDNF, an VEGF, HGF, TGFβ1, TGFβ2, TPA, or PAI-1), or a cytokine (e.g., GM-CSF, IL-2, IL-3, IL-4, IL-5, IL-7, IL-12, interferon, TNF-α, a CC chemokine, or a CXC chemokine), a protein associated with energy balance (e.g., ApoB, ApoE, or lipoprotein lipase), a complement or coagulation protein (e.g., antithrombin II, tissue factor (TF), pathway inhibitor, Factor IX, Factor X, Factor XI, or Factor XII), or a viral or parasite coat protein (e.g., HIV-1-tat, HIV-1 gp41, HIV-1 gp120, HSV gB, HSV gC, HSV gD, a coat protein of HHV-6 or HHV-8, or G-protein of RSV).

CD138 expressed on the cell surface can be cleaved by specific proteases and the shed CD138 is responsible for mediating paracrine and autocrine functions. Shed CD138 is soluble and secreted ectodomain (ECD) in blood and matrix. Shed CD138 is an indicator of poor prognosis in multiple myeloma patients and enhanced tumor progression in myeloma mouse models. Typically, shed CD138 is not considered to be primarily responsible for the disease manifestation. Translocation of CD138 to the cell nucleus can correlate to the differentiation and proliferation of certain tumor cells. In an embodiment, the anti-CD138 antibody molecules described herein preferentially target membrane-associated CD138 over soluble CD138.

CD138 is generally not present on B lymphocytes and it is expressed after the onset of plasma cell differentiation. CD138 is highly expressed on malignant plasma cells (myeloma) and has a causal role in disease progression. CD138 is implicated in various biological functions. For example, it can bind to extracellular proteins, growth factors, and chemokines; engage and activate the αVβ3 and αVβ5 integrin when clustered; regulate the biogenesis of exosomes; and regulate bone marrow microenvironment that supports myeloma growth and metastasis. Multiple signals can be attenuated by targeting CD138.

CD138 is upregulated in multiple myeloma (Tassone et al. Blood. 104(12): 3688-3696). It is overexpressed on malignant plasma cells. Multiple myeloma cells typically express between 50-200 fold higher levels of CD138. Soluble CD138 (sCD138) levels are generally from less than 60 ng/mL in normal serum to 200-1500 ng/mL in sera of multiple myeloma patients. CD138 is overexpressed in about 80% multiple myeloma patients.

CD138 can be used as a primary diagnostic marker for multiple myeloma. Increased levels of shed CD138 in serum correlated to increased tumor burden and poorer outcomes. CD138+ myeloma cells show higher proliferation and CD138+ myeloma patients have lower overall survival rates. CD138+ myeloma cells aberrantly express angiogenic factors, e.g., HGF, IL-15, ANG, APRIL, CTGF, or TGFA (Hose et al. Blood. 2009; 114(1): 128-143). Expression levels of CD138 and its released extracellular domain correlate with tumor malignancy, phenotype, and metastatic potential for both solid and hematological tumors. CD138 expression varies among cancer types, but the differential expression signatures between normal and cancer cells in epithelial and stromal compartments are directly associated with aggressiveness of tumors and patient’s clinical outcome and survival.

Exemplary amino acid and nucleotide sequences of human CD138 are described, e.g., in Mali et al. J Biol Chem. 1990; 265(12): 6884-6889; Lories et al. J Biol Chem. 1992; 267(2): 1116-1122.

The amino acid sequence of an exemplary human CD138 precursor (SEQ ID NO: 1) is provided as follows.

MRRAALWLWLCALALSLQPALPQIVATNLPPEDQDGSGDDSDNFSGSGAG
ALQDITLSQQTPSTWKDTQLLTAIPTSPEPTGLEATAASTSTLPAGEGPK
EGEAVVLPEVEPGLTAREQEATPRPRETTQLPTTHQASTTTATTAQEPAT
SHPHRDMQPGHHETSTPAGPSQADLHTPHTEDGGPSATERAAEDGASSQL
PAAEGSGEQDFTFETSGENTAVVAVEPDRRNQSPVDQGATGASQGLLDRK
EVLGGVIAGGLVGLIFAVCLVGFMLYRMKKKDEGSYSLEEPKQANGGAYQ
KPTKQEEFYA

The amino acid sequence of an exemplary human CD138 precursor variant (Q136L) (SEQ ID NO: 2) is provided as follows.

MRRAALWLWLCALALSLQPALPQIVATNLPPEDQDGSGDDSDNFSGSGAG
ALQDITLSQQTPSTWKDTQLLTAIPTSPEPTGLEATAASTSTLPAGEGPK
EGEAVVLPEVEPGLTAREQEATPRPRETTQLPTTHLASTTTATTAQEPAT
SHPHRDMQPGHHETSTPAGPSQADLHTPHTEDGGPSATERAAEDGASSQL
PAAEGSGEQDFTFETSGENTAVVAVEPDRRNQSPVDQGATGASQGLLDRK
EVLGGVIAGGLVGLIFAVCLVGFMLYRMKKKDEGSYSLEEPKQANGGAYQ
KPTKQEEFYA

The amino acid sequence of an exemplary human CD138 precursor variant (T76M) (SEQ ID NO: 3) is provided as follows.

MRRAALWLWLCALALSLQPALPQIVATNLPPEDQDGSGDDSDNFSGSGAG
ALQDITLSQQTPSTWKDTQLLTAIPMSPEPTGLEATAASTSTLPAGEGPK
EGEAVVLPEVEPGLTAREQEATPRPRETTQLPTTHQASTTTATTAQEPAT
SHPHRDMQPGHHETSTPAGPSQADLHTPHTEDGGPSATERAAEDGASSQL
PAAEGSGEQDFTFETSGENTAVVAVEPDRRNQSPVDQGATGASQGLLDRK
EVLGGVIAGGLVGLIFAVCLVGFMLYRMKKKDEGSYSLEEPKQANGGAYQ
KPTKQEEFYA

The signal peptide includes amino acids 1-22 of any of SEQ ID NOs: 1-3. The mature peptide includes amino acids 23-310 of any of SEQ ID NOs: 1-3. The extracellular domain includes amino acids 23-254 of any of SEQ ID NOs: 1-3. The transmembrane domain includes amino acids 255-275 of any of SEQ ID NOs: 1-3. The cytoplasmic domain includes amino acids 276-310 of any of SEQ ID NOs: 1-3.

An exemplary coding nucleotide sequence of human CD138 (SEQ ID NO: 4) is provided as follows. This nucleotide sequence encodes the amino acid sequence of SEQ ID NO: 1.

ATGAGGCGCGCGGCGCTCTGGCTCTGGCTGTGCGCGCTGGCGCTGAGCCT
GCAGCCGGCCCTGCCGCAAATTGTGGCTACTAATTTGCCCCCTGAAGATC
AAGATGGCTCTGGGGATGACTCTGACAACTTCTCCGGCTCAGGTGCAGGT
GCTTTGCAAGATATCACCTTGTCACAGCAGACCCCCTCCACTTGGAAGGA
CACGCAGCTCCTGACGGCTATTCCCACGTCTCCAGAACCCACCGGCCTGG
AGGCTACAGCTGCCTCCACCTCCACCCTGCCGGCTGGAGAGGGGCCCAAG
GAGGGAGAGGCTGTAGTCCTGCCAGAAGTGGAGCCTGGCCTCACCGCCCG
GGAGCAGGAGGCCACCCCCCGACCCAGGGAGACCACACAGCTCCCGACCA
CTCATCAGGCCTCAACGACCACAGCCACCACGGCCCAGGAGCCCGCCACC
TCCCACCCCCACAGGGACATGCAGCCTGGCCACCATGAGACCTCAACCCC
TGCAGGACCCAGCCAAGCTGACCTTCACACTCCCCACACAGAGGATGGAG
GTCCTTCTGCCACCGAGAGGGCTGCTGAGGATGGAGCCTCCAGTCAGCTC
CCAGCAGCAGAGGGCTCTGGGGAGCAGGACTTCACCTTTGAAACCTCGGG
GGAGAATACGGCTGTAGTGGCCGTGGAGCCTGACCGCCGGAACCAGTCCC
CAGTGGATCAGGGGGCCACGGGGGCCTCACAGGGCCTCCTGGACAGGAAA
GAGGTGCTGGGAGGGGTCATTGCCGGAGGCCTCGTGGGGCTCATCTTTGC
TGTGTGCCTGGTGGGTTTCATGCTGTACCGCATGAAGAAGAAGGACGAAG
GCAGCTACTCCTTGGAGGAGCCGAAACAAGCCAACGGCGGGGCCTACCAG
AAGCCCACCAAACAGGAGGAATTCTATGCCTGA

Another exemplary coding nucleotide sequence of human CD138 (SEQ ID NO: 5) is provided as follows. This nucleotide sequence also encodes the amino acid sequence of SEQ ID NO: 1.

ATGAGGCGCGCGGCGCTCTGGCTCTGGCTGTGCGCGCTGGCGCTGAGCCT
GCAGCCGGCCCTGCCGCAAATTGTGGCTACTAATTTGCCCCCTGAAGATC
AAGATGGCTCTGGGGATGACTCTGACAACTTCTCCGGCTCAGGTGCAGGT
GCTTTGCAAGATATCACCTTGTCACAGCAGACCCCCTCCACTTGGAAGGA
CACGCAGCTCCTGACGGCTATTCCCACGTCTCCAGAACCCACCGGCCTGG
AGGCTACAGCTGCCTCCACCTCCACCCTGCCGGCTGGAGAGGGGCCCAAG
GAGGGAGAGGCTGTAGTCCTGCCAGAAGTGGAGCCTGGCCTCACCGCCCG
GGAGCAGGAGGCCACCCCCCGACCCAGGGAGACCACACAGCTCCCGACCA
CTCATCAGGCCTCAACGACCACAGCCACCACGGCCCAGGAGCCCGCCACC
TCCCACCCCCACAGGGACATGCAGCCTGGCCACCATGAGACCTCAACCCC
TGCAGGACCCAGCCAAGCTGACCTTCACACTCCCCACACAGAGGATGGAG
GTCCTTCTGCCACCGAGAGGGCTGCTGAGGATGGAGCCTCCAGTCAGCTC
CCAGCAGCAGAGGGCTCTGGGGAGCAGGACTTCACCTTTGAAACCTCGGG
GGAGAATACGGCTGTAGTGGCCGTGGAGCCTGACCGCCGGAACCAGTCCC
CAGTGGATCAGGGGGCCACGGGGGCCTCACAGGGCCTCCTGGACAGGAAA
GAGGTGCTGGGAGGGGTCATTGCCGGAGGCCTCGTGGGGCTCATCTTTGC
TGTGTGCCTGGTGGGTTTCATGCTGTACCGCATGAAGAAGAAGGACGAAG
GCAGCTACTCCTTGGAGGAGCCGAAACAAGCCAACGGCGGGGCCTACCAG
AAGCCCACCAAACAGGAGGAATTCTATGCCTGA

As used herein, when an anti-CD138 antibody molecule binds, or substantially binds, to human CD138, it binds, or substantially binds, to one or more isoforms of human CD138. In an embodiment, the antibody molecule binds or substantially binds to human CD138 having an amino acid sequence described herein, or encoded by a nucleotide sequence described herein. In an embodiment, the antibody molecule binds or substantially binds to human CD138 comprising amino acids 23-254 of any of SEQ ID NOs: 1-3.

Exemplary amino acid and nucleotide sequences of mouse CD138 are described, e.g., in Saunders et al. J Cell Biol. 1989; 108(4): 1547-1556; and Vihinen et al. J Biol Chem. 1993; 268(23): 17261-17269.

The amino acid sequence of an exemplary mouse CD138 precursor (SEQ ID NO: 6) is provided as follows.

MRRAALWLWLCALALRLQPALPQIVAVNVPPEDQDGSGDDSDNFSGSGTG
ALPDTLSRQTPSTWKDVWLLTATPTAPEPTSSNTETAFTSVLPAGEKPEE
GEPVLHVEAEPGFTARDKEKEVTTRPRETVQLPITQRASTVRVTTAQAAV
TSHPHGGMQPGLHETSAPTAPGQPDHQPPRVEGGGTSVIKEVVEDGTANQ
LPAGEGSGEQDFTFETSGENTAVAAVEPGLRNQPPVDEGATGASQSLLDR
KEVLGGVIAGGLVGLIFAVCLVAFMLYRMKKKDEGSYSLEEPKQANGGAY
QKPTKQEEFYA

The signal peptide includes amino acids 1-22 of SEQ ID NO: 6. The mature peptide includes amino acids 23-311 of SEQ ID NO: 6. The extracellular domain includes amino acids 23-255 of SEQ ID NO: 6. The transmembrane domain includes amino acids 256-276 of SEQ ID NO: 4. The cytoplasmic domain includes amino acids 277-311 of SEQ ID NO: 6.

An exemplary coding nucleotide sequence of mouse CD138 (SEQ ID NO: 7) is provided as follows.

ATGAGACGCGCGGCGCTCTGGCTCTGGCTCTGCGCGCTGGCGCTGCGCCT
GCAGCCTGCCCTCCCGCAAATTGTGGCTGTAAATGTTCCTCCTGAAGATC
AGGATGGCTCTGGGGATGACTCTGACAACTTCTCTGGCTCTGGCACAGGT
GCTTTGCCAGATACTTTGTCACGGCAGACACCTTCCACTTGGAAGGACGT
GTGGCTGTTGACAGCCACGCCCACAGCTCCAGAGCCCACCAGCAGCAACA
CCGAGACTGCTTTTACCTCTGTCCTGCCAGCCGGAGAGAAGCCCGAGGAG
GGAGAGCCTGTGCTCCATGTAGAAGCAGAGCCTGGCTTCACTGCTCGGGA
CAAGGAAAAGGAGGTCACCACCAGGCCCAGGGAGACCGTGCAGCTCCCCA
TCACCCAACGGGCCTCAACAGTCAGAGTCACCACAGCCCAGGCAGCTGTC
ACATCTCATCCGCACGGGGGCATGCAACCTGGCCTCCATGAGACCTCGGC
TCCCACAGCACCTGGTCAACCTGACCATCAGCCTCCACGTGTGGAGGGTG
GCGGCACTTCTGTCATCAAAGAGGTTGTCGAGGATGGAACTGCCAATCAG
CTTCCCGCAGGAGAGGGCTCTGGAGAACAAGACTTCACCTTTGAAACATC
TGGGGAGAACACAGCTGTGGCTGCCGTAGAGCCCGGCCTGCGGAATCAGC
CCCCGGTGGACGAAGGAGCCACAGGTGCTTCTCAGAGCCTTTTGGACAGG
AAGGAAGTGCTGGGAGGTGTCATTGCCGGAGGCCTAGTGGGCCTCATCTT
TGCTGTGTGCCTGGTGGCTTTCATGCTGTACCGGATGAAGAAGAAGGACG
AAGGCAGCTACTCCTTGGAGGAGCCCAAACAAGCCAATGGCGGTGCCTAC
CAGAAACCCACCAAGCAGGAGGAGTTCTACGCCTGA

As used herein, when an anti-CD138 antibody molecule binds, or substantially binds, to mouse CD138, it binds, or substantially binds, to one or more isoforms of mouse CD138. In an embodiment, the antibody molecule binds or substantially binds to human CD138 having an amino acid sequence described herein, or encoded by a nucleotide sequence described herein. In an embodiment, the antibody molecule binds or substantially binds to mouse CD138 comprising amino acids 23-255 of SEQ ID NO: 6.

### pH-Selective Binding to CD138

The antibody molecules described herein can have pH-selective binding to CD138.

In an embodiment, the antibody molecule binds to CD138 more strongly under conditions in which the pH is more acidic than conditions of physiological pH (e.g., pH 7.4). In an embodiment, the antibody molecule binds to CD138 more strongly at a pH of about 6.0-6.5 than at a physiological pH (e.g., pH 7.4). In some embodiments, the antibody molecule binds to CD138 more strongly at a pH of about 6.5-7.0 than at a physiological pH (e.g., pH 7.4). In an embodiment, the antibody molecule binds to CD138 more strongly at a pH of about 7.0-7.3 than at a physiological pH (e.g., pH 7.4). In an embodiment, the antibody molecule binds to CD138 more strongly at a pH of about 6.0, 6.1, 6.2, 6.3, 6.4, 6.5, 6.6, 6.7, 6.8, 6.9, 7.0, 7.1, 7.2, or 7.3 than at a physiological pH (e.g., pH 7.4). In an embodiment, the antibody molecule binds to CD138 at acidic pH (e.g., as described herein, e.g., about pH 6.0-6.5, 6.5-7.0, or 7.0-7.3, or pH of about 6.0, 6.1, 6.2, 6.3, 6.4, 6.5, 6.6, 6.7, 6.8, 6.9, 7.0, 7.1, 7.2, or 7.3) at least 2, 3, 4, 5, 6, 7, 8, 9, 10, 15, 20, 25, 30, 40, 50, 60, 70, 80, 90, 100, 150, 200, 300, 400, 500, 600, 700, 800, 900, or 1000-fold more strongly than at physiological pH (e.g., pH 7.4).

In an embodiment, the antibody molecule has a KD for CD138 at acidic pH (e.g., as described herein, e.g., about pH 6.0-6.5, 6.5-7.0, or 7.0-7.3, or pH of about 6.0, 6.1, 6.2, 6.3, 6.4, 6.5, 6.6, 6.7, 6.8, 6.9, 7.0, 7.1, 7.2, or 7.3) at least 2, 3, 4, 5, 6, 7, 8, 9, 10, 15, 20, 25, 30, 40, 50, 60, 70, 80, 90, 100, 150, 200, 300, 400, 500, 600, 700, 800, 900, or 1000-fold lower than the KD for CD138 at physiological pH (e.g., pH 7.4). In an embodiment, the antibody molecule has a KD for CD138 at acidic pH (e.g., as described herein, e.g., about pH 6.0-6.5, 6.5-7.0, or 7.0-7.3, or pH of about 6.0, 6.1, 6.2, 6.3, 6.4, 6.5, 6.6, 6.7, 6.8, 6.9, 7.0, 7.1, 7.2, or 7.3) at least 4-fold lower than the KD for CD138 at physiological pH (e.g., pH 7.4). In an embodiment, binding affinity of the antibody molecule for CD138 at a particular pH is determined by biolayer interferometry, e.g., as described herein. In an embodiment, binding affinity of the antibody molecule for CD138 at a particular pH is determined using a cell-based assay (e.g., measuring the level or activity of a downstream effector or reporter of CD138 activity in a cell, e.g., as described herein).

### Epitope

The antibody molecules described herein (e.g., pH-selective antibody molecules described herein) can bind to an epitope on CD138 (e.g., human CD138). For example, an epitope bound by an antibody molecule described herein can include one or more epitope contact points described herein.

In an embodiment, the antibody molecule binds to a plurality of (e.g., two) epitopes on CD138 (e.g., human CD138). In an embodiment, the antibody molecule binds to a first epitope on CD138 and to a second epitope on CD138. In an embodiment, an epitope on CD138 bound by an antibody molecule described herein is comprised in an integrin binding region of CD138. In an embodiment, an epitope on CD138 bound by an antibody molecule described herein is comprised in a membrane proximal region of CD138. In an embodiment, an epitope on CD138 bound by an antibody molecule described herein comprises the amino acid sequence VEP. In an embodiment, an epitope on CD138 bound by an antibody molecule described herein comprises the amino acid sequence hhxVEP, wherein h indicates a hydrophobic residue, and x can be any amino acid (e.g., a noncharged residue or any amino acid other than Asp, Glu, Lys, Arg, Tyr, or Cys). The epitope on CD138 bound by an antibody molecule described herein optionally further comprises a noncontiguous arginine residue, e.g., on the C-terminal side of the VEP motif, e.g., within about 1, 2, 3, 4, 5, 6, 7, 8, 9, or 10 amino acid residues of the VEP motif.

In an embodiment, the first epitope on CD138 comprises the amino acid sequence VEP. In an embodiment, the first epitope on CD138 comprises the amino acid sequence LPEVEP, or an amino acid sequence having no more than 1, 2, or 3 amino acid sequence differences (e.g., substitutions, additions, or deletions) therefrom, or an amino acid sequence having at least 75%, 80%, 85%, 90%, 95%, 96%, 97%, 98%, or 99% sequence identity thereto. In an embodiment, the first epitope on CD138 comprises the amino acid sequence GEAVVLPEVEPGLTAR, or an amino acid sequence having no more than 1, 2, 3, 4, or 5 amino acid sequence differences (e.g., substitutions, additions, or deletions) therefrom, or an amino acid sequence having at least 75%, 80%, 85%, 90%, 95%, 96%, 97%, 98%, or 99% sequence identity thereto.

In an embodiment, the first epitope on CD138 comprises the amino acid sequence VEP. In an embodiment, the first epitope on CD138 comprises the amino acid sequence VVAVEP, or an amino acid sequence having no more than 1, 2, or 3 amino acid sequence differences (e.g., substitutions, additions, or deletions) therefrom, or an amino acid sequence having at least 75%, 80%, 85%, 90%, 95%, 96%, 97%, 98%, or 99% sequence identity thereto. In an embodiment, the first epitope on CD138 comprises the amino acid sequence ENTAVVAVEPDRRNQ, or an amino acid sequence having no more than 1, 2, 3, 4, or 5 amino acid sequence differences (e.g., substitutions, additions, or deletions) therefrom, or an amino acid sequence having at least 75%, 80%, 85%, 90%, 95%, 96%, 97%, 98%, or 99% sequence identity thereto.

In an embodiment, the second epitope on CD138 comprises the amino acid sequence VEP. In an embodiment, the second epitope on CD138 comprises the amino acid sequence LPEVEP, or an amino acid sequence having no more than 1, 2, or 3 amino acid sequence differences (e.g., substitutions, additions, or deletions) therefrom, or an amino acid sequence having at least 75%, 80%, 85%, 90%, 95%, 96%, 97%, 98%, or 99% sequence identity thereto. In an embodiment, the second epitope on CD138 comprises the amino acid sequence GEAVVLPEVEPGLTAR, or an amino acid sequence having no more than 1, 2, 3, 4, or 5 amino acid sequence differences (e.g., substitutions, additions, or deletions) therefrom, or an amino acid sequence having at least 75%, 80%, 85%, 90%, 95%, 96%, 97%, 98%, or 99% sequence identity thereto.

In an embodiment, the second epitope on CD138 comprises the amino acid sequence VEP. In an embodiment, the second epitope on CD138 comprises the amino acid sequence VVAVEP, or an amino acid sequence having no more than 1, 2, or 3 amino acid sequence differences (e.g., substitutions, additions, or deletions) therefrom, or an amino acid sequence having at least 75%, 80%, 85%, 90%, 95%, 96%, 97%, 98%, or 99% sequence identity thereto. In an embodiment, the second epitope on CD138 comprises the amino acid sequence ENTAVVAVEPDRRNQ, or an amino acid sequence having no more than 1, 2, 3, 4, or 5 amino acid sequence differences (e.g., substitutions, additions, or deletions) therefrom, or an amino acid sequence having at least 75%, 80%, 85%, 90%, 95%, 96%, 97%, 98%, or 99% sequence identity thereto.

In an embodiment, an antibody molecule described herein can bind to a first epitope on a first copy of CD138 and to a second epitope on a second copy of CD138. In an embodiment, the first epitope on the first copy of CD138 and the second epitope on the second copy of CD138 are the same epitope. In an embodiment, the first epitope on the first copy of CD138 and the second epitope on the second copy of CD138 are different epitopes.

In an embodiment, the antibody molecule binds to an epitope of CD138 comprising a membrane proximal region. In an embodiment, the antibody molecule binds to an epitope of CD138 comprising at least two distinct peptide regions (e.g., comprising peptide 2A and/or 6A, and/or portions thereof). In an embodiment, the antibody molecule binds to an epitope of CD138 distinct from the epitope bound by antibody BB4.

In an embodiment, the antibody molecule binds to CD138 (e.g., human CD138) with at least 10% (e.g., at least 10%, 15%, 20%, 25%, 30%, 35%, 40%, 50%, 55%, 60%, 70%, 80%, or 90%) greater affinity relative to a reference anti-CD138 antibody (e.g., antibody BB4), e.g., as determined by a cell-binding assay described herein. In an embodiment, the CD138 is membrane-associated. In an embodiment, the antibody molecule binds to a soluble CD138,e.g., the extracellular domain of soluble CD138 (e.g., having the sequence of amino acids 18-251 of SEQ ID NO: 1). In an embodiment, the antibody molecule binds to peptide 2A of human CD138. In certain embodiments, the antibody molecule binds to peptide 6A of human CD138.

In an embodiment, the anti-CD138 antibody molecules described herein have one, two, or all of the following properties: optimal distance of epitope from the cell membrane (e.g., not on the N-terminal of IDB); appropriate orientation of the Fc region for CD16 engagement; or proper CD138 engagement that allows for CD16 clustering on NK cells (e.g., to overcome the effect of high amount of glycosylation on CD138 molecules that may restrict the access of NK cells).

Without wishing to be bound by theory, it is believed that in an embodiment altering the position of the antibody epitope can change certain effector mechanisms engaged. For example, antibody-dependent cellular cytotoxicity (ADCC) and complement-dependent cytotoxicity (CDC) may favor a membrane-proximal epitope versus a membrane-distal epitope (Cleary et al. J Immunol. 2017; 198(10): 3999-4011). In an embodiment, antibodies designed to delete target cells through specific effector mechanisms can be selected by altering the position of the antibody epitope (e.g., the distance of epitope from membrane).

In an embodiment, the mode of engagement can affect the ability of the antibody to mediate effector functions. For example, the angle of antibody binding to the extracellular loop with regard to the membrane surface may be different (e.g., parallel or perpendicular to the membrane surface) between antibodies that bind to the same peptide epitopes.

In an embodiment, the anti-CD138 antibody molecules described herein bind to an epitope that has one, two, or all of the following properties: proximal to the cell membrane; not restricted or occluded by the glycosaminoglycan (GAG) chains; or preferentially present on membrane-associated CD138. In an embodiment, the anti-CD138 antibody molecules described herein can bind to a desired epitope region and engage with the optimal pose relative to the membrane. In an embodiment, the epitope is a linear epitope. In an embodiment, the antibody molecule binds to an extracellular region of CD138 distant from the transmembrane region. In an embodiment, the epitope is a non-contiguous or conformational epitope.

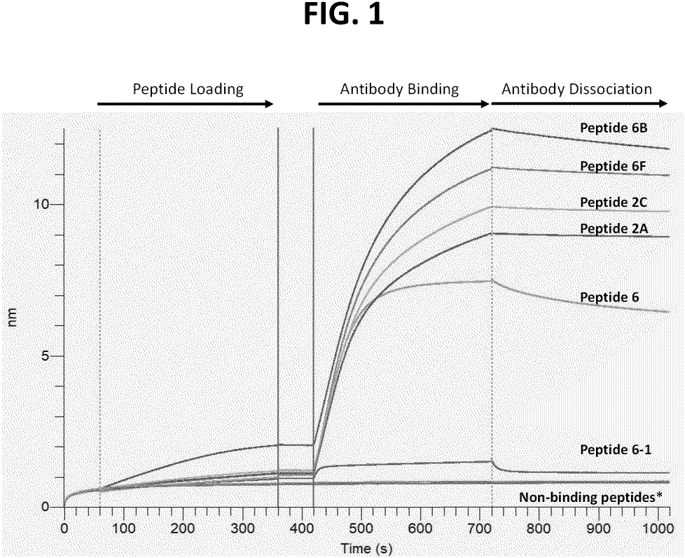

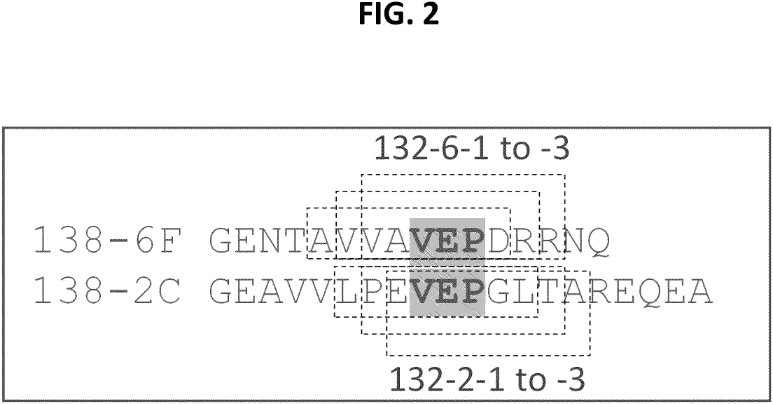

Peptides for identification of desired epitopes for anti-CD 138 antibodies are shown, e.g., in FIG. 2 of PCT Publication No. WO 2019/070726 or U.S. Pat. Application Publication No. US 2019/0100588. Without wishing to be bound by theory, it is believed that in an embodiment, the anti-CD138 antibody molecules described herein target a peptide region between residues Gly217 to Glu251 of human CD138, e.g., as shown in FIG. 1 of PCT Publication No. WO 2019/070726 or U.S. Pat. Application Publication No. US 2019/0100588. This region is expected to have a linear random coil conformation. In an embodiment, the anti-CD138 antibody molecule binds to at least one linear tetrapeptide in the aforesaid region. In an embodiment, the anti-CD138 antibody molecule binds to a combination of linear tetrapeptides (e.g., two, three, four, or more adjacent tetrapeptides) in the aforesaid region.

The amino acid sequences of the aforesaid peptides are shown in Table 3.

TABLE 3

Peptides for Identification of CD138 Epitopes

| Peptide | Region | Amino Acid Sequence | SEQ ID NO | Length |
|---|---|---|---|---|
| Pep1a | 23-50 | QIVATNLPPEDQDGSGDDSDNFSGSGAGALQDITLSQQT | 8 | 39 |
| Pep1b | 51-95 | ALQDITLSQQTPSTWKDTQLLTAIPTSPEPTGLEATAASTS TLPA | 9 | 45 |
| Pep2a | 88-121 | ASTSTLPAGEGPKEGEAVVLPEVEPGLTAREQEA | 10 | 34 |
| Pep2b | 88-102 | ASTSTLPAGEGPKEG | 11 | 15 |
| Pep3 | 111-150 | EPGLTAREQEATPRPRETTQLPTTHQASTTTATTAQEPAT | 12 | 40 |
| Pep4 | 146-180 | QEPATSHPHRDMQPGHHETSTPAGPSQADLHTPHT | 13 | 35 |
| Pep5-6 | 176-250 | HTPHTEDGGPSATERAAEDGASSQLPAAEGSGEQDFTFE TSGENTAVVAVEPDRRNQSPVDQGATGASQGLLDRK | 14 | 75 |
| Pep5 | 176-214 | HTPHTEDGGPSATERAAEDGASSQLPAAEGSGEQDFTFE | 15 | 39 |
| Pep6 | 210-250 | DFTFETSGENTAVVAVEPDRRNQSPVDQGATGASQGLLD RK | 16 | 41 |
| Pep6a | 220-245 | TAVVAVEPDRRNQSPVDQGATGASQG | 17 | 26 |

In Table 3, the overlapping amino acids among the peptides are shown in bold; the BB4 epitope residues are shown in italic; the glycosaminoglycan (heparan sulfate, chondroitin sulfate) chain carrying serine residues are underlined. The terms “Peptide” and “Pep” are used interchangeably herein. For peptide designations, the lower case and upper-case letters are intended to have the same meaning. For example, the terms “Peptide 1A,” “Peptide 1a,” “Pep1A,” and “Pepla” can be used to refer to the same peptide.

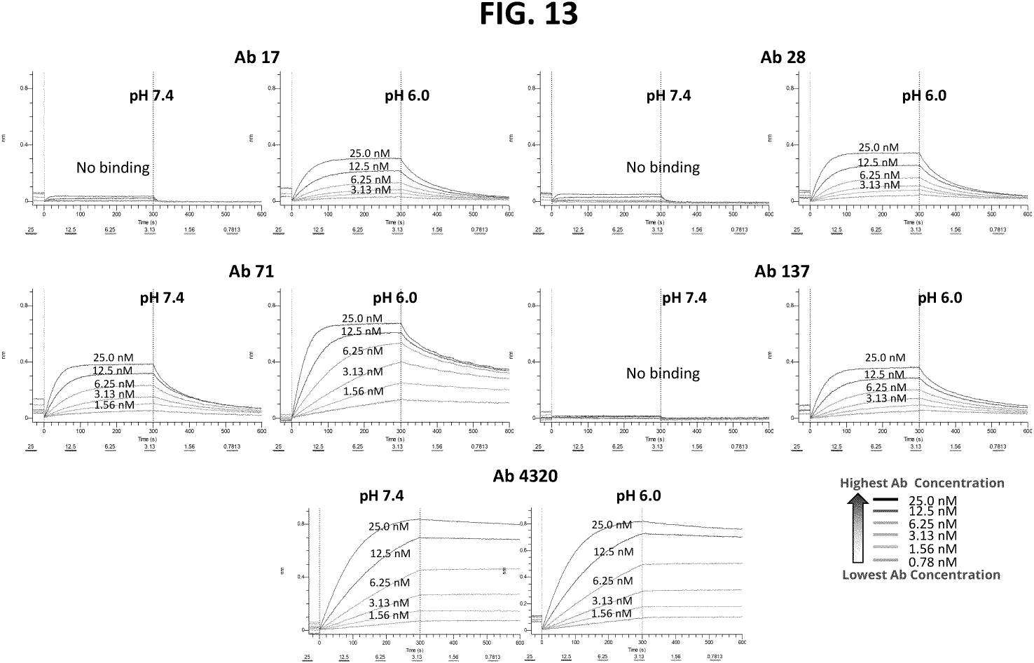

Other exemplary peptides used for identification of desired epitopes for anti-CD138 antibodies are described herein, e.g., in FIG. 13 and 22C of PCT Publication No. WO 2019/070726 or U.S. Pat. Application Publication No. US 2019/0100588.

In an embodiment, the antibody molecule contacts (e.g., binds, or substantially binds, to) a region in CD138 corresponding to one or more peptides as described in Table 3, or in FIG. 13 or 22C of PCT Publication No. WO 2019/070726 or U.S. Pat. Application Publication No. US 2019/0100588. In an embodiment, the peptide is Pep6. In an embodiment, the peptide is Pep6a. In an embodiment, the peptide is Pep5. In an embodiment, the peptide is Pep4. In an embodiment, the antibody molecule contacts Pep6 or Pep6a and does not contact Pep4. In an embodiment, the antibody molecule does not contact any of Pep1a, Pep1b, Pep2a, Pep2b, Pep3, Pep4, or Pep5. In an embodiment, the antibody molecule does not contact Pep2a. In an embodiment, the antibody molecule contacts Pep2a but does not bind to the same epitope as BB4.

In an embodiment, the antibody molecule contacts Pep2a and Pep6. In an embodiment, the antibody molecule contacts Pep2a and Pep2c. In an embodiment, the antibody molecule contacts Pep6b. In an embodiment, the antibody molecule contacts Pep2a, Pep2c, and Pep6b. In an embodiment, the antibody molecule does not contact Pep6e. In an embodiment, the antibody molecule contacts Pep6b and does not contact Pep6e. In an embodiment, the antibody molecule contacts Pep2a and Pep2c and does not contact Pep6e. In an embodiment, the antibody molecule contacts Pep2a, Pep2c, and Pep6b and does not contact Pep6e.

In an embodiment, the antibody molecule contacts Pep2a and Pep2d. In an embodiment, the antibody molecule contacts Pep6b and Pep6f. In an embodiment, the antibody molecule contacts Pep2a, Pep2d, Pep6b, and Pep6f.

In an embodiment, the antibody molecule binds, or substantially binds, to CD138 in an extracellular region proximal to the transmembrane domain of CD138. In an embodiment, the C-terminus of the extracellular region proximal to the transmembrane domain is within 70, 65, 60, 55, 50, 45, 40, 35, 30, 25, 20, 15, 10, or 5 amino acids from the N-terminus of the transmembrane domain. In an embodiment, the N-terminus of the extracellular region proximal to the transmembrane domain is within 75, 70, 65, 60, 55, 50, 45, 40, 35, 30, 25, 20, 15, 10, or 5 amino acids from the N-terminus of the transmembrane domain.

In an embodiment, the antibody molecule binds to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in the extracellular region proximal to the transmembrane domain.

In an embodiment, the antibody molecule binds to an epitope on CD138 comprising five or more consecutive amino acid residues in the extracellular region proximal to the transmembrane domain. In an embodiment, the antibody molecule binds to an epitope on CD138 comprising six or more consecutive amino acid residues in the extracellular region proximal to the transmembrane domain. In an embodiment, the antibody molecule binds to an epitope on CD138 comprising seven or more consecutive amino acid residues in the extracellular region proximal to the transmembrane domain. In an embodiment, the antibody molecule binds to an epitope on CD138 comprising eight or more consecutive amino acid residues in the extracellular region proximal to the transmembrane domain. In an embodiment, the antibody molecule binds to an epitope on CD138 comprising nine or more consecutive amino acid residues in the extracellular region proximal to the transmembrane domain. In an embodiment, the antibody molecule binds to an epitope on CD138 comprising ten or more consecutive amino acid residues in the extracellular region proximal to the transmembrane domain. In an embodiment, the antibody molecule binds to an epitope on CD138 comprising eleven or more consecutive amino acid residues in the extracellular region proximal to the transmembrane domain. In an embodiment, the antibody molecule binds to an epitope on CD138 comprising twelve or more consecutive amino acid residues in the extracellular region proximal to the transmembrane domain.

In an embodiment, the extracellular region proximal to the transmembrane domain corresponds to (e.g., comprises or consists of) Pep6. In an embodiment, the extracellular region proximal to the transmembrane domain corresponds to (e.g., comprises or consists of) Pep6a, 6b, 6e, and/or 6f. In an embodiment, the extracellular region proximal to the transmembrane domain corresponds to (e.g., comprises or consists of) Pep5.

In an embodiment, the antibody molecule contacts four or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41) consecutive amino acid residues in Pep6. In an embodiment, the antibody molecule contacts four or more (e.g., 5, 6, 7, 8, 9, 10, 11, 12, 13, 14, 15, 16, 17, 18, 19, 20, 21, 22, 23, 24, 25, or 26) consecutive amino acid residues in Pep6a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38) of the following peptides (e.g., from Pep6a): DFTF (SEQ ID NO: 18); FTFE (SEQ ID NO: 19); TFET (SEQ ID NO: 20); FETS (SEQ ID NO: 21); ETSG (SEQ ID NO: 22); TSGE (SEQ ID NO: 23); SGEN (SEQ ID NO: 24); GENT (SEQ ID NO: 25); ENTA (SEQ ID NO: 26); NTAV (SEQ ID NO: 27); TAVV (SEQ ID NO: 28); AVVA (SEQ ID NO: 29); VVAV (SEQ ID NO: 30); VAVE (SEQ ID NO: 31); AVEP (SEQ ID NO: 32); VEPD (SEQ ID NO: 33); EPDR (SEQ ID NO: 34); PDRR (SEQ ID NO: 35); DRRN (SEQ ID NO: 36); RRNQ (SEQ ID NO: 37); RNQS (SEQ ID NO: 38); NQSP (SEQ ID NO: 39); QSPV (SEQ ID NO: 40); SPVD (SEQ ID NO: 41); PVDQ (SEQ ID NO: 42); VDQG (SEQ ID NO: 43); DQGA (SEQ ID NO: 44); QGAT (SEQ ID NO: 45); GATG (SEQ ID NO: 46); ATGA (SEQ ID NO: 47); TGAS (SEQ ID NO: 48); GASQ (SEQ ID NO: 49); ASQG (SEQ ID NO: 50); SQGL (SEQ ID NO: 51); QGLL (SEQ ID NO: 52); GLLD (SEQ ID NO: 53); LLDR (SEQ ID NO: 54); or LDRK (SEQ ID NO: 55).

In an embodiment, the antibody molecule contacts five or more (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41) consecutive amino acid residues in Pep6a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37) of the following peptides (e.g., from Pep6a): DFTFE (SEQ ID NO: 56); FTFET (SEQ ID NO: 57); TFETS (SEQ ID NO: 58); FETSG (SEQ ID NO: 59); ETSGE (SEQ ID NO: 60); TSGEN (SEQ ID NO: 61); SGENT (SEQ ID NO: 62); GENTA (SEQ ID NO: 63); ENTAV (SEQ ID NO: 64); NTAVV (SEQ ID NO: 65); TAVVA (SEQ ID NO: 66); AVVAV (SEQ ID NO: 67); VVAVE (SEQ ID NO: 68); VAVEP (SEQ ID NO: 69); AVEPD (SEQ ID NO: 70); VEPDR (SEQ ID NO: 71); EPDRR (SEQ ID NO: 72); PDRRN (SEQ ID NO: 73); DRRNQ (SEQ ID NO: 74); RRNQS (SEQ ID NO: 75); RNQSP (SEQ ID NO: 76); NQSPV (SEQ ID NO: 77); QSPVD (SEQ ID NO: 78); SPVDQ (SEQ ID NO: 79); PVDQG (SEQ ID NO: 80); VDQGA (SEQ ID NO: 81); DQGAT (SEQ ID NO: 82); QGATG (SEQ ID NO: 83); GATGA (SEQ ID NO: 84); ATGAS (SEQ ID NO: 85); TGASQ (SEQ ID NO: 86); GASQG (SEQ ID NO: 87); ASQGL (SEQ ID NO: 88); SQGLL (SEQ ID NO: 89); QGLLD (SEQ ID NO: 90); GLLDR (SEQ ID NO: 91); or LLDRK (SEQ ID NO: 92).

In an embodiment, the antibody molecule contacts six or more (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or 41) consecutive amino acid residues in Pep6a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) of the following peptides (e.g., from Pep6a): DFTFET (SEQ ID NO: 93); FTFETS (SEQ ID NO: 94); TFETSG (SEQ ID NO: 95); FETSGE (SEQ ID NO: 96); ETSGEN (SEQ ID NO: 97); TSGENT (SEQ ID NO: 98); SGENTA (SEQ ID NO: 99); GENTAV (SEQ ID NO: 100); ENTAVV (SEQ ID NO: 101); NTAVVA (SEQ ID NO: 102); TAVVAV (SEQ ID NO: 103); AVVAVE (SEQ ID NO: 104); VVAVEP (SEQ ID NO: 105); VAVEPD (SEQ ID NO: 106); AVEPDR (SEQ ID NO: 107); VEPDRR (SEQ ID NO: 108); EPDRRN (SEQ ID NO: 109); PDRRNQ (SEQ ID NO: 110); DRRNQS (SEQ ID NO: 111); RRNQSP (SEQ ID NO: 112); RNQSPV (SEQ ID NO: 113); NQSPVD (SEQ ID NO: 114); QSPVDQ (SEQ ID NO: 115); SPVDQG (SEQ ID NO: 116); PVDQGA (SEQ ID NO: 117); VDQGAT (SEQ ID NO: 118); DQGATG (SEQ ID NO: 119); QGATGA (SEQ ID NO: 120); GATGAS (SEQ ID NO: 121); ATGASQ (SEQ ID NO: 122); TGASQG (SEQ ID NO: 123); GASQGL (SEQ ID NO: 124); ASQGLL (SEQ ID NO: 125); SQGLLD (SEQ ID NO: 126); QGLLDR (SEQ ID NO: 127); or GLLDRK (SEQ ID NO: 128).

In an embodiment, the antibody molecule contacts four or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) consecutive amino acid residues in Pep5.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36) of the following peptides (e.g., from Pep5): HTPH (SEQ ID NO: 129), TPHT (SEQ ID NO: 130), PHTE (SEQ ID NO: 131), HTED (SEQ ID NO: 132), TEDG (SEQ ID NO: 133), EDGG (SEQ ID NO: 134), DGGP (SEQ ID NO: 135), GGPS (SEQ ID NO: 136), GPSA (SEQ ID NO: 137), PSAT (SEQ ID NO: 138), SATE (SEQ ID NO: 139), ATER (SEQ ID NO: 140), TERA (SEQ ID NO: 141), ERAA (SEQ ID NO: 142), RAAE (SEQ ID NO: 143), AAED (SEQ ID NO: 144), AEDG (SEQ ID NO: 145), EDGA (SEQ ID NO: 146), DGAS (SEQ ID NO: 147), GASS (SEQ ID NO: 148), ASSQ (SEQ ID NO: 149), SSQL (SEQ ID NO: 150), SQLP (SEQ ID NO: 151), QLPA (SEQ ID NO: 152), LPAA (SEQ ID NO: 153), PAAE (SEQ ID NO: 154), AAEG (SEQ ID NO: 155), AEGS (SEQ ID NO: 156), EGSG (SEQ ID NO: 157), GSGE (SEQ ID NO: 158), SGEQ (SEQ ID NO: 159), GEQD (SEQ ID NO: 160), EQDF (SEQ ID NO: 161), QDFT (SEQ ID NO: 162), DFTF (SEQ ID NO: 18), or FTFE (SEQ ID NO: 19).

In an embodiment, the antibody molecule contacts five or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) consecutive amino acid residues in Pep5.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) of the following peptides (e.g., from Pep5): HTPHT (SEQ ID NO: 163), TPHTE (SEQ ID NO: 164), PHTED (SEQ ID NO: 165), HTEDG (SEQ ID NO: 166), TEDGG (SEQ ID NO: 167), EDGGP (SEQ ID NO: 168), DGGPS (SEQ ID NO: 169), GGPSA (SEQ ID NO: 170), GPSAT (SEQ ID NO: 171), PSATE (SEQ ID NO: 172), SATER (SEQ ID NO: 173), ATERA (SEQ ID NO: 174), TERAA (SEQ ID NO: 175), ERAAE (SEQ ID NO: 176), RAAED (SEQ ID NO: 177), AAEDG (SEQ ID NO: 178), AEDGA (SEQ ID NO: 179), EDGAS (SEQ ID NO: 180), DGASS (SEQ ID NO: 181), GASSQ (SEQ ID NO: 182), ASSQL (SEQ ID NO: 183), SSQLP (SEQ ID NO: 184), SQLPA (SEQ ID NO: 185), QLPAA (SEQ ID NO: 186), LPAAE (SEQ ID NO: 187), PAAEG (SEQ ID NO: 188), AAEGS (SEQ ID NO: 189), AEGSG (SEQ ID NO: 190), EGSGE (SEQ ID NO: 191), GSGEQ (SEQ ID NO: 192), SGEQD (SEQ ID NO: 193), GEQDF (SEQ ID NO: 194), EQDFT (SEQ ID NO: 195), QDFTF (SEQ ID NO: 196), or DFTFE (SEQ ID NO: 56).

In an embodiment, the antibody molecule contacts six or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34) consecutive amino acid residues in Pep5.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34) of the following peptides (e.g., from Pep5): HTPHTE (SEQ ID NO: 197), TPHTED (SEQ ID NO: 198), PHTEDG (SEQ ID NO: 199), HTEDGG (SEQ ID NO: 200), TEDGGP (SEQ ID NO: 201), EDGGPS (SEQ ID NO: 202), DGGPSA (SEQ ID NO: 203), GGPSAT (SEQ ID NO: 204), GPSATE (SEQ ID NO: 205), PSATER (SEQ ID NO: 206), SATERA (SEQ ID NO: 207), ATERAA (SEQ ID NO: 208), TERAAE (SEQ ID NO: 209), ERAAED (SEQ ID NO: 210), RAAEDG (SEQ ID NO: 211), AAEDGA (SEQ ID NO: 212), AEDGAS (SEQ ID NO: 213), EDGASS (SEQ ID NO: 214), DGASSQ (SEQ ID NO: 215), GASSQL (SEQ ID NO: 216), ASSQLP (SEQ ID NO: 217), SSQLPA (SEQ ID NO: 218), SQLPAA (SEQ ID NO: 219), QLPAAE (SEQ ID NO: 220), LPAAEG (SEQ ID NO: 221), PAAEGS (SEQ ID NO: 222), AAEGSG (SEQ ID NO: 223), AEGSGE (SEQ ID NO: 224), EGSGEQ (SEQ ID NO: 225), GSGEQD (SEQ ID NO: 226), SGEQDF (SEQ ID NO: 227), GEQDFT (SEQ ID NO: 228), EQDFTF (SEQ ID NO: 229), or QDFTFE (SEQ ID NO: 230).

In an embodiment, the antibody molecule does not bind, or binds with low affinity, to an extracellular region of CD138 distant from the transmembrane domain. In an embodiment, the antibody molecule does not bind to an epitope on CD138 comprising four or more (e.g., 5, 6, 7, 8, 9, 10, 11, 12, 13, 14, 15, 20, 25, 30, 35, or more) consecutive amino acid residues in an extracellular region distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain corresponds to Pep1a, Peplb, Pep2a, Pep2b, Pep2c, Pep2d, Pep3, Pep4, or a combination thereof. In an embodiment, the antibody molecule does not bind, or binds with low affinity, to the integrin binding domain (IBD) of CD138. In an embodiment, the antibody molecule does not bind, or binds with low affinity, to a region N-terminal to the IBD of CD138.

In an embodiment, the antibody molecule binds, or substantially binds, to an extracellular region of CD138 distant from the transmembrane domain. In an embodiment, the C-terminus of the extracellular region distant from the transmembrane domain is at least 100, 110, 120, 130, 140, 150, 160, 170, 180, 190, or 200 amino acids away from the N-terminus of the transmembrane domain. In an embodiment, the extracellular region distant from the transmembrane domain corresponds to Pep1a, Pep1b, Pep2a, Pep2b, Pep2c, Pep2d, Pep3, Pep4, or a combination thereof. In an embodiment, the antibody molecule binds, or substantially binds, to the integrin binding domain (IBD) of CD138. In an embodiment, the antibody molecule binds, or substantially binds, to a region N-terminal to the IBD of CD138. In an embodiment, the antibody molecule does not bind, or binds with low affinity, to the epitope of BB4.

In an embodiment, the antibody molecule binds to an epitope on CD138 comprising four or more (e.g., 5, 6, 7, 8, 9, 10, 11, 12, 13, 14, 15, 20, 25, 30, 35, 40, 45, 50, 55, 60, 65, 70, 75, or more) consecutive amino acid residues in the extracellular region distant from the transmembrane domain.

In an embodiment, the antibody molecule binds to an epitope on CD138 comprising five or more consecutive amino acid residues in the extracellular region distant to the transmembrane domain. In an embodiment, the antibody molecule binds to an epitope on CD138 comprising six or more consecutive amino acid residues in the extracellular region distant to the transmembrane domain. In an embodiment, the antibody molecule binds to an epitope on CD138 comprising seven or more consecutive amino acid residues in the extracellular region distant to the transmembrane domain. In an embodiment, the antibody molecule binds to an epitope on CD138 comprising eight or more consecutive amino acid residues in the extracellular region distant to the transmembrane domain. In an embodiment, the antibody molecule binds to an epitope on CD138 comprising nine or more consecutive amino acid residues in the extracellular region distant to the transmembrane domain. In an embodiment, the antibody molecule binds to an epitope on CD138 comprising ten or more consecutive amino acid residues in the extracellular region distant to the transmembrane domain. In an embodiment, the antibody molecule binds to an epitope on CD138 comprising eleven or more consecutive amino acid residues in the extracellular region distant to the transmembrane domain. In an embodiment, the antibody molecule binds to an epitope on CD138 comprising twelve or more consecutive amino acid residues in the extracellular region distant to the transmembrane domain.

In an embodiment, the extracellular region distant to the transmembrane domain corresponds to (e.g., comprises or consists of) Pep2a.

In an embodiment, the antibody molecule contacts four or more (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34) consecutive amino acid residues in Pep2a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31) of the following peptides (e.g., from Pep2a): ASTS (SEQ ID NO: 231), STST (SEQ ID NO: 232), TSTL (SEQ ID NO: 233), STLP (SEQ ID NO: 234), TLPA (SEQ ID NO: 235), LPAG (SEQ ID NO: 236), PAGE (SEQ ID NO: 237), AGEG (SEQ ID NO: 238), GEGP (SEQ ID NO: 239), EGPK (SEQ ID NO: 240), GPKE (SEQ ID NO: 241), PKEG (SEQ ID NO: 242), KEGE (SEQ ID NO: 243), EGEA (SEQ ID NO: 244), GEAV (SEQ ID NO: 245), EAVV (SEQ ID NO: 246), AVVL (SEQ ID NO: 247), VVLP (SEQ ID NO: 248), VLPE (SEQ ID NO: 249), LPEV (SEQ ID NO: 250), PEVE (SEQ ID NO: 251), EVEP (SEQ ID NO: 252), VEPG (SEQ ID NO: 253), EPGL (SEQ ID NO: 254), PGLT (SEQ ID NO: 255), GLTA (SEQ ID NO: 256), LTAR (SEQ ID NO: 257), TARE (SEQ ID NO: 258), AREQ (SEQ ID NO: 259), REQE (SEQ ID NO: 260), or EQEA (SEQ ID NO: 261). In an embodiment, the antibody molecule does not contact LPEV (SEQ ID NO: 250).

In an embodiment, the antibody molecule contacts five or more (e.g., 6, 7, 8, 9, 10, 11, 12, 13, 14, 15, 16, 17, 18, 19, 20, 21, 22, 23, 24, 25, 26, 27, 28, 29, or 30) consecutive amino acid residues in Pep2a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33) of the following peptides (e.g., from Pep2a): ASTS (SEQ ID NO: 231), STST (SEQ ID NO: 232), TSTL (SEQ ID NO: 233), STLP (SEQ ID NO: 234), TLPA (SEQ ID NO: 235), LPAG (SEQ ID NO: 236), PAGE (SEQ ID NO: 237), AGEG (SEQ ID NO: 238), GEGP (SEQ ID NO: 239), EGPK (SEQ ID NO: 240), GPKE (SEQ ID NO: 241), PKEG (SEQ ID NO: 242), KEGE (SEQ ID NO: 243), EGEA (SEQ ID NO: 244), GEAV (SEQ ID NO: 245), EAVV (SEQ ID NO: 246), AVVL (SEQ ID NO: 247), VVLP (SEQ ID NO: 248), VLPE (SEQ ID NO: 249), LPEV (SEQ ID NO: 250), PEVE (SEQ ID NO: 251), EVEP (SEQ ID NO: 252), VEPG (SEQ ID NO: 253), EPGL (SEQ ID NO: 254), PGLT (SEQ ID NO: 255), GLTA (SEQ ID NO: 256), LTAR (SEQ ID NO: 257), TARE (SEQ ID NO: 258), AREQ (SEQ ID NO: 259), REQE (SEQ ID NO: 260), or EQEA (SEQ ID NO: 261). In an embodiment, the antibody molecule does not contact a peptide comprising LPEV (SEQ ID NO: 250).

In an embodiment, the antibody molecule contacts six or more (e.g., 7, 8, 9, 10, 11, 12, 13, 14, 15, 16, 17, 18, 19, 20, 21, 22, 23, 24, 25, 26, 27, 28, or 29) consecutive amino acid residues in Pep2a.

In an embodiment, the antibody molecule contacts one or more (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32) of the following peptides (e.g., from Pep2a): ASTS (SEQ ID NO: 231), STST (SEQ ID NO: 232), TSTL (SEQ ID NO: 233), STLP (SEQ ID NO: 234), TLPA (SEQ ID NO: 235), LPAG (SEQ ID NO: 236), PAGE (SEQ ID NO: 237), AGEG (SEQ ID NO: 238), GEGP (SEQ ID NO: 239), EGPK (SEQ ID NO: 240), GPKE (SEQ ID NO: 241), PKEG (SEQ ID NO: 242), KEGE (SEQ ID NO: 243), EGEA (SEQ ID NO: 244), GEAV (SEQ ID NO: 245), EAVV (SEQ ID NO: 246), AVVL (SEQ ID NO: 247), VVLP (SEQ ID NO: 248), VLPE (SEQ ID NO: 249), LPEV (SEQ ID NO: 250), PEVE (SEQ ID NO: 251), EVEP (SEQ ID NO: 252), VEPG (SEQ ID NO: 253), EPGL (SEQ ID NO: 254), PGLT (SEQ ID NO: 255), GLTA (SEQ ID NO: 256), LTAR (SEQ ID NO: 257), TARE (SEQ ID NO: 258), AREQ (SEQ ID NO: 259), REQE (SEQ ID NO: 260), EQEA (SEQ ID NO: 261). In an embodiment, the antibody molecule does not contact a peptide comprising LPEV (SEQ ID NO: 250).

In an embodiment, the antibody molecule binds, or substantially binds, to an extracellular region of CD138 proximal to the transmembrane domain (e.g., an extracellular region described herein) and an extracellular region of CD138 distant from the transmembrane domain (e.g., an extracellular region described herein). In an embodiment, the antibody molecule binds to the extracellular region of CD138 proximal to the transmembrane domain with a binding affinity that is higher (e.g., at least 2, 3, 4, 5, 6, 7, 8, 9, 10, 20, 30, 40, 50, 100, 200, 300, 400, or 500-fold higher) than the binding affinity to the extracellular region of CD138 distant from the transmembrane domain. In an embodiment, the antibody molecule binds to the extracellular region of CD138 distant from the transmembrane domain with a binding affinity that is higher (e.g., at least 2, 3, 4, 5, 6, 7, 8, 9, 10, 20, 30, 40, 50, 100, 200, 300, 400, or 500-fold higher) than the binding affinity to the extracellular region of CD138 proximal to the transmembrane domain.

### Antibody Molecules

Disclosed herein are antibody molecules that bind to CD138, e.g., an anti-CD138 molecule described herein (e.g., a pH-selective anti-CD138 antibody molecule described herein).

As used herein, the term “antibody molecule” refers to a protein, e.g., an immunoglobulin chain or a fragment thereof, comprising at least one immunoglobulin variable domain sequence. The term “antibody molecule” includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. The antibody molecule can also be a human, humanized, CDR-grafted, or in vitro generated antibody. The antibody molecule can have a heavy chain constant region chosen from, e.g., IgG1, IgG2, IgG3, or IgG4. The antibody molecule can also have a light chain chosen from, e.g., kappa or lambda. The term “immunoglobulin” (Ig) is used interchangeably with the term “antibody” herein.

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

The antibody molecule can be a single chain antibody. A single-chain antibody (scFv) may be engineered (see, e.g., Colcher et al. (1999) Ann N Y Acad Sci 880: 263-280; and Reiter & Pastan (1996) Clin Cancer Res 2: 245-252). The single chain antibody can be dimerized or multimerized to generate multivalent antibodies having specificities for different epitopes of the same target protein.

The antibody molecules disclosed herein can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. According to some aspects, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also contemplated.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular’s AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). In an embodiment, the following definitions are used: AbM definition of CDR1 of the heavy chain variable domain and Kabat definitions for the other CDRs. In an embodiment, Kabat definitions are used for all CDRs. In addition, embodiments described with respect to Kabat or AbM CDRs may also be implemented using Chothia hypervariable loops. Each VH and VL typically includes three CDRs and four FRs, arranged from amino-terminus to carboxy-terminus in the following order: FR1, CDR1, FR2, CDR2, FR3, CDR3, and FR4.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody molecule that comprises determinants that form an interface that binds to an antigen, e.g., CD138, or an epitope thereof. With respect to proteins (or protein mimetics), the antigen-binding region typically includes one or more loops (of at least, e.g., four amino acids or amino acid mimics) that form an interface that binds to the antigen, e.g., CD138. Typically, the antigen-binding region of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-CD138 antibody molecule, e.g., an anti-CD138 antibody molecule provided herein, to a target, e.g., CD138. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA or BIACORE assay. In an embodiment, a competition binding assay is a quantitative competition assay. In an embodiment, a first anti-CD138 antibody molecule is said to compete for binding to the target with a second anti-CD138 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum (see e.g., Saleh et al., Cancer Immunol. Immunother., 32:180-190 (1990)) and also because of potential allergic reactions (see e.g., LoBuglio et al., Hybridoma, 5:5117-5123 (1986)).

The antibody molecule can be a polyclonal or a monoclonal antibody. In an embodiment, the antibody can be recombinantly produced, e.g., produced by any suitable phage display or combinatorial methods.

Various phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246: 1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In an embodiment, the antibody molecule is a fully human antibody (e.g., an antibody made in a mouse which has been genetically engineered to produce an antibody from a human immunoglobulin sequence), or a non-human antibody, e.g., a rodent (mouse or rat), goat, primate (e.g., monkey), camel antibody. In an embodiment, the non-human antibody is a rodent (mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L.L. et al. 1994 Nature Genet. 7:13-21; Morrison, S.L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by any suitable recombinant DNA technique. Several are known in the art (see Robinson et al., International Patent Application Publication No. WO1987/002671; Akira, et al., European Patent Application Publication No. 184,187; Taniguchi, M., European Patent Application Publication No. 171,496; Morrison et al., European Patent Application Publication No. 173,494; Neuberger et al., International Patent Application Publication No. WO 86/01533; Cabilly et al. U.S. Pat. No. 4,816,567; Cabilly et al., European Patent Application Publication No. 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In an embodiment, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In an embodiment, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (see, e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by any suitable method, and several such methods known in the art (see, e.g., Morrison, S. L., 1985, Science 229:1202-1207, by Oi et al., 1986, BioTechniques 4:214, and by Queen et al. US 5,585,089, US 5,693,761 and US 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See, e.g., U.S. Pat. 5,225,539; Jones et al. 1986 Nature 321:552-525; Verhoeyan et al. 1988 Science 239:1534; Beidler et al. 1988 J. Immunol. 141:4053-4060; Winter US 5,225,539, the contents of all of which are hereby expressly incorporated by reference. Winter describes a CDR-grafting method which may be used to prepare humanized antibodies (UK Patent Application GB 2188638A, filed on Mar. 26, 1987; Winter US 5,225,539), the contents of which is expressly incorporated by reference.

Also provided are humanized antibodies in which specific amino acids have been substituted, deleted or added. Criteria for selecting amino acids from the donor are described in, e.g., US 5,585,089, e.g., columns 12-16 of US 5,585,089, the contents of which are hereby incorporated by reference. Other techniques for humanizing antibodies are described in Padlan et al. EP 519596 A1, published on Dec. 23, 1992.

In an embodiment, the antibody molecule has a heavy chain constant region chosen from, e.g., the heavy chain constant regions of IgG1, IgG2 (e.g., IgG2a), IgG3, IgG4, IgM, IgA1, IgA2, IgD, and IgE; particularly, chosen from, e.g., the (e.g., human) heavy chain constant regions of IgG1, IgG2, IgG3, and IgG4. In an embodiment, the antibody molecule comprises a heavy chain constant region of IgG1 (e.g., m3 allotype). In another embodiment, the antibody molecule has a light chain constant region chosen from, e.g., the (e.g., human) light chain constant regions of kappa or lambda. In an embodiment, the antibody molecule comprises a light chain constant region of kappa (e.g., kappa constant *01). In an embodiment, the antibody molecule comprises a heavy chain constant region of IgG1 and a light chain constant region of kappa. The constant region can be altered, e.g., mutated, to modify the properties of the antibody molecule (e.g., to increase or decrease one or more of: Fc receptor binding, antibody glycosylation, the number of cysteine residues, effector cell function, and/or complement function). In an embodiment, the antibody molecule has effector function and can fix complement. In another embodiment, the antibody molecule does not recruit effector cells or fix complement. In certain embodiments, the antibody molecule has reduced or no ability to bind an Fc receptor. For example, it may be an isotype or subtype, fragment or other mutant, which does not support binding to an Fc receptor, e.g., it has a mutagenized or deleted Fc receptor binding region.

In an embodiment, a constant region of the antibody molecule is altered. Methods for altering an antibody constant region are known in the art. Antibody molecules s with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

In an embodiment, the antibody molecule comprises an Fc region that comprise one or more (e.g., 2, 3, 4, 5, 6, 7, 8, 9, 10, 11, 12, 13, 14, 15, 16, 17, 18, 19, 20, 21, 22, 23, 24, 25, or more) of mutations or combinations of mutations described in Table 9.

TABLE 9

Exemplary Fc mutations

| | |
|---|---|
| Name | Mutation (according to EU numbering) |
| FcMut001 | I253M |
| FcMut002 | L309H_D312A_N315D |
| FcMut003 | L309N |
| FcMut004 | M252E_S254R |
| FcMut005 | M252E_S254R_R255Y |
| FcMut006 | S254H |
| FcMut007 | S254M |
| FcMut008 | T256D_T307R |
| FcMut009 | T256L_N286I_T307I |
| FcMut010 | T256I_N286I_T307I |
| FcMut011 | K248S_D376Q |
| FcMut012 | K248S_D376N |
| FcMut013 | D376Q_E380A |
| FcMut014 | D376N_E380A |
| FcMut015 | D376Q_M428L |
| FcMut016 | K248S_A378I |
| FcMut017 | L314K |
| FcMut018 | T250Q_M428L |
| FcMut019 | M428L_N434A |
| FcMut020 | N434A |
| FcMut021 | T307A_E380A_N434A |
| FcMut022 | M252W |
| FcMut023 | V308F |
| FcMut024 | V308F_N434Y |
| FcMut026 | T256D_T307R_D376N |
| FcMut027 | L309R_D312E |
| FcMut028 | L309R_Q311P_D312E |
| FcMut029 | K246N_P247A |
| FcMut030 | K246N_P247A _D376N |
| FcMut031 | T256E_T307R |
| FcMut032 | T256R_T307D |
| FcMut033 | T256R_T307E |
| FcMut034 | Q311P |
| FcMut035 | D376Q |
| FcMut036 | L234A_L235A |
| FcMut037 | L235V_G236A |
| FcMut038 | L234P_L235P |
| FcMut039 | L235P |
| FcMut040 | P329G |
| FcMut041 | P329E |
| FcMut042 | E233K |
| FcMut043 | T256D_N286D_A287S_T307R |
| FcMut044 | T256D_P257L_T307R |
| FcMut045 | T256D_T307R_Q311V |
| FcMut046 | P247D_T256D_T307R |
| FcMut047 | P247D_N286D_A287S_Q311V |
| FcMut048 | P257M_V308N |
| FcMut049 | V279I_Q311L_N315T |
| FcMut050 | M428L_N434S |
| FcMut051 | N434S |
| FcMut052 | H433G_N434P |
| FcMut053 | V259I_V308F_M428L |
| FcMut067 | T256D_N286D_T307R |
| FcMut068 | T256D_N286E_T307R |
| FcMut069 | T256D_N286Q_T307R |
| FcMut070 | T256D_P257T_T307R |
| FcMut071 | T256D_P257V_T307R |
| FcMut072 | T256D_T307R_Q311I |
| FcMut073 | T256D_T307R_Q311L |
| FcMut074 | T256D_T307R_Q311M |
| FcMut075 | T256D_P257L_N286D_T307R_Q311V |
| FcMut076 | T256D_T307R_M428L |
| FcMut077 | M428L |
| FcMut078 | M252Y_S254T_T256Q |
| FcMut079 | M252Y_S254T_T256E_K288E |
| FcMut080 | T256K_K288E |
| FcMut081 | T256D_E258T |
| FcMut082 | E283Q_H285E |
| FcMut083 | R344D_D401R |
| FcMut084 | K248E_E380K |
| FcMut085 | K248E_E380R |
| FcMut086 | K246H |
| FcMut087 | K248H |
| FcMut088 | T250I |
| FcMut089 | T250V |
| FcMut090 | L251F |
| FcMut091 | L251M |
| FcMut093 | P257V |
| FcMut094 | N276D |
| FcMut095 | H285N |
| FcMut096 | H285D |
| FcMut097 | K288H |
| FcMut098 | K288Q |
| FcMut099 | K288E |
| FcMut100 | T307E |
| FcMut101 | T307Q |
| FcMut102 | V308P |
| FcMut103 | V308I |
| FcMut104 | V308L |
| FcMut105 | L309H |
| FcMut106 | L309M |
| FcMut107 | Q311H |
| FcMut108 | L314F |
| FcMut109 | Y319H |
| FcMut110 | I336T |
| FcMut111 | P343D |
| FcMut112 | P343V |
| FcMut113 | E345Q |
| FcMut114 | P346V |
| FcMut115 | P374T |
| FcMut116 | D376N |
| FcMut117 | A378S |
| FcMut118 | A431T |
| FcMut119 | A431P |
| FcMut120 | A431G |
| FcMut121 | L432V |
| FcMut122 | L432I |
| FcMut123 | L432Q |
| FcMut124 | N434T |
| FcMut125 | H435N |
| FcMut126 | Y436H |
| FcMut127 | K439Q |
| FcMut128 | T256D |
| FcMut129 | T307R |
| FcMut130 | A378T |
| FcMut131 | A378D |
| FcMut132 | A378H |
| FcMut133 | A378Y |
| FcMut134 | A378V |
| FcMut135 | D376R |
| FcMut136 | D376F |
| FcMut137 | D376W |
| FcMut138 | L314H |
| FcMut139 | L432E_T437Q |
| FcMut140 | D376Q_A378T |
| FcMut141 | D376Q_I377M_A378T |
| FcMut142 | P244Q_D376Q |
| FcMut143 | P247T_A378T |
| FcMut144 | P247N_A378T |
| FcMut145 | T256D_T307R_L309T |
| FcMut146 | A339T_S375E_F404Y |
| FcMut147 | L235V_G236A_T256D_T307R |
| FcMut148 | L235V_G236A_D376Q_M428L |
| FcMut149 | L314N |
| FcMut150 | N315D |
| FcMut151 | A378T |
| FcMut152 | T437Q |
| FcMut153 | L432E |
| FcMut154 | Y436R |
| FcMut155 | L314M |
| FcMut156 | L234A_L235A_T256D_T307R_Q311V |
| FcMut157 | L234A_L235A_T256D_P257V_T307R |
| FcMut158 | L234A_L235A_T256D_P257L_N286D_T307RQ311V |
| FcMut159 | L235V_G236A_T256D_T307R_Q311V |
| FcMut160 | L235V_G236A_T256D_P257V_T307R |
| FcMut161 | L235V_G236A_T256D_P257L_N286D_T307R_Q311V |
| FcMut162 | S267T_A327N_A330M |
| FcMut163 | S267T_A327N |
| FcMut164 | L235V_G236A_S267T_A327N_A330M |
| FcMut165 | L235V_G236A_S267T_A327N |
| FcMut166 | M252Y_S254T |
| FcMut167 | T256E |
| FcMut168 | G236A_I332E |
| FcMut169 | S239D_I332E |
| FcMut170 | G236A_S239D_I332E |
| FcMut171 | T256D_N286D_T307R_Q311V |
| FcMut172 | T256D_E258T_T307R |
| FcMut173 | T256D_E258T_T307R_Q311V |
| FcMut174 | T256D_P257V_E258T_T307R |
| FcMut175 | T256D_P257L_E258T_N286D_T307R_Q311V |
| FcMut176 | T256D_E258T_N286D_T307R_Q311V |
| FcMut177 | A378V_M428L |
| FcMut178 | A378V_M428I |
| FcMut179 | A378V_M428V |
| FcMut180 | T256D _N286D |
| FcMut181 | T256D_A378V |
| FcMut182 | T256D_Q311V |
| FcMut183 | T256D_Q311V_A378V |
| FcMut184 | T256D_T307R_A378V |
| FcMut185 | T256D_N286D_T307R_A378V |
| FcMut186 | T256D_T307R_Q311V_A378V |
| FcMut187 | H285D_A378V |
| FcMut188 | H285D_Q311V |
| FcMut189 | T256D_H285D |
| FcMut190 | T256D_H285D_Q311V |
| FcMut191 | T256D_H285D_T307R |
| FcMut192 | T256D_H285D_T307R_ A378V |
| FcMut193 | H285D_L314M_A378V |
| FcMut194 | T256D_E258T_H285D_Q311H |
| FcMut195 | T256D_E258T_H285D |
| FcMut196 | H285D_N315D |
| FcMut197 | H285N_T307Q_N315D |
| FcMut198 | H285D_L432E_T437Q |
| FcMut199 | T256D_E258T_N315D |
| FcMut200 | P257V_H285N |
| FcMut201 | H285N_L432F |
| FcMut202 | H285N_T437I |
| FcMut203 | T256D_E258T_L314M |
| FcMut204 | T256D_E258T_T307Q |
| FcMut205 | T256D_E258T_A378V |
| FcMut206 | V308P_A378V |
| FcMut207 | P257V_A378T |
| FcMut208 | P257V_V308P_A378V |
| FcMut209 | N315D_A378T |
| FcMut210 | H285N_L314M |
| FcMut211 | L314M_L432E_T437Q |
| FcMut212 | T307Q_N315D |
| FcMut213 | H285D_T307Q_A378V |
| FcMut214 | L314M_N315D |
| FcMut215 | T307Q_Q311V_A378V |
| FcMut216 | H285D_Q311V_A378V |
| FcMut217 | Q311V_N315D_A378V |
| FcMut218 | T256D_E258T_Q311V |
| FcMut219 | T256D_N315D_A378V |
| FcMut220 | T256D_Q311V_N315D |
| FcMut221 | T256D_T307Q_A378V |
| FcMut222 | T256D_T307Q_Q311V |
| FcMut223 | T256D_H285D_A378V |
| FcMut224 | T256D_H285D_T307RQ311V |
| FcMut225 | T256D_H285D_N286D_T307R |
| FcMut226 | T256D_H285D_N286D_T307R_Q311V |
| FcMut227 | T256D_H285D_N286D_T307R_A378V |
| FcMut228 | T256D_N286D_T307R_Q311V_A378V |
| FcMut229 | T256D_H285D_T307R_Q311V_A378V |
| FcMut230 | V308P_Q311V_A378V |
| FcMut231 | T256D_V308P_A378V |
| FcMut232 | T256D_V308P_Q311V |
| FcMut233 | T256D_E258T_V308P |
| FcMut234 | H285D_V308P_Q311V |
| FcMut242 | E258T |
| FcMut243 | N286D |
| FcMut244 | Q311V |
| YTE | M252Y_S254T_T256E |

In an embodiment, the Fc region comprises FcMut001. In an embodiment, the Fc region comprises FcMut002. In an embodiment, the Fc region comprises FcMut003. In an embodiment, the Fc region comprises FcMut004. In an embodiment, the Fc region comprises FcMut005. In an embodiment, the Fc region comprises FcMut006. In an embodiment, the Fc region comprises FcMut007. In an embodiment, the Fc region comprises FcMut008. In an embodiment, the Fc region comprises FcMut009. In an embodiment, the Fc region comprises FcMut010. In an embodiment, the Fc region comprises FcMut011. In an embodiment, the Fc region comprises FcMut012. In an embodiment, the Fc region comprises FcMut013. In an embodiment, the Fc region comprises FcMut014. In an embodiment, the Fc region comprises FcMut015. In an embodiment, the Fc region comprises FcMut016. In an embodiment, the Fc region comprises FcMut017. In an embodiment, the Fc region comprises FcMut018. In an embodiment, the Fc region comprises FcMut019. In an embodiment, the Fc region comprises FcMut020. In an embodiment, the Fc region comprises FcMut021. In an embodiment, the Fc region comprises FcMut022. In an embodiment, the Fc region comprises FcMut023. In an embodiment, the Fc region comprises FcMut024. In an embodiment, the Fc region comprises FcMut026. In an embodiment, the Fc region comprises FcMut027. In an embodiment, the Fc region comprises FcMut028. In an embodiment, the Fc region comprises FcMut029. In an embodiment, the Fc region comprises FcMut030. In an embodiment, the Fc region comprises FcMut031. In an embodiment, the Fc region comprises FcMut032. In an embodiment, the Fc region comprises FcMut033. In an embodiment, the Fc region comprises FcMut034. In an embodiment, the Fc region comprises FcMut035. In an embodiment, the Fc region comprises FcMut036. In an embodiment, the Fc region comprises FcMut037. In an embodiment, the Fc region comprises FcMut038. In an embodiment, the Fc region comprises FcMut039. In an embodiment, the Fc region comprises FcMut040. In an embodiment, the Fc region comprises FcMut041. In an embodiment, the Fc region comprises FcMut042. In an embodiment, the Fc region comprises FcMut043. In an embodiment, the Fc region comprises FcMut044. In an embodiment, the Fc region comprises FcMut045. In an embodiment, the Fc region comprises FcMut046. In an embodiment, the Fc region comprises FcMut047. In an embodiment, the Fc region comprises FcMut048. In an embodiment, the Fc region comprises FcMut049. In an embodiment, the Fc region comprises FcMut050. In an embodiment, the Fc region comprises FcMut051. In an embodiment, the Fc region comprises FcMut052. In an embodiment, the Fc region comprises FcMut053. In an embodiment, the Fc region comprises FcMut067. In an embodiment, the Fc region comprises FcMut068. In an embodiment, the Fc region comprises FcMut069. In an embodiment, the Fc region comprises FcMut070. In an embodiment, the Fc region comprises FcMut071. In an embodiment, the Fc region comprises FcMut072. In an embodiment, the Fc region comprises FcMut073. In an embodiment, the Fc region comprises FcMut074. In an embodiment, the Fc region comprises FcMut075. In an embodiment, the Fc region comprises FcMut076. In an embodiment, the Fc region comprises FcMut077. In an embodiment, the Fc region comprises FcMut078. In an embodiment, the Fc region comprises FcMut079. In an embodiment, the Fc region comprises FcMut080. In an embodiment, the Fc region comprises FcMut081. In an embodiment, the Fc region comprises FcMut082. In an embodiment, the Fc region comprises FcMut083. In an embodiment, the Fc region comprises FcMut084. In an embodiment, the Fc region comprises FcMut085. In an embodiment, the Fc region comprises FcMut086. In an embodiment, the Fc region comprises FcMut087. In an embodiment, the Fc region comprises FcMut088. In an embodiment, the Fc region comprises FcMut089. In an embodiment, the Fc region comprises FcMut090. In an embodiment, the Fc region comprises FcMut091. In an embodiment, the Fc region comprises FcMut093. In an embodiment, the Fc region comprises FcMut094. In an embodiment, the Fc region comprises FcMut095. In an embodiment, the Fc region comprises FcMut096. In an embodiment, the Fc region comprises FcMut097. In an embodiment, the Fc region comprises FcMut098. In an embodiment, the Fc region comprises FcMut099. In an embodiment, the Fc region comprises FcMut100. In an embodiment, the Fc region comprises FcMut101. In an embodiment, the Fc region comprises FcMut102. In an embodiment, the Fc region comprises FcMut103. In an embodiment, the Fc region comprises FcMut104. In an embodiment, the Fc region comprises FcMut105. In an embodiment, the Fc region comprises FcMut106. In an embodiment, the Fc region comprises FcMut107. In an embodiment, the Fc region comprises FcMut108. In an embodiment, the Fc region comprises FcMut109. In an embodiment, the Fc region comprises FcMut110. In an embodiment, the Fc region comprises FcMut111. In an embodiment, the Fc region comprises FcMut112. In an embodiment, the Fc region comprises FcMut113. In an embodiment, the Fc region comprises FcMut114. In an embodiment, the Fc region comprises FcMut115. In an embodiment, the Fc region comprises FcMut116. In an embodiment, the Fc region comprises FcMut117. In an embodiment, the Fc region comprises FcMut118. In an embodiment, the Fc region comprises FcMut119. In an embodiment, the Fc region comprises FcMut120. In an embodiment, the Fc region comprises FcMut121. In an embodiment, the Fc region comprises FcMut122. In an embodiment, the Fc region comprises FcMut123. In an embodiment, the Fc region comprises FcMut124. In an embodiment, the Fc region comprises FcMut125. In an embodiment, the Fc region comprises FcMut126. In an embodiment, the Fc region comprises FcMut127. In an embodiment, the Fc region comprises FcMut128. In an embodiment, the Fc region comprises FcMut129. In an embodiment, the Fc region comprises FcMut130. In an embodiment, the Fc region comprises FcMut131. In an embodiment, the Fc region comprises FcMut132. In an embodiment, the Fc region comprises FcMut133. In an embodiment, the Fc region comprises FcMut134. In an embodiment, the Fc region comprises FcMut135. In an embodiment, the Fc region comprises FcMut136. In an embodiment, the Fc region comprises FcMut137. In an embodiment, the Fc region comprises FcMut138. In an embodiment, the Fc region comprises FcMut139. In an embodiment, the Fc region comprises FcMut140. In an embodiment, the Fc region comprises FcMut141. In an embodiment, the Fc region comprises FcMut142. In an embodiment, the Fc region comprises FcMut143. In an embodiment, the Fc region comprises FcMut144. In an embodiment, the Fc region comprises FcMut145. In an embodiment, the Fc region comprises FcMut146. In an embodiment, the Fc region comprises FcMut147. In an embodiment, the Fc region comprises FcMut148. In an embodiment, the Fc region comprises FcMut149. In an embodiment, the Fc region comprises FcMut150. In an embodiment, the Fc region comprises FcMut151. In an embodiment, the Fc region comprises FcMut152. In an embodiment, the Fc region comprises FcMut153. In an embodiment, the Fc region comprises FcMut154. In an embodiment, the Fc region comprises FcMut155. In an embodiment, the Fc region comprises FcMut156. In an embodiment, the Fc region comprises FcMut157. In an embodiment, the Fc region comprises FcMut158. In an embodiment, the Fc region comprises FcMut159. In an embodiment, the Fc region comprises FcMut160. In an embodiment, the Fc region comprises FcMut161. In an embodiment, the Fc region comprises FcMut162. In an embodiment, the Fc region comprises FcMut163. In an embodiment, the Fc region comprises FcMut164. In an embodiment, the Fc region comprises FcMut165. In an embodiment, the Fc region comprises FcMut166. In an embodiment, the Fc region comprises FcMut167. In an embodiment, the Fc region comprises FcMut168. In an embodiment, the Fc region comprises FcMut169. In an embodiment, the Fc region comprises FcMut170. In an embodiment, the Fc region comprises FcMut171. In an embodiment, the Fc region comprises FcMut172. In an embodiment, the Fc region comprises FcMut173. In an embodiment, the Fc region comprises FcMut174. In an embodiment, the Fc region comprises FcMut175. In an embodiment, the Fc region comprises FcMut176. In an embodiment, the Fc region comprises FcMut177. In an embodiment, the Fc region comprises FcMut178. In an embodiment, the Fc region comprises FcMut179. In an embodiment, the Fc region comprises FcMut180. In an embodiment, the Fc region comprises FcMut181. In an embodiment, the Fc region comprises FcMut182. In an embodiment, the Fc region comprises FcMut183. In an embodiment, the Fc region comprises FcMut184. In an embodiment, the Fc region comprises FcMut185. In an embodiment, the Fc region comprises FcMut186. In an embodiment, the Fc region comprises FcMut187. In an embodiment, the Fc region comprises FcMut188. In an embodiment, the Fc region comprises FcMut189. In an embodiment, the Fc region comprises FcMut190. In an embodiment, the Fc region comprises FcMut191. In an embodiment, the Fc region comprises FcMut192. In an embodiment, the Fc region comprises FcMut193. In an embodiment, the Fc region comprises FcMut194. In an embodiment, the Fc region comprises FcMut195. In an embodiment, the Fc region comprises FcMut196. In an embodiment, the Fc region comprises FcMut197. In an embodiment, the Fc region comprises FcMut198. In an embodiment, the Fc region comprises FcMut199. In an embodiment, the Fc region comprises FcMut200. In an embodiment, the Fc region comprises FcMut201. In an embodiment, the Fc region comprises FcMut202. In an embodiment, the Fc region comprises FcMut203. In an embodiment, the Fc region comprises FcMut204. In an embodiment, the Fc region comprises FcMut205. In an embodiment, the Fc region comprises FcMut206. In an embodiment, the Fc region comprises FcMut207. In an embodiment, the Fc region comprises FcMut208. In an embodiment, the Fc region comprises FcMut209. In an embodiment, the Fc region comprises FcMut210. In an embodiment, the Fc region comprises FcMut211. In an embodiment, the Fc region comprises FcMut212. In an embodiment, the Fc region comprises FcMut213. In an embodiment, the Fc region comprises FcMut214. In an embodiment, the Fc region comprises FcMut215. In an embodiment, the Fc region comprises FcMut216. In an embodiment, the Fc region comprises FcMut217. In an embodiment, the Fc region comprises FcMut218. In an embodiment, the Fc region comprises FcMut219. In an embodiment, the Fc region comprises FcMut220. In an embodiment, the Fc region comprises FcMut221. In an embodiment, the Fc region comprises FcMut222. In an embodiment, the Fc region comprises FcMut223. In an embodiment, the Fc region comprises FcMut224. In an embodiment, the Fc region comprises FcMut225. In an embodiment, the Fc region comprises FcMut226. In an embodiment, the Fc region comprises FcMut227. In an embodiment, the Fc region comprises FcMut228. In an embodiment, the Fc region comprises FcMut229. In an embodiment, the Fc region comprises FcMut230. In an embodiment, the Fc region comprises FcMut231. In an embodiment, the Fc region comprises FcMut232. In an embodiment, the Fc region comprises FcMut233. In an embodiment, the Fc region comprises FcMut234. In an embodiment, the Fc region comprises FcMut242. In an embodiment, the Fc region comprises FcMut243. In an embodiment, the Fc region comprises FcMut244.

Other exemplary Fc mutations are described, e.g., in International Application Publication No. WO2018/052556, U.S. Pat. Application Publication No. US2018/0037634, and Booth et al., MAbs. 2018; 10(7): 1098-1110, the contents of which are incorporated by reference in their entirety.

In an embodiment, the Fc region is altered to extend half-life. For example, the Fc region can contain one or more of: FcMut183 (T256D-Q311V-A378V), FcMut197 (H285N-T307Q-N315D), FcMut213 (H285D-T307Q-A378V), FcMut215 (T307Q-Q311V-A378V), or FcMut228 (T256D-N286D-T307R-Q311V-A378V) (all according to EU numbering).

In an embodiment, the Fc region is altered to enhance ADCC. For example, the Fc region can contain one or more of: A330L-I332E-S239D, F243L-R292P-Y300L-V305I-P396L, or S298A-E333A-K334A. In an embodiment, afucosylation can be achieved by expression in a cell line such as CHO in which fucosyltransferase (FucT8) is knocked out.

In an embodiment, the Fc region is altered to enhance CDC. For example, the Fc region contains S267E-H268F-S324T.

In an embodiment, the Fc region is altered to enhance antibody-dependent cellular phagocytosis (ADCP). For example, the Fc region contains S239D-I332E-A330L.

In an embodiment, the only amino acids in the antibody molecule are canonical amino acids. In an embodiment, the antibody molecule comprises naturally-occurring amino acids; analogs, derivatives and congeners thereof; amino acid analogs having variant side chains; and/or all stereoisomers of any of any of the foregoing. The antibody molecule may comprise the D- or L- optical isomers of amino acids and peptidomimetics.

A polypeptide of an antibody molecule described herein may be linear or branched, it may comprise modified amino acids, and it may be interrupted by non-amino acids. The antibody molecule may also be modified; for example, by disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The antibody molecule described herein can be used alone in unconjugated form, or can be bound to a substance, e.g., a toxin or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-CD138 antibody can be coupled to a radioactive isotope such as an α-, β-, or γ-emitter, or a β-and γ-emitter.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a toxin, a pharmaceutical agent, and/or a protein or peptide that can mediate association of the antibody or antibody portion with another molecule (such as a streptavidin core region or a polyhistidine tag).

Some types of derivatized antibody molecule are produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-CD138 antibody molecule may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecules can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule may be conjugated to another molecular entity, typically a label or a therapeutic (e.g., antimicrobial (e.g., antibacterial or bactericidal), immunomodulatory, immunostimularoty, cytotoxic, or cytostatic) agent or moiety. Radioactive isotopes can be used in diagnostic or therapeutic applications. Radioactive isotopes that can be coupled to the antibody molecules include, but are not limited to α-, β-, or γ-emitters, or β-and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹ mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷CO or ⁵⁸CO), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The present disclosure provides radiolabeled antibody molecules and methods of labeling the same. In an embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

In an aspect, this disclosure provides a method of making an antibody molecule disclosed herein (e.g., pH-selective anti-CD138 antibody molecules described herein). The method includes: providing an antigen, e.g., CD138 or a fragment thereof; obtaining an antibody molecule that specifically binds to the antigen; evaluating efficacy of the antibody molecule in modulating activity of the antigen and/or organism expressing the antigen, e.g., CD138. The method can further include administering the antibody molecule, including a derivative thereof (e.g., an antibody molecule) to a subject, e.g., a human.

This disclosure provides an isolated nucleic acid molecule encoding the above antibody molecule, vectors and host cells thereof. The nucleic acid molecule includes, but is not limited to, RNA, genomic DNA and cDNA.

Amino acid sequences of exemplary antibody molecules are described in Table 1. Amino acid and nucleotide sequences of exemplary VHs and VLs are described in Table 2. Any VH described in Table 2 can be paired with any VL described in Table 2 to form an exemplary anti-CD138 antibody molecule. Antibodies 28-0, 29-0, Ab17, Ab18, Ab29, Ab28, Ab30, Ab32, Ab34, Ab43, Ab71, Ab137, Ab173, Ab174, or Ab175 are also sometimes referred to as mAbs 28-0, 29-0, Ab17, Ab18, Ab29, Ab28, Ab30, Ab32, Ab34, Ab43, Ab71, Ab137, Ab173, Ab174, or Ab175 herein.

TABLE 1

Exemplary anti-CD138 antibodies.

| Antibody | Chain | Amino Acid Sequence | SEQ ID NO | Chothia CDR | | SEQ ID NO | Kabat CDR | | SEQ ID NO |
|---|---|---|---|---|---|---|---|---|---|
| 28-0 | VH | EVQLVQSGAEVKKPGASVKVSC KASGYSFSSYYMHWVRQAPGQG LEWMGTIHPSDSTTNYNQKFQG RVTMTVDTSTRTAYMELSSLRS EDTAVYYCADFVYWGQGTTVTV SS | 458 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 470 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| 29-0 | VH | EVQLVQSGAEVKKPGASVKVSC KASGYSFSSYYMHWVRQAPGQG LEWMGTIYPGASTTNYNQKFQG RVTMTVDTSTRTAYMELSSLRS EDTAVYYCADFVYWGQGTTVTV SS | 459 | HCDR1 | GYSFSSY | 301 | HCDR1 | SYYMH | 307 |
| | | | | HCDR2 | YPGAST | 309 | HCDR2 | TIYPGASTTNYNQK FQG | 310 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 471 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab17 | VH | EVQLVQSGAEVKKPGASVKVSC KASGYSFSSYYMHWVRQAPGQG LEWMGTIHPSDSTTNYNQKFQG RVTMTVDTSTRTAYMELSSLRS EDTAVYYCADFVYWGQGTTVTV SS | 460 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FVY | 303 | HCDR3 | FVY | 303 |
| | VL | DIVMTQTPLSLSVTPGQPASIS CKSSKSLLYKDGHTYLNWFLQK PGQSPQLLIYVVSTRASGVPDR FSGSGSGTDFTLKISRVEAEDV GVYYCQQLVEYPYTFGQGTKLE IK | 472 | LCDR1 | KSSKSLLYKDGHTY LN | 311 | LCDR1 | KSSKSLLYKDGHTY LN | 311 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab18 | VH | EVQLVQSGAEVKKPGASVKVSC KASGYSFSSYYMHWVRQAPGQG LEWMGTIHPSDSTTNYNQKFQG RVTMTVDTSTRTAYMELSSLRS EDTAVYYCADFVYWGQGTTVTV SS | 461 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCEQLVEYPYTFGQGTKLE IK | 473 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | EQLVEYPYT | 312 | LCDR3 | EQLVEYPYT | 312 |
| Ab29 | VH | EVQLVQSGAEVKKPGASVKVSC KASGYSFSSYYMHWVRQAPGQG LEWMGTIYPEDSTTNYNQKFQG RVTMTVDTSTRTAYMELSSLRS EDTAVYYCADFVYWGQGTTVTV SS | 462 | HCDR1 | GYSFSSY | 301 | HCDR1 | SYYMH | 307 |
| | | | | HCDR2 | YPEDST | 313 | HCDR2 | TIYPEDSTTNYNQK FQG | 314 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 474 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab28 | VH | EVQLVQSGAEVKKPGASVKVSC KASGYSFSSYYMHWVRQAPGQG LEWMGTIYPGDSTTNYNQKFQG RVTMTVDTSTRTAYMELSSLRS EDTAVYYCADFVYWGQGTTVTV SS | 463 | HCDR1 | GYSFSSY | 301 | HCDR1 | SYYMH | 307 |
| | | | | HCDR2 | YPGDST | 321 | HCDR2 | TIYPGDSTTNYNQK FQG | 322 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 475 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab30 | VH | EVQLVQSGAEVKKPGASVKVSC KASGYSFSSYYMHWVRQAPGQG LEWMGTIHPEDSTTNYNQKFQG RVTMTVDTSTRTAYMELSSLRS EDTAVYYCADFVYWGQGTTVTV SS | 464 | HCDR1 | GYSFSSY | 301 | HCDR1 | SYYMH | 307 |
| | | | | HCDR2 | HPEDST | 315 | HCDR2 | TIHPEDSTTNYNQK FQG | 316 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 476 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab32 | VH | EVQLVQSGAEVKKPGASVKVSC KASGYSFSSYYMHWVRQAPGQG LEWMGTIHPSDSTTNYNQKFQG RVTMTVDTSTRTAYMELSSLRS EDTAVYYCADFLYWGQGTTVTV SS | 465 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FLY | 317 | HCDR3 | FLY | 317 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 477 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab34 | VH | EVQLVQSGAEVKKPGASVKVSC KASGYSFSSYYMHWVRQAPGQG LEWMGTIHPSDSTTNYNQKFQG RVTMTIDTSTRTAYMELSSLRS EDTAVYYCADFVYWGQGTTVTV SS | 466 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 478 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab43 | VH | EVQLVQSGAEVKKPGASVKVSC KASGYSFSSYYMHWVRQAPGQG LEWMGTIHPSDSTTNYNQKFQG RVTMTVDTSTRTAYMELSSLRS EDTAVYYCADFVYWGQGTTVTV SS | 467 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FVY | 303 | HCDR3 | FVY | 303 |
| | VL | DIVMTQTPLSLSVTPGQPASIS CKSSKSLLYKDGKTYLLWFLQK PGQSPQLLIYVVSTRASGVPDR FSGSGSGTDFTLKISRVEAEDV GVYYCQQLVEYPYTFGQGTKLE IK | 479 | LCDR1 | KSSKSLLYKDGKTY LL | 318 | LCDR1 | KSSKSLLYKDGKTY LL | 318 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab71 | VH | QVQLVQSGAEVKKPGASVKVSC KASGYSFSSYYMHWVRQAPGQG LEWMGDIHPSDSTTNYNQKFQG RVTMTVDTSTRTAYMELSSLRS EDTAVYYCANFVYWGQGTTVTV SS | 468 | HCDR1 | GYSFSSY | 301 | HCDR1 | SYYMH | 307 |
| | | | | HCDR2 | HPSDST | 302 | HCDR2 | DIHPSDSTTNYNQK FQG | 319 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIYVVSTRASGVPDR FSGSGSGTDFTLKISRVEAEDV GVYYCQQLVEYPYTFGQGTKLE IK | 480 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab137 | VH | QVQLVQSGAEVKKPGASVKVSC KASGYSFSSYYMHWVRQAPGQG LEWMGTIHPSDSTTNYNQKFQG RVTMTVDTSTRTAYMELSSLRS EDTAVYYCANFVYWGQGTTVTV SS | 469 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FVY | 303 | HCDR3 | FVY | 303 |
| | VL | DIVMTQTPLSLSVTPGQPASIS CKSSKSLLYKDGKTYLNWFLQK PGQSPQLLIDVVSTRASGVPDR FSGSGSGTDFTLKISRVEAEDV GVYYCQQLVEYPYTFGQGTKLE IK | 481 | LCDR1 | KSSKSLLYKDGKTY LN | 304 | LCDR1 | KSSKSLLYKDGKTY LN | 304 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab173 | VH | EVQLVQSGAEVKKPGASVKVSC KASGYSFSSYYMHWVRQAPGQG LEWMGTIYPGDSTTNYNQKFQG RVTMTVDTSTRTAYMELSSLRS EDTAVYYCADFVYWGQGTTVTV SS | 507 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FVY | 303 | HCDR3 | FVY | 303 |
| | VL | DIVMTQTPLSLSVTPGQPASIS CKSSKSLLYKDGHTYLNWFLQK PGQSPQLLIYVVSTRASGVPDR FSGSGSGTDFTLKISRVEAEDV GVYYCQQLVEYPYTFGQGTKLE IK | 508 | LCDR1 | KSSKSLLYKDGHTY LN | 311 | LCDR1 | KSSKSLLYKDGHTY LN | 311 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab174 | VH | EVQLVQSGAEVKKPGASVKVSC KASGYSFSSYYMHWVRQAPGQG LEWMGTIHPEDSTTNYNQKFQG RVTMTVDTSTRTAYMELSSLRS EDTAVYYCADFVYWGQGTTVTV SS | 509 | HCDR1 | GYSFSSY | 301 | HCDR1 | SYYMH | 307 |
| | | | | HCDR2 | HPEDST | 315 | HCDR2 | TIHPEDSTTNYNQK FQG | 316 |
| | | | | HCDR3 | FVY | 303 | HCDR3 | FVY | 303 |
| | VL | DIVMTQTPLSLSVTPGQPASIS CKSSKSLLYKDGHTYLNWFLQK PGQSPQLLIYVVSTRASGVPDR FSGSGSGTDFTLKISRVEAEDV GVYYCQQLVEYPYTFGQGTKLE IK | 510 | LCDR1 | KSSKSLLYKDGHTY LN | 311 | LCDR1 | KSSKSLLYKDGHTY LN | 311 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |
| Ab175 | VH | EVQLVQSGAEVKKPGASVKVSC KASGYSFSSYYMHWVRQAPGQG LEWMGTIHPSDSTTNYNQKFQG RVTMTVDTSTRTAYMELSSLRS EDTAVYYCADFLYWGQGTTVTV SS | 511 | HCDR1 | GYSFSSY | 301 | HCDR1 | SYYMH | 307 |
| | | | | HCDR2 | HPSDST | 302 | HCDR2 | TIHPSDSTTNYNQK FQG | 308 |
| | | | | HCDR3 | FLY | 317 | HCDR3 | FLY | 317 |
| | VL | DIVMTQTPLSLSVTPGQPASIS CKSSKSLLYKDGHTYLNWFLQK PGQSPQLLIYVVSTRASGVPDR FSGSGSGTDFTLKISRVEAEDV GVYYCQQLVEYPYTFGQGTKLE IK | 512 | LCDR1 | KSSKSLLYKDGHTY LN | 311 | LCDR1 | KSSKSLLYKDGHTY LN | 311 |
| | | | | LCDR2 | VVSTRAS | 305 | LCDR2 | VVSTRAS | 305 |
| | | | | LCDR3 | QQLVEYPYT | 306 | LCDR3 | QQLVEYPYT | 306 |

The amino acid sequences of the heavy chain variable region (VH) and light chain variable region (VL) are provided as follows. CDRs, defined according to the Kabat or Chothia system, are indicated.

TABLE 2

Amino acid and nucleotide sequences of exemplary heavy chain variable regions (VH) and light chain variable regions (VL).

| Antibody | Chain | Amino Acid Sequence | SEQ ID NO | Exemplary Nucleotide Sequence | SEQ ID NO |
|---|---|---|---|---|---|
| 28-0 | VH | EVQLVQSGAEVKKPGASVKVSCKASG YSFSSYYMHWVRQAPGQGLEWMGTIH PSDSTTNYNQKFQGRVTMTVDTSTRT AYMELSSLRSEDTAVYYCADFVYWGQ GTTVTVSS | 458 | GAAGTGCAACTTGTCCAGTCTGGAGCAGAAGTAA AGAAGCCAGGCGCATCAGTGAAAGTAAGCTGTAA AGCCTCTGGCTACAGTTTTTCCTCATACTACATG CACTGGGTTAGGCAGGCTCCTGGGCAAGGCCTGG AATGGATGGGTACTATCCATCCCTCAGATTCAAC CACAAACTACAACCAAAAGTTTCAAGGAAGAGTG ACAATGACCGTTGATACCTCTACACGCACAGCCT ATATGGAACTTAGCAGCCTGCGAAGCGAGGATAC AGCCGTATACTACTGCGCCGACTTCGTTTATTGG GGTCAAGGTACTACTGTCACCGTCAGCAGTGCCT CAACTAA | 483 |
| 29-0 | VH | EVQLVQSGAEVKKPGASVKVSCKASG YSFSSYYMHWVRQAPGQGLEWMGTIY PGASTTNYNQKFQGRVTMTVDTSTRT AYMELSSLRSEDTAVYYCADFVYWGQ GTTVTVSS | 459 | GAAGTTCAGTTGGTGCAGTCTGGCGCAGAAGTTA AGAAGCCCGGGGCATCAGTTAAAGTCAGTTGCAA AGCATCAGGTTATTCCTTTAGTAGCTATTATATG CACTGGGTGCGACAAGCCCCTGGTCAGGGTCTGG AGTGGATGGGGACTATTTATCCTGGGGCTTCTAC TACAAATTACAATCAGAAATTCCAGGGGCGAGTT ACCATGACTGTAGATACTAGCACTCGAACCGCAT ATATGGAATTGTCATCCCTCCGCTCAGAAGATAC AGCAGTGTACTATTGTGCCGACTTTGTCTACTGG GGTCAGGGTACAACTGTTACAGTCTCTAGTGCCT CAACTAA | 484 |
| Ab17 | VH | EVQLVQSGAEVKKPGASVKVSCKASG YSFSSYYMHWVRQAPGQGLEWMGTIH PSDSTTNYNQKFQGRVTMTVDTSTRT AYMELSSLRSEDTAVYYCADFVYWGQ GTTVTVSS | 460 | GAAGTGCAACTTGTCCAGTCTGGAGCAGAAGTAA AGAAGCCAGGCGCATCAGTGAAAGTAAGCTGTAA AGCCTCTGGCTACAGTTTTTCCTCATACTACATG CACTGGGTTAGGCAGGCTCCTGGGCAAGGCCTGG AATGGATGGGTACTATCCATCCCTCAGATTCAAC CACAAACTACAACCAAAAGTTTCAAGGAAGAGTG ACAATGACCGTTGATACCTCTACACGCACAGCCT ATATGGAACTTAGCAGCCTGCGAAGCGAGGATAC AGCCGTATACTACTGCGCCGACTTCGTTTATTGG GGTCAAGGTACTACTGTCACCGTCAGCAGTGCCT CAACTAA | 485 |
| Ab18 | VH | EVQLVQSGAEVKKPGASVKVSCKASG YSFSSYYMHWVRQAPGQGLEWMGTIH PSDSTTNYNQKFQGRVTMTVDTSTRT AYMELSSLRSEDTAVYYCADFVYWGQ GTTVTVSS | 461 | GAAGTGCAACTTGTCCAGTCTGGAGCAGAAGTAA AGAAGCCAGGCGCATCAGTGAAAGTAAGCTGTAA AGCCTCTGGCTACAGTTTTTCCTCATACTACATG CACTGGGTTAGGCAGGCTCCTGGGCAAGGCCTGG AATGGATGGGTACTATCCATCCCTCAGATTCAAC CACAAACTACAACCAAAAGTTTCAAGGAAGAGTG ACAATGACCGTTGATACCTCTACACGCACAGCCT ATATGGAACTTAGCAGCCTGCGAAGCGAGGATAC AGCCGTATACTACTGCGCCGACTTCGTTTATTGG GGTCAAGGTACTACTGTCACCGTCAGCAGTGCCT CAACTAA | 486 |
| Ab29 | VH | EVQLVQSGAEVKKPGASVKVSCKASG YSFSSYYMHWVRQAPGQGLEWMGTIY PEDSTTNYNQKFQGRVTMTVDTSTRT AYMELSSLRSEDTAVYYCADFVYWGQ GTTVTVSS | 462 | GAGGTACAGCTGGTACAAAGTGGGGCGGAAGTTA AAAAGCCGGGGGCTTCAGTGAAGGTCAGCTGTAA GGCGAGTGGGTACTCTTTCTCCTCCTATTATATG CACTGGGTCAGGCAGGCCCCAGGACAAGGTCTCG AATGGATGGGCACGATCTACCCAGAGGACTCCAC GACCAACTACAATCAGAAGTTTCAGGGTCGAGTG ACTATGACCGTTGATACCTCAACGAGAACCGCAT ACATGGAACTCTCCAGCTTGAGAAGTGAAGATAC AGCCGTATATTATTGCGCGGATTTCGTCTATTGG GGGCAAGGTACGACTGTCACAGTTAGCTCAGCCT CAACTAA | 487 |
| Ab28 | VH | EVQLVQSGAEVKKPGASVKVSCKASG YSFSSYYMHWVRQAPGQGLEWMGTIY PGDSTTNYNQKFQGRVTMTVDTSTRT AYMELSSLRSEDTAVYYCADFVYWGQ GTTVTVSS | 463 | GAAGTCCAATTGGTCCAGAGCGGAGCAGAGGTGA AGAAGCCAGGGGCCTCAGTTAAAGTGTCCTGTAA AGCATCTGGGTATTCCTTCAGCAGCTATTATATG CACTGGGTCCGCCAGGCTCCAGGTCAGGGGCTTG AATGGATGGGCACCATCTACCCCGGAGACTCTAC GACGAACTATAACCAAAAATTTCAGGGGCGCGTT ACCATGACGGTAGACACTTCTACACGGACGGCGT ATATGGAGCTCAGCAGTCTTAGGTCTGAGGATAC GGCTGTTTATTATTGCGCGGATTTCGTCTATTGG GGACAGGGAACAACGGTTACGGTTAGCAGTGCCT CAACTAA | 488 |
| Ab30 | VH | EVQLVQSGAEVKKPGASVKVSCKASG YSFSSYYMHWVRQAPGQGLEWMGTIH PEDSTTNYNQKFQGRVTMTVDTSTRT | 464 | GAGGTACAACTTGTTCAGTCTGGCGCCGAAGTCA AAAAACCTGGGGCAAGCGTAAAAGTGTCTTGCAA AGCGAGCGGGTATTCTTTTAGTAGTTACTATATG CACTGGGTTAGGCAAGCGCCTGGTCAAGGTCTGG | 489 |
| | | AYMELSSLRSEDTAVYYCADFVYWGQ GTTVTVSS | | AGTGGATGGGAACAATTCATCCCGAAGACAGTAC CACCAATTACAACCAGAAATTCCAAGGCAGAGTT ACCATGACCGTAGATACGTCAACGCGGACGGCAT ATATGGAACTGTCCTCTCTCAGATCAGAAGACAC GGCTGTTTATTACTGCGCCGATTTCGTGTACTGG GGCCAAGGCACTACTGTCACCGTTTCCAGCGCCT CAACTAA | |
| Ab32 | VH | EVQLVQSGAEVKKPGASVKVSCKASG YSFSSYYMHWVRQAPGQGLEWMGTIH PSDSTTNYNQKFQGRVTMTVDTSTRT AYMELSSLRSEDTAVYYCADFLYWGQ GTTVTVSS | 465 | GAAGTACAGCTGGTACAGTCTGGTGCTGAGGTTA AGAAACCGGGAGCGTCCGTTAAGGTAAGCTGTAA GGCTAGTGGCTACTCCTTTTCCTCATACTATATG CACTGGGTCAGGCAGGCACCGGGACAAGGTCTGG AATGGATGGGTACAATACATCCGTCAGACAGCAC TACGAACTATAATCAGAAGTTTCAAGGACGGGTC ACTATGACTGTGGACACGAGTACGAGAACAGCCT ACATGGAACTCTCTTCTCTTCGGAGCGAGGACAC GGCAGTGTACTATTGTGCTGATTTCCTTTACTGG GGCCAGGGTACGACTGTGACGGTCAGTTCCGCCT CAACTAA | 490 |
| Ab34 | VH | EVQLVQSGAEVKKPGASVKVSCKASG YSFSSYYMHWVRQAPGQGLEWMGTIH PSDSTTNYNQKFQGRVTMTIDTSTRT AYMELSSLRSEDTAVYYCADFVYWGQ GTTVTVSS | 466 | GAAGTTCAACTTGTTCAGAGCGGCGCAGAGGTAA AAAAGCCGGGCGCAAGTGTTAAGGTTAGCTGCAA AGCATCTGGGTATAGCTTCTCTAGCTATTATATG CACTGGGTGCGACAAGCTCCTGGTCAGGGTCTGG AATGGATGGGAACCATCCACCCGAGCGACTCTAC GACGAACTATAATCAAAAATTCCAGGGCAGAGTC ACAATGACAATAGACACGTCCACCCGGACCGCTT ATATGGAACTTTCCTCCCTGCGATCCGAGGATAC GGCCGTTTATTATTGCGCCGACTTTGTGTACTGG GGGCAAGGGACCACGGTAACGGTGAGTTCAGCCT CAACTAA | 491 |
| Ab43 | VH | EVQLVQSGAEVKKPGASVKVSCKASG YSFSSYYMHWVRQAPGQGLEWMGTIH PSDSTTNYNQKFQGRVTMTVDTSTRT AYMELSSLRSEDTAVYYCADFVYWGQ GTTVTVSSAST | 467 | GAAGTGCAACTTGTCCAGTCTGGAGCAGAAGTAA AGAAGCCAGGCGCATCAGTGAAAGTAAGCTGTAA AGCCTCTGGCTACAGTTTTTCCTCATACTACATG CACTGGGTTAGGCAGGCTCCTGGGCAAGGCCTGG AATGGATGGGTACTATCCATCCCTCAGATTCAAC CACAAACTACAACCAAAAGTTTCAAGGAAGAGTG ACAATGACCGTTGATACCTCTACACGCACAGCCT ATATGGAACTTAGCAGCCTGCGAAGCGAGGATAC | 492 |
| | | | | AGCCGTATACTACTGCGCCGACTTCGTTTATTGG GGTCAAGGTACTACTGTCACCGTCAGCAGTGCCT CAACTAA | |
| Ab71 | VH | QVQLVQSGAEVKKPGASVKVSCKASG YSFSSYYMHWVRQAPGQGLEWMGDIH PSDSTTNYNQKFQGRVTMTVDTSTRT AYMELSSLRSEDTAVYYCANFVYWGQ GTTVTVSS | 468 | TTCTGCTCTGCCTGGCCGGGCGCGCCTTGGCCCA AGTACAGCTTGTCCAGTCAGGTGCAGAGGTAAAA AAGCCCGGCGCATCAGTGAAGGTATCTTGTAAAG CGTCCGGTTATTCATTTTCATCTTACTACATGCA TTGGGTTCGGCAGGCACCGGGACAGGGCCTGGAA TGGATGGGGGACATCCATCCATCTGACAGCACAA CAAATTACAATCAGAAATTTCAAGGTCGGGTCAC AATGACCGTGGATACAAGCACAAGAACAGCATAT ATGGAACTGAGCTCACTTCGGAGTGAAGATACTG CCGTGTATTATTGTGCTAATTTCGTCTATTGGGG GCAGGGGACGACGGTGACAGTAAGTAGTGCCTCA ACTAAGGGCCCCTCCGTGTTCCCGTT | 493 |
| Ab137 | VH | QVQLVQSGAEVKKPGASVKVSCKASG YSFSSYYMHWVRQAPGQGLEWMGTIH PSDSTTNYNQKFQGRVTMTVDTSTRT AYMELSSLRSEDTAVYYCANFVYWGQ GTTVTVSS | 469 | AAGTACAGCTTGTCCAGTCAGGTGCAGAGGTAAA AAAGCCCGGCGCATCAGTGAAGGTATCTTGTAAA GCGTCCGGTTATTCATTTTCATCTTACTACATGC ATTGGGTTCGGCAGGCACCGGGACAGGGCCTGGA ATGGATGGGGACGATCCATCCATCTGACAGCACA ACAAATTACAATCAGAAATTTCAAGGTCGGGTCA CAATGACCGTGGATACAAGCACAAGAACAGCATA TATGGAACTGAGCTCACTTCGGAGTGAAGATACT GCCGTGTATTATTGTGCTAATTTCGTCTATTGGG GGCAGGGGACGACGGTGACAGTAAGTAGT | 494 |
| Ab173 | VH | EVQLVQSGAEVKKPGASVKVSCKASG YSFSSYYMHWVRQAPGQGLEWMGTIY PGDSTTNYNQKFQGRVTMTVDTSTRT AYMELSSLRSEDTAVYYCADFVYWGQ GTTVTVSS | 507 | GAAGTCCAATTGGTCCAGAGCGGAGCAGAGGTGA AGAAGCCAGGGGCCTCAGTTAAAGTGTCCTGTAA AGCATCTGGGTATTCCTTCAGCAGCTATTATATG CACTGGGTCCGCCAGGCTCCAGGTCAGGGGCTTG AATGGATGGGCACCATCTACCCCGGAGACTCTAC GACGAACTATAACCAAAAATTTCAGGGGCGCGTT ACCATGACGGTAGACACTTCTACACGGACGGCGT ATATGGAGCTCAGCAGTCTTAGGTCTGAGGATAC GGCTGTTTATTATTGCGCGGATTTCGTCTATTGG GGACAGGGAACAACGGTTACGGTTAGCAGT | |
| Ab174 | VH | EVQLVQSGAEVKKPGASVKVSCKASG YSFSSYYMHWVRQAPGQGLEWMGTIH PEDSTTNYNQKFQGRVTMTVDTSTRT AYMELSSLRSEDTAVYYCADFVYWGQ GTTVTVSS | 509 | GAGGTACAACTTGTTCAGTCTGGCGCCGAAGTCA AAAAACCTGGGGCAAGCGTAAAAGTGTCTTGCAA AGCGAGCGGGTATTCTTTTAGTAGTTACTATATG CACTGGGTTAGGCAAGCGCCTGGTCAAGGTCTGG AGTGGATGGGAACAATTCATCCCGAAGACAGTAC CACCAATTACAACCAGAAATTCCAAGGCAGAGTT ACCATGACCGTAGATACGTCAACGCGGACGGCAT ATATGGAACTGTCCTCTCTCAGATCAGAAGACAC GGCTGTTTATTACTGCGCCGATTTCGTGTACTGG GGCCAAGGCACTACTGTCACCGTTTCCAGC | |
| Ab175 | VH | EVQLVQSGAEVKKPGASVKVSCKASG YSFSSYYMHWVRQAPGQGLEWMGTIH PSDSTTNYNQKFQGRVTMTVDTSTRT AYMELSSLRSEDTAVYYCADFLYWGQ GTTVTVSS | 512 | GAAGTACAGCTGGTACAGTCTGGTGCTGAGGTTA AGAAACCGGGAGCGTCCGTTAAGGTAAGCTGTAA GGCTAGTGGCTACTCCTTTTCCTCATACTATATG CACTGGGTCAGGCAGGCACCGGGACAAGGTCTGG AATGGATGGGTACAATACATCCGTCAGACAGCAC TACGAACTATAATCAGAAGTTTCAAGGACGGGTC ACTATGACTGTGGACACGAGTACGAGAACAGCCT ACATGGAACTCTCTTCTCTTCGGAGCGAGGACAC GGCAGTGTACTATTGTGCTGATTTCCTTTACTGG GGCCAGGGTACGACTGTGACGGTCAGTTCC | |
| 28-0 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCQQLVEYPYTFG QGTKLEIK | 470 | GATATTGTCATGACACAAACCCCCCTGAGTCTTA GCGTCACCCCTGGGCAGCCCGCTTCAATAAGTTG TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA CTACTGTCAGCAGCTCGTAGAATATCCATACACA TTTGGACAAGGTACGAA.ACTGGAGATAAA.A | 495 |
| 29-0 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCQQLVEYPYTFG QGTKLEIK | 471 | GATATTGTCATGACACAAACCCCCCTGAGTCTTA GCGTCACCCCTGGGCAGCCCGCTTCAATAAGTTG TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG | 496 |
| 70 | | | | | |
| | | | | CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA CTACTGTCAGCAGCTCGTAGAATATCCATACACA TTTGGACAAGGTACGAAACTGGAGATAAAA | |
| Ab17 | VL | DIVMTQTPLSLSVTPGQPASISCKSS KSLLYKDGHTYLNWFLQKPGQSPQLL IYVVSTRASGVPDRFSGSGSGTDFTL KISRVEAEDVGVYYCQQLVEYPYTFG QGTKLEIK | 472 | GACATCGTAATGACTCAAACCCCTCTGTCTTTGA GTGTCACCCCTGGTCAACCTGCATCTATTAGCTG CAAATCCAGTAAGTCTTTGCTTTACAAAGATGGA CATACATACTTGAACTGGTTTTTGCAGAAACCCG GTCAGAGTCCTCAGCTTCTGATCTATGTTGTCAG TACCCGCGCGTCTGGGGTACCTGACCGCTTTAGC GGAAGTGGAAGCGGTACGGACTTTACGCTCAAAA TTTCAAGAGTGGAGGCGGAGGATGTAGGGGTCTA CTACTGTCAACAGCTTGTAGAATATCCTTATACC TTCGGACAGGGTACAAA.ACTTGAAATTAA.A | 497 |
| Ab18 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCEQLVEYPYTFG QGTKLEIK | 473 | GACATCGTTATGACCCAAACTCCCCTCTCCCTTA GTGTGACTCCTGGGCAGCCTGCATCTATAAGTTG TAAGAGTTCAAAGTCACTGTTGTATAAAGATGGA AAAACTTACCTCAACTGGTTCTTGCAGAAGCCTG GCCAGTCACCCCAACTTCTGATATACGTCGTCTC AACCCGAGCATCTGGTGTACCGGACAGGTTTTCT GGTAGTGGCAGTGGAACGGACTTTACGTTGAAAA TATCCCGAGTTGAAGCGGAAGATGTAGGCGTCTA TTACTGCGAGCAGCTCGTAGAGTATCCCTACACG TTTGGACAGGGTACGAAACTTGAGATTAAG | 498 |
| Ab29 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCQQLVEYPYTFG QGTKLEIK | 474 | GATATTGTCATGACACAAACCCCCCTGAGTCTTA GCGTCACCCCTGGGCAGCCCGCTTCAATAAGTTG TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA CTACTGTCAGCAGCTCGTAGAATATCCATACACA TTTGGACAAGGTACGAAACTGGAGATAAAA | 499 |
| Ab28 | VL | DIVMTQTPLSLSVTPGQPASISCKSS KSLLYKDGKTYLNWFLQKPGQSPQLL | 475 | GATATTGTCATGACACAAACCCCCCTGAGTCTTA GCGTCACCCCTGGGCAGCCCGCTTCAATAAGTTG | 500 |
| | | IYVVSTRASGVPDRFSGSGSGTDFTL KISRVEAEDVGVYYCQQLVEYPYTFG QGTKLEIK | | TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA CTACTGTCAGCAGCTCGTAGAATATCCATACACA TTTGGACAAGGTACGAAACTGGAGATAAAA | |
| Ab30 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCQQLVEYPYTFG QGTKLEIK | 476 | GATATTGTCATGACACAAACCCCCCTGAGTCTTA GCGTCACCCCTGGGCAGCCCGCTTCAATAAGTTG TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA CTACTGTCAGCAGCTCGTAGAATATCCATACACA TTTGGACAAGGTACGAAACTGGAGATAAAA | 501 |
| Ab32 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCQQLVEYPYTFG QGTKLEIK | 477 | GATATTGTCATGACACAAACCCCCCTGAGTCTTA GCGTCACCCCTGGGCAGCCCGCTTCAATAAGTTG TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA CTACTGTCAGCAGCTCGTAGAATATCCATACACA TTTGGACAAGGTACGAAACTGGAGATAAAA | 502 |
| Ab34 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCQQLVEYPYTFG QGTKLEIK | 478 | GATATTGTCATGACACAAACCCCCCTGAGTCTTA GCGTCACCCCTGGGCAGCCCGCTTCAATAAGTTG TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA | 503 |
| | | | | CTACTGTCAGCAGCTCGTAGAATATCCATACACA TTTGGACAAGGTACGAAACTGGAGATAAAA | |
| Ab43 | VL | DIVMTQTPLSLSVTPGQPASISCKSS KSLLYKDGKTYLLWFLQKPGQSPQLL IYVVSTRASGVPDRFSGSGSGTDFTL KISRVEAEDVGVYYCQQLVEYPYTFG QGTKLEIK | 479 | GATATAGTAATGACTCAGACCCCGCTCAGCCTCA GCGTAACACCGGGACAACCTGCCTCAATATCATG CAAATCCTCCAAAAGCCTCCTCTACAAAGACGGA AAGACGTATCTGCTGTGGTTTCTTCAGAAACCGG GGCAATCCCCGCAACTTCTTATCTATGTCGTATC AACCAGAGCTTCCGGTGTTCCCGATCGCTTCTCT GGTAGCGGGAGTGGAACTGACTTCACACTCAAAA TATCTAGGGTCGAGGCTGAGGACGTGGGTGTTTA CTATTGTCAACAGTTGGTGGAGTATCCCTACACA TTCGGGCAGGGTACTAAACTTGAGATTAAG | 504 |
| Ab71 | VL | DIVMTQTPLSLSVTPGQPASISCKSS KSLLYKDGKTYLNWFLQKPGQSPQLL IYVVSTRASGVPDRFSGSGSGTDFTL KISRVEAEDVGVYYCQQLVEYPYTFG QGTKLEIK | 480 | GATATTGTCATGACACAAACCCCCCTGAGTCTTA GCGTCACCCCTGGGCAGCCCGCTTCAATAAGTTG TAAGTCCTCTAAATCATTGCTGTATAAAGATGGG AAGACCTATCTTAACTGGTTTCTCCAAAAGCCAG GGCAGTCACCACAACTGTTGATCTACGTTGTAAG CACCAGGGCGAGTGGAGTCCCCGACAGATTCAGT GGTAGCGGCTCTGGTACAGATTTTACCTTGAAAA TATCTAGGGTGGAAGCGGAGGATGTCGGTGTCTA CTACTGTCAGCAGCTCGTAGAATATCCATACACA TTTGGACAAGGTACGAAACTGGAGATAAAA | 505 |
| Ab137 | VL | DIVMTQTPLSLSVTPGQPASISCKSS KSLLYKDGKTYLNWFLQKPGQSPQLL IDVVSTRASGVPDRFSGSGSGTDFTL KISRVEAEDVGVYYCQQLVEYPYTFG QGTKLEIK | 481 | TTCTGCTCTGCCTGGCCGGGCGCGCCTTGGCCGA TATTGTCATGACACAAACCCCCCTGAGTCTTAGC GTCACCCCTGGGCAGCCCGCTTCAATAAGTTGTA AGTCCTCTAAATCATTGCTGTATAAAGATGGGAA GACCTATCTTAACTGGTTTCTCCAAAAGCCAGGG CAGTCACCACAACTGTTGATCGATGTTGTAAGCA CCAGGGCGAGTGGAGTCCCCGACAGATTCAGTGG TAGCGGCTCTGGTACAGATTTTACCTTGAAAATA TCTAGGGTGGAAGCGGAGGATGTCGGTGTCTACT ACTGTCAGCAGCTCGTAGAATATCCATACACATT TGGACAAGGTACGAA.ACTGGAGATAAAACGTACG GTGGCAGCGCCTTCCGTGT | 506 |
| Ab173 | VL | DIVMTQTPLSLSVTPGQPASISCKSS KSLLYKDGHTYLNWFLQKPGQSPQLL IYVVSTRASGVPDRFSGSGSGTDFTL | 508 | GACATCGTAATGACTCAAACCCCTCTGTCTTTGA GTGTCACCCCTGGTCAACCTGCATCTATTAGCTG CAAATCCAGTAAGTCTTTGCTTTACAAAGATGGA | |
| | | KISRVEAEDVGVYYCQQLVEYPYTFG QGTKLEIK | | CATACATACTTGAACTGGTTTTTGCAGAAACCCG GTCAGAGTCCTCAGCTTCTGATCTATGTTGTCAG TACCCGCGCGTCTGGGGTACCTGACCGCTTTAGC GGAAGTGGAAGCGGTACGGACTTTACGCTCAAAA TTTCAAGAGTGGAGGCGGAGGATGTAGGGGTCTA CTACTGTCAACAGCTTGTAGAATATCCTTATACC TTCGGACAGGGTACAAAACTTGAAATTAAA | |
| Ab174 | VL | DIVMTQTPLSLSVTPGQPASISCKSS KSLLYKDGHTYLNWFLQKPGQSPQLL IYVVSTRASGVPDRFSGSGSGTDFTL KISRVEAEDVGVYYCQQLVEYPYTFG QGTKLEIK | 510 | GACATCGTAATGACTCAAACCCCTCTGTCTTTGA GTGTCACCCCTGGTCAACCTGCATCTATTAGCTG CAAATCCAGTAAGTCTTTGCTTTACAAAGATGGA CATACATACTTGAACTGGTTTTTGCAGAAACCCG GTCAGAGTCCTCAGCTTCTGATCTATGTTGTCAG TACCCGCGCGTCTGGGGTACCTGACCGCTTTAGC GGAAGTGGAAGCGGTACGGACTTTACGCTCAAAA TTTCAAGAGTGGAGGCGGAGGATGTAGGGGTCTA CTACTGTCAACAGCTTGTAGAATATCCTTATACC TTCGGACAGGGTACAAAACTTGAAATTAAA | |
| Ab175 | VL | DIVMTQTPLSLSVTPGQPASISCKSS KSLLYKDGHTYLNWFLQKPGQSPQLL IYVVSTRASGVPDRFSGSGSGTDFTL KISRVEAEDVGVYYCQQLVEYPYTFG QGTKLEIK | 512 | GACATCGTAATGACTCAAACCCCTCTGTCTTTGA GTGTCACCCCTGGTCAACCTGCATCTATTAGCTG CAAATCCAGTAAGTCTTTGCTTTACAAAGATGGA CATACATACTTGAACTGGTTTTTGCAGAAACCCG GTCAGAGTCCTCAGCTTCTGATCTATGTTGTCAG TACCCGCGCGTCTGGGGTACCTGACCGCTTTAGC GGAAGTGGAAGCGGTACGGACTTTACGCTCAAAA TTTCAAGAGTGGAGGCGGAGGATGTAGGGGTCTA CTACTGTCAACAGCTTGTAGAATATCCTTATACC TTCGGACAGGGTACAAAACTTGAAATTAAA | |

In an embodiment, the antibody molecule comprises one, two, or three CDRs of the VH region of an antibody molecule described herein, e.g., in Table 1 or 2, using the Kabat or Chothia definitions of CDRs. In an embodiment, the antibody molecule comprises one, two, or three CDRs of the VL region of an antibody molecule described herein, e.g., in Table 1 or 2, using the Kabat or Chothia definitions of CDRs. In an embodiment, the antibody molecule comprises one or more (e.g., two or three) CDRs of the VH region and one or more (e.g., two or three) CDRs of the VL region of an antibody molecule described herein, e.g., in Table 1 or 2, using the Kabat or Chothia definitions of CDRs.

In an embodiment, the antibody molecule comprises one, two, or three HCDRs described in Table 1 or 2. In an embodiment, the antibody molecule comprises one, two, or three LCDRs described in Table 1 or 2. In an embodiment, the antibody molecule comprises one or more (e.g., two or three) HCDRs and one or more (e.g., two or three) LCDRs described in Table 1 or 2.

In an embodiment, the antibody molecule comprises one, two, three, or four frameworks of the VH region of an antibody molecule described in Table 1 or 2. In an embodiment, the antibody molecule comprises one, two, three, or four frameworks of the VL region of an antibody molecule described in Table 1 or 2. In an embodiment, the antibody molecule comprises one or more (e.g., two, three, or four) frameworks of the VH region and one or more (e.g., two, three, or four) frameworks of the VL region of an antibody molecule described in Table 1 or 2.

In an embodiment, the antibody molecule comprises a VH of an antibody molecule described herein, e.g., in Table 1 or 2. In an embodiment, the antibody molecule comprises a VL of an antibody molecule described herein, e.g., in Table 1 or 2. In an embodiment, the antibody molecule comprises a VH and a VL of an antibody molecule described herein, e.g., in Table 1 or 2.

In an embodiment, the antibody molecule comprises a VH having an amino acid sequence described in Table 1 or 2, or an amino acid sequence substantially identical thereof (e.g., differing by no more than 1, 2, 3, 4, 5, 6, 7, 8, 9, 10, 11, 12, 13, 14, or 15 amino acid residues therefrom, or at least 85, 90, 95, or 99% identical thereto). In an embodiment, the antibody molecule comprises a VL having an amino acid sequence described in Table 1 or 2, or an amino acid sequence substantially identical thereof (e.g., differing by no more than 1, 2, 3, 4, 5, 6, 7, 8, 9, 10, 11, 12, 13, 14, or 15 amino acid residues therefrom, or at least 85, 90, 95, or 99% identical thereto). In an embodiment, the antibody molecule comprises a VH having an amino acid sequence described in Table 1 or 2 (or an amino acid sequence substantially identical thereof) and a VL having an amino acid sequences described in Table 1 or 2 (or an amino acid sequence substantially identical thereof).

In an embodiment, the antibody molecule comprises a VH encoded by a nucleotide sequence described in Table 2, or a nucleotide sequence substantially identical thereof (e.g., differing by no more than 3, 6, 15, 30, or 45 nucleotides therefrom, or at least about 85%, 90%, 95%, or 99% identical thereto). In an embodiment, the antibody molecule comprises a VL encoded by a nucleotide sequence described in Table 2, or a nucleotide sequence substantially identical thereof (e.g., differing by no more than 3, 6, 15, 30, or 45 nucleotides therefrom, or at least about 85%, 90%, 95%, or 99% identical thereto). In an embodiment, the antibody molecule comprises a VH encoded by a nucleotide sequence described in Table 2 (or a nucleotide sequence substantially identical thereof) and a VL encoded by a nucleotide sequence described in Table 2 (or a nucleotide sequence substantially identical thereof).

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 458. In an embodiment, the VL comprises the amino acid sequence of SEQ ID NO: 470. In an embodiment, the VH comprises the amino acid sequence of SEQ ID NO: 458 and the VL comprises the amino acid sequence of SEQ ID NO: 470.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 309; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 309; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 310; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 310; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 459. In an embodiment, the VL comprises the amino acid sequence of SEQ ID NO: 471. In an embodiment, the VH comprises the amino acid sequence of SEQ ID NO: 459 and the VL comprises the amino acid sequence of SEQ ID NO: 471.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 303; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 303; and the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 460. In an embodiment, the VL comprises the amino acid sequence of SEQ ID NO: 472. In an embodiment, the VH comprises the amino acid sequence of SEQ ID NO: 460 and the VL comprises the amino acid sequence of SEQ ID NO: 472.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 312. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 312.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 312. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 312.

In an embodiment, the VH comprises the amino acid sequence of SEQ ID NO: 461. In an embodiment, the VL comprises the amino acid sequence of SEQ ID NO: 473. In an embodiment, the VH comprises the amino acid sequence of SEQ ID NO: 461 and the VL comprises the amino acid sequence of SEQ ID NO: 473.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 313; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 313; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 314; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 314; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 462. In an embodiment, the VL comprises the amino acid sequence of SEQ ID NO: 474. In an embodiment, the VH comprises the amino acid sequence of SEQ ID NO: 462 and the VL comprises the amino acid sequence of SEQ ID NO: 474.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 321; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 321; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 322; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 322; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 463. In an embodiment, the VL comprises the amino acid sequence of SEQ ID NO: 475. In an embodiment, the VH comprises the amino acid sequence of SEQ ID NO: 463 and the VL comprises the amino acid sequence of SEQ ID NO: 475.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 315; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 315; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 316; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 316; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 464. In an embodiment, the VL comprises the amino acid sequence of SEQ ID NO: 476. In an embodiment, the VH comprises the amino acid sequence of SEQ ID NO: 464 and the VL comprises the amino acid sequence of SEQ ID NO: 476.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 317; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 317; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 317; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 317; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 465. In an embodiment, the VL comprises the amino acid sequence of SEQ ID NO: 477. In an embodiment, the VH comprises the amino acid sequence of SEQ ID NO: 465 and the VL comprises the amino acid sequence of SEQ ID NO: 477.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 466. In an embodiment, the VL comprises the amino acid sequence of SEQ ID NO: 478. In an embodiment, the VH comprises the amino acid sequence of SEQ ID NO: 466 and the VL comprises the amino acid sequence of SEQ ID NO: 478.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 318; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 318; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 303; and/or the VL comprises one, two, or all of: (i) an LCDR1 comprising an amino acid sequence of SEQ ID NO: 318; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 303; and the VL comprises: (i) an LCDR1 comprising an amino acid sequence of SEQ ID NO: 318; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 467. In an embodiment, the VL comprises the amino acid sequence of SEQ ID NO: 479. In an embodiment, the VH comprises the amino acid sequence of SEQ ID NO: 467 and the VL comprises the amino acid sequence of SEQ ID NO: 479.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 319; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 319; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 468. In an embodiment, the VL comprises the amino acid sequence of SEQ ID NO: 480. In an embodiment, the VH comprises the amino acid sequence of SEQ ID NO: 468 and the VL comprises the amino acid sequence of SEQ ID NO: 480.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 303; and/or the VL comprises one, two, or all of: (i) an LCDR1 comprising an amino acid sequence of SEQ ID NO: 304; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 303; and the VL comprises: (i) an LCDR1 comprising an amino acid sequence of SEQ ID NO: 304; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 469. In an embodiment, the VL comprises the amino acid sequence of SEQ ID NO: 481. In an embodiment, the VH comprises the amino acid sequence of SEQ ID NO: 469 and the VL comprises the amino acid sequence of SEQ ID NO: 481.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 303; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 303; and/or the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 303; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 303; and the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 507. In an embodiment, the VL comprises the amino acid sequence of SEQ ID NO: 508. In an embodiment, the VH comprises the amino acid sequence of SEQ ID NO: 507 and the VL comprises the amino acid sequence of SEQ ID NO: 508.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 315; or (iii) an HCDR3 comprising an amino acid sequence of SEQ ID NO: 303; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 315; and (iii) an HCDR3 comprising an amino acid sequence of SEQ ID NO: 303; and/or the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 316; or (iii) an HCDR3 comprising an amino acid sequence of SEQ ID NO: 303; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 316; and (iii) an HCDR3 comprising an amino acid sequence of SEQ ID NO: 303; and the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 509. In an embodiment, the VL comprises the amino acid sequence of SEQ ID NO: 510. In an embodiment, the VH comprises the amino acid sequence of SEQ ID NO: 509 and the VL comprises the amino acid sequence of SEQ ID NO: 510.

In an embodiment, the VH comprises one, two, or all of: (i) an HCDR1 comprising an amino acid sequence of SEQ ID NO: 301; (ii) an HCDR2 comprising an amino acid sequence of SEQ ID NO: 302; or (iii) an HCDR3 comprising an amino acid sequence of SEQ ID NO: 317; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 301; (ii) an HCDR2 comprising an amino acid sequence of SEQ ID NO: 302; and (iii) an HCDR3 comprising an amino acid sequence of SEQ ID NO: 317; and/or the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises one, two, or all of: (i) an HCDR1 comprising an amino acid sequence of SEQ ID NO: 307; (ii) an HCDR2 comprising an amino acid sequence of SEQ ID NO: 308; or (iii) an HCDR3 comprising an amino acid sequence of SEQ ID NO: 317; and/or the VL comprises one, two, or all of: (i) an LCDR1 comprising an amino acid sequence of SEQ ID NO: 311; (ii) an LCDR2 comprising an amino acid sequence of SEQ ID NO: 305; or (iii) an LCDR3 comprising an amino acid sequence of SEQ ID NO: 306. In an embodiment, the VH comprises: (i) an HCDR1 comprising an amino acid sequence of SEQ ID NO: 307; (ii) an HCDR2 comprising an amino acid sequence of SEQ ID NO: 308; and (iii) an HCDR3 comprising an amino acid sequence of SEQ ID NO: 317; and the VL comprises: (i) an LCDR1 comprising an amino acid sequence of SEQ ID NO: 311; (ii) an LCDR2 comprising an amino acid sequence of SEQ ID NO: 305; and (iii) an LCDR3 comprising an amino acid sequence of SEQ ID NO: 306.

In an embodiment, the VH comprises the amino acid sequence of SEQ ID NO: 511. In an embodiment, the VL comprises the amino acid sequence of SEQ ID NO: 512. In an embodiment, the VH comprises the amino acid sequence of SEQ ID NO: 511 and the VL comprises the amino acid sequence of SEQ ID NO: 512.

In an aspect, the disclosure features an anti-CD138 antibody molecule (e.g., a Ph-selective anti-CD138 antibody molecule) comprising one or both of:
(a) a heavy chain variable region (VH), wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of 28-0, 29-0, Ab17, Ab18, Ab29, Ab28, Ab30, Ab32, Ab34, Ab43, Ab71, Ab137, Ab173, Ab174, or Ab175, e.g., as listed in Table 1 or 2); (ii) an HCDR2 comprising an amino acid sequence that differs by no more than 1, 2, or 3 amino acid residues from, or has at least 85, 90, 95, 99 or 100% homology with, the amino acid sequence of the HCDR2 of the VH; or (iii) an HCDR3 comprising an amino acid sequence that differs by no more than 1, 2, or 3 amino acid residues from, or has at least 85, 90, 95, 99 or 100% homology with, the amino acid sequence of the HCDR3 of the VH; or
(b) a light chain variable region (VL), wherein the VL comprises three light chain complementarity determining regions (LCDR1, LCDR2, and LCDR3), wherein the VL comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of 28-0, 29-0, Ab17, Ab18, Ab29, Ab28, Ab30, Ab32, Ab34, Ab43, Ab71, Ab137, Ab173, Ab174, or Ab175; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the VL; or (iii) an LCDR3 comprising an amino acid sequence that differs by no more than 1, 2, or 3 amino acid residues from, or has at least 85, 90, 95, 99 or 100% homology with, the amino acid sequence of the VL.

In an embodiment, the VH comprises: (i) an HCDR1 comprising an amino acid sequence that differs by no more than 1, 2, or 3 amino acid residues from, or has at least 85, 90, 95, 99 or 100% homology with, the amino acid sequence of the HCDR1 of the VH; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the VH; and (iii) an HCDR3 comprising an amino acid sequence that differs by no more than 1, 2, or 3 amino acid residues from, or has at least 85, 90, 95, 99 or 100% homology with, the amino acid sequence of the HCDR3 of the VH.

In an embodiment, the VH comprises: (i) an HCDR1 comprising the amino acid sequence of the HCDR1 of the VH; (ii) an HCDR2 comprising the amino acid sequence of the HCDR2 of the VH; and (iii) an HCDR3 comprising the amino acid sequence of the HCDR3 of the VH.

In an embodiment, the VL comprises: (i) an LCDR1 comprising an amino acid sequence that differs by no more than 1, 2, or 3 amino acid residues from, or has at least 85, 90, 95, 99 or 100% homology with, the amino acid sequence of the LCDR1 of the VL; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the VL; and (iii) an LCDR3 comprising an amino acid sequence that differs by no more than 1, 2, or 3 amino acid residues from, or has at least 85, 90, 95, 99 or 100% homology with, the amino acid sequence of the LCDR3 of the VL.

In an embodiment, the VL comprises: (i) an LCDR1 comprising the amino acid sequence of the LCDR1 of the VL; (ii) an LCDR2 comprising the amino acid sequence of the LCDR2 of the VL; and (iii) an LCDR3 comprising the amino acid sequence of the LCDR3 of the VL.

In an embodiment, the antibody molecule comprises:
(a) a VH comprising: (i) an HCDR1 comprising an amino acid sequence that differs by no more than 1, 2, or 3 amino acid residues from, or has at least 85, 90, 95, 99 or 100% homology with, the amino acid sequence of the HCDR1 of the VH; (ii) an HCDR2 comprising an amino acid sequence that differs by no more than 1, 2, or 3 amino acid residues from, or has at least 85, 90, 95, 99 or 100% homology with, the amino acid sequence of the HCDR2 of the VH; and (iii) an HCDR3 comprising an amino acid sequence that differs by no more than 1, 2, or 3 amino acid residues from, or has at least 85, 90, 95, 99 or 100% homology with, the amino acid sequence of the HCDR3 of the VH, and
(b) a VL comprising: (i) an LCDR1 comprising an amino acid sequence that differs by no more than 1, 2, or 3 amino acid residues from, or has at least 85, 90, 95, 99 or 100% homology with, the amino acid sequence of the LCDR1 of the VL; (ii) an LCDR2 comprising an amino acid sequence that differs by no more than 1, 2, or 3 amino acid residues from, or has at least 85, 90, 95, 99 or 100% homology with, the amino acid sequence of the LCDR2 of the VL; and (iii) an LCDR3 comprising an amino acid sequence that differs by no more than 1, 2, or 3 amino acid residues from, or has at least 85, 90, 95, 99 or 100% homology with, the amino acid sequence of the LCDR3 of the VL.

In an embodiment, the antibody molecule comprises: (a) a VH comprising: (i) an HCDR1 comprising the amino acid sequence of the HCDR1 of the VH; (ii) an HCDR2 comprising the amino acid sequence of the HCDR2 of the VH; and (iii) an HCDR3 comprising the amino acid sequence of the HCDR3 of the VH, and (b) a VL comprising: (i) an LCDR1 comprising the amino acid sequence of the LCDR1 of the VL; (ii) an LCDR2 comprising the amino acid sequence of the LCDR2 of the VL; and (iii) an LCDR3 comprising the amino acid sequence of the LCDR3 of the VL.

In an embodiment, the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of 28-0, 29-0, Ab17, Ab18, Ab29, Ab28, Ab30, Ab32, Ab34, Ab43, Ab71, Ab137, Ab173, Ab174, or Ab175. In an embodiment, the VH comprises the amino acid sequence of the VH of 28-0, 29-0, Ab17, Ab18, Ab29, Ab28, Ab30, Ab32, Ab34, Ab43, Ab71, Ab137, Ab173, Ab174, or Ab175.

In an embodiment, the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VL of 28-0, 29-0, Ab17, Ab18, Ab29, Ab28, Ab30, Ab32, Ab34, Ab43, Ab71, Ab137, Ab173, Ab174, or Ab175. In an embodiment, the VL comprises the amino acid sequence of the VL of 28-0, 29-0, Ab17, Ab18, Ab29, Ab28, Ab30, Ab32, Ab34, Ab43, Ab71, Ab137, Ab173, Ab174, or Ab175.

In an embodiment, (a) the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of 28-0, 29-0, Ab17, Ab18, Ab29, Ab28, Ab30, Ab32, Ab34, Ab43, Ab71, Ab137, Ab173, Ab174, or Ab175; and (b) the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VL of 28-0, 29-0, Ab17, Ab18, Ab29, Ab28, Ab30, Ab32, Ab34, Ab43, Ab71, Ab137, Ab173, Ab174, or Ab175.

In an embodiment, the VH comprises the amino acid sequence of the VH of 28-0, 29-0, Ab17, Ab18, Ab29, Ab28, Ab30, Ab32, Ab34, Ab43, Ab71, Ab137, Ab173, Ab174, or Ab175and the VL comprises the amino acid sequence of the VL of 28-0, 29-0, Ab17, Ab18, Ab29, Ab28, Ab30, Ab32, Ab34, Ab43, Ab71, Ab137, Ab173, Ab174, or Ab175.

In an embodiment, the antibody molecule comprises an Fc region.

In an embodiment, the antibody molecule is fucosylated. In an embodiment, the antibody molecule is afucosylated.

In an embodiment, the antibody molecule comprises: (a) a VH comprising: (i) an HCDR1 comprising the amino acid sequence of the HCDR1 of an anti-CD138 antibody described herein, e.g., chosen from antibodies 28-0, 29-0, Ab17, Ab18, Ab29, Ab28, Ab30, Ab32, Ab34, Ab43, Ab71, Ab137, Ab173, Ab174, or Ab175; (ii) an HCDR2 comprising the amino acid sequence of the HCDR2 of the anti-CD138 antibody; and (iii) an HCDR3 comprising the amino acid sequence of the HCDR3 of the anti-CD138 antibody, and (b) a VL comprising: (i) an LCDR1 comprising the amino acid sequence of the LCDR1 of the anti-CD 138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

In an embodiment, the VH comprises the amino acid sequence of the VH of the anti-CD 138 antibody and the VL comprises the amino acid sequence of the VL of the anti-CD 138 antibody.

In an embodiment, the antibody molecule comprises two VHs and two VLs.

In an embodiment, the antibody molecule is a synthetic antibody molecule. In an embodiment, the antibody molecule is an isolated antibody molecule. In an embodiment, the antibody molecule is a pH-selective antibody molecule. In an embodiment, the antibody molecule comprises one or more framework regions derived from human framework germline sequence.

In an embodiment, the antibody molecule comprises a VH region comprising one or more mutations relative to an anti-CD138 antibody described herein (e.g., antibody 28-0, 29-0, Ab17, Ab18, Ab29, Ab28, Ab30, Ab32, Ab34, Ab43, Ab71, Ab137, Ab173, Ab174, or Ab175).

In an embodiment, the antibody molecule binds to the extracellular domain of CD138. In an embodiment, the antibody molecule binds to an extracellular region of CD138 proximal to the transmembrane domain. In an embodiment, the antibody molecule is capable of binding to one or more (e.g., two, three, or all) of the following peptides: a peptide comprising the amino acid sequence of ENTAVVAVEPDRRNQSPVDQGATGASQGLLDRKEVLG (SEQ ID NO: 440), a peptide comprising the amino acid sequence of TAVVAVEPDRRNQSPVDQGATGASQ (SEQ ID NO: 441), a peptide comprising the amino acid sequence of ENTAVVAVEPDRRNQSPVDQGATG (SEQ ID NO: 442), or a peptide comprising the amino acid sequence of ENTAVVAVEPDRRNQ (SEQ ID NO: 443). In an embodiment, the antibody molecule is capable of binding to one or more (e.g., two or all) of the following peptides: a peptide comprising the amino acid sequence of ENTAVVAVEPDRRNQSPVDQGATGASQGLLDRKEVLG (SEQ ID NO: 440), a peptide comprising the amino acid sequence of RNQSPVDQGATGASQGLLDRKEVLG (SEQ ID NO: 444), or a peptide comprising the amino acid sequence of ENTAVVAVEPDRRNQ (SEQ ID NO: 443).

In an embodiment, the antibody molecule further binds to an extracellular region of CD138 distal to the transmembrane domain, e.g., a region corresponding to or proximal to the integrin binding domain (IBD) of CD138. In an embodiment, the antibody molecule is capable of binding to one or both the following peptides: a peptide comprising the amino acid sequence of ASTSTLPAGEGPKEGEAVVLPEVEPGLTAREQEA (SEQ ID NO: 10) or a peptide comprising the amino acid sequence of GEAVVLPEVEPGLTA (SEQ ID NO: 445).

In an embodiment, the antibody molecule is a synthetic antibody molecule. In an embodiment, the antibody molecule is an isolated antibody molecule. In an embodiment, the antibody molecule is a pH-selective antibody molecule. In an embodiment, the antibody molecule comprises one or more framework regions derived from human framework germline sequence.

In an embodiment, the antibody molecule is an IgG antibody. In an embodiment, the antibody molecule comprises a heavy chain constant region of IgG chosen from IgG1, IgG2, IgG3, or IgG4. In an embodiment, the antibody molecule comprises a light chain constant region of kappa or lambda light chain.

In an embodiment, the antibody molecule comprises an Fc region comprising one or more mutations to increase the binding affinity to neonatal receptor FcRn and/or the half-life of the antibody molecule. In an embodiment, the antibody molecule comprises an Fc region comprising one or more mutations described herein, e.g., to increase one or more of half-life, ADCC, CDC, or ADCP.

In an embodiment, the antibody molecule is an IgG antibody. In an embodiment, the antibody molecule comprises a heavy chain constant region of IgG chosen from IgG1, IgG2, IgG3, or IgG4. In an embodiment, the antibody molecule comprises a light chain constant region of kappa or lambda light chain.

In an embodiment, the antibody molecule comprises an Fc region comprising one or more mutations to increase the binding affinity to neonatal receptor FcRn and/or the half-life of the antibody molecule. In an embodiment, the antibody molecule comprises an Fc region comprising one or more mutations described herein, e.g., to increase one or more of half-life, ADCC, CDC, or ADCP. In an embodiment, the antibody molecule induces at least 10% (e.g., at least 15%, 20%, 25%, 30%, 35%, or 40%) greater ADCC activity relative to a reference anti-CD138 antibody (e.g., antibody BB4), e.g., as determined by a method described herein.

In an embodiment, the antibody molecule further comprises a heavy chain constant region. In an embodiment, the heavy chain constant region is an IgG1 constant region or a functional portion thereof. In another embodiment, the heavy chain constant region is an IgG2 constant region or a functional portion thereof. In an embodiment, the antibody molecule further comprises a light chain constant region. In an embodiment, the antibody molecule further comprises a heavy chain constant region and a light chain constant region. In an embodiment, the antibody molecule comprises a heavy chain constant region, a light chain constant region, and heavy and light chain variable regions of an antibody molecule described in Table 1 or 2. In certain embodiments, the antibody molecule comprises a heavy chain constant region, a light chain constant region, and variable regions that comprise one, two, three, four, five, or six CDRs of an antibody molecule described in Table 1 or 2.

Exemplary heavy chain constant regions are described below.

IgG1 HC constant region:

ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK (SEQ ID NO: 446)

IgG2 HC constant region:

ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK (SEQ ID NO: 447)

In an embodiment, the antibody molecule is derived from a parental antibody molecule described herein. In an embodiment, the parental antibody molecule comprises a VH comprising the amino acid sequence of SEQ ID NO: 471 and a VL comprising the amino acid sequence of SEQ ID NO: 475. In an embodiment, the parental antibody molecule comprises a heavy chain comprising the amino acid sequence of SEQ ID NO: 527 and a light chain comprising the amino acid sequence of SEQ ID NO: 528. The antibody molecule derived from the parental antibody molecule may have one or more of the structural and/or functional properties described herein. The antibody molecule may typically differ from the parental antibody molecule by at least 1, but no more than 2, 3, 4, 5, 6, 7, 8, 9, 10, 11, 12, 13, 14, 15, 16, 17, 18, 19, or 20 amino acids.

In an embodiment, the antibody molecule comprises the heavy chain sequence listed in Table 6 (or an amino acid sequence having at least 75%, 80%, 85%, 90%, 95%, 96%, 97%, 98%, 99%, or 100% sequence identity thereto). In an embodiment, the antibody molecule comprises a heavy chain comprising one or more (e.g., 1, 2, 3, 4, or 5) of the sequences listed in Table 7, or one or more amino acid sequences having at least 75%, 80%, 85%, 90%, 95%, 96%, 97%, 98%, 99%, or 100% sequence identity thereto. In an embodiment, the antibody molecule comprises the light chain sequence listed in Table 6 (or an amino acid sequence having at least 75%, 80%, 85%, 90%, 95%, 96%, 97%, 98%, 99%, or 100% sequence identity thereto). In an embodiment, the antibody molecule comprises a light chain comprising one or more (e.g., 1, 2, or 3) of the sequences listed in Table 8, or one or more amino acid sequences having at least 75%, 80%, 85%, 90%, 95%, 96%, 97%, 98%, 99%, or 100% sequence identity thereto.

In an embodiment, the antibody molecule comprises one, two, or all of the heavy chain constant region sequences (e.g., CH1, CH2, or CH3) listed in Table 7 (or an amino acid sequence having at least 75%, 80%, 85%, 90%, 95%, 96%, 97%, 98%, 99%, or 100% sequence identity thereto). In an embodiment, the antibody molecule comprises all of the CH1, CH2, and CH3 sequences listed in Table 7 (or an amino acid sequence having at least 75%, 80%, 85%, 90%, 95%, 96%, 97%, 98%, 99%, or 100% sequence identity thereto). In an embodiment, the antibody molecule further comprises the heavy chain constant hinge region sequence list in Table 7 (or an amino acid sequence having at least 75%, 80%, 85%, 90%, 95%, 96%, 97%, 98%, 99%, or 100% sequence identity thereto).

In an embodiment, the antibody molecule comprises the light chain constant region sequence (e.g., CL) listed in Table 8 (or an amino acid sequence having at least 75%, 80%, 85%, 90%, 95%, 96%, 97%, 98%, 99%, or 100% sequence identity thereto). In an embodiment, the antibody molecule further comprises the light chain constant hinge region sequence list in Table 8 (or an amino acid sequence having at least 75%, 80%, 85%, 90%, 95%, 96%, 97%, 98%, 99%, or 100% sequence identity thereto).

In an embodiment, the antibody molecule comprises one, two, or all of the heavy chain constant region sequences (e.g., CH1, CH2, or CH3) listed in Table 7 (or an amino acid sequence having at least 75%, 80%, 85%, 90%, 95%, 96%, 97%, 98%, 99%, or 100% sequence identity thereto) and the light chain constant region sequence (e.g., CL) listed in Table 8 (or an amino acid sequence having at least 75%, 80%, 85%, 90%, 95%, 96%, 97%, 98%, 99%, or 100% sequence identity thereto). In an embodiment, the antibody molecule comprises all of the CH1, CH2, and CH3 sequences listed in Table 7 (or an amino acid sequence having at least 75%, 80%, 85%, 90%, 95%, 96%, 97%, 98%, 99%, or 100% sequence identity thereto) and the light chain constant hinge region sequence list in Table 8 (or an amino acid sequence having at least 75%, 80%, 85%, 90%, 95%, 96%, 97%, 98%, 99%, or 100% sequence identity thereto). In an embodiment, the antibody molecule further comprises the heavy chain constant hinge region sequence list in Table 7 (or an amino acid sequence having at least 75%, 80%, 85%, 90%, 95%, 96%, 97%, 98%, 99%, or 100% sequence identity thereto) and/or the light chain constant hinge region sequence list in Table 8 (or an amino acid sequence having at least 75%, 80%, 85%, 90%, 95%, 96%, 97%, 98%, 99%, or 100% sequence identity thereto).

In an embodiment, the antibody molecule comprises one, two, or all of the amino acid sequences of SEQ ID NOs: 529, 531, or 532 (or an amino acid sequence having at least 75%, 80%, 85%, 90%, 95%, 96%, 97%, 98%, 99%, or 100% sequence identity thereto). In an embodiment, the antibody molecule comprises all of the amino acid sequences of SEQ ID NOs: 529, 531, and 532 (or an amino acid sequence having at least 75%, 80%, 85%, 90%, 95%, 96%, 97%, 98%, 99%, or 100% sequence identity thereto). In an embodiment, the antibody molecule further comprises the amino acid sequence of SEQ ID NO: 530 (or an amino acid sequence having at least 75%, 80%, 85%, 90%, 95%, 96%, 97%, 98%, 99%, or 100% sequence identity thereto).

In an embodiment, the antibody molecule comprises the amino acid sequence of SEQ ID NO: 534 (or an amino acid sequence having at least 75%, 80%, 85%, 90%, 95%, 96%, 97%, 98%, 99%, or 100% sequence identity thereto). In an embodiment, the antibody molecule further comprises the amino acid sequence of SEQ ID NO: 533 (or an amino acid sequence having at least 75%, 80%, 85%, 90%, 95%, 96%, 97%, 98%, 99%, or 100% sequence identity thereto).

In an embodiment, the antibody molecule comprises one, two, or all of the amino acid sequences of SEQ ID NOs: 529, 531, or 532 (or an amino acid sequence having at least 75%, 80%, 85%, 90%, 95%, 96%, 97%, 98%, 99%, or 100% sequence identity thereto) and the amino acid sequence of SEQ ID NO: 534 (or an amino acid sequence having at least 75%, 80%, 85%, 90%, 95%, 96%, 97%, 98%, 99%, or 100% sequence identity thereto). In an embodiment, the antibody molecule comprises all of the amino acid sequences of SEQ ID NOs: 529, 531, and 532 (or an amino acid sequence having at least 75%, 80%, 85%, 90%, 95%, 96%, 97%, 98%, 99%, or 100% sequence identity thereto) and the amino acid sequence of SEQ ID NO: 534 (or an amino acid sequence having at least 75%, 80%, 85%, 90%, 95%, 96%, 97%, 98%, 99%, or 100% sequence identity thereto). In an embodiment, the antibody molecule further comprises the amino acid sequence of SEQ ID NO: 530 (or an amino acid sequence having at least 75%, 80%, 85%, 90%, 95%, 96%, 97%, 98%, 99%, or 100% sequence identity thereto) and/or the amino acid sequence of SEQ ID NO: 533 (or an amino acid sequence having at least 75%, 80%, 85%, 90%, 95%, 96%, 97%, 98%, 99%, or 100% sequence identity thereto).

In an embodiment, the antibody molecule comprises one, two, three, four, five, or all of the amino acid sequences of SEQ ID NOs: 529-534. In an embodiment, the antibody molecule comprises the amino acid sequences of SEQ ID NOs: 529-534.

In an embodiment, the antibody molecule comprises (a) a heavy chain (HC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of SEQ ID NO: 527; and/or (b) a light chain (LC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of SEQ ID NO: 528. In an embodiment, the HC comprises the amino acid sequence of SEQ ID NO: 527 or the LC comprises the amino acid sequence of SEQ ID NO: 528.

In an embodiment, the VH of an antibody molecule described herein does not comprise the amino acid sequence of SEQ ID NO: 471. In an embodiment, the VL of an antibody molecule described herein does not comprise the amino acid sequence of SEQ ID NO: 475. In an embodiment, the VH of an antibody molecule described herein does not comprise the amino acid sequence of SEQ ID NO: 471, and the VL of an antibody molecule described herein does not comprise the amino acid sequence of SEQ ID NO: 475. In an embodiment, the heavy chain of an antibody molecule described herein does not comprise the amino acid sequence of SEQ ID NO: 527. In an embodiment, the light chain of an antibody molecule described herein does not comprise the amino acid sequence of SEQ ID NO: 528. In an embodiment, the heavy chain of an antibody molecule described herein does not comprise the amino acid sequence of SEQ ID NO: 527, and the light chain of an antibody molecule described herein does not comprise the amino acid sequence of SEQ ID NO: 528.

TABLE 6

Parental mAb 4320 heavy chain and light chain sequences

| | |
|---|---|
| | Amino Acid Sequence |
| Heavy Chain | QVQLVQSGAEVKKPGASVKVSCKASGYSFSSYYMHWVRQAPGQGLEWMG TIHPSDSTTNYNQKFQGRVTMTVDTSTRTAYMELSSLRSEDTAVYYCANFV YWGQGTTVTVSSASTKGPSVFPLAPSSKSTSGGTAALGCLVKDYFPEPVTVS WNSGALTSGVHTFPAVLQSSGLYSLSSVVTVPSSSLGTQTYICNVNHKPSNT KVDKRVEPKSCDKTHTCPPCPAPELLGGPSVFLFPPKPKDTLMISRTPEVTCV VVDVSHEDPEVKFNWYVDGVEVHNAKTKPREEQYNSTYRVVSVLTVLHQD WLNGKEYKCKVSNKALPAPIEKTISKAKGQPREPQVYTLPPSREEMTKNQVS LTCLVKGFYPSDIAVEWESNGQPENNYKTTPPVLDSDGSFFLYSKLTVDKSR WQQGNVFSCSVMHEALHNHYTQKSLSLSPGK (SEQ ID NO: 527) |
| Light Chain | DIVMTQTPLSLSVTPGQPASISCKSSKSLLYKDGKTYLNWFLQKPGQSPQLLI YVVSTRASGVPDRFSGSGSGTDFTLKISRVEAEDVGVYYCQQLVEYPYTFGQ GTKLEIKRTVAAPSVFIFPPSDEQLKSGTASVVCLLNNFYPREAKVQWKVDN ALQSGNSQESVTEQDSKDSTYSLSSTLTLSKADYEKHKVYACEVTHQGLSSP VTKSFNRGEC (SEQ ID NO: 528) |

TABLE 7

Annotation of parental mAb 4320 heavy chain sequence

| Feature | Position | Sequence |
|---|---|---|
| Leader Sequence | -- | N/A |
| Variable (VH) | 1-112 | QVQLVQSGAEVKKPGASVKVSCKASGYSFSSYYMHWVRQAPGQGLEWMG TIHPSDSTTNYNQKFQGRVTMTVDTSTRTAYMELSSLRSEDTAVYYCAN FVYWGQGTTVTVSS (SEQ ID NO: 471) |
| Constant-CH1 | 113-210 | ASTKGPSVFPLAPSSKSTSGGTAALGCLVKDYFPEPVTVSWNSGALTSG VHTFPAVLQSSGLYSLSSVVTVPSSSLGTQTYICNVNHKPSNTKVDKRV (SEQ ID NO: 529) |
| Constant-Hinge | 211-225 | EPKSCDKTHTCPPCP(SEQ ID NO: 530) |
| Constant-CH2 | 226-335 | APELLGGPSVFLFPPKPKDTLMISRTPEVTCVVVDVSHEDPEVKFNWYV DGVEVHNAKTKPREEQYNSTYRVVSVLTVLHQDWLNGKEYKCKVSNKAL PAPIEKTISKAK (SEQ ID NO: 531) |
| Constant-CH3 | 336-442 | GQPREPQVYTLPPSREEMTKNQVSLTCLVKGFYPSDIAVEWESNGQPEN NYKTTPPVLDSDGSFFLYSKLTVDKSRWQQGNVFSCSVMHEALHNHYTQ KSLSLSPGK(SEQ ID NO: 532) |

CDRs in variable region (VH) and as defined by Chothia are underlined. Heavy chain constant region is Homo sapiens immunoglobulin heavy constant gamma 1 (m3 allotype). Any of the anti-CD 138 antibody molecules described herein (e.g., antibodies 28-0, 29-0, Ab17, Ab18, Ab29, Ab28, Ab30, Ab32, Ab34, Ab43, Ab71, Ab137, Ab173, Ab174, or Ab175) can comprise the constant CH1, hinge, CH2, and/or CH3 sequences described in Table 7.

TABLE 8

Annotation of mAb 4320 light chain sequence

| Feature | Position | Sequence |
|---|---|---|
| Leader Sequence | -- | N/A |
| Variable (VK) | 1-112 | DIVMTQTPLSLSVTPGQPASISCKSSKSLLYKDGKTYLNWFLQKPGQSP QLLIYVVSTRASGVPDRFSGSGSGTDFTLKISRVEAEDVGVYYCQQLVE YPYTFGQGTKLEIK(SEQ ID NO: 475) |
| Constant-Hinge | 113-118 | RTVAAP(SEQ ID NO: 533) |
| Constant-CL | 119-219 | SVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNR GEC(SEQ ID NO: 534) |

CDRs in variable region (VL) and as defined by Chothia are underlined. Light chain is Homo sapiens kappa constant*01. Any of the humanized anti-CD138 antibody molecules described herein (e.g., antibodies 28-0, 29-0, Ab17, Ab18, Ab29, Ab28, Ab30, Ab32, Ab34, Ab43, Ab71, Ab137, Ab173, Ab174, or Ab175) can comprise the constant CL and/or hinge sequences described in Table 8.

In an embodiment, the antibody molecule is a multivalent (e.g., bivalent, trivalent, or tetravalent) antibody molecule. In an embodiment, the antibody molecule binds to two or more (e.g., three or four) different regions in CD138. For example, the antibody molecule can comprise two or more sets of identical, or substantially identical, VH-VL pairs, wherein each VH-VL pair binds to two or more different regions in CD138. As another example, the antibody molecule can comprise two or more sets of different VH-VL pairs, wherein each VH-VL pair binds to a different region in CD138.

In an embodiment, the antibody molecule is a multispecific (e.g., bispecific, trispecific, or tetraspecific) antibody molecule. In an embodiment, the antibody molecule has a first binding specificity to CD138 and a second binding specificity other than CD138. For example, the antibody molecule can comprise two or more sets of identical, or substantially identical, VH-VL pairs, wherein each VH-VL pair has both the first binding specificity and the second binding specificity. As another example, the antibody molecule can comprise two or more sets of different VH-VL pairs, wherein each VH-VL pair has a different binding specificity.

In an embodiment, the antibody molecule is at least 10%, 20%, 30%, 40%, 50%, 60%, 70%, 80%, or 90%, or at least 1, 2, 3, 4, 5, 6, 7, 8, 9, or 10-fold, more stable in vitro, than a reference antibody molecule, e.g., a related non-pH-selective antibody molecule, e.g., as determined by a method described herein. In an embodiment, the antibody molecule has a first thermal transition temperature of at least about 62° C., e.g., between about 65° C. and about 70° C. (e.g., at about 65° C., 66° C., 67° C., 68° C., 69° C., or 70° C.), e.g., as determined by differential scanning fluorescence (DSF). In an embodiment, the antibody molecule has a second thermal transition temperature of at least about 70° C., e.g., between about 75° C. and about 80° C. (e.g., at about 75° C., 76° C., 77° C., 78° C., 79° C., or 80° C.), e.g., as determined by DSF.

In an embodiment, the antibody molecule is at least 10%, 20%, 30%, 40%, 50%, 60%, 70%, 80%, or 90%, or at least 1, 2, 3, 4, 5, 6, 7, 8, 9, or 10-fold, more stable in vivo, than a reference antibody molecule, e.g., a related non-pH-selective antibody molecule, as determined by a method described herein. In an embodiment, the antibody molecule has a serum half-life (in human or in an animal model) that is at least 10%, 20%, 30%, 40%, 50%, 60%, 70%, 80%, or 90%, or at least 1, 2, 3, 4, 5, 6, 7, 8, 9, or 10-fold, higher than a related non-pH-selective antibody molecule, e.g., as determined by ELISA.

### Antibody Molecule-Drug Conjugates

As used herein, the term “antibody molecule-drug conjugate” or ADC refers to an antibody molecule that is coupled to a non-antibody moiety, e.g., a therapeutic agent or label, e.g., a cytotoxic agent. The antibody molecule can be coupled to the non-antibody moiety directly, or indirectly, e.g., through a linker.

In an embodiment, the antibody molecule is coupled to the non-antibody moiety by a covalent bond. In an embodiment, the antibody molecule is coupled to the non-antibody moiety by a peptide bond. In an embodiment, the antibody molecule is coupled to the non-antibody moiety by a non-peptide bond. In an embodiment, the antibody molecule is not coupled to the non-antibody moiety by a non-peptide bond. In an embodiment, a non-antibody moiety is also referred to as a “payload.”

In an embodiment, the non-antibody moiety is coupled to the backbone of the antibody molecule. In another embodiment, the non-antibody moiety is coupled to a side chain of the antibody molecule. In an embodiment, two or more (e.g., three, four, five, six, seven, eight, or more) non-antibody moieties are coupled to the antibody molecule.

In an embodiment, the ADC comprises an antibody molecule that binds to CD138, e.g., an anti-CD138 antibody molecule described herein (e.g., pH-selective anti-CD138 antibody molecules described herein).

In an embodiment, the ADC comprises one, two, or three CDRs of the VH region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175), using the Kabat or Chothia definitions of CDRs. In an embodiment, the ADC comprises one, two, or three CDRs of the VL region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175), using the Kabat or Chothia definitions of CDRs. In an embodiment, the ADC comprises one or more (e.g., two or three) CDRs of the VH region and/or one or more (e.g., two or three) CDRs of the VL region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175), using the Kabat or Chothia definitions of CDRs.

In an embodiment, the ADC comprises one, two, or three VH CDRs described in Table 1 or 2. In an embodiment, the ADC comprises one, two, or three VL CDRs described in Table 1 or 2. In an embodiment, the ADC comprises one or more (e.g., two or three) VH CDRs and/or one or more (e.g., two or three) VL CDRs described in Table 1 or 2.

In an embodiment, the ADC comprises one, two, three, or four frameworks of the VH region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175). In an embodiment, the ADC comprises one, two, three, or four frameworks of the VL region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175). In an embodiment, the ADC comprises one or more (e.g., two, three, or four) frameworks of the VH region and/or one or more (e.g., two, three, or four) frameworks of the VL region 28-0, 29-0, Ab17, Ab18, Ab29, Ab28, Ab30, Ab32, Ab34, Ab43, Ab71, Ab137, Ab173, Ab174, or Ab175).

In an embodiment, the ADC comprises a heavy chain variable region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175). In an embodiment, the ADC comprises a light chain variable region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175). In an embodiment, the ADC comprises a heavy chain variable region and a light chain variable region of an antibody molecule described in Table 1 or 2 (e.g., any of antibodies 28-0, 29-0, Ab17, Ab18, Ab29, Ab28, Ab30, Ab32, Ab34, Ab43, Ab71, Ab137, Ab173, Ab174, or Ab175).

In an embodiment, the ADC comprises a heavy chain variable region having an amino acid sequence described in Table 1 or 2. In an embodiment, the ADC comprises a light chain variable region having an amino acid sequence described in Table 1 or 2. In an embodiment, the ADC comprises a heavy chain variable region having an amino acid sequence described in Table 1 or 2 and a light chain variable region having an amino acid sequence described in Table 1 or 2.

In an embodiment, the antibody molecule comprises a heavy chain variable region encoded by a nucleotide sequence described in Table 2. In an embodiment, the antibody molecule comprises a light chain variable region encoded by a nucleotide sequence described in Table 2. In an embodiment, the antibody molecule comprises a heavy chain variable region encoded by a nucleotide sequence described in Table 2 and a light chain variable region encoded by a nucleotide sequence described in Table 2.

In an embodiment, the ADC comprises a heavy chain constant region. In an embodiment, the ADC comprises a light chain constant region. In an embodiment, the ADC comprises a heavy chain constant region and a light chain constant region. In an embodiment, the ADC comprises a heavy chain constant region, a light chain constant region, and heavy and light chain variable regions of an antibody molecule described in Table 1 or 2. In certain embodiments, the ADC comprises a heavy chain constant region, a light chain constant region, and variable regions that comprise one, two, three, four, five, or six CDRs of antibody molecule described in Table 1 or 2.

In an embodiment, the ADC comprises one, two, or all of the heavy chain constant region sequences (e.g., CH1, CH2, or CH3) listed in Table 7. In an embodiment, the ADC comprises all of the CH1, CH2, and CH3 sequences listed in Table 7. In an embodiment, the ADC further comprises the heavy chain constant hinge region sequence list in Table 7. In an embodiment, the ADC comprises the light chain constant region sequence (e.g., CL) listed in Table 8. In an embodiment, the ADC further comprises the light chain constant hinge region sequence list in Table 8.

In an embodiment, the ADC comprises one, two, or all of the heavy chain constant region sequences (e.g., CH1, CH2, or CH3) listed in Table 7 and the light chain constant region sequence (e.g., CL) listed in Table 8. In an embodiment, the ADC comprises all of the CH1, CH2, and CH3 sequences listed in Table 7 and the light chain constant hinge region sequence list in Table 8. In an embodiment, the ADC further comprises the heavy chain constant hinge region sequence list in Table 7 and/or the light chain constant hinge region sequence list in Table 8.

In an embodiment, the ADC comprises a heavy chain comprising an amino acid sequence described in Table 6 or 8. In an embodiment, the ADC comprises a light chain comprising an amino acid sequence described in Table 6 or 7. In an embodiment, the ADC comprises a heavy chain comprising an amino acid sequence described in Table 6 or 8 and a light chain comprising an amino acid sequence described in Table 6 or 7.

In an embodiment, the non-antibody molecule comprises a cytotoxic agent (e.g., any cytotoxic agent that is active against a cancer). In an embodiment, the cytotoxic agent is chosen from a tubulin polymerase inhibitor (e.g., an auristatin), an agent associated with tubulin depolymerization (e.g., a maytansine), an agent associated with DNA cleavage (e.g., a calicheamicin), a DNA minor groove alkylating agent (e.g., a duocarymycin), a DNA minor groove cross-linker (e.g., a PBD dimers), or an RNA polymerase II inhibitor (e.g., α-amanitin).

In an embodiment, the cytotoxic agent is α-amanitin. α-amanitin is a bicyclic octapeptide which belongs to a large group of protoplasmic mushroom toxins known as amatoxins. α-Amanitin binds to the bridging helix of RNA polymerase II inhibiting the translocation of RNA and DNA needed to empty the site for the next round of synthesis, thereby reducing the rate of transcription. α-amanitin and its use in ADCs are described, e.g., in Moldenhauer et al. J Natl Cancer Inst. 2012; 104(8): 622-634. The structure of α-amanitin is as follows:

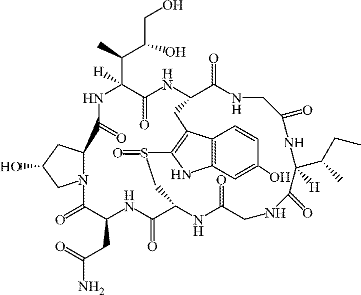 [SMILES: CC[C@H](C)[C@@H]1NC(=O)CNC(=O)[C@@H]2Cc3c([nH]c4cc(O)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]

In an embodiment, the cytotoxic agent is a cryptophycin analog. The cryptophycins are a group of cyanobacterial depsipeptides with a remarkable biological activity against multi-drug-resistant (MDR) cancer cells. Cryptophycins deplete microtubules through interaction with tubulin, thereby preventing cell division. They are capable of inducing apoptosis, possibly through other mechanisms in addition to that mediated by microtubule inhibition. Cryptophycin, analogues, and their uses in ADCs are described, e.g., in Shih & Teicher. Curr Pharm Des. 2001; 7(13): 1259-1276; Eggen & Georg. Med Res Rev. 2002; 22(2): 85-101. The structure of a cryptophycin analog is as follows:

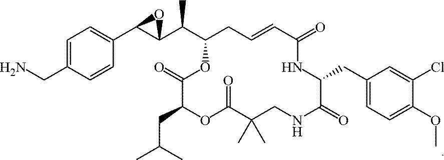

In an embodiment, the cytotoxic agent is calicheamicin (also known as LL-E33288). Calicheamicin contacts DNA and causes the Bergman cyclization, which results in cleaving the DNA and thus destroying cells. Calicheamicin and its use in ADCs is described, e.g., in Maiese et al. J Antibiot (Tokyo). 1989; 42(4): 558-563; Watanabe et al. Chem Biol. 2002; 9(2): 245-251; Ricart & Tolcher. Nat Clin Pract Oncol. 2007; 4: 245-255. The structure of calicheamicin is as follows.

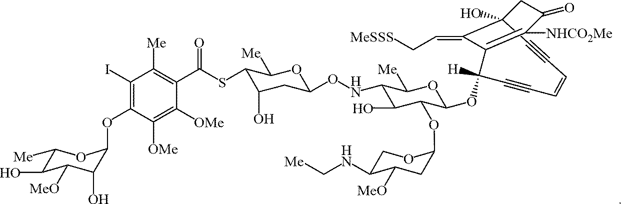

In an embodiment, the cytotoxic agent is centanamycin. Centanamycin is also known as ML-970, AS-I-145, NSC 716970, or N-[4-Amino-1-(2-chloroethyl)-2-naphthyl]-5,6,7-trimethoxy-1H-indole-2-carboxamide). Centanamycin binds the A-T-rich DNA minor groove and alkylates DNA. Centanamycin and its use in ADCs is described, e.g., in Rayburn et al. Cancer Chemother Pharmacol. 2012; 69(6): 1423-31.

In an embodiment, the cytotoxic agent is a dolastatin. In an embodiment, the dolastatin is dolastatin 10 or dolastatin 15. Dolastatins noncompetitively inhibit binding of vincristine to tubulin at the vinca/peptide region). Analogues of dolastatins include, e.g., symplostatin 1, symplostatin 3, and auristatin. Dolastatins, analogues, and their uses are described, e.g., in Amador et al. Annals of Oncology. 2003; 14: 1607-1615; Kijjoa & Sawangwong. Mar Drugs. 2004; 2(2): 73-82; Luesch et al. J NatProd. 2001; 64(7): 907-910; Luesch et al. J Nat Prod. 2002; 65(1): 16-20. The structure of dolastatin 10 is as follows:

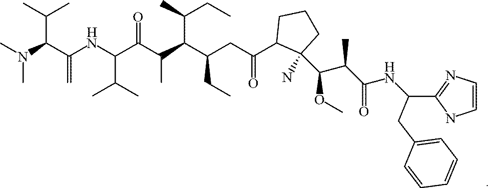

The structure of dolastatin 15 is as follows:

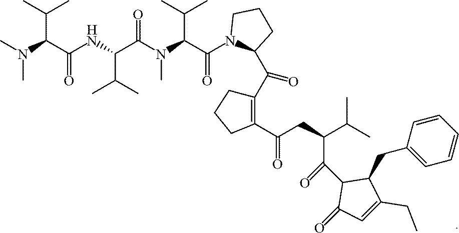

In an embodiment, the cytotoxic agent is a duocarmycin analogue. Duocarmycin analogues are DNA minor groove, AT-sequence selective, and adenine-N3 alkylating agents. Duocarmycin, analogues, and their uses in ADCs are described, e.g., in Tietze & Krewer. Chem Biol Drug Des. 2009; 74(3):205-211; Cacciari et al. Expert Opinion on Therapeutic Patents. 2000; 10 (12): 1853-1871; Tercel et al. Angew Chem Int Ed Engl. 2013; 52(21): 5442-5446. Exemplary duocarmycin and analogues include, e.g., duocarmycin A, duocarmycin B1, duocarmycin B2, duocarmycin C1, duocarmycin C2, duocarmycin D, duocarmycin SA, and CC-1065. The structure of duocarymycin A is as follows:

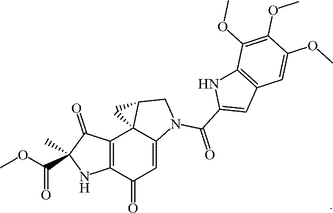

In an embodiment, the cytotoxic agent is maytansine. Maytansine, a benzoansamacrolide, is a highly potent microtubule-targeted compound that induces mitotic arrest and kills tumor cells at subnanomolar concentrations. Maytansine and its analogs (maytansinoids DM1 and DM4) are potent microtubule-targeted compounds that inhibit proliferation of cells at mitosis. Maytansine is described, e.g., in Lopus et al. Mol Cancer Ther. 2010; 9(10): 2689-2699; Widdison et al. J Med Chem. 2006; 49(14): 4392-4408; Liu et al. J Mass Spectrom. 2005; 40(3): 389-399; Tassone et al. Cancer Res. 2004; 64(13): 4629-4636; Sawada et al. Bioconjug Chem. 1993; 4(4):284-289. The structure of maytansine is as follows:

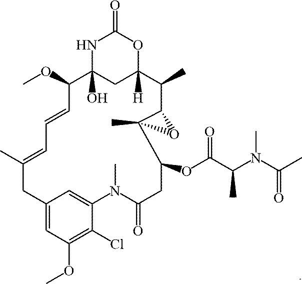

In an embodiment, the cytotoxic agent is monomethyl auristatin E (MMAE, vedotin). MMAE is a highly potent antimitotic agent that inhibits cell division by blocking the polymerization of tubulin. MMAE and its use in ADCs are described, e.g., in Francisco et al. Blood. 2003; 102(4): 1458-1465; Junutula et al. Nat Biotechnol. 2008; 26(8):925-932; Asundi et al. Clin Cancer Res. 2011; 17(5): 965-975; Younes et al. J Clin Oncol. 2012; 30(18):2183-2189; Pettit et al. Anticancer Drug Des. 1995; 10(7): 529-544; Doronina et al. Nat Biotechnol. 2003; 21(7): 778-784. The structure of MMAE is as follows:

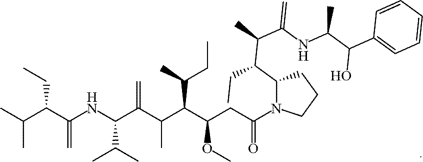

In an embodiment, the cytotoxic agent is monomethyl auristatin F (MMAF). MMAF is an antitubulin agent that inhibits cell division by blocking the polymerization of tubulin. It is an auristatin derivative with a charged C-terminal phenylalanine that attenuates its cytotoxic activity compared to its uncharged counterpart, monomethyl auristatin E (MMAE). MMAF can induce potent antitumor effects when conjugated via protease cleavable linkers to a monoclonal antibody targeting internalizing, tumor-specific cell surface antigens. For example, the linker to the monoclonal antibody is stable in extracellular fluid, but can be cleaved by cathepsin once the conjugate has entered a tumor cell, thus activating the anti-mitotic mechanism. MMAF and its use in ADCs are described, e.g., in Smith et al. Mol Cancer Ther. 2006 5; 1474-1482; Doronina et al., Bioconjug Chem. 2006; 17(1):114-24; Oflazoglu et al. Clin Cancer Res. 2008; 14(19): 6171-6180; Nilsson et al. Cancer. 2010; 116(4 Suppl): 1033-1042. The structure of MMAF is as follows:

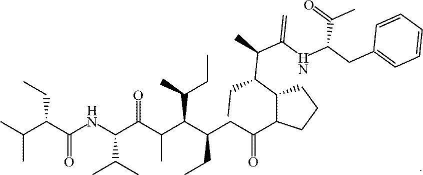

In an embodiment, the cytotoxic agent is a pyrrolobenzodiazepine (PBD). PBDs are a class of sequence-selective DNA minor-groove binding crosslinking agents. The mechanism of action of the PBDs is associated with their ability to form an adduct in the minor groove, thus interfering with DNA processing. Exemplary agents that belong to the pyrrolobenzodiazepine antibiotic group include, but are not limited to, anthramycin. abbeymycin, chicamycin, DC-81, mazethramycin, neothramycin A, neothramycin B, porothramycin, prothracarcin, sibanomicin (DC-102), sibiromycin, and tomamycin. PBDs and their use in ADCs are described, e.g., in Antonow & Thurston DE. Chem Rev. 2011; 111: 2815-2864; Cipolla et al. Anticancer Agents Med Chem. 2009; 9: 1-31; Gerratana. Med Res Rev. 2012; 32: 254-293; Li et al. Appl Environ Microbiol. 2009; 75(9):2869-2878; Rahman et al. Org. Biomol. Chem. 2011; 9: 1632-1641; Saunders et al. Sci Transl Med. 2015; 7(302): 302ra136; Hu et al. Chem Biol. 2007; 14(6):691-701. The structure of PBD is as follows:

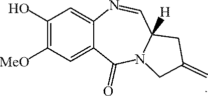

In an embodiment, the ADC further comprises a linker, e.g., a linker that couples an antibody molecule to a non-antibody moiety. In an embodiment, the linker comprises a hydrazone, a disulfide bond, a peptide, or a thioether bond.

In an embodiment, the linker is a non-cleavable linker. Exemplary non-cleavable linkers include, e.g., a non-cleavable thioether linker (e.g., N-succinimidyl-4-(N-maleimidomethyl)cyclohexane-1-carboxylate (SMCC)) or a non-cleavable maleimidocaproyl linker.

In an embodiment, the liner is a cleavable linker. In an embodiment, the cleavable linker is a chemically labile linker, e.g., an acid-cleavable linker (e.g., an acid-cleavable hydrazone) or a reducible linker (e.g., a disulfide linker). In an embodiment, the cleavable linker is an enzyme cleavable linker, e.g., a peptide-based linker (e.g., a dipeptide linker (e.g., a valine-citrulline (Val-Cit) linker or a phenylalanine-lysine (Phe-Lys) dipeptide linker)) or a β-glucuronide linker. Other linkers and their use in ADCs are described, e.g., in Lu et al. Int J Mol Sci. 2016; 17(4): 561, the content of which is incorporated by reference in its entirety.

In an embodiment, the linker is a poly(ethylene glycol) (PEG) linker.

### Animal Models

The anti-CD138 antibody molecules described herein (e.g., pH-selective anti-CD138 antibody molecules described herein) can be evaluated in vivo, e.g., using various animal models. For example, an animal model can be used to test the efficacy of an antibody molecule described herein in inhibiting CD138 and/or in treating or preventing a disorder described herein, e.g., a myeloma (e.g., multiple myeloma). Animal models can also be used, e.g., to investigate for side effects, measure concentrations of antibody molecules in situ, demonstrate correlations between a CD138 function and a disorder described herein, e.g., a myeloma (e.g., multiple myeloma). Exemplary types of animals that can be used to evaluate the antibody molecules described herein include, but are not limited to, mice, rats, rabbits, guinea pigs, and monkeys.

Exemplary animal models for myelomas (e.g., multiple myeloma) that can be used for evaluating an antibody molecule described herein include, but are not limited to, immunocompetent murine models, e.g., 5TMM (5T Radl), 5T2, 5T33, and 5TGMA models (Radl et al. Am J Pathol. 1988; 132: 593-597); immunocompromised murine models, e.g., RAG-2 model (Fowler et al. Dis Model Mech. 2009; 2: 604-611), xenograft murine myeloma models, e.g., SCID and NOD/SCID models (Huang et al. Cancer Res. 1993; 53: 1392-1396; Tsunenari et al. Blood. 1997; 90: 2437-2444; Torcia et al. Exp Hematol. 1996; 24: 868-874; Hjorth-Hansen et al. J Bone Miner Res. 1999; 14: 256-263); SCID-Hu and SCID-Rab models (Urashima et al. Blood. 1997; 90: 754-765; Yaccoby et al. Blood. 1998; 92: 2908-2913; Yata & Yaccoby. Leukemia. 2004; 18: 1891-1897); genetically engineered models, e.g., IL-6- and MYC-driven models (Kovalchuk et al. Proc Natl Acad Sci USA. 2002; 99: 1509-1514; Adams et al. Nature. 1985; 318: 533-538; Chesi et al. Blood. 2012; 120: 376-385); Eµ-xbp-1s model (Carrasco et al. Cancer Cell. 2007; 11(4):349-360); L-GP130 model (Dechow et al. J Clin Invest. 2014; 124(12): 5263-5274).

Various murine and human myeloma cell lines and primary human myeloma cells can be used in preclinical in vivo models. Exemplary murine and human myeloma cell lines that can be used for engraftment include, but are not limited to, 5T myeloma cells (Radl et al. Am J Pathol. 1988; 132: 593-597), human lymphoblastoid ARH-77 cells (Huang et al. Cancer Res. 1993; 53(6):1392-1396), the human JJN3 myeloma cell line (Hjorth-Hansen et al. J Bone Miner Res. 1999; 14(2): 256-263), and IL-6-dependent myeloma cell lines (Tsunenari et al. Blood. 1997; 90(6):2437-2444). A desired cell line can be selected based on, e.g., the pace of tumor engraftment, characteristics of the particular tumor type (e.g., propensity to develop lytic bone lesions), or the type of monoclonal protein that is produced.

Other animal models for myelomas (e.g., multiple myeloma) are described, e.g., in Lwin et al. Bonekey Rep. 2016; 5: 772; Libouban et al. Morphologie. 2015; 99(325): 63-72; Campbell et al. Curr Protoc Pharmacol. 2008; Chapter 14: Unit 14.9.

### Pharmaceutical Compositions and Kits

In an aspect, this disclosure provides compositions, e.g., pharmaceutically acceptable compositions, which include an anti-CD138 antibody molecule described herein (e.g., pH-selective anti-CD138 antibody molecules described herein) or an ADC comprising an anti-CD138 antibody molecule described herein (e.g., pH-selective anti-CD138 antibody molecules described herein), formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). In certain embodiments, less than about 5%, e.g., less than about 4%, 3%, 2%, or 1% of the antibody molecules in the pharmaceutical composition are present as aggregates. In other embodiments, at least about 95%, e.g., at least about 96%, 97%, 98%, 98.5%, 99%, 99.5%, 99.8%, or more of the antibody molecules in the pharmaceutical composition are present as monomers. In an embodiment, the level of aggregates or monomers is determined by chromatography, e.g., high performance size exclusion chromatography (HP-SEC).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In an embodiment, the antibody molecule is administered by intravenous infusion or injection. In certain embodiments, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules described herein can be administered by a variety of methods. Several are known in the art, and for many therapeutic, prophylactic, or diagnostic applications, an appropriate route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, an antibody molecule can be orally administered, for example, with an inert diluent or an assimilable edible carrier. The antibody molecule (and other ingredients, if desired) may also be enclosed in a hard or soft shell gelatin capsule, compressed into tablets, or incorporated directly into the subject’s diet. For oral therapeutic administration, the antibody molecule may be incorporated with excipients and used in the form of ingestible tablets, buccal tablets, troches, capsules, elixirs, suspensions, syrups, wafers, and the like. To administer an antibody molecule by other than parenteral administration, it may be necessary to coat the compound with, or co-administer the compound with, a material to prevent its inactivation. Therapeutic, prophylactic, or diagnostic compositions can also be administered with medical devices, and several are known in the art.

Dosage regimens are adjusted to provide the desired response (e.g., a therapeutic, prophylactic, or diagnostic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the antibody molecule and the particular therapeutic, prophylactic, or diagnostic effect to be achieved, and (b) the limitations inherent in the art of compounding such an antibody molecule for the treatment of sensitivity in individuals.

An exemplary, non-limiting range for a therapeutically, prophylactically, or diagnostically effective amount of an antibody molecule is about 0.1-50 mg/kg body weight of a subject, e.g., about 0.1-30 mg/kg, e.g., about 1-30, 1-15, 1-10, 1-5, 5-10, or 1-3 mg/kg, e.g., about 1, 2, 3, 4, 5, 6, 7, 8, 9, 10, 15, 20, 30, 40, or 50 mg/kg. The antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², e.g., about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount,” “prophylactically effective amount,” or “diagnostically effectively amount” of an antibody molecule described herein.

A “therapeutically effective amount” refers to an amount effective, at dosages and for periods of time necessary, to achieve the desired therapeutic result. A therapeutically effective amount of the antibody molecule may vary according to factors such as the disease state, age, sex, and weight of the individual, and the ability of the antibody or antibody portion to elicit a desired response in the individual. A therapeutically effective amount is also one in which any toxic or detrimental effect of the antibody molecule is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody molecule to inhibit a measurable parameter can be evaluated in an animal model system predictive of efficacy in treating or preventing a myeloma. Alternatively, this property of a composition can be evaluated by examining the ability of the antibody molecule to inhibit CD138, e.g., by an in vitro assay.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder, e.g., a disorder described herein, e.g., A myeloma, can be diagnosed in vitro, ex vivo, or in vivo.

Also within this disclosure is a kit that comprises an antibody molecule, described herein. The kit can include one or more other elements including: instructions for use; other reagents, e.g., a label, a therapeutic agent, or an agent useful for chelating, or otherwise coupling, an antibody molecule to a label or therapeutic agent, or a radioprotective composition; devices or other materials for preparing the antibody molecule for administration; pharmaceutically acceptable carriers; and devices or other materials for administration to a subject.

### Nucleic Acids

The present disclosure also features nucleic acids comprising nucleotide sequences that encode the anti-CD138 antibody molecules (e.g., pH-selective anti-CD138 antibody molecules described herein, e.g., heavy and light chain variable regions and CDRs of the antibody molecules), as described herein.

For example, the present disclosure features a first and second nucleic acid encoding heavy and light chain variable regions, respectively, of an antibody molecule chosen from one or more of the antibody molecules disclosed herein, e.g., an antibody molecule of Table 1, 2, or 6, or a portion of an antibody molecule, e.g., the variable regions of Table 1 or 2. The nucleic acid can comprise a nucleotide sequence encoding any one of the amino acid sequences in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein).

In certain embodiments, the nucleic acid can comprise a nucleotide sequence encoding at least one, two, or three CDRs from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, or three CDRs from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, three, four, five, or six CDRs from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding a heavy chain variable region and a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding a heavy chain having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding a light chain having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding a heavy chain and a light chain having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In certain embodiments, the nucleic acid can comprise a nucleotide sequence encoding at least one, two, or three CDRs from a heavy chain variable region having the nucleotide sequence as set forth in Table 2, a sequence substantially homologous thereto (e.g., a sequence at least about 85%, 90%, 95%, 99% or more identical thereto, and/or capable of hybridizing under the stringency conditions described herein). In an embodiment, the nucleic acid can comprise a nucleotide sequence encoding at least one, two, or three CDRs from a light chain variable region having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein). In certain embodiments, the nucleic acid can comprise a nucleotide sequence encoding at least one, two, three, four, five, or six CDRs from heavy and light chain variable regions having the nucleotide sequence as set forth in Table 2, or a sequence substantially homologous thereto (e.g., a sequence at least about 85%, 90%, 95%, 99% or more identical thereto, and/or capable of hybridizing under the stringency conditions described herein).

In certain embodiments, the nucleic acid comprises a nucleotide sequence as set forth in Table 2 or a sequence substantially homologous thereto (e.g., a sequence at least about 85%, 90%, 95%, 99% or more identical thereto, and/or capable of hybridizing under the stringency conditions described herein). In an embodiment, the nucleic acid comprises a portion of a nucleotide sequence as set forth in Table 2 or a sequence substantially homologous thereto (e.g., a sequence at least about 85%, 90%, 95%, 99% or more identical thereto, and/or capable of hybridizing under the stringency conditions described herein). The portion may encode, for example, a variable region (e.g., VH or VL); one, two, or three or more CDRs; or one, two, three, or four or more framework regions.

The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In an aspect, the application features host cells and vectors containing the nucleic acids described herein. The nucleic acids may be present in a single vector or separate vectors present in the same host cell or separate host cell, as described in more detail below.

### Vectors

Further provided herein are vectors that comprise nucleotide sequences encoding an anti-CD 138 antibody molecule described herein (e.g., a pH-selective anti-CD138 antibody molecule described herein).

In an embodiment, the vector comprises a nucleotide sequence encoding an antibody molecule described herein, e.g., as described in Table 1, 2, or 6. In another embodiment, the vector comprises a nucleotide sequence described herein, e.g., in Table 2. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

The present disclosure also provides cells (e.g., host cells) comprising a nucleic acid encoding an anti-CD138 antibody molecule described herein (e.g., a pH-selective anti-CD138 antibody molecule described herein). For example, the host cells may comprise a nucleic acid molecule having a nucleotide sequence described in Table 2, a sequence substantially homologous thereto (e.g., a sequence at least about 85%, 90%, 95%, 99% or more identical thereto, and/or capable of hybridizing under the stringency conditions described herein), or a portion of one of said nucleic acids. Additionally, the host cells may comprise a nucleic acid molecule encoding an amino acid sequence of Table 2, a sequence substantially homologous thereto (e.g., a sequence at least about 80%, 85%, 90%, 95%, 99% or more identical thereto), or a portion of one of said sequences.

In an embodiment, the host cells are genetically engineered to comprise nucleic acids encoding the antibody molecule described herein.

In certain embodiments, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. In an embodiment, the cell (e.g., host cell) is an isolated cell.

### Uses of Antibody Molecules

The anti-CD138 antibody molecules (e.g., pH-selective anti-CD138 antibody molecules described herein) disclosed herein, as well as the pharmaceutical compositions disclosed herein, have in vitro, ex vivo, and in vivo therapeutic, prophylactic, and/or diagnostic utilities.

In an embodiment, the antibody molecule causes (e.g., induces or increases) an effector function on a cell expressing CD138. For example, the antibody molecules can be administered to a subject, e.g., a human subject, to cause an antibody-dependent cellular cytotoxicity activity on a diseased cell (e.g., a cancer cell or a precancerous cell) that it binds to. In an embodiment, the antibody molecule causes a complement-dependent cytotoxicity activity on a cell expressing CD138. In an embodiment, the antibody molecule reduces (e.g., inhibits, blocks, or neutralizes) one or more biological activities of a cell expressing CD138. In an embodiment, the antibody molecule inhibits the action of a protease on a membrane-bound CD138, e.g., to reduce shedding of CD138. For example, these antibodies molecules can be administered to cells in culture, in vitro or ex vivo, or to a subject, e.g., a human subject, e.g., in vivo, to reduce (e.g., inhibits, blocks, or neutralizes) one or more biological activities of the cell.

Accordingly, in an aspect, the disclosure provides a method of treating, preventing, or diagnosing a disorder, e.g., a disorder described herein (e.g., multiple myeloma), in a subject, comprising administering to the subject an anti-CD138 antibody molecule described herein, such that the disorder is treated, prevented, or diagnosed. For example, the disclosure provides a method comprising contacting the antibody molecule described herein with cells in culture, e.g. in vitro or ex vivo, or administering the antibody molecule described herein to a subject, e.g., in vivo, to treat, prevent, or diagnose a disorder, e.g., a disorder associated with CD138 (e.g., multiple myeloma).

As used herein, the term “subject” is intended to include human and non-human animals. In an embodiment, the subject is a human subject, e.g., a human patient having a disorder described herein (e.g., multiple myeloma), or at risk of having a disorder described herein (e.g., multiple myeloma). The term “non-human animals” includes mammals and non-mammals, such as non-human primates. In an embodiment, the subject is a human. The methods and compositions described herein are suitable for treating human patients a disorder described herein (e.g., multiple myeloma). Patients having a disorder described herein include, e.g., those who have developed a disorder described herein but are (at least temporarily) asymptomatic, patients who have exhibited a symptom of a disorder described herein, and patients having a disorder related to or associated with a disorder described herein.

### Methods of Treating or Preventing Disorders

The antibody molecules described herein (e.g., pH-selective anti-CD138 antibody molecules described herein) can be used to treat or prevent disorders associated with CD138 or symptoms thereof.

Exemplary disorders or conditions that can be associated with CD138 include, but are not limited to cancer (e.g., hematological cancer (e.g., a myeloma, e.g., multiple myeloma) or solid tumors, and precancerous conditions (e.g., smoldering myeloma or monoclonal gammopathy of undetermined significance (MGUS)). In an embodiment, the disorder is associated with aberrant expression of CD138. In an embodiment, the antibody molecule is used to treat a subject having a disorder described herein, or is at risk of developing a disorder described herein. In an embodiment, the antibody molecule is used to reduce progression of the disorder, e.g., to reduce progression of a precancerous condition to cancer.

In an embodiment, the disorder is a hematological cancer. In an embodiment, the disorder is a solid tumor. In an embodiment, the cancer is a pancreatic cancer (e.g., pancreatic ductal adenoma carcinoma (PDAC)), a breast cancer, a lung cancer, a urogenital cancer, or a prostate cancer.

In an embodiment, the antibody molecule has an increased efficacy for treating a disorder described herein, compared to a reference antibody molecule, e.g., a related non-pH-selective antibody molecule, e.g., as determined by a method described herein. In an embodiment, the antibody molecule results in at least 10%, 20%, 30%, 40%, 50%, 60%, 70%, 80%, or 90% lower tumor burden, e.g., within a predetermined period of time. In an embodiment, the antibody molecule results in at least 10%, 20%, 30%, 40%, 50%, 60%, 70%, 80%, or 90%, or at least 1, 2, 3, 4, or 5-fold greater overall survival, e.g., the length of time from the date of diagnosis or the start of treatment. In an embodiment, the disorder is a cancer, e.g., a multiple myeloma. In an embodiment, the tumor burden is determined by imaging. In an embodiment, the tumor burden is determined in a tissue, e.g., a skeletal tissue.

The antibody molecules described herein are typically administered at a frequency that keeps a therapeutically effective level of antibody molecules in the patient’s system until the patient recovers. For example, the antibody molecules may be administered at a frequency that achieves a serum concentration sufficient for at least about 1, 2, 5, 10, 20, 30, or 40 antibody molecules to bind each CD138 molecule. In an embodiment, the antibody molecules are administered every 1, 2, 3, 4, 5, 6, or 7 days, every 1, 2, 3, 4, 5, or 6 weeks, or every 1, 2, 3, 4, 5, or 6 months.

Methods of administering various antibody molecules are known in the art and are described below. Suitable dosages of the antibody molecules used will depend on the age and weight of the subject and the particular drug used.

The antibody molecules can be used by themselves or conjugated to a second agent, e.g., a bacterial agent, toxin, or protein, e.g., a second anti-CD138 antibody molecule. This method includes: administering the antibody molecule, alone or conjugated to a second agent, to a subject requiring such treatment. The antibody molecules can be used to deliver a variety of therapeutic agents, e.g., a toxin, or mixtures thereof.

### Cancer

The anti-CD138 antibody molecules described herein (e.g., pH-selective anti-CD138 antibody molecules described herein) can be used to treat or prevent a cancer or a precancerous condition.

CD138 expression is dysregulated in many cancers, e.g., prostate cancer, breast cancer, pancreatic cancer, ovarian cancer, colon cancer, lung cancer, and myeloma (Kiviniemi et al. APMIS. 2004; 112(2): 89-97; Lendorf et al. J Histochem Cytochem. 2011; 59(6): 615-629; Juuti et al. Oncology. 2005; 68(2-3): 97-106; Kusumoto et al. Oncol Rep. 2010; 23(4): 917-25; Hashimoto et al. BMC Cancer. 2008; 8: 185; Joensuu et al. Cancer Res. 2002; 62(18):5210-5217; Seidel et al. Blood. 2000; 95(2): 388-392). CD138 can modulate several key processes of tumorigenesis, e.g., cancer cell proliferation, apoptosis, and angiogenesis (Teng et al. Matrix Biol. 2012; 31(1): 3-16). The molecular and clinical profiles of CD138 in solid and hematological cancers are described, e.g., in Akl et al. Oncotarget. 2015; 6(30):28693-28715.

CD138 can affect tumorigenesis by regulating mediators of tumor cell survival and proliferation (e.g., oncogenes or growth factors). For example, Sdcl-/- mice were protected against Wnt-1 induced mammary tumorigenesis (Alexander et al. Nat Genet. 2000; 25(3): 329-32). Hepatocyte growth factor (HGF) binds to CD138 on myeloma cells (Derksen et al. Blood. 2002; 99(4): 1405-1410). The interaction of HGF with CD138 potentiated Met signaling, which is involved in the growth, survival, and spread of a number of cancers (Birchmeier et al. Nat Rev Mol Cell Biol. 2003; 4(12): 915-925; Derksen et al. Blood. 2002; 99(4):1405-1410). CD138 expression is elevated in the reactive stroma of breast carcinoma tissue (Stanley et al. Am J Clin Pathol. 1999; 112(3): 377-383). MEFs expressing CD138 enhanced the growth of breast cancer cell lines in co-culture and promoted breast carcinoma progression in vivo (Maeda et al. Cancer Res. 2004; 64(2):612-621).

CD138 can regulate tumor cell apoptosis. Knock-down of CD138 in myeloma cells induced growth arrest and apoptosis (Khotskaya et al. J Biol Chem. 2009; 284(38): 26085-26095). Recombinant CD138 ectodomains induced apoptosis in MCF-7 breast cancer cells and cultured human prostate cancer cells (Sun et al. Cancer Res. 2008; 68(8):2912-2919; Hu et al. Neoplasia. 2010; 12(10): 826-836).

CD138 can bind to pro-angiogenic factors (e.g., FGF-2 and VEGF) and present these factors to their respective receptors on endothelial cells to initiate endothelial invasion and budding (Teng et al. Matrix Biol. 2012; 31(1): 3-16). Increased CD138 expression in stromal fibroblasts was observed in several carcinomas, such as those of the breast, stomach, and thyroid (Stanley et al. Am J Clin Pathol. 1999; 112(3): 377-383; Wiksten et al. Int J Cancer. 2001; 95(1): 1-6; Barbareschi et al. Cancer. 2003; 98(3): 474-483). In a xenograft model of human breast carcinoma cells and CD138-transfected fibroblasts implantation into mice, stromal CD138 expression was associated with significantly elevated microvessel density and larger vessel area (Maeda et al. Oncogene. 2006; 25(9): 1408-1412).

Exemplary cancers that can be treated or prevented by the antibody molecules described herein include, but are not limited to, acute lymphoblastic leukemia (ALL), acute myeloid leukemia (AML), adrenocortical carcinoma, Kaposi sarcoma, an AIDS-related lymphoma, primary central nervous system (CNS) lymphoma, anal cancer, appendix cancer, astrocytoma, atypical teratoid/rhabdoid tumor, basal cell carcinoma, bile duct cancer, bladder cancer, bone cancer (e.g., Ewing sarcoma or osteosarcoma and malignant fibrous histiocytoma), brain tumor (e.g., astrocytomas, brain stem glioma, central nervous system atypical teratoid/rhabdoid tumor, central nervous system embryonal tumor, central nervous system germ cell tumor, craniopharyngioma, or ependymoma), breast cancer, bronchial tumor, Burkitt lymphoma, carcinoid tumor (e.g., gastrointestinal carcinoid tumor), cardiac (heart) tumor, embryonal tumor, germ cell tumor, lymphoma, cervical cancer, cholangiocarcinoma, chordoma, chronic lymphocytic leukemia (CLL), chronic myelogenous leukemia (CML), chronic myeloproliferative neoplasm, colon cancer, colorectal cancer, craniopharyngioma, cutaneous T-cell lymphoma, ductal carcinoma in situ (DCIS), endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric (stomach) cancer, gastrointestinal carcinoid tumor, gastrointestinal stromal tumors (GIST), germ cell tumor (e.g., central nervous system tumor, extracranial tumor, extragonadal tumor, ovarian cancer, or testicular cancer), gestational trophoblastic disease, glioma, hairy cell leukemia, head and neck cancer, hepatocellular (liver) cancer, Hodgkin lymphoma, hypopharyngeal cancer, intraocular melanoma, islet cell tumor, pancreatic neuroendocrine tumor, Kaposi sarcoma, kidney cancer (e.g., renal cell cancer or Wilms tumor), Langerhans cell histiocytosis (LCH), laryngeal cancer, leukemia (e.g., acute lymphoblastic leukemia (ALL), acute myeloid leukemia (AML), chronic lymphocytic leukemia (CLL), chronic myelogenous leukemia (CML), or hairy cell leukemia), lip and oral cavity cancer, liver cancer, lung cancer (e.g., non-small cell lung cancer (NSCLC) or small cell lung cancer), lymphoma (e.g., aids-related, Burkitt lymphoma, cutaneous T-cell lymphoma, Hodgkin lymphoma, non-Hodgkin lymphoma, or primary central nervous system (CNS) lymphoma), Waldenström macroglobulinemia, male breast cancer, malignant fibrous histiocytoma of bone and osteosarcoma, melanoma (e.g., intraocular (eye) melanoma), Merkel cell carcinoma, mesothelioma, metastatic squamous neck cancer, midline tract carcinoma, mouth cancer, multiple endocrine neoplasia syndrome, multiple myeloma/plasma cell neoplasm, mycosis fungoides, myelodysplastic syndrome, myelodysplastic/myeloproliferative neoplasm, chronic myeloproliferative neoplasm, nasal cavity and paranasal sinus cancer, nasopharyngeal cancer, neuroblastoma, oral cancer, lip and oral cavity cancer, oropharyngeal cancer, osteosarcoma and malignant fibrous histiocytoma of bone, ovarian cancer (e.g., epithelial ovarian cancer or germ cell ovarian tumor), pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, pleuropulmonary blastoma, peritoneal cancer, prostate cancer, rectal cancer, retinoblastoma, rhabdomyosarcoma, salivary gland cancer, sarcoma (e.g., Ewing sarcoma, Kaposi sarcoma, osteosarcoma, rhabdomyosarcoma, soft tissue sarcoma, or uterine sarcoma), Sézary syndrome, skin cancer (e.g., melanoma, Merkel cell carcinoma, or nonmelanoma skin cancer), small intestine cancer, squamous cell carcinoma, testicular cancer, throat cancer, thymoma and thymic carcinoma, thyroid cancer, transitional cell cancer of the renal pelvis and ureter, urethral cancer, endometrial uterine cancer, vaginal cancer, vulvar cancer, or a metastatic lesion thereof.

In an embodiment, the cancer is a hematological cancer, e.g., a myeloma, lymphoma, or leukemia. In an embodiment, the cancer is a myeloma. In an embodiment, the cancer is a multiple myeloma.

In another embodiment, the cancer is a solid tumor. In an embodiment, the cancer is a cervical cancer (e.g., a cervical squamous cell carcinoma or an endocervical adenocarcinoma), a uterine cancer (e.g., a uterine corpus endometrioid carcinoma), a brain cancer (e.g., a glioblastoma), a lung cancer (e.g., a lung squamous cell carcinoma), or a breast cancer (e.g., a breast invasive carcinoma).

In an embodiment, the cancer is chosen from a bladder cancer, a breast cancer, a cervical cancer, a colorectal cancer, an endometrial cancer, a gallbladder cancer, a gastric cancer, a glioma, a head and neck cancer, a laryngeal cancer, a liver cancer, a lung cancer, a mesothelioma, a nasopharyngeal cancer, an oral cancer, an ovarian cancer, a pancreatic cancer, a prostate cancer, or a thyroid cancer.

In an embodiment, the cancer is a bladder cancer. CD138 is expressed in bladder cancer (Kim & Park. Hum Pathol. 2014; 45: 1830-1838). In an embodiment, the bladder cancer is a urothelial carcinoma, a squamous cell carcinoma, or an adenocarcinoma. In an embodiment, the bladder cancer is a noninvasive, non-muscle-invasive, or muscle-invasive. The anti-CD138 antibody molecules described herein can be used alone or in combination with a second therapeutic agent, procedure, or modality to treat a bladder cancer. In an embodiment, the anti-CD138 antibody molecule is used in combination with a surgery (e.g., transurethral resection of bladder tumor (TURBT) or cystectomy), an intravesical therapy (e.g., an intravesical immunotherapy (e.g., Bacillus Calmette-Guerin (BCG) therapy) or an intravesical chemotherapy (e.g., mitomycin, valrubicin, docetaxel, thiotepa, or gemcitabine)), a chemotherapy (e.g., an intravesical chemotherapy or a systemic chemotherapy (e.g., cisplatin, fluorouracil (5-FU), mitomycin, gemcitabine, methotrexate, vinblastine, doxorubicin, carboplatin, paclitaxel, docetaxel, ifosfamide, or pemetrexed), a radiation therapy, or an immunotherapy (e.g., intravesical BCG, an immune checkpoint inhibitor (e.g., a PD-L1 inhibitor (e.g., atezolizumab, durvalumab, or avelumab) or a PD-1 inhibitor (e.g., nivolumab or pembrolizumab).

In an embodiment, the cancer is a breast cancer. CD138 is expressed in breast cancer (Akl et al. Oncotarget. 2015; 6(30):28693-28715; Barbareschi et al. Cancer. 2003; 98: 474-483; Lim et al. Singapore Med J. 2014; 55: 468-472; Nguyen et al. Am J Clin Pathol. 2013; 140: 468-474; Lendorf et al. J Histochem Cytochem. 2011; 59: 615-629; Gotte et al. Breast Cancer Res. 2007; 9(1):R8; Tsanou et al. J Exp Clin Cancer Res. 2004; 23(4):641-650). In an embodiment, the breast cancer is a ductal carcinoma (e.g., ductal carcinoma in situ (DCIS), or invasive ductal carcinoma (IDC) (e.g., a tubular carcinoma, a medullary carcinoma, a mucinous carcinoma, a papillary carcinoma, or a cribriform carcinoma), a lobular carcinoma (e.g., a lobular carcinoma in situ (LCIS) or an invasive lobular carcinoma (ILC)), or an inflammatory breast cancer. In an embodiment, the breast cancer is ER-positive, PR-positive, HER2-positive, or triple-negative (ER-, PR- and HER2-). The anti-CD138 antibody molecules described herein can be used alone or in combination with a second therapeutic agent, procedure, or modality to treat a bladder cancer. In an embodiment, the anti-CD138 antibody molecule is used in combination with a surgery (e.g., a breast-conserving surgery or a mastectomy), a radiation therapy, a chemotherapy (e.g., an anthracycline (e.g., doxorubicin, liposomal doxorubicin, epirubicin), a taxane (e.g., paclitaxel, albumin-bound paclitaxel (e.g., nab-paclitaxel) or docetaxel), 5-fluorouracil (5-FU), cyclophosphamide, a platinum agent (e.g., cisplatin or carboplatin), vinorelbine, capecitabine, gemcitabine, mitoxantrone, ixabepilone, or eribulin), a hormone therapy (e.g., tamoxifen, toremifene, fulvestrant, an aromatase inhibitor (e.g., letrozole, anastrozole, or exemestane), ovarian ablation (e.g., oophorectomy, a luteinizing hormone-releasing hormone (LHRH) analog, or a chemotherapy drug)), a targeted therapy (e.g., trastuzumab, pertuzumab, ado-trastuzumab emtansine, lapatinib, neratinib, a CDK4/6 inhibitor (e.g., palbociclib or ribociclib), an mTOR inhibitor (e.g., everolimus), or a combination thereof.

In an embodiment, the cancer is a cervical cancer. CD138 is expressed in cervical cancer (Akl et al. Oncotarget. 2015; 6(30):28693-28715). In an embodiment, the cervical cancer is a microinvasive cervical cancer or invasive cervical cancer. In an embodiment, the cervical cancer is a squamous cell carcinoma or an adenocarcinoma. The anti-CD138 antibody molecules described herein can be used alone or in combination with a second therapeutic agent, procedure, or modality to treat a cervical cancer. In an embodiment, the anti-CD138 antibody molecule is used in combination with a surgery (e.g., a cryosurgery, a laser surgery, a conization, a simple hysterectomy, a radical hysterectomy, a trachelectomy, or a pelvic exenteration), a radiation therapy, a chemotherapy (e.g., cisplatin, carboplatin, paclitaxel, topotecan, gemcitabine, docetaxel, ifosfamide, 5-fluorouracil (5-FU), irinotecan, or mitomycin), a targeted therapy (e.g., an angiogenesis inhibitor (e.g., bevacizumab)), or a combination thereof.

In an embodiment, the cancer is an endometrial cancer. CD138 is expressed in endometrial cancer (Hasengaowa et al. Ann Oncol. 2005; 16:1109-1115). In an embodiment, the endometrial cancer is an endometrioid carcinoma, a serous carcinoma, a clear cell carcinoma, a mucinous carcinoma, a mixed or undifferentiated carcinoma, a squamous cell carcinoma, a transitional cell carcinoma, or an endometrial stromal sarcoma. The anti-CD 138 antibody molecules described herein can be used alone or in combination with a second therapeutic agent, procedure, or modality to treat an endometrial cancer. In an embodiment, the anti-CD138 antibody molecule is used in combination with a surgery, a radiation therapy, a hormone therapy (e.g., a progestin (e.g., medroxyprogesterone acetate) or megestrol acetate), tamoxifen, a luteinizing hormone-releasing hormone (LHRH) agonist (e.g., goserelin or leuprolide), an aromatase inhibitor (e.g., letrozole, anastrozole, or exemestane), a chemotherapy (e.g., paclitaxel, carboplatin, doxorubicin, liposomal doxorubicin, or cisplatin), or a combination thereof.

In an embodiment, the cancer is a gallbladder cancer. CD138 is overexpressed in gallbladder cancer (Roh et al. Eur Surg Res. 2008; 41(2): 245-250). In an embodiment, the gallbladder cancer is an adenocarcinoma or a papillary adenocarcinoma. The anti-CD138 antibody molecules described herein can be used alone or in combination with a second therapeutic agent, procedure, or modality to treat a gallbladder cancer. In an embodiment, the anti-CD138 antibody molecule is used in combination with a surgery, a radiation therapy, a chemotherapy (e.g., gemcitabine, cisplatin, 5-fluorouracil (5-FU), capecitabine, or oxaliplatin), or a palliative therapy (e.g., a biliary stent, a biliary catheter, a biliary bypass, an alcohol injection, a pain medicine, or a combination thereof.

In an embodiment, the cancer is a gastric cancer. Strong stromal CD138 expression is associated with gastric cancer (Wiksten et al. Int J Cancer. 2001; 95(1):1-6). In an embodiment, the gastric cancer is an adenocarcinoma (ACA). The anti-CD138 antibody molecules described herein can be used alone or in combination with a second therapeutic agent, procedure, or modality to treat a gastric cancer. In an embodiment, the anti-CD138 antibody molecule is used in combination with a surgery, a chemotherapy (e.g., 5-FU (fluorouracil), capecitabine, carboplatin, cisplatin, docetaxel, epirubicin, irinotecan, oxaliplatin, or paclitaxel), or a combination thereof.

In an embodiment, the cancer is a brain cancer (e.g., a glioma). CD138 is expressed in glioma (Xu et al. Mol Biol Rep. 2012; 39(9): 8979-8985). In an embodiment, the glioma is an astrocytoma, an en ependymoma, or an oligodendroglioma. The anti-CD138 antibody molecules described herein can be used alone or in combination with a second therapeutic agent, procedure, or modality to treat a glioma. In an embodiment, the anti-CD138 antibody molecule is used in combination with a surgery, a radiation therapy, a chemotherapy (e.g., carboplatin, carmustine (BCNU), cisplatin, cyclophosphamide, etoposide, irinotecan, lomustine (CCNU), methotrexate, procarbazine, temozolomide, or vincristine), a targeted therapy (e.g., bevacizumab or everolimus), a corticosteroid (e.g., dexamethasone), an anti-seizure drug, or a hormone, or a combination thereof.

In an embodiment, the cancer is a laryngeal cancer. CD138 expression is in laryngeal cancer (Klatka et al. Otolaryngol Pol. 2004; 58: 933-940). In an embodiment, the laryngeal cancer is a squamous cell carcinoma or an adenocarcinoma. The anti-CD138 antibody molecules described herein can be used alone or in combination with a second therapeutic agent, procedure, or modality to treat a laryngeal cancer. In an embodiment, the anti-CD138 antibody molecule is used in combination with a surgery, a radiation therapy, a chemotherapy (e.g., cisplatin, carboplatin, 5-fluorouracil (5-FU), docetaxel, paclitaxel, bleomycin, methotrexate, or ifosfamide), a targeted therapy (e.g., an EGFR inhibitor (e.g., cetuximab)), or a combination thereof. In an embodiment, the cancer is a liver cancer. In an embodiment, the liver cancer is a hepatocellular carcinoma (HCC), a cholangiocarcinoma, an angiosarcoma, or a secondary liver cancer. The anti-CD138 antibody molecules described herein can be used alone or in combination with a second therapeutic agent, procedure, or modality to treat a liver cancer. In an embodiment, the anti-CD138 antibody molecule is used in combination with a surgery, tumor ablation, tumor embolization, a radiation therapy, a targeted therapy (e.g., sorafenib or regorafenib), a chemotherapy (e.g., doxorubicin, 5-fluorouracil (5-FU), or cisplatin), or a combination thereof.

In an embodiment, the cancer is a lung cancer. CD138 is expressed in lung cancer (Anttonen et al. Lung Cancer. 2001; 32:297-305). In an embodiment, the lung cancer is a non-small cell lung cancer (NSCLC) (e.g., an adenocarcinoma, a squamous cell carcinoma, a large cell carcinoma, or a large cell neuroendocrine tumor) or a small cell lung cancer (SCLC). The anti-CD138 antibody molecules described herein can be used alone or in combination with a second therapeutic agent, procedure, or modality to treat a lung cancer. In an embodiment, the anti-CD138 antibody molecule is used in combination with a surgery, radiofrequency ablation (RFA), a radiation therapy, a chemotherapy (cisplatin, carboplatin, paclitaxel, albumin-bound paclitaxel (nab-paclitaxel), docetaxel, gemcitabine, vinorelbine, irinotecan, etoposide, vinblastine, or pemetrexed), a targeted therapy (an angiogenesis inhibitor (e.g., bevacizumab or ramucirumab), an EGFR inhibitor (e.g., erlotinib, afatinib, gefitinib, osimertinib, or necitumumab), an ALK inhibitor (e.g., crizotinib, ceritinib, alectinib, or brigatinib), a BRAF inhibitor (e.g., dabrafenib or trametinib), an immunotherapy (e.g., a PD-1 inhibitor (e.g., nivolumab or pembrolizumab) or a PD-L1 inhibitor (e.g., atezolizumab), or a combination thereof, e.g., to treat a non-small cell lung cancer. In an embodiment, the anti-CD138 antibody molecule is used in combination with a surgery, a radiation therapy, a chemotherapy (cisplatin, etoposide, carboplatin, or irinotecan), or a combination thereof, e.g., to treat a small cell lung cancer.

In an embodiment, the cancer is a mesothelioma. CD138 is expressed in mesothelioma (Kumar-singh et al. J Pathol. 1998; 186:300-305). In an embodiment, the mesothelioma is an epithelioid mesothelioma, a sarcomatoid mesothelioma, or abiphasic mesothelioma. In an embodiment, the mesothelioma is a pleural mesothelioma, a peritoneal mesothelioma, or a pericardial mesothelioma. The anti-CD138 antibody molecules described herein can be used alone or in combination with a second therapeutic agent, procedure, or modality to treat a mesothelioma. In an embodiment, the anti-CD138 antibody molecule is used in combination with a surgery, a radiation therapy, a chemotherapy (e.g., pemetrexed, cisplatin, carboplatin, gemcitabine, methotrexate, vinorelbine, mitomycin, or doxorubicin), or a combination thereof.

In an embodiment, the cancer is a nasopharyngeal cancer. CD138 is expressed in nasopharyngeal cancer (Kim et al. Head Neck. 2011; 33:1458-1466). In an embodiment, the nasopharyngeal cancer is a keratinizing squamous cell carcinoma, a non-keratinizing differentiated carcinoma, or an undifferentiated carcinoma. The anti-CD138 antibody molecules described herein can be used alone or in combination with a second therapeutic agent, procedure, or modality to treat a nasopharyngeal cancer. In an embodiment, the anti-CD138 antibody molecule is used in combination with a surgery, a radiation therapy, a chemotherapy (e.g., carboplatin, doxorubicin, epirubicin, paclitaxel, docetaxel, gemcitabine, bleomycin, or methotrexate), a targeted therapy (e.g., cetuximab), or a combination thereof.

In an embodiment, the cancer is a nasopharyngeal cancer. CD138 is expressed in oral cancer (Al-Otaibi et al. J Oral Pathol Med. 2013; 42: 186-193). In an embodiment, the oral cancer is a squamous cell carcinoma, a verrucous carcinoma, or a minor salivary gland carcinoma. The anti-CD138 antibody molecules described herein can be used alone or in combination with a second therapeutic agent, procedure, or modality to treat an oral cancer. In an embodiment, the anti-CD138 antibody molecule is used in combination with a surgery, a radiation therapy, a chemotherapy (e.g., cisplatin, carboplatin, 5-fluorouracil (5-FU), paclitaxel, docetaxel, methotrexate, ifosfamide, or bleomycin), a targeted therapy (e.g., cetuximab), or a combination thereof.

In an embodiment, the cancer is an ovarian cancer. CD138 is expressed in ovarian cancer (Kusumoto et al. Oncol Rep. 2010; 23: 917-925; Davies et al. Clin Cancer Res. 2004; 10: 5178-5186). In an embodiment, the ovarian cancer is an epithelial cancer, a germ cell carcinoma, a stromal carcinoma, or a small cell carcinoma. The anti-CD138 antibody molecules described herein can be used alone or in combination with a second therapeutic agent, procedure, or modality to treat an ovarian cancer. In an embodiment, the anti-CD138 antibody molecule is used in combination with a surgery, a chemotherapy (e.g., cisplatin, carboplatin, paclitaxel, albumin bound paclitaxel (nab-paclitaxel), docetaxel, altretamine, capecitabine, cyclophosphamide, etoposide, gemcitabine, ifosfamide, irinotecan, liposomal doxorubicin, melphalan, pemetrexed, topotecan, or vinorelbine), a hormone therapy (e.g., a luteinizing-hormone-releasing hormone (LHRH) agonist (e.g., goserelin or leuprolide), tamoxifen, or aromatase inhibitor (e.g., letrozole, anastrozole, or exemestane), a targeted therapy (e.g., an angiogenesis inhibitor (e.g., bevacizumab), a PARP inhibitor (e.g., olaparib, rucaparib, or niraparib), a radiation therapy, or a combination thereof.

In an embodiment, the cancer is a pancreatic cancer. CD138 is expressed in pancreatic cancer (Juuti et al. Oncology. 2005; 68: 97-106). In an embodiment, the pancreatic cancer is an exocrine tumor or an endocrine tumor. The anti-CD138 antibody molecules described herein can be used alone or in combination with a second therapeutic agent, procedure, or modality to treat a pancreatic cancer. In an embodiment, the anti-CD138 antibody molecule is used in combination with a surgery, ablation, embolization, a radiation therapy, or a chemotherapy (cemcitabine, 5-fluorouracil (5-FU), irinotecan, oxaliplatin, albumin-bound paclitaxel, capecitabine, cisplatin, paclitaxel, docetaxel, or irinotecan liposome.

In an embodiment, the cancer is a prostate cancer. CD138 is expressed in prostate cancer (Ledezma et al. Asian J Androl. 2011; 13: 476-480; Shariat et al. BJU Int. 2008; 101:232-237; Kiviniemi et al. Apmis. 2004; 112: 89-97; Zellweger et al. Prostate. 2003; 55: 20-29). In an embodiment, the prostate cancer is an adenocarcinoma, a transitional cell (or urothelial) cancer, a squamous cell cancer, or a small cell prostate cancer. The anti-CD138 antibody molecules described herein can be used alone or in combination with a second therapeutic agent, procedure, or modality to treat a prostate cancer. In an embodiment, the anti-CD138 antibody molecule is used in combination with a surgery, a radiation therapy, a cryotherapy, a hormone therapy (e.g., orchiectomy, an LHRH agonist (e.g., leuprolide, goserelin, triptorelin, or histrelin), an LHRH antagonist (e.g., degarelix), a CYP17 inhibitor (e.g., abiraterone), an anti-androgen (e.g., flutamide, bicalutamide, nilutamide, or enzalutamide), an estrogen, or ketoconazole), a chemotherapy (e.g., docetaxel, cabazitaxel, mitoxantrone, or estramustine), a vaccine treatment (e.g., Sipuleucel-T), or a bone-directed treatment (e.g., a bisphosphonate (e.g., zoledronic acid), denosumab, a corticosteroid (e.g., prednisone or dexamethasone), an external radiation therapy, a radiopharmaceutical (e.g., Strontium-89, Samarium-153, or Radium-223), or a combination thereof.

In an embodiment, the cancer is a head and neck cancer. CD138 is expressed in head and neck cancer (Anttonen et al. Br J Cancer. 1999; 79: 558-564; Inki et al. Br J Cancer. 1994; 70: 319-323). In an embodiment, the head and neck cancer is a squamous cell carcinoma. The anti-CD138 antibody molecules described herein can be used alone or in combination with a second therapeutic agent, procedure, or modality to treat a head and neck cancer. In an embodiment, the anti-CD138 antibody molecule is used in combination with a surgery, a radiation therapy, a chemotherapy (e.g., methotrexate, bleomycin, or docetaxel), a targeted therapy (e.g., cetuximab), an immunotherapy (e.g., a PD-1 inhibitor (e.g., nivolumab or pembrolizumab)), or a combination thereof.

In an embodiment, the cancer is a thyroid cancer. CD138 is expressed in thyroid cancer (Oh & Park. J Korean Med Sci. 2006; 21: 397-405). In an embodiment, the thyroid cancer is a papillary carcinoma, a follicular carcinoma, a Hürthle cell carcinoma, a medullary thyroid carcinoma, or an anaplastic carcinoma. The anti-CD138 antibody molecules described herein can be used alone or in combination with a second therapeutic agent, procedure, or modality to treat a thyroid cancer. In an embodiment, the anti-CD138 antibody molecule is used in combination with a surgery, a radioactive iodine treatment, a thyroid hormone therapy, a radiation therapy, a chemotherapy, a targeted therapy (e.g., a kinase inhibitor (e.g., sorafenib or lenvatinib), or a combination thereof.

In an embodiment, the cancer is a chronic lymphocytic leukemia (CLL). CD138 is expressed in chronic lymphocytic leukemia cancer (Jilani et al. Int J Lab Hematol. 2009; 31:97-105). The anti-CD138 antibody molecules described herein can be used alone or in combination with a second therapeutic agent, procedure, or modality to treat a thyroid cancer. In an embodiment, the anti-CD 138 antibody molecule is used in combination with a chemotherapy (e.g., a purine analog (e.g., fludarabine, pentostatin, or cladribine), an alkylating agent (e.g., chlorambucil, cyclophosphamide, or bendamustine), a corticosteroid (e.g., prednisone, methylprednisolone, or dexamethasone), doxorubicin, methotrexate, oxaliplatin, vincristine, etoposide, and cytarabine), an anti-CD20 antibody (rituximab, obinutuzumab, or ofatumumab), an anti-CD52 antibody (e.g., alemtuzumab), a targeted therapy (e.g., ibrutinib, idelalisib, or venetoclax), a stem cell transplant (SCT), or a combination thereof.

In an embodiment, the cancer is a lymphoma (e.g., a diffuse large B-cell lymphoma (DLBCL)). CD138 is expressed in DLBCL (Oh & Park. J Korean Med Sci. 2006; 21: 397-405; Bodoor et al. Asian Pac J Cancer Prev. 2012; 13: 3037-3046). The anti-CD138 antibody molecules described herein can be used alone or in combination with a second therapeutic agent, procedure, or modality to treat a DLBCL. In an embodiment, the anti-CD138 antibody molecule is used in combination with a chemotherapy (e.g., an alkylating agent (e.g., cyclophosphamide, chlorambucil, bendamustine, or ifosfamide), a corticosteroid (e.g., prednisone or dexamethasone), a platinum drug (cisplatin, carboplatin, or oxaliplatin), a purine analog (e.g., fludarabine, pentostatin, or cladribine), an anti-metabolite (e.g., cytarabine, gemcitabine, methotrexate, or pralatrexate), vincristine, doxorubicin, mitoxantrone, etoposide, or bleomycin), an immunotherapy (e.g., an anti-CD20 antibody (rituximab, obinutuzumab, or ofatumumab), an anti-CD52 antibody (e.g., alemtuzumab), an anti-CD30 antibody (e.g., brentuximab vedotin), interferon, an immunomodulating drug (e.g., thalidomide or lenalidomide), a targeted therapy (e.g., a proteasome inhibitor (e.g., bortezomib), a histone deacetylase (HDAC) inhibitor (e.g., romidepsin or belinostat), or a kinase inhibitor (e.g., ibrutinib or idelalisib)), a radiation therapy, a stem cell transplant (SCT), or a combination thereof.

In an embodiment, the cancer is a Hodgkin’s lymphoma. CD138 is expressed in Hodgkin’s lymphoma (Gharbaran et al. J Hematol Oncol. 2013; 6:62; Vassilakopoulos et al. Anticancer Res. 2005; 25: 4743-4746). The anti-CD138 antibody molecules described herein can be used alone or in combination with a second therapeutic agent, procedure, or modality to treat a Hodgkin’s lymphoma. In an embodiment, the anti-CD138 antibody molecule is used in combination with a chemotherapy (e.g., doxorubicin, bleomycin, vinblastine, dacarbazine, etoposide, cyclophosphamide, vincristine, procarbazine, prednisone, mechlorethamine, vincristine, or vinblastine), a radiation therapy, an immunotherapy (e.g., an anti-CD30 antibody (e.g., brentuximab vedotin)), a stem cell transplant, or a combination thereof.

In an embodiment, the antibody molecule is used to treat or prevent a precancerous condition. Precancerous condition, also known as premalignant condition, potentially precancerous condition, or potentially premalignant condition, refers to a state of disordered morphology of cells that is associated with an increased risk of cancer. If left untreated, precancerous conditions may lead to cancer. In an embodiment, the premalignant lesion is morphologically atypical tissue which appears abnormal under microscopic examination, and in which cancer is more likely to occur than in its apparently normal counterpart. In an embodiment, the precancerous condition is smoldering myeloma or asymptomatic myeloma. In an embodiment, the precancerous condition is monoclonal gammopathy of undetermined significance (MGUS). Other examples of precancerous conditions include, but are not limited to, actinic keratosis, Barrett’s esophagus, atrophic gastritis, ductal carcinoma in situ, dyskeratosis congenital, sideropenic dysphagia, lichen planus, oral submucous fibrosis, solar elastosis, cervical dysplasia, leukoplakia, and erythroplakia.

### Multiple Myeloma

The antibody molecules described herein (e.g., pH-selective anti-CD138 antibody molecules described herein) can be used to treat or prevent multiple myeloma.

Multiple myeloma (MM), also known as plasma cell myeloma, is a cancer of plasma cells, which are normally responsible for producing antibodies (Raab et al. Lancet. 2009; 374(9686): 324-39). Multiple myeloma is typically considered as a malignant disorder of uncontrolled proliferation of monoclonal plasma cells (PCs) in the bone marrow. This hematological malignancy is clinically characterized for example, by hyperproduction of monoclonal immunoglobulins, osteolytic bone disease, anemia, immunosuppression and end-organ damage, predominantly occurring in the kidney. Encouraging therapeutic advancements in the treatment of MM patients have occurred over the last several decades. Despite such advances, multiple myeloma remains an incurable disease in most patients due to a high incidence of relapse or treatment resistance. MM represents the second leading hematological cancer (globally) accounting for about 2% of all newly diagnosed cancers and approximately 13% of hematological malignancies in the U.S. with conservative estimates of greater than 30,000 new cases in 2018 leading to approximately 13,000 deaths. There is a high unmet need for developing new approaches for treating, preventing and diagnosing multiple myeloma and other plasma cell disorders that share similar disease mechanisms. Without wishing to be bound by theory, it is believed that in an embodiment, the prospect of earlier intervention in disease treatment through the use of safe and efficacious agents is also an emerging and attractive strategy. Novel targeted therapies, such as the therapies using the anti-CD138 antibody molecules described herein, can be beneficial at least in this respect.

Signs or symptoms of multiple myeloma include, e.g., bone pain, anemia (e.g., normocytic and/or normochromic anemia), kidney failure (e.g., acute or chronical kidney failure), infection (e.g., pneumonias or pyelonephritis), a neurological symptom (e.g., weakness, confusion, fatigue, headache, visual change, retinopathy, radicular pain, loss of bowel or bladder control, carpal tunnel syndrome, or paraplegia).

Risk factors for multiple myeloma include, e.g., smoldering myeloma (also known as asymptomatic myeloma), monoclonal gammopathy of undetermined significance (MGUS), obesity, or familial predisposition. In an embodiment, the anti-CD138 antibody molecules described herein can be used to reduce (e.g., prevent) the progression of smoldering myeloma or MGUS to multiple myeloma.

Diagnostic criteria for symptomatic myeloma, asymptomatic myeloma and MGUS are described, e.g., in Kyle & Rajkumar Leukemia. 2009; 23(1): 3-9.

Diagnostic criteria for symptomatic myeloma (all three criteria must be met) include, e.g., clonal plasma cells >10% on bone marrow biopsy or (in any quantity) in a biopsy from other tissues (plasmacytoma), a monoclonal protein (Myeloma protein) in either serum or urine (except in cases of true non-secretory myeloma), and evidence of end-organ damage felt related to the plasma cell disorder (related organ or tissue impairment, commonly referred to by the acronym “CRAB”): hypercalcemia (corrected calcium >2.75 mmol/l, >11 mg/dL), renal insufficiency attributable to myeloma, anemia (hemoglobin <10 g/dl), bone lesions (lytic lesions or osteoporosis with compression fractures). Diagnostic criteria for asymptomatic/smoldering myeloma include, e.g., serum M protein >30 g/l (3 g/dL) and/or clonal plasma cells >10% on bone marrow biopsy and no myeloma-related organ or tissue impairment). Diagnostic criteria for monoclonal gammopathy of undetermined significance (MGUS) include, e.g., serum paraprotein <30 g/l (3 g/dL) and clonal plasma cells <10% on bone marrow biopsy and no myeloma-related organ or tissue impairment or a related B-cell lymphoproliferative disorder

Related conditions include, e.g., solitary plasmacytoma, plasma cell dyscrasia (e.g., AL amyloidosis), and peripheral neuropathy, organomegaly, endocrinopathy, monoclonal plasma cell disorder, and skin changes.

The International Staging System (ISS) for myeloma is described, e.g., in Greipp et al. J Clin Oncol. 2005; 23(15): 3412-20. For example, the ISS includes the following: Stage I: β2 microglobulin (β2M) < 3.5 mg/L, albumin ≥ 3.5 g/dL; Stage II: β2M < 3.5 mg/L and albumin < 3.5 g/dL; or β2M 3.5-5.5 mg/L irrespective of the serum albumin; Stage III: β2M ≥ 5.5 mg/L.

The ISS can be used along with the Durie-Salmon Staging System. The Durie-Salmon Staging System is described, e.g., in Durie & Salmon Cancer. 1975; 36(3):842-54. For example, the Durie-Salmon Staging System include the following: Stage I (all of Hb > 10 g/dL, normal calcium, skeletal survey: normal or single plasmacytoma or osteoporosis, serum paraprotein level < 5 g/dL if IgG, < 3 g/dL if IgA, urinary light chain excretion < 4 g/24h); Stage II (fulfilling the criteria of neither I nor III); Stage III (one or more of Hb < 8.5 g/dL, high calcium > 12 mg/dL, skeletal survey: three or more lytic bone lesions, serum paraprotein > 7 g/dL if IgG, > 5 g/dL if IgA, urinary light chain excretion > 12 g/24h). Stages I, II, and III of the Durie-Salmon Staging System can be divided into A or B depending on serum creatinine: A: serum creatinine < 2 mg/dL (< 177 µmol/L); B: serum creatinine > 2 mg/dL (> 177 µmol/L).

Other treatments for multiple myeloma that can be used in combination with an anti-CD 138 antibody molecule described herein include, e.g., a protease inhibitor (e.g., bortezomib (VELCADE®), carfilzomib (KYPROLIS®), or ixazomib (NINLARO®)), an immunomodulating agent (e.g., thalidomide (THALOMID®), lenalidomide (REVLIMID®), or pomalidomide (POMALYST®)), a chemotherapy (e.g., melphalan, vincristine (ONCOVIN®), cyclophosphamide, etoposide, doxorubicin (ADRIAMYCIN®), liposomal doxorubicin (DOXIL®), or bendamustine (TREANDA®)), a corticosteroid (e.g., prednisone or dexamethasone), a histone deacetylase (HDAC) inhibitor (e.g., panobinostat (FARYDAK®), an anti-CD38 antibody (e.g., daratumumab (DARZALEX®)), an anti-SLAMF7 antibody (e.g., elotuzumab (EMPLICITI®)), an interferon, or a bone marrow transplantation (e.g., autologous stem cell transplantation (ASCT) or allogeneic stem cell transplantation), a bisphosphonate (e.g., pamidronate (AREDIA®) and zoledronic acid (ZOMETA®), a radiation therapy, a surgery, an intravenous immunoglobulin (IVIG), a treatment for low blood cell count (e.g., erythropoietin (PROCRIT®) or darbepoietin (ARANESP®), plasmapheresis, or a combination thereof.

Exemplary combination therapies that can be used in combination with an anti-CD138 antibody molecule described herein for treating multiple myeloma include, but are not limited to, melphalan and prednisone (MP), with or without thalidomide or bortezomib; vincristine, doxorubicin (ADRIAMYCIN®), and dexamethasone (VAD); thalidomide (or lenalidomide) and dexamethasone; bortezomib, doxorubicin, and dexamethasone; bortezomib, dexamethasone, and thalidomide (or lenalidomide); liposomal doxorubicin, vincristine, and dexamethasone; carfilzomib, lenalidomide, and dexamethasone; dexamethasone, cyclophosphamide, etoposide, and cisplatin (DCEP); dexamethasone, thalidomide, cisplatin, doxorubicin, cyclophosphamide, and etoposide (DT-PACE), with or without bortezomib; panobinostat, bortezomib, and dexamethasone; ixazomib, lenalidomide; and dexamethasone, and elotuzumab, lenalidomide, and dexamethasone.

### Combination Therapies

The antibody molecules described herein (e.g., pH-selective anti-CD138 antibody molecules described herein) can be used in combination with other therapies. For example, the combination therapy can include an antibody molecule co-formulated with, and/or co-administered with, one or more additional therapeutic agents, e.g., one or more additional therapeutic agents described herein. In other embodiments, the antibody molecules are administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject’s affliction with a disorder. In an embodiment, two or more treatments are delivered prophylactically, e.g., before the subject has the disorder or is diagnosed with the disorder. In another embodiment, the two or more treatments are delivered after the subject has developed or diagnosed with the disorder. In an embodiment, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In an embodiment of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In an embodiment, delivery is such that the reduction in a symptom, or other parameter related to the disorder is greater than what would be observed with one treatment delivered in the absence of the other. The effect of the two or more treatments can be partially additive, wholly additive, or greater than additive. In an embodiment, the effect of the two or more treatments can be synergistic. The delivery can be such that an effect of the first treatment delivered is still detectable when the second is delivered.

In certain embodiments, the additional agent is a second antibody molecule, e.g., an antibody molecule different from a first antibody molecule. Exemplary antibody molecules that can be used in combination include, but are not limited to, any combination of the antibody molecules listed in Table 1, 2, or 6.

In an embodiment, the antibody molecule is administered in combination with a second therapy to treat or prevent a myeloma, e.g., multiple myeloma.

In an embodiment, the antibody molecule is administered in combination with a protease inhibitor. Exemplary protease inhibitors include, e.g., bortezomib (VELCADE®), carfilzomib (KYPROLIS®), and ixazomib (NINLARO®).

In an embodiment, the antibody molecule is administered in combination with an immunomodulating agent. Exemplary immunomodulating agents include, e.g., thalidomide (THALOMID®), lenalidomide (REVLIMID®), and pomalidomide (POMALYST®).

In an embodiment, the antibody molecule is administered in combination with a chemotherapeutic agent. Exemplary chemotherapeutic agents include, e.g., melphalan, vincristine (ONCOVIN®), cyclophosphamide, etoposide, doxorubicin (ADRIAMYCIN®), liposomal doxorubicin (DOXIL®), and bendamustine (TREANDA®).

In an embodiment, the antibody molecule is administered in combination with a corticosteroid, e.g., prednisone and dexamethasone.

In an embodiment, the antibody molecule is administered in combination with a histone deacetylase (HDAC) inhibitor, e.g., panobinostat (FARYDAK®).

In an embodiment, the antibody molecule is administered in combination with an anti-CD38 antibody, e.g., daratumumab (DARZALEX®).

In an embodiment, the antibody molecule is administered in combination with an anti-SLAMF7 antibody, e.g., elotuzumab (EMPLICITI®).

In an embodiment, the antibody molecule is administered in combination with an interferon.

In an embodiment, the antibody molecule is administered in combination with bone marrow transplantation (e.g., autologous stem cell transplantation (ASCT) or allogeneic stem cell transplantation).

In an embodiment, the antibody molecule is administered in combination with a bisphosphonate, e.g., pamidronate (AREDIA®) or zoledronic acid (ZOMETA®).

In an embodiment, the antibody molecule is administered in combination with a radiation therapy.

In an embodiment, the antibody molecule is administered in combination with a surgery.

In an embodiment, the antibody molecule is administered in combination with an intravenous immunoglobulin (IVIG).

In an embodiment, the antibody molecule is administered in combination with a treatment for low blood cell count, e.g., erythropoietin (PROCRIT®) or darbepoietin (ARANESP®).

In an embodiment, the antibody molecule is administered in combination with plasmapheresis.

In an embodiment, the antibody molecule is administered in combination with melphalan and prednisone (MP), with or without thalidomide or bortezomib.

In an embodiment, the antibody molecule is administered in combination with vincristine, doxorubicin (ADRIAMYCIN®), and dexamethasone (VAD).

In an embodiment, the antibody molecule is administered in combination with thalidomide (or lenalidomide) and dexamethasone.

In an embodiment, the antibody molecule is administered in combination with bortezomib, doxorubicin, and dexamethasone.

In an embodiment, the antibody molecule is administered in combination with bortezomib, dexamethasone, and thalidomide (or lenalidomide).

In an embodiment, the antibody molecule is administered in combination with liposomal doxorubicin, vincristine, and dexamethasone;

In an embodiment, the antibody molecule is administered in combination with carfilzomib, lenalidomide, and dexamethasone.

In an embodiment, the antibody molecule is administered in combination with dexamethasone, cyclophosphamide, etoposide, and cisplatin (DCEP).

In an embodiment, the antibody molecule is administered in combination with dexamethasone, thalidomide, cisplatin, doxorubicin, cyclophosphamide, and etoposide (DT-PACE), with or without bortezomib.

In an embodiment, the antibody molecule is administered in combination with panobinostat, bortezomib, and dexamethasone.

In an embodiment, the antibody molecule is administered in combination with ixazomib, lenalidomide, and dexamethasone.

In an embodiment, the antibody molecule is administered in combination with elotuzumab, lenalidomide, and dexamethasone.

In an embodiment, the antibody molecule is administered in combination with a second agent that targets the CD138 pathway. Exemplary agents that target the CD138 pathway include, e.g., an agent that targets the extracellular domain of CD138 (e.g., synstatin, BT-062-DM4 (indatuximab ravtansine), BB4 conjugated to ¹³¹I, OC-46F2, or GLVGLIFAV (SEQ ID NO: 448)), an agent that targets shed CD138 (e.g., NSC 405020, BB-94, PI-88, PG545, M402, SST00001, or Pentraxin-3), and an agent that targets genetic expression of CD138 (e.g., an all-trans retinoic acid, nimesulide, zoledronic acid, or imatinib). Other agents that target the CD138 pathway are described, e.g., Akl et al. Oncotarget. 2015; 6(30):28693-28715, the content of which in incorporated by reference in its entirety.

In an embodiment, the antibody molecule is administered in combination with lenalidomide and/or dexamethasone, e.g., to treat a multiple myeloma (e.g., a relapsed multiple myeloma).

In an embodiment, the antibody molecule is administered in combination with an FGFR2 antagonist (e.g., an anti-FGFR2 antibody, e.g., FPA144) to treat a solid tumor (e.g., an advanced solid tumor).

In an embodiment, the antibody molecule is administered in combination with a αᵥβ₃ inhibitor (e.g., an ADC against integrin αᵥβ₃, e.g., brentuximab vedotin), e.g., to treat Hodgkin lymphoma (e.g., relapsed or refractory Hodgkin lymphoma).

In an embodiment, the antibody molecule is administered in combination with a heparin or heparanase inhibitor (e.g., roneparstat (SST0001)), e.g., to treat a multiple myeloma (e.g., an advanced multiple myeloma).

In an embodiment, the antibody molecule is administered in combination with a VEGFR inhibitor (e.g., bevacizumab or cediranib), e.g., to treat a cancer (e.g., an advanced cancer).

In an embodiment, the antibody molecule is administered in combination with a Wnt signaling pathway inhibitor (e.g., ipafricept (OMP-54F28)), e.g., to treat a solid tumor.

In an embodiment, the antibody molecule is administered in combination with an FAK inhibitor (e.g., defactinib (VS-6063) or GSK2256098), e.g., to treat a solid tumor, e.g., a lung cancer (e.g., a non-small cell lung cancer, e.g., with a KRAS mutation).

In an embodiment, the antibody molecule is administered in combination with a glysoaminoglycan or heparanase inhibitor (e.g., necuparanib (M402)), optionally, further in combination with a chemotherapeutic agent (e.g., nab-paclitaxel or gemcitabine), e.g., to treat a pancreatic cancer (e.g., a metastatic pancreatic cancer).

In an embodiment, the antibody molecule is administered in combination with a mannose oligosaccharide, or a FGF, heparanase, and/or VEGF inhibitor (e.g., muparfostat (PI-88)), e.g., to treat a cancer (e.g., a melanoma).

In an embodiment, the antibody molecule is administered in combination with a chemically modified heparin sulfate/heparanase inhibitor (e.g., PG545), e.g., to treat a solid tumor (e.g., an advanced solid tumor).

In an embodiment, the antibody molecule is administered in combination with an amino acid or matrix metalloprotease inhibitor (e.g., intrapleural batimastat (BB-94)), e.g., to treat a malignant pleural effusion.

In an embodiment, the antibody molecule is administered in combination with a chimeric anti-CD138 antigen receptor-modified T cells, e.g., to treat a multiple myeloma (e.g., a relapsed and/or refractory multiple myeloma).

In an embodiment, the antibody molecule is administered in combination with a proteasome inhibitor. In an embodiment, the proteasome inhibitor comprises bortezomib. In an embodiment, the proteasome inhibitor comprises a compound having a formula of

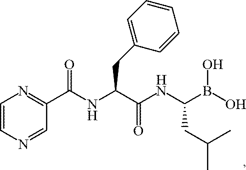

or a pharmaceutically acceptable salt, hydrate, solvate, prodrug, stereoisomer, or tautomer thereof.

Bortezomib (also known as VELCADE®, LDP 341, MLN341, or PS-341) is an anti-cancer drug and the first therapeutic proteasome inhibitor to be used in humans. Proteasomes are higher order enzymatic complexes that degrade misfolded, damaged, or potentially toxic proteins and constitute a metabolic and homeostatic mechanism by which cells regulate the concentration and turnover of such proteins. In some cancers, this homeostasis is imbalanced leading to the inappropriate degradation of proteins that normally function to kill cancer cells or control cellular growth (e.g., the immunoproteasome). Bortezomib modulates this process and promotes a pro-apoptotic or immune-based killing of the cancer cells. It does so in part by promoting the unfolded protein response (UPR). In an embodiment, bortezomib has the chemical structure of [(1R)-3-Methyl-1-[[(2S)-1-oxo-3-phenyl-2-[(pyrazinylcarbonyl)amino]propyl]amino]butyl]boronic Acid.

Bortezomib is approved in the U.S. and Europe for treating relapsed multiple myeloma and mantle cell lymphoma. Clinical studies have shown partial benefit in the use of bortezomib as an initial therapy, as maintenance therapy, or as second line therapy for previously treated multiple myeloma. The drug is more typically used in combination with dexamethasone (VD) or as part of a three-drug combination, e.g., VELCADE-REVLIMID (lenalidomide)-dexamethasone (VRD).

Exemplary therapies that can be used in combination with an antibody molecule or composition described herein to treat or prevent other disorders are also described in the section of “Methods of Treating or Preventing Disorders” herein.

### Methods of Diagnosis

The antibody molecules described herein (e.g., pH-selective anti-CD138 antibody molecules described herein) can be used to detect the presence of CD 138 and/or to diagnose disorders associated with CD138 or symptoms thereof.

In an aspect, the present disclosure provides a diagnostic method for detecting the presence of CD138 in vitro (e.g., in a biological sample, such as a biopsy or blood sample) or in vivo (e.g., in vivo imaging in a subject). The method includes: (i) contacting the sample with an anti-CD138 antibody molecule described herein, or administering to the subject, the antibody molecule; (optionally) (ii) contacting a reference sample, e.g., a control sample (e.g., a control biological sample, such as a biopsy or blood sample) or a control subject with an antibody molecule described herein; and (iii) detecting formation of a complex between the antibody molecule and CD138 in the sample or subject, or the control sample or subject, wherein a change, e.g., a statistically significant change, in the formation of the complex in the sample or subject relative to the control sample or subject is indicative of the presence of CD138 in the sample. The antibody molecule can be directly or indirectly labeled with a detectable substance to facilitate detection of the bound or unbound antibody molecule. Suitable detectable substances include various enzymes, prosthetic groups, fluorescent materials, luminescent materials and radioactive materials, as described above and described in more detail below.

The term “sample,” as it refers to samples used for detecting a polypeptide (e.g., CD138) or a nucleic acid encoding the polypeptide includes, but is not limited to, cells, cell lysates, proteins or membrane extracts of cells, body fluids such as blood, or tissue samples such as biopsies.

Complex formation between the antibody molecule, and CD138, can be detected by measuring or visualizing either the antibody molecule bound to CD138 or unbound antibody molecule. Any suitable detection assays can be used, and conventional detection assays include an enzyme-linked immunosorbent assays (ELISA), a radioimmunoassay (RIA) or tissue immunohistochemistry. Alternative to labeling the antibody molecule, the presence of CD138 can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of CD138 in the sample is inversely proportional to the amount of labeled standard bound to the antibody molecule.

The anti-CD138 antibody molecules described herein can be used to diagnose disorders that can be treated or prevented by the anti-CD138 antibody molecules described herein. The detection or diagnostic methods described herein can be used in combination with other methods described herein to treat or prevent a disorder described herein.

### Enumerated Embodiments

1. An anti-CD138 antibody molecule that selectively binds to CD138 in a tumor microenvironment.

2. The antibody molecule of embodiment 1, which preferentially disassociates from CD138 in a normal tissue.

3. The antibody molecule of embodiment 1 or 2, wherein the tumor microenvironment is an acidic tumor microenvironment.

4. The antibody molecule of any of embodiments 1-3, wherein the tumor microenvironment is a solid tumor microenvironment.

5. The antibody molecule of any of embodiments 1-4, wherein the tumor microenvironment has a pH of 5.5-7.0 (e.g., a pH of 5.5-6.0, 6.0-6.5, or 6.5-7.0).

6. The antibody molecule of any of embodiments 1-5, wherein the normal tissue has a physiological pH.

7. The antibody molecule of any of embodiments 1-6, wherein the normal tissue has a pH of 7.4.

8. The antibody molecule of any of embodiments 1-7, which binds to CD138 with a higher affinity in a tumor microenvironment than in a normal tissue.

9. The antibody molecule of any of embodiments 1-8, wherein the binding affinity is at least 2, 3, 4, 5, 6, 7, 8, 9, 10, 50, 100, 500, 1,000, 2,000, 3,000, 4,000, 5,000, 6,000, 7,000, 8,000, 9,000, or 10,000-fold higher in a tumor microenvironment than in a normal tissue.

10. The antibody molecule of any of embodiments 1-9, wherein the binding affinity is at least 2-10,000, 3-5,000, 4-1,000, 5-500, 6-100, 7-50, 8-10, 2-5,000, 2-1,000, 2-500, 2-100, 2-50, 2-10, 1,000-5,000, 500-5,000, 100-5,000, 50-5,000, 10-5,000, 5-5,000, 2-4, 3-5, 4-6, 5-7, 6-8, 7-9, 8-10, 9-11, 10-12, 10-100, 50-500, 100-1,000, or 500-5,000-fold higher in a tumor microenvironment than in a normal tissue.

11. The antibody molecule of any of embodiments 1-10, wherein the binding affinity is 2, 3, 4, 5, 6, 7, 8, 9, 10, 50, 100, 500, 1,000, 2,000, 3,000, 4,000, 5,000, 6,000, 7,000, 8,000, 9,000, or 10,000-fold higher in a tumor microenvironment than in a normal tissue.

12. The antibody molecule of any of embodiments 1-11, which binds to CD138 with lower binding affinity than a reference anti-CD 138 antibody in a normal tissue.

13. The antibody molecule of any of embodiments 1-12, which does not substantially bind to CD138 in non-acidic conditions (e.g., at physiological pH, e.g., in a normal tissue).

14. The antibody molecule of any of embodiments 1-13, wherein the binding affinity of the antibody molecule is at least 2, 3, 4, 5, 6, 7, 8, 9, 10, 50, 100, 500, 1,000, 2,000, 3,000, 4,000, 5,000, 6,000, 7,000, 8,000, 9,000, or 10,000-fold lower than the binding affinity of the reference anti-CD 138 antibody in a normal tissue.

15. The antibody molecule of any of embodiments 1-14, wherein the binding affinity of the antibody molecule is at least 2-10,000, 3-5,000, 4-1,000, 5-500, 6-100, 7-50, 8-10, 2-5,000, 2-1,000, 2-500, 2-100, 2-50, 2-10, 1,000-5,000, 500-5,000, 100-5,000, 50-5,000, 10-5,000, 5-5,000, 2-4, 3-5, 4-6, 5-7, 6-8, 7-9, 8-10, 9-11, 10-12, 10-100, 50-500, 100-1,000, or 500-5,000-fold lower than the binding affinity of the reference anti-CD 138 antibody in a normal tissue.

16. The antibody molecule of any of embodiments 1-15, wherein the binding affinity of the antibody molecule is 2, 3, 4, 5, 6, 7, 8, 9, 10, 50, 100, 500, 1,000, 2,000, 3,000, 4,000, 5,000, 6,000, 7,000, 8,000, 9,000, or 10,000-fold lower than the binding affinity of the reference anti-CD138 antibody in a normal tissue.

17. The antibody molecule of any of embodiments 1-16, which binds to CD138 with the same, or substantially the same, binding affinity as a reference anti-CD138 antibody in a tumor microenvironment.

18. The antibody molecule of any of embodiments 1-17, wherein the binding affinity of the antibody molecule is no more than 2, 3, 4, 5, 6, 7, 8, 9, or 10-fold higher than the binding affinity of a reference anti-CD 138 antibody in a tumor microenvironment.

19. The antibody molecule of any of embodiments 1-18, wherein the binding affinity of the antibody molecule is no more than 2, 3, 4, 5, 6, 7, 8, 9, or 10-fold lower than the binding affinity of a reference anti-CD 138 antibody in a tumor microenvironment.

20. The antibody molecule of any of embodiments 1-19, wherein the binding affinity is determined by Octet assay.

21. The antibody molecule of any of embodiments 1-20, wherein the binding affinity is determined by surface plasmon resonance (SPR).

22. The antibody molecule of any of embodiments 1-21, wherein the binding affinity is determined by ELISA.

23. The antibody molecule of any of embodiments 1-22, wherein the binding affinity is determined by isothermal calorimetry (ITC).

24. The antibody molecule of any of embodiments 1-23, wherein the binding affinity is determined by a kinetic exclusion assay.

25. The antibody molecule of any of embodiments 1-24, wherein the binding affinity is determined using a CD138 protein or a fragment thereof.

26. The antibody molecule of any of embodiments 1-25, wherein the binding affinity is determined using a cell that expresses CD138.

27. The antibody molecule of any of embodiments 1-26, comprising a mutation in a complementarity determining region (CDR) compared to a reference anti-CD138 antibody.

28. The antibody molecule of any of embodiments 1-27, wherein the mutation results in selective binding to CD138 at an acidic pH.

29. The antibody molecule of any of embodiments 1-28, wherein the mutation does not significantly alter binding to CD138 at pH 7.4.

30. The antibody molecule of any of embodiments 1-29, comprising a plurality of mutations.

31. The antibody molecule of any of embodiments 1-30, wherein the plurality of mutations are in a single CDR.

32. The antibody molecule of any of embodiments 1-31, wherein at least two of the plurality of mutations are in two different CDRs.

33. The antibody molecule of any of embodiments 1-32, comprising a mutation in a heavy chain CDR (HCDR).

34. The antibody molecule of any of embodiments 1-33, wherein the mutation is in HCDR1.

35. The antibody molecule of any of embodiments 1-34, wherein the mutation is an H35 mutation (e.g., an H35N substitution).

36. The antibody molecule of any of embodiments 1-35, wherein the mutation is in HCDR2.

37. The antibody molecule of any of embodiments 1-36, wherein the mutation is a T50 mutation (e.g., a T50I substitution), an H52 mutation (e.g., an H52N or H52Y substitution), an S54 mutation (e.g., an S54G substitution), a D55 mutation (e.g., a D55A substitution), or a combination thereof.

38. The antibody molecule of any of embodiments 1-37, comprising an H52 mutation (e.g., an H52Y substitution) and an S54 mutation (e.g., an S54G substitution).

39. The antibody molecule of any of embodiments 1-38, comprising an H52 mutation (e.g., an H52Y substitution), an S54 mutation (e.g., an S54G substitution), and a D55 mutation (e.g., a D55A substitution).

40. The antibody molecule of any of embodiments 1-39, comprising a T50 mutation (e.g., a T50I substitution), an H52 mutation (e.g., an H52Y substitution), an S54 mutation (e.g., an S54G substitution), and a D55 mutation (e.g., a D55A substitution).

41. The antibody molecule of any of embodiments 1-40, comprising an H52 mutation (e.g., an H52N mutation).

42. The antibody molecule of any of embodiments 1-41, wherein the mutation is in HCDR3.

43. The antibody molecule of any of embodiments 1-42, wherein the mutation is an N98 mutation (e.g., an N98D substitution), a V100 mutation (e.g., a V100F substitution), or a combination thereof.

44. The antibody molecule of any of embodiments 1-43, comprising an N98 mutation (e.g., an N98D substitution).

45. The antibody molecule of any of embodiments 1-44, comprising a V100 mutation (e.g., a V100F substitution).

46. The antibody molecule of any of embodiments 1-45, comprising a mutation in HCDR1 and a mutation in HCDR2.

47. The antibody molecule of any of embodiments 1-46, comprising an H35 mutation (e.g., an H35N substitution) and an H52 mutation (e.g., an H52N mutation).

48. The antibody molecule of any of embodiments 1-47, comprising a mutation in HCDR2 and a mutation in HCDR3.

49. The antibody molecule of any of embodiments 1-48, comprising an H52 mutation (e.g., an H52Y substitution), an S54 mutation (e.g., an S54G substitution), a D55 mutation (e.g., a D55A substitution), and an N98 mutation (e.g., an N98D substitution).

50. The antibody molecule of any of embodiments 1-49, comprising a T50 mutation (e.g., a T50I substitution), an H52 mutation (e.g., an H52Y substitution), an S54 mutation (e.g., an S54G substitution), a D55 mutation (e.g., a D55A substitution), and an N98 mutation (e.g., an N98D substitution).

51. The antibody molecule of any of embodiments 1-50, comprising a T50 mutation (e.g., a T50I substitution), an H52 mutation (e.g., an H52Y substitution), an S54 mutation (e.g., an S54G substitution), a D55 mutation (e.g., a D55A substitution), an N98 mutation (e.g., an N98D substitution), and a V100 mutation (e.g., a V100F substitution).

52. The antibody molecule of any of embodiments 1-51, which further comprises a mutation in a heavy chain framework region (FR).

53. The antibody molecule of any of embodiments 1-52, comprising a mutation in a light chain CDR (LCDR).

54. The antibody molecule of any of embodiments 1-53, wherein the mutation is in LCDR1.

55. The antibody molecule of any of embodiments 1-54, wherein the mutation is a K35 mutation (e.g., a K35H substitution).

56. The antibody molecule of any of embodiments 1-55, wherein the mutation is in LCDR2.

57. The antibody molecule of any of embodiments 1-56, wherein the mutation is a Y45 mutation (e.g., a Y45D substitution).

58. The antibody molecule of any of embodiments 1-57, wherein the mutation is in LCDR2.

59. The antibody molecule of any of embodiments 1-58, wherein the mutation is an N39 mutation (e.g., an N39L substitution).

60. The antibody molecule of any of embodiments 1-59, wherein the mutation is in LCDR1.

61. The antibody molecule of any of embodiments 1-60, which further comprises a mutation in a light chain framework region (FR).

62. The antibody molecule of any of embodiments 1-61, comprising the HCDR1-3 and LCDR1-3 of a reference anti-CD138 antibody other than an N98 mutation (e.g., an N98D substitution) in HCDR3.

63. The antibody molecule of any of embodiments 1-62, comprising the HCDR1-3 and LCDR1-3 of a reference anti-CD 138 antibody other than an H52 mutation (e.g., an H52Y substitution), an S54 mutation (e.g., an S54G substitution), and a D55 mutation (e.g., a D55A substitution) in HCDR2, and an N98 mutation (e.g., an N98D substitution) in HCDR3.

64. The antibody molecule of any of embodiments 1-63, comprising the HCDR1-3 and LCDR1-3 of a reference anti-CD138 antibody other than an N98 mutation (e.g., an N98D substitution) in HCDR3, and a K35Hmutation (e.g., a K35H substitution) in LCDR1.

65. The antibody molecule of any of embodiments 1-64, comprising the HCDR1-3 and LCDR1-3 of a reference anti-CD 138 antibody other than an H52 mutation (e.g., an H52Y substitution) and an S54 mutation (e.g., an S54G substitution) in HCDR2, and an N98 mutation (e.g., an N98D substitution) in HCDR3.

66. The antibody molecule of any of embodiments 1-65, comprising the HCDR1-3 and LCDR1-3 of a reference anti-CD138 antibody other than a Y54 mutation (e.g., a Y54D substitution) in LCDR2.

67. The antibody molecule of any of embodiments 1-66, comprising the HCDR1-3 and LCDR1-3 of a reference anti-CD138 antibody other than an S54 mutation (e.g., an S54E substitution) in HCDR2.

68. The antibody molecule of any of embodiments 1-67, comprising the HCDR1-3 and LCDR1-3 of a reference anti-CD138 antibody other than a V100 mutation (e.g., a V100L substitution) in HCDR3.

69. The antibody molecule of any of embodiments 1-68, comprising the HCDR1-3 and LCDR1-3 of a reference anti-CD138 antibody other than an N39 mutation (e.g., an N39L substitution) in LCDR1.

70. The antibody molecule of any of embodiments 1-69, comprising the HCDR1-3 and LCDR1-3 of a reference anti-CD138 antibody other than a T50 mutation (e.g., a T50D substitution) in HCDR2.

71. The antibody molecule of any of embodiments 1-70, which binds to at least a first epitope and a second epitope of CD138, wherein the first epitope and the second epitope each comprise the amino acid sequence VEP.

72. The antibody molecule of embodiment 71, wherein the first epitope and the second epitope are located in different regions of CD138.

73. The antibody molecule of any of embodiments 1-72, wherein the first epitope comprises an arginine residue positioned 1, 2, 3, 4, 5, 6, 7, or 8 amino acids (e.g., 5 amino acids) C-terminal relative to the VEP.

74. The antibody molecule of any of embodiments 1-73, wherein the second epitope comprises an arginine residue positioned 1, 2, 3, 4, 5, 6, 7, or 8 amino acids (e.g., 2 amino acids) C-terminal relative to the VEP.

75. The antibody molecule of any of embodiments 1-74, wherein the first epitope comprises the amino acid sequence ENTAVVAVEPDRRNQ, or an amino acid sequence differing by no more than 1, 2, 3, 4, or 5 amino acids (e.g., substitution, deletion, or insertion) therefrom.

76. The antibody molecule of any of embodiments 1-75, wherein the first epitope is comprised in a membrane-proximal region of CD138.

77. The antibody molecule of any of embodiments 1-76, wherein the second epitope comprises the amino acid sequence GEAVVLPEVEPGLTAR, or an amino acid sequence differing by no more than 1, 2, 3, 4, or 5 amino acids (e.g., substitution, deletion, or insertion) therefrom.

78. The antibody molecule of any of embodiments 1-77, wherein the second epitope comprises the amino acid sequence GEAVVLPEVEPGLTAREQEA, or an amino acid sequence differing by no more than 1, 2, 3, 4, or 5 amino acids (e.g., substitution, deletion, or insertion) therefrom.

79. The antibody molecule of any of embodiments 1-78, wherein the second epitope is comprised in an integrin-binding region of CD138.

80. The antibody molecule of any of embodiments 1-79, wherein the antibody molecule comprises an HCDR1 that binds to the first epitope, e.g., at a histidine residue of the HCDR1 (e.g., HCDR1:H35).

81. The antibody molecule of any of embodiments 1-80, wherein the antibody molecule comprises an HCDR1 that binds to the second epitope, e.g., at a histidine residue of the HCDR1 (e.g., HCDR1:H35).

82. The antibody molecule of any of embodiments 1-81, wherein the antibody molecule comprises an HCDR2 that binds to the first epitope, e.g., at a histidine residue of the HCDR2 (e.g., HCDR2:H52).

83. The antibody molecule of any of embodiments 1-82, wherein the antibody molecule comprises an HCDR2 that binds to the second epitope, e.g., at a histidine residue of the HCDR2 (e.g., HCDR2:H52).

84. An anti-CD138 antibody molecule having a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2 (e.g., at least 2, 3, 4, 5, 6, 7, 8, 9, or 10).

85. An anti-CD138 antibody molecule having an IC50 value for CD138 at pH 7.4 and an IC50 value for CD138 at pH of 5.5-7.0 (e.g., a pH of 5.5-6.0, 6.0-6.5, or 6.5-7.0), wherein the ratio of IC50 for CD138 at pH 7.4 and IC50 for CD138 at pH of 5.5-7.0 (e.g., a pH of 5.5-6.0, 6.0-6.5, or 6.5-7.0) is at least 2 (e.g., at least 2, 3, 4, 5, 6, 7, 8, 9, or 10).

86. The antibody molecule of any of embodiments 1-85, wherein the KD value for CD138 at pH 5.5-7.0 is a KD value for CD138 at pH 5.5-5.7, 5.7-6.1, 6.1 to 6.4, 6.2 to 6.3, 6.0 to 6.4, 6.0 to 6.3, 6.0 to 6.2, 6.3 to 6.5, 6.2 to 6.5, 6.1 to 6.5, 6.1 to 6.3, 6.2 to 6.4, 6.3 to 6.5, or 6.5-7.0.

87. The antibody molecule of any of embodiments 1-86, wherein the KD value for CD138 at pH 5.5 to 7.0 is a KD value for CD138 at pH 5.5, 5.6, 5.7, 5.8, 5.9, 6.0, 6.1, 6.2, 6.3, 6.4, 6.5, 6.6, 6.7, 6.8, 6.9, or 7.0.

88. The antibody molecule of any of embodiments 1-87, wherein the ratio of KD for CD138 at pH 7.4 and KD for CD138 at pH of 5.5-7.0 (e.g., a pH of 5.5-6.0, 6.0-6.5, or 6.5-7.0) is at least 3, 4, 5, 6, 7, 8, 9, 10, 50, 100, 500, 1,000, 2,000, 3,000, 4,000, 5,000, 6,000, 7,000, 8,000, 9,000, or 10,000.

89. The antibody molecule of any of embodiments 1-88, wherein the ratio of KD for CD138 at pH 7.4 and KD for CD138 at pH of 5.5-7.0 (e.g., a pH of 5.5-6.0, 6.0-6.5, or 6.5-7.0) is 2-10,000, 3-5,000, 4-1,000, 5-500, 6-100, 7-50, 8-10, 2-5,000, 2-1,000, 2-500, 2-100, 2-50, 2-10, 1,000-5,000, 500-5,000, 100-5,000, 50-5,000, 10-5,000, 5-5,000, 2-4, 3-5, 4-6, 5-7, 6-8, 7-9, 8-10, 9-11, 10-12, 10-100, 50-500, 100-1,000, or 500-5,000.

90. The antibody molecule of any of embodiments 1-89, wherein the ratio of KD for CD138 at pH 7.4 and KD for CD138 at pH of 5.5-7.0 (e.g., a pH of 5.5-6.0, 6.0-6.5, or 6.5-7.0) is 2, 3, 4, 5, 6, 7, 8, 9, 10, 50, 100, 500, 1,000, 2,000, 3,000, 4,000, 5,000, 6,000, 7,000, 8,000, 9,000, or 10,000.

91. The antibody molecule of any of embodiments 1-90, which binds to CD138 with a higher KD value than a reference anti-CD138 antibody at pH 7.4.

92. The antibody molecule of any of embodiments 1-91, wherein the KD value of the antibody molecule is at least 2, 3, 4, 5, 6, 7, 8, 9, 10, 50, 100, 500, 1,000, 2,000, 3,000, 4,000, 5,000, 6,000, 7,000, 8,000, 9,000, or 10,000-fold higher than the KD value of the reference anti-CD 138 antibody at pH 7.4.

93. The antibody molecule of any of embodiments 1-92, wherein the KD value of the antibody molecule is at least 2-10,000, 3-5,000, 4-1,000, 5-500, 6-100, 7-50, 8-10, 2-5,000, 2-1,000, 2-500, 2-100, 2-50, 2-10, 1,000-5,000, 500-5,000, 100-5,000, 50-5,000, 10-5,000, 5-5,000, 2-4, 3-5, 4-6, 5-7, 6-8, 7-9, 8-10, 9-11, 10-12, 10-100, 50-500, 100-1,000, or 500-5,000-fold higher than the binding affinity of the reference anti-CD138 antibody at pH 7.4.

94. The antibody molecule of any of embodiments 1-93, wherein the KD value of the antibody molecule is 2, 3, 4, 5, 6, 7, 8, 9, 10, 50, 100, 500, 1,000, 2,000, 3,000, 4,000, 5,000, 6,000, 7,000, 8,000, 9,000, or 10,000-fold higher than the KD value of the reference anti-CD138 antibody at pH 7.4.

95. The antibody molecule of any of embodiments 1-94, which binds to CD138 with the same, or substantially the same, binding affinity as a reference anti-CD 138 antibody at pH of 5.5-7.0 (e.g., a pH of 5.5-6.0, 6.0-6.5, or 6.5-7.0).

96. The antibody molecule of any of embodiments 1-95, wherein the KD value of the antibody molecule is no more than 2, 3, 4, 5, 6, 7, 8, 9, or 10-fold lower than the KD value of a reference anti-CD138 antibody at pH of 5.5-7.0 (e.g., a pH of 5.5-6.0, 6.0-6.5, or 6.5-7.0).

97. The antibody molecule of any of embodiments 1-96, wherein the KD value of the antibody molecule is no more than 2, 3, 4, 5, 6, 7, 8, 9, or 10-fold higher than the KD of a reference anti-CD 138 antibody at pH of 5.5-7.0 (e.g., a pH of 5.5-6.0, 6.0-6.5, or 6.5-7.0).

98. The antibody molecule of any of embodiments 1-97, wherein the binding affinity is determined by Octet assay.

99. The antibody molecule of any of embodiments 1-98, wherein the binding affinity is determined by surface plasmon resonance (SPR).

100. The antibody molecule of any of embodiments 1-99, wherein the binding affinity is determined by ELISA.

101. The antibody molecule of any of embodiments 1-100, wherein the binding affinity is determined by isothermal calorimetry (ITC).

102. The antibody molecule of any of embodiments 1-101, wherein the binding affinity is determined by a kinetic exclusion assay.

103. The antibody molecule of any of embodiments 1-102, wherein the binding affinity is determined using a CD138 protein or a fragment thereof.

104. The antibody molecule of any of embodiments 1-103, wherein the binding affinity is determined using a cell that expresses CD138.

105. The antibody molecule of any of embodiments 1-104, comprising a mutation in a complementarity determining region (CDR) compared to a reference anti-CD138 antibody.

106. The antibody molecule of any of embodiments 1-105, wherein the mutation results in selective binding to CD138 at an acidic pH.

107. The antibody molecule of any of embodiments 1-106, wherein the mutation does not significantly alter binding to CD138 at pH 7.4.

108. The antibody molecule of any of embodiments 1-107, comprising a plurality of mutations.

109. The antibody molecule of any of embodiments 1-108, wherein the plurality of mutations are in a single CDR.

110. The antibody molecule of any of embodiments 1-109, wherein at least two of the plurality of mutations are in two different CDRs.

111. The antibody molecule of any of embodiments 1-110, comprising a mutation in a heavy chain CDR (HCDR).

112. The antibody molecule of any of embodiments 1-111, wherein the mutation is in HCDR1.

113. The antibody molecule of any of embodiments 1-112, wherein the mutation is an H35 mutation (e.g., an H35N substitution).

114. The antibody molecule of any of embodiments 1-113, wherein the mutation is in HCDR2.

115. The antibody molecule of any of embodiments 1-114, wherein the mutation is a T50 mutation (e.g., a T50I substitution), an H52 mutation (e.g., an H52N or H52Y substitution), an S54 mutation (e.g., an S54G substitution), a D55 mutation (e.g., a D55A substitution), or a combination thereof.

116. The antibody molecule of any of embodiments 1-115, comprising an H52 mutation (e.g., an H52Y substitution) and an S54 mutation (e.g., an S54G substitution).

117. The antibody molecule of any of embodiments 1-116, comprising an H52 mutation (e.g., an H52Y substitution), an S54 mutation (e.g., an S54G substitution), and a D55 mutation (e.g., a D55A substitution).

118. The antibody molecule of any of embodiments 1-117, comprising a T50 mutation (e.g., a T50I substitution), an H52 mutation (e.g., an H52Y substitution), an S54 mutation (e.g., an S54G substitution), and a D55 mutation (e.g., a D55A substitution).

119. The antibody molecule of any of embodiments 1-118, comprising an H52 mutation (e.g., an H52N mutation).

120. The antibody molecule of any of embodiments 1-119, wherein the mutation is in HCDR3.

121. The antibody molecule of any of embodiments 1-120, wherein the mutation is an N98 mutation (e.g., an N98D substitution), a V100 mutation (e.g., a V100F substitution), or a combination thereof.

122. The antibody molecule of any of embodiments 1-121, comprising an N98 mutation (e.g., an N98D substitution).

123. The antibody molecule of any of embodiments 1-122, comprising a V100 mutation (e.g., a V100F substitution).

124. The antibody molecule of any of embodiments 1-123, comprising a mutation in HCDR1 and a mutation in HCDR2.

125. The antibody molecule of any of embodiments 1-124, comprising an H35 mutation (e.g., an H35N substitution) and an H52 mutation (e.g., an H52N mutation).

126. The antibody molecule of any of embodiments 1-125, comprising a mutation in HCDR2 and a mutation in HCDR3.

127. The antibody molecule of any of embodiments 1-126, comprising an H52 mutation (e.g., an H52Y substitution), an S54 mutation (e.g., an S54G substitution), a D55 mutation (e.g., a D55A substitution), and an N98 mutation (e.g., an N98D substitution).

128. The antibody molecule of any of embodiments 1-127, comprising a T50 mutation (e.g., a T50I substitution), an H52 mutation (e.g., an H52Y substitution), an S54 mutation (e.g., an S54G substitution), a D55 mutation (e.g., a D55A substitution), and an N98 mutation (e.g., an N98D substitution).

129. The antibody molecule of any of embodiments 1-128, comprising a T50 mutation (e.g., a T50I substitution), an H52 mutation (e.g., an H52Y substitution), an S54 mutation (e.g., an S54G substitution), a D55 mutation (e.g., a D55A substitution), an N98 mutation (e.g., an N98D substitution), and a V100 mutation (e.g., a V100F substitution).

130. The antibody molecule of any of embodiments 1-129, which further comprises a mutation in a heavy chain framework region (FR).

131. The antibody molecule of any of embodiments 1-130, comprising a mutation in a light chain CDR (LCDR).

132. The antibody molecule of any of embodiments 1-131, wherein the mutation is in LCDR1.

133. The antibody molecule of any of embodiments 1-132, wherein the mutation is a K35 mutation (e.g., a K35H substitution).

134. The antibody molecule of any of embodiments 1-133, wherein the mutation is in LCDR2.

135. The antibody molecule of any of embodiments 1-134, wherein the mutation is a Y45 mutation (e.g., a Y45D substitution).

136. The antibody molecule of any of embodiments 1-135, wherein the mutation is in LCDR2.

137. The antibody molecule of any of embodiments 1-136, wherein the mutation is an N39 mutation (e.g., an N39L substitution).

138. The antibody molecule of any of embodiments 1-137, wherein the mutation is in LCDR1.

139. The antibody molecule of any of embodiments 1-138, which further comprises a mutation in a light chain framework region (FR).

140. The antibody molecule of any of embodiments 1-139, comprising the HCDR1-3 and LCDR1-3 of a reference anti-CD138 antibody other than an N98 mutation (e.g., an N98D substitution) in HCDR3.

141. The antibody molecule of any of embodiments 1-140, comprising the HCDR1-3 and LCDR1-3 of a reference anti-CD138 antibody other than an H52 mutation (e.g., an H52Y substitution), an S54 mutation (e.g., an S54G substitution), and a D55 mutation (e.g., a D55A substitution) in HCDR2, and an N98 mutation (e.g., an N98D substitution) in HCDR3.

142. The antibody molecule of any of embodiments 1-141, comprising the HCDR1-3 and LCDR1-3 of a reference anti-CD138 antibody other than an N98 mutation (e.g., an N98D substitution) in HCDR3, and a K35 mutation (e.g., a K35H substitution) in LCDR1.

143. The antibody molecule of any of embodiments 1-142, comprising the HCDR1-3 and LCDR1-3 of a reference anti-CD138 antibody other than an H52 mutation (e.g., an H52Y substitution) and an S54 mutation (e.g., an S54G substitution) in HCDR2, and an N98 mutation (e.g., an N98D substitution) in HCDR3.

144. The antibody molecule of any of embodiments 1-143, comprising the HCDR1-3 and LCDR1-3 of a reference anti-CD138 antibody other than a Y54 mutation (e.g., a Y54D substitution) in LCDR2.

145. The antibody molecule of any of embodiments 1-144, comprising the HCDR1-3 and LCDR1-3 of a reference anti-CD138 antibody other than an S54 mutation (e.g., an S54E substitution) in HCDR2.

146. The antibody molecule of any of embodiments 1-145, comprising the HCDR1-3 and LCDR1-3 of a reference anti-CD 138 antibody other than a V100 mutation (e.g., a V100L substitution) in HCDR3.

147. The antibody molecule of any of embodiments 1-146, comprising the HCDR1-3 and LCDR1-3 of a reference anti-CD138 antibody other than an N39 mutation (e.g., an N39L substitution) in LCDR2.

148. The antibody molecule of any of embodiments 1-147, comprising the HCDR1-3 and LCDR1-3 of a reference anti-CD138 antibody other than a T50 mutation (e.g., a T50D substitution) in HCDR2.

149. The antibody molecule of any of embodiments 1-148, which binds to at least a first epitope and a second epitope of CD138, wherein the first epitope and the second epitope each comprise the amino acid sequence VEP.

150. The antibody molecule of embodiment 149, wherein the first epitope and the second epitope are located in different regions of CD138.

151. The antibody molecule of any of embodiments 1-150, wherein the first epitope comprises an arginine residue positioned 1, 2, 3, 4, 5, 6, 7, or 8 amino acids (e.g., 5 amino acids) C-terminal relative to the VEP.

152. The antibody molecule of any of embodiments 1-151, wherein the second epitope comprises an arginine residue positioned 1, 2, 3, 4, 5, 6, 7, or 8 amino acids (e.g., 2 amino acids) C-terminal relative to the VEP.

153. The antibody molecule of any of embodiments 1-152, wherein the first epitope comprises the amino acid sequence ENTAVVAVEPDRRNQ, or an amino acid sequence differing by no more than 1, 2, 3, 4, or 5 amino acids (e.g., substitution, deletion, or insertion) therefrom.

154. The antibody molecule of any of embodiments 1-153, wherein the first epitope is comprised in a membrane-proximal region of CD138.

155. The antibody molecule of any of embodiments 1-154, wherein the second epitope comprises the amino acid sequence GEAVVLPEVEPGLTAR, or an amino acid sequence differing by no more than 1, 2, 3, 4, or 5 amino acids (e.g., substitution, deletion, or insertion) therefrom.

156. The antibody molecule of any of embodiments 1-155, wherein the second epitope comprises the amino acid sequence GEAVVLPEVEPGLTAREQEA, or an amino acid sequence differing by no more than 1, 2, 3, 4, or 5 amino acids (e.g., substitution, deletion, or insertion) therefrom.

157. The antibody molecule of any of embodiments 1-156, wherein the second epitope is comprised in an integrin-binding region of CD138.

158. The antibody molecule of any of embodiments 1-157, wherein the antibody molecule comprises an HCDR1 that binds to the first epitope, e.g., at a histidine residue of the HCDR1 (e.g., HCDR1:H35).

159. The antibody molecule of any of embodiments 1-158, wherein the antibody molecule comprises an HCDR1 that binds to the second epitope, e.g., at a histidine residue of the HCDR1 (e.g., HCDR1:H35).

160. The antibody molecule of any of embodiments 1-159, wherein the antibody molecule comprises an HCDR2 that binds to the first epitope, e.g., at a histidine residue of the HCDR2 (e.g., HCDR2:H52).

161. The antibody molecule of any of embodiments 1-160, wherein the antibody molecule comprises an HCDR2 that binds to the second epitope, e.g., at a histidine residue of the HCDR2 (e.g., HCDR2:H52).

162. An anti-CD138 antibody molecule comprising:
(a) a heavy chain variable region (VH), wherein the VH comprises three heavy chain complementarity determining regions (HCDR1, HCDR2, and HCDR3), wherein the VH comprises 1, 2, or all 3 of:
   (i) an HCDR1 comprising an amino acid sequence that differs by no more than 1, 2, or 3 amino acid residues from, or has at least 85, 90, 95, 99 or 100% homology with, the amino acid sequence of the HCDR1 of an anti-CD138 antibody as listed in Table 1;
   (ii) an HCDR2 comprising an amino acid sequence that differs by no more than 1, 2, or 3 amino acid residues from, or has at least 85, 90, 95, 99 or 100% homology with, the amino acid sequence of the HCDR2 of the anti-CD138 antibody; and/or
   (iii) an HCDR3 comprising an amino acid sequence that differs by no more than 1, 2, or 3 amino acid residues from, or has at least 85, 90, 95, 99 or 100% homology with, the amino acid sequence of the HCDR3 of the anti-CD138 antibody; and/or
(b) a light chain variable region (VL), wherein the VL comprises three light chain complementarity determining regions (LCDR1, LCDR2, and LCDR3), wherein the VL comprises 1, 2, or all 3 of:
   (i) an LCDR1 comprising an amino acid sequence that differs by no more than 1, 2, or 3 amino acid residues from, or has at least 85, 90, 95, 99 or 100% homology with, the amino acid sequence of the LCDR1 of the anti-CD 138 antibody;
   (ii) an LCDR2 comprising an amino acid sequence that differs by no more than 1, 2, or 3 amino acid residues from, or has at least 85, 90, 95, 99 or 100% homology with, the amino acid sequence of the LCDR2 of the anti-CD138 antibody; and/or
   (iii) an LCDR3 comprising an amino acid sequence that differs by no more than 1, 2, or 3 amino acid residues from, or has at least 85, 90, 95, 99 or 100% homology with, the amino acid sequence of the LCDR3 of the anti-CD138 antibody.

163. The antibody molecule of embodiment 162, comprising:
(a) a VH comprising: (i) an HCDR1 comprising the amino acid sequence of the HCDR1 of the anti-CD138 antibody; (ii) an HCDR2 comprising the amino acid sequence of the HCDR2 of the anti-CD138 antibody; and (iii) an HCDR3 comprising the amino acid sequence of the HCDR3 of the anti-CD138 antibody, and
(b) a VL comprising: (i) an LCDR1 comprising the amino acid sequence of the LCDR1 of the anti-CD138 antibody; (ii) an LCDR2 comprising the amino acid sequence of the LCDR2 of the anti-CD138 antibody; and (iii) an LCDR3 comprising the amino acid sequence of the LCDR3 of the anti-CD138 antibody.

164. The antibody molecule of embodiment 162 or 163, wherein the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody.

165. The antibody molecule of any of embodiments 162-164, wherein the VH comprises the amino acid sequence of the VH of the anti-CD138 antibody.

166. The antibody molecule of any of embodiments 162-165, wherein the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VL of the anti-CD 138 antibody.

167. The antibody molecule of any of embodiments 162-166, wherein the VL comprises the amino acid sequence of the VL of the anti-CD138 antibody.

168. The antibody molecule of any of embodiments 162-167, wherein:
(a) the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody; and
(b) the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody.

169. The antibody molecule of any of embodiments 162-168, wherein the VH comprises the amino acid sequence of the VH of the anti-CD138 antibody and the VL comprises the amino acid sequence of the VL of the anti-CD138 antibody.

170. The antibody molecule of any of embodiments 162-169, comprising an Fc region.

171. The antibody molecule of any of embodiments 162-170, comprising a heavy chain (HC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the HC of the anti-CD 138 antibody.

172. The antibody molecule of embodiment 171, wherein the HC comprises the amino acid sequence of the HC of the anti-CD 138 antibody.

173. The antibody molecule of any of embodiments 162-172, comprising a light chain (LC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the LC of the anti-CD138 antibody.

174. The antibody molecule of embodiment 173, wherein the LC comprises the amino acid sequence of the LC of the anti-CD138 antibody.

175. The antibody molecule of any of embodiments 162-174, comprising:
(a) a heavy chain (HC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the HC of the anti-CD 138 antibody; and
(b) a light chain (LC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the LC of the anti-CD 138 antibody.

176. The antibody molecule of embodiment 175, wherein the HC comprises the amino acid sequence of the HC of the anti-CD138 antibody and the LC comprises the amino acid sequence of the LC of the anti-CD138 antibody.

177. The antibody molecule of any of embodiments 162-176, wherein the antibody molecule is 28-0, as listed in Table 1 or 2.

178. The antibody molecule of any of embodiments 162-176, wherein the antibody molecule is 29-0, as listed in Table 1 or 2.

179. The antibody molecule of any of embodiments 162-176, wherein the antibody molecule is Ab17, as listed in Table 1 or 2.

180. The antibody molecule of any of embodiments 162-176, wherein the antibody molecule is Ab18, as listed in Table 1 or 2.

181. The antibody molecule of any of embodiments 162-176, wherein the antibody molecule is Ab29, as listed in Table 1 or 2.

182. The antibody molecule of any of embodiments 162-176, wherein the antibody molecule is Ab28, as listed in Table 1 or 2.

183. The antibody molecule of any of embodiments 162-176, wherein the antibody molecule is Ab30, as listed in Table 1 or 2.

184. The antibody molecule of any of embodiments 162-176, wherein the antibody molecule is Ab32, as listed in Table 1 or 2.

185. The antibody molecule of any of embodiments 162-176, wherein the antibody molecule is Ab34, as listed in Table 1 or 2.

186. The antibody molecule of any of embodiments 162-176, wherein the antibody molecule is Ab43, as listed in Table 1 or 2.

187. The antibody molecule of any of embodiments 162-176, wherein the antibody molecule is Ab71, as listed in Table 1 or 2.

188. The antibody molecule of any of embodiments 162-176, wherein the antibody molecule is Ab137, as listed in Table 1 or 2.

189. The antibody molecule of any of embodiments 162-176, wherein the antibody molecule is Ab173, as listed in Table 1 or 2.

190. The antibody molecule of any of embodiments 162-176, wherein the antibody molecule is Ab174, as listed in Table 1 or 2.

191. The antibody molecule of any of embodiments 162-176, wherein the antibody molecule is Ab175, as listed in Table 1 or 2.

192. An anti-CD138 antibody molecule that binds to at least a first epitope and a second epitope of CD138, wherein the first epitope and the second epitope each comprise the amino acid sequence VEP.

193. The antibody molecule of embodiment 192, wherein the first epitope and the second epitope are located in different regions of CD138.

194. The antibody molecule of embodiment 192 or 193, wherein the first epitope comprises an arginine residue positioned 1, 2, 3, 4, 5, 6, 7, or 8 amino acids (e.g., 5 amino acids) C-terminal relative to the VEP.

195. The antibody molecule of any of embodiments 192-194, wherein the second epitope comprises an arginine residue positioned 1, 2, 3, 4, 5, 6, 7, or 8 amino acids (e.g., 2 amino acids) C-terminal relative to the VEP.

196. The antibody molecule of any of embodiments 192-195, wherein the first epitope comprises the amino acid sequence ENTAVVAVEPDRRNQ, or an amino acid sequence differing by no more than 1, 2, 3, 4, or 5 amino acids (e.g., substitution, deletion, or insertion) therefrom.

197. The antibody molecule of any of embodiments 192-196, wherein the first epitope is comprised in a membrane-proximal region of CD138.

198. The antibody molecule of any of embodiments 192-197, wherein the second epitope comprises the amino acid sequence GEAVVLPEVEPGLTAR, or an amino acid sequence differing by no more than 1, 2, 3, 4, or 5 amino acids (e.g., substitution, deletion, or insertion) therefrom.

199. The antibody molecule of any of embodiments 192-198, wherein the second epitope comprises the amino acid sequence GEAVVLPEVEPGLTAREQEA, or an amino acid sequence differing by no more than 1, 2, 3, 4, or 5 amino acids (e.g., substitution, deletion, or insertion) therefrom.

200. The antibody molecule of any of embodiments 192-199, wherein the second epitope is comprised in an integrin-binding region of CD138.

201. The antibody molecule of any of embodiments 192-200, wherein the antibody molecule comprises an HCDR1 that binds to the first epitope, e.g., at a histidine residue of the HCDR1 (e.g., HCDR1:H35).

202. The antibody molecule of any of embodiments 192-201, wherein the antibody molecule comprises an HCDR1 that binds to the second epitope, e.g., at a histidine residue of the HCDR1 (e.g., HCDR1:H35).

203. The antibody molecule of any of embodiments 192-202, wherein the antibody molecule comprises an HCDR2 that binds to the first epitope, e.g., at a histidine residue of the HCDR2 (e.g., HCDR2:H52).

204. The antibody molecule of any of embodiments 192-203, wherein the antibody molecule comprises an HCDR2 that binds to the second epitope, e.g., at a histidine residue of the HCDR2 (e.g., HCDR2:H52).

205. An antibody molecule, which binds, or substantially binds, to an epitope that completely or partially overlaps with the epitope of an anti-CD 138 antibody molecule as listed in Table 1.

206. An antibody molecule, which competes with an anti-CD138 antibody molecule as listed in Table 1 for binding to CD138.

207. The antibody molecule of any of embodiments 1-206, comprising two VHs and two VLs.

208. The antibody molecule of any of embodiments 1-207, which is a synthetic antibody molecule or an isolated antibody molecule.

209. The antibody molecule of any of embodiments 1-208, which is a monovalent antibody molecule, a multivalent (e.g., bivalent, trivalent, or tetravalent) antibody molecule, a monospecific molecule, or a multispecific (e.g., bispecific, trispecific, or tetraspecific) antibody molecule.

210. The antibody molecule of any of embodiments 1-209, which is a humanized antibody molecule.

211. The antibody molecule of any of embodiments 1-210, comprising one or more framework regions derived from human framework germline sequence.

212. The antibody molecule of any of embodiments 1-211, which is an IgG antibody.

213. The antibody molecule of any of embodiments 1-212, comprising a heavy chain constant region of IgG chosen from IgG1 (e.g., IgGm3), IgG2, IgG3, or IgG4.

214. The antibody molecule of any of embodiments 1-213, comprising a light chain constant region of kappa or lambda light chain.

215. The antibody molecule of any of embodiments 1-214, comprising a heavy chain constant region of IgG1 and a light chain constant region of kappa.

216. The antibody molecule of any of embodiments 1-215, comprising an Fc region comprising one or more mutations to increase the binding affinity to neonatal receptor FcRn and/or the half-life of the antibody molecule.

217. The antibody molecule of any of embodiments 1-216, comprising an Fc region comprising one or more mutations described herein, e.g., to increase one or more of half-life, ADCC, CDC, or ADCP.

218. The antibody molecule of any of embodiments 1-217, which is afucosylated ( e.g., at N297 of an Fc region of the antibody molecule).

219. An antibody-molecule drug conjugate (ADC) comprising an antibody molecule of any of the preceding embodiments, optionally comprising a cytotoxic agent, further optionally comprising a linker.

220. A composition comprising an antibody molecule of any of embodiments 1-218, or an ADC of embodiment 29, optionally, wherein the composition is a pharmaceutical composition.

221. The composition of embodiment 220, further comprising a pharmaceutically acceptable carrier.

222. A nucleic acid molecule encoding a heavy chain variable region (VH), a light chain variable region (VL), or both, of an antibody molecule of any of embodiments 1-218.

223. A vector comprising a nucleic acid molecule of embodiment 222, optionally wherein the vector is an expression vector.

224. A cell comprising a nucleic acid molecule of embodiment 222, or a vector of embodiment 223, optionally, wherein the cell is an isolated cell.

225. A kit comprising an antibody molecule of any of embodiments 1-218, an ADC of embodiment 219, or a composition of embodiment 220 or 221, and instructions to use of the antibody molecule or composition.

226. A container comprising an antibody molecule of any of embodiments 1-218, an ADC of embodiment 219, or a composition of embodiment 220 or 221.

227. A method of producing an anti-CD138 antibody molecule, the method comprising culturing a cell of embodiment 224 under conditions that allow production of an antibody molecule, thereby producing the antibody molecule.

228. The method of embodiment 227, further comprising isolating or purifying the antibody molecule.

229. A method of producing an ADC, the method comprising coupling an antibody molecule of any of embodiments 1-218 with a non-antibody moiety, thereby producing the ADC.

230. The method of embodiment 229, wherein the non-antibody moiety is a cytotoxic agent.

231. An antibody molecule of any of embodiments 1-218, an ADC of embodiment 219, or a composition of embodiment 220 or 221, for use in a method of treating a cancer in a subject.

232. A method of treating a cancer, the method comprising administering an effective amount of an antibody molecule of any of embodiments 1-218, an ADC of embodiment 219, or a composition of embodiment 220 or 221, to a subject in need thereof, thereby treating the cancer.

233. An antibody molecule of any of embodiments 1-218, an ADC of embodiment 219, or a composition of embodiment 220 or 221, for use in a method of treating an osteolytic disease in a subject.

234. The antibody molecule, ADC, composition for use, or method of any of embodiments 231-233, wherein the cancer comprises a tumor microenvironment, and the antibody molecule or ADC is enriched in the microenvironment.

235. The antibody molecule, ADC, composition for use, or method of any of embodiments 231-233, wherein the cancer comprises a tumor microenvironment having a pH less than the pH of a tissue surrounding the tumor microenvironment.

236. The antibody molecule, ADC, composition for use, or method of any of embodiments 231-233, wherein the cancer comprises a tumor microenvironment having a pH less than the pH of a normal tissue, e.g., pH 7.4.

237. The antibody molecule, ADC, composition for use, or method of any of embodiments 231-233, wherein the cancer comprises a tumor microenvironment having a pH of 6.0 to 6.5.

238. The antibody molecule, ADC, composition for use, or method of any of embodiments 231-233, wherein the antibody molecule is not enriched in a tissue surrounding the tumor microenvironment.

239. The antibody molecule, ADC, composition for use, or method of any of embodiments 231-233, wherein the antibody molecule is not enriched in a normal tissue.

240. The antibody molecule, ADC, composition for use, or method of any of embodiments 231-233, wherein the cancer is a solid tumor.

241. The antibody molecule, ADC, composition for use, or method of any of embodiments 231-233, wherein the cancer is a pancreatic cancer (e.g., pancreatic ductal adenoma carcinoma (PDAC)), a breast cancer, renal carcinoma, a lung cancer, a urogenital cancer, a prostate cancer, an ovarian cancer, an endometrial cancer, a uterine cancer, an esophageal cancer, a gastric cancer, a colorectal cancer, a dermal cancer (e.g., melanoma), squamous cell carcinoma, adenocarcinoma, basal cell carcinoma, mucinous carcinoma, or sarcoma (e.g., kaposa sarcoma or Ewing’s sarcoma).

242. The antibody molecule, ADC, composition for use, or method of any of embodiments 231-233, wherein the cancer is a hematological cancer (e.g., multiple myeloma, smoldering myeloma, plasma cell dyscrasias (light chain amyloidosis), lymphoma, or leukemia).

243. The antibody molecule, ADC, composition for use, or method of any of embodiments 231-233, wherein the cancer is a myeloma, e.g., a multiple myeloma.

244. The antibody molecule, ADC, composition for use, or method of any of embodiments 231-233, wherein the antibody molecule, ADC, or composition is administered to the subject intravenously.

245. The antibody molecule, ADC, composition for use, or method of any of embodiments 231-233, wherein the antibody molecule, ADC, or composition is administered once a week, twice a week, once every two weeks, once every three weeks, or once every four weeks.

246. The antibody molecule, ADC, composition for use, or method of any of embodiments 231-233, further comprising determining the level of CD138 in a sample from the subject.

247. The antibody molecule, ADC, composition for use, or method of any of embodiments 231-233, further comprising administering to the subject a second therapy for cancer.

248. The antibody molecule, ADC, composition for use, or method of any of embodiments 231-233, wherein the second therapeutic agent or modality comprises a proteasome inhibitor.

249. The antibody molecule, ADC, composition for use, or method of any of embodiments 231-233, wherein the proteasome inhibitor comprises bortezomib.

250. A method of targeting an anti-CD138 antibody molecule to a cancer, the method comprising:
providing an anti-CD138 antibody molecule that selectively binds to CD138 in a tumor microenvironment; and
administering the antibody molecule to a subject in need thereof,
thereby targeting the anti-CD138 antibody molecule to the cancer.

251. A method of targeting an ADC to a cancer, the method comprising:
providing an ADC comprising an anti-CD138 antibody molecule that selectively binds to CD138 in a tumor microenvironment, or an ADC comprising the anti-CD138 antibody molecule; and
administering the ADC to a subject in need thereof,
thereby targeting the ADC to the cancer.

252. A method of targeting an anti-CD138 antibody molecule to a cancer, the method comprising:
providing an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the antibody molecule to a subject in need thereof,
thereby targeting the anti-CD138 antibody molecule to the cancer.

253. A method of targeting an ADC to a cancer, the method comprising:
providing an ADC comprising an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the ADC to a subject in need thereof,
thereby targeting the ADC to the cancer.

254. A method of targeting an anti-CD138 antibody molecule to a cancer, the method comprising:
providing an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2, or an ADC comprising the antibody molecule; and
administering the antibody molecule to a subject in need thereof,
thereby targeting the anti-CD138 antibody molecule to the cancer.

255. A method of targeting an ADC to a cancer, the method comprising:
providing an ADC comprising an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the ADC to a subject in need thereof,
thereby targeting the ADC to the cancer.

256. A method of improving the delivery of an anti-CD138 antibody molecule to a cancer, the method comprising:
providing an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the antibody molecule to a subject in need thereof,
thereby improving the delivery of the anti-CD138 antibody molecule to the cancer.

257. A method of improving the delivery of an ADC to a cancer, the method comprising:
providing an ADC comprising an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2, or an ADC comprising the antibody molecule; and
administering the ADC to a subject in need thereof,
thereby improving the delivery of the ADC to the cancer.

258. A method of increasing the efficacy of a cancer therapy, the method comprising:
providing an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the antibody molecule to a subject in need thereof,
thereby increasing the efficacy of the cancer therapy.

259. A method of reducing the toxicity of a cancer therapy, the method comprising:
providing an ADC comprising an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the ADC to a subject in need thereof,
thereby reducing the toxicity of the cancer therapy.

260. A method of reducing the toxicity of an anti-CD138 antibody molecule, the method comprising:
providing an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the antibody molecule to a subject in need thereof,
thereby reducing the toxicity of the cancer therapy.

261. A method of reducing the toxicity of a cancer therapy, the method comprising:
providing an ADC comprising an anti-CD138 antibody molecule that has a KD value for CD138 at pH 7.4 and a KD value for CD138 at pH of 5.5-7.0 (e.g., a pH of 5.5-6.0, 6.0-6.5, or 6.5-7.0), wherein the ratio of KD for CD138 at pH 7.4 and KD for CD138 at pH of 5.5-7.0 (e.g., a pH of 5.5-6.0, 6.0-6.5, or 6.5-7.0) is at least 2; and
administering the ADC to a subject in need thereof,
thereby reducing the toxicity of the cancer therapy.

262. An antibody molecule of any of embodiments 1-218, an ADC of embodiment 219, or a composition of embodiment 220 or 221, for use in a method of treating a precancerous condition or preventing a cancer.

263. A method of treating a precancerous condition or preventing a cancer, the method comprising administering to a subject in need thereof an effective amount of an antibody molecule of any of embodiments 1-218, an ADC of embodiment 219, or a composition of embodiment 220 or 221, thereby treating the precancerous condition or preventing the cancer.

264. An antibody molecule of any of embodiments 1-218, an ADC of embodiment 219, or a composition of embodiment 220 or 221, for use in a method of inhibiting CD138 in a cell or subject.

265. A method of inhibiting CD138, the method comprising contacting a cell or subject an antibody molecule of any of embodiments 1-218, an ADC of embodiment 219, or a composition of embodiment 220 or 221, thereby inhibiting CD138.

266. An antibody molecule of any of embodiments 1-218, an ADC of embodiment 219, or a composition of embodiment 220 or 221, for use in a method of causing an ADCC activity in a cell or subject.

267. A method of causing an ADCC activity, the method comprising contacting a cell or subject an antibody molecule of any of embodiments 1-218, an ADC of embodiment 219, or a composition of embodiment 220 or 221, thereby causing the ADCC activity.

268. A method of inhibiting CD138, comprising contacting a cell that expresses CD138 with an antibody molecule of any of embodiments 1-218, thereby inhibiting CD138.

269. The method of embodiment 268, wherein the cell is contacted with the antibody molecule in vitro, ex vivo, or in vivo.

270. A method of detecting a CD138 molecule, the method comprising contacting a cell or a subject with an antibody molecule of any of embodiments 1-218, thereby detecting the CD138 molecule.

271. The method of embodiment 270, wherein the antibody molecule is coupled with a detectable label.

272. The method of embodiment 270 or 271, wherein the CD138 molecule is detected in vitro, ex vivo, or in vivo.

### EXAMPLES

### Example 1: Peptide Mapping of mAb 4320 Binding Site

The binding kinetics for antibody mAb 4320 binding human CD138 peptides, representing two distinct regions of CD138 as described, was evaluated by bio-layer interferometry (aka “Octet). Bio-Layer Interferometry (BLI) is a label-free technology for measuring biomolecular interactions. It is an optical analytical technique that analyzes the interference pattern of white light reflected from two surfaces: a layer of immobilized protein on the biosensor tip, and an internal reference layer. Any change in the number of molecules bound to the biosensor tip causes a shift in the interference pattern. Interactions are measured in real time, providing the ability to monitor key parameter such as binding specificity, rates of association and dissociation, or analyte concentration.

Chemically synthesized peptides included covalently modified, amino-terminal biotinylation separated by an intervening dPEG8 linker and followed by human CD138 amino acid sequences of varying length and as described in Error! Reference source not found..

TABLE 11

List of human CD138 peptides used for epitope mapping

| Peptide ID | SEQ ID | Region | Peptide Sequence | Length | Binding |
|---|---|---|---|---|---|
| Peptide 2A | 602 | IBD | Biot-LC-ASTSTLPAGEGPKEGEAVVLPEVEPGLTAREQEA-OH | 34 | + |
| Peptide 2C | 603 | IBD | Biot-LCGEAVVLPEVEPGLTAREQEA-OH | 20 | + |
| Peptide 2D | 604 | IBD | Biot-LC-GEAVVLPEVEPGLTA-OH | 15 | + |
| 138-2-1 | 605 | IBD | Biot-LC-VLPEVEPG-OH | 8 | - |
| 138-2-2 | 606 | IBD | Biot- LC-LPEVEPG-OH | 7 | - |
| 138-2-3 | 607 | IBD | Biot-LC- PEVEPGLT-OH | 8 | - |
| Peptide 6 | 608 | MPER | Biot-LC-DFTFETSGENTAVVAVEPDRRNQSPVDQGATGASQGLLDRK-OH | 41 | + |
| Peptide 6B | 609 | MPER | Biot-LC-ENTAVVAVEPDRRNQSPVDQGATGASQGLLDRK-OH | 33 | + |
| Peptide 6F | 610 | MPER | Biot-LC-ENTAVVAVEPDRRNQ-OH | 15 | + |
| 138-6-1 | 611 | MPER | Biot-LC-VVAVEPDR-OH | 8 | + |
| 138-6-2 | 612 | MPER | Biot-LC-VAVEPDRR-OH | 8 | - |
| 138-6-3 | 613 | MPER | Biot-LC-AVEPDRR-OH | 7 | - |

Approximate region within human CD138 (extracellular domain) is generally noted. IBD, integrin binding region. MPER, membrane proximal region. VEP sequence common to all peptides is noted by boldface and underline. See also SEQ ID NO: 601; LC-long chain (dPEG8) n-terminal linker. Relative mAb 4320 binding is qualitatively scored as positive (+) or negative (-) based on data presented in FIG. 1 and methods described above.

An exemplary human CD138 amino acid sequence is shown below as SEQ ID NO: 601. Italics indicate the signal sequence. Bolded underlining indicates the heparan sulfate and chondroitin sulfate attachment sites (SG). Bolded double-underlining indicates the transmembrane domain. Bolding without underlining indicates VEP motifs. Box indicates a minimal integrin-binding site (corresponding to peptide 2 region, as described herein). Dark gray highlighting indicates an extended membrane proximal region, which includes a second chondroitin sulfate attachment site, and which corresponds to peptide 6 region, as described herein.

MRRAALWIWLCALALSLOPALPQIVATNLPPEDQDGSG DDSDNFSGSG AG
ALQDITLSQQTPSTWKDTQLLTAIPTSPEPTGLEATAASTSTLPAGEGPK EGEAVVLPEVEPGLTAREQEATPRPRETTQLPTTHLASTTTATTAQEPAT
SHPHRDMQPGHHETSTPAGPSQADLHTPHTEDGGPSATERAAEDGASSQL
PAAEGSG EQDFTFETSG ENTAVVAVEPDRRNQSPVDQGATGASGLLDRKE
VLGGVIA GGLVGLIFAVCLVGFMLY RMKKKDEGSYSLEEPKQANGGAYQK
PTKQEEFYA (SEQ ID NO:601)

In this example, human syndecan-1 (sdc1; CD138) is based on UniProt accession number (P18827) https://www.uniprot.org/uniprot/P18827. Proposed signal sequence is italicized. Heparan sulfate and chondroitin sulfate attachment sites (SG) are underlined in bold. Transmembrane domain is double underlined and in bold. Minimal integrin binding site corresponding elsewhere to “peptide 2 region” is boxed. Extended membrane proximal region inclusive of second chondroitin sulfate attachment site and corresponding elsewhere to “peptide 6 region” is shaded in dark gray. VEP is highlighted in bold and in larger font.

Biotinylated peptides were bound on to streptavidin SA biosensors (Sartorius Cat. No. 18-5019) at 50 nM concentrations for 360 seconds (loading step) followed by washing for 60 seconds (wash step). MAb 4320 was subsequently bound for 300 seconds (association step) followed by a 300 second dissociation step in buffer (dissociation step). All steps were completed in binding buffer comprised of phosphate buffered saline (PBS), pH 7.4 and 0.02% Tween-20 to minimize non-specific interactions. Binding and dissociation of MAb 4320 to CD138 derived peptides are visually summarized in FIG. 1.

MAb 4320 bound peptide 2 related peptides 2A and truncated nested peptides 2C, and 2D. Loss of binding is observed for minimal peptide sequences 138-2-1, 138-2-2, and 138-2-3. Similarly, MAb 4320 binds the larger peptide 6 as well as truncated, nested peptides 6B and 6F. Binding of mAb 4320 was observed for minimal peptides 138-6-2 and 138-6-3 and greatly reduced binding to peptide 138-6-1. 132-6-1 differs from 132-6-2 by the absence of one N-terminal residue and one C terminal residue illustrating the criticality of these terminal amino acids. A sequence alignment of overlapping peptides is summarized in FIG. 2.

A comparison of these peptides as they correspond to separate regions of human CD138 highlight the common core linear sequence comprised of amino acids VEP. The respective binding profiles strongly suggest VEP as comprising a “core” epitope motif. These data also point to contributions of sequences within the respective “peptide 2” and “peptide 6” regions both N and C terminal to this core epitope to the larger definition of the mAb 4320. Importantly, these adjoining sequences are not identical; moreover, their separation in the primary Sdc1 sequence points to two distinct regions within CD138 (sdc1) comprising the mAb 4320 epitope.

Based on these data, peptides H2N-GEAVVLPEVEPGGLTAR-OH (peptide 138-2-5) and H2N-ENTAVVAVEPDRRNQ-OH (peptide 138-6-5), corresponding to peptide region 2 and peptide region 6, respectively were synthesized for use as substrates in x-ray crystallography as described in Example 3.

### Example 2. Peptide Mapping of CD138 Epitope by Alanine Scanning Mutagenesis

The epitope of mAb 4320 was further evaluated using alanine scanning mutagenesis in conjunction with the use of mammalian-based antigen display. Mammalian-display platform allows hundreds of specified mutations to be introduced into membrane target proteins such as syndecan-1 and recombinantly expressed in mammalian cells followed by evaluation of target binding by antibodies and other proteins. A common human derived cell line for such purposes is HEK293 cells. Conditions for binding and screening of mAb 4320 and related Fab (variable domain) using high-throughput flow cytometry were optimized using full length SDC1 sequence cloned into a vector and heterologously expressed in HEK-293T cells as described. A carboxy-terminal epitope tag comprised of the V5 epitope in tandem with a 6X histidine sequence was included in the design to validate and quantify target membrane expression independent of antibody binding. Anti-CD138 antibodies used as positive controls include DL-101 (APC-conjugated; BioLegend, Cat No. 352308) and unconjugated (unlabeled) antibody clone 359103 (R&D Systems, Cat No. MAB2780). DL-101 represents a commercially available anti-CD138 antibody pre-validated for use in flow cytometry and commonly cited in the literature. The anti-CD138 antibody BB4 was used as an additional control and for comparative purposes. Isotype matched-secondary antibodies were obtained from Jackson Immuno Research and included anti human IgG and anti-rat IgG chosen based on primary anti-CD138 antibody used in this evaluation.

In brief, cells were transfected with a wild-type (WT) CD138 (SDC1) gene sequence construct of the target protein or with vector alone in 384-well format, followed by detection of cellular expression via high-throughput flow cytometry. Serial dilutions of each MAb were tested for immunoreactivity against cells expressing target protein (WT) or vector alone. Validation of target expression and optimization of detection and binding of anti-CD138 antibodies both by flow cytometry was completed in replicate (N=4). The optimal screening concentration for each MAb was determined based on the raw signal values and signal-to-background calculations specifically optimized for each test article. Optimized parameters included the use of 10% goat serum as blocking reagent. Primary antibody staining was for 60 minutes at 25° C. Wash buffer include phosphate buffered saline (divalent metal ion free), pH 7.4. Optimized concentrations of respective anti-CD138 antibodies included 2.0 µg/mL (mAb 4320), 0.5 µg/mL (antibody 359103), 1.0 µg/mL(DL-101), and 0.50 µg/ml (BB4). Secondary antibodies were diluted 1:400 and incubated for 30 minutes at 25° C. These optimization experiments confirmed robust expression of CD138 on the cell surface of HEK293 and titrated binding of all antibodies used.

Library screens of very-high-affinity mAbs can fail to yield critical residues for antibody binding due to deficiencies of such an assay to detect and differentiate critical vs. less critical binding based on affinity and quantification of cell binding. One common approach in the literature is the use of monovalent antibody derived fragments such as Fab usually weakens binding sufficiently to allow identification of critical residues for binding. For cases where Fab screens under standard conditions are still insufficient to identify critical residues for binding, high stringency conditions are implemented. These conditions include combinations of increased pH, increased salinity, increased temperature, and/or increased wash time. mAb 4320 was therefore evaluated for syndecan-1 cell surface binding analogously to described above but as a Fab. Optimized experimental parameters were largely as described for the full-length IgG but with experimental modifications. These included 0.50 ug/mL 4320 Fab concentration, 60 minute incubation time at 25° C. and the use of goat anti-human F(ab)2 antibody, conjugated with Alexa Fluor 488 and used at a dilution of 1:200. Binding of each test Ab to each mutant clone in the alanine scanning library was determined, in duplicate, and likewise evaluated by flow cytometry. For each point, background fluorescence was subtracted from the raw data, which were then normalized to Ab reactivity with WT target protein. For each mutant clone, the mean binding value was normalized as a function of expression (represented by control reactivity). The control antibody (BB4) was also analyzed for comparative purposes.

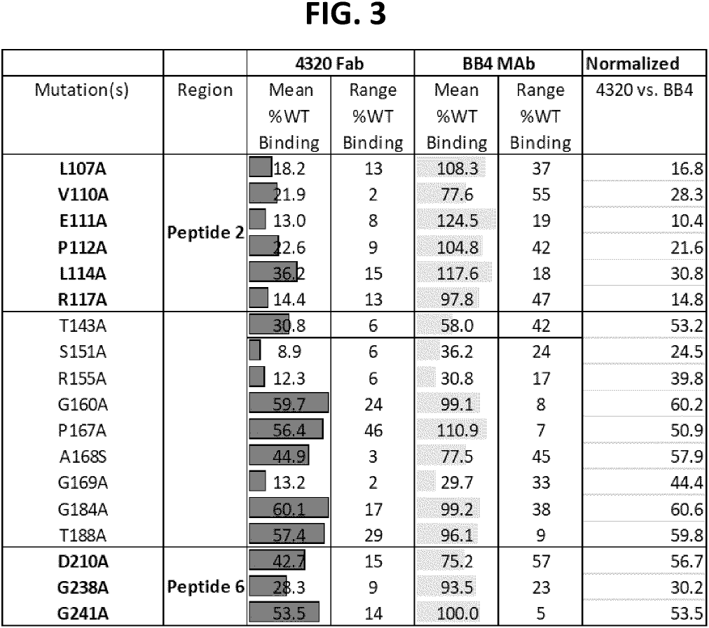

Binding analysis is summarized in FIG. 3. For this example, stringency was set at ≤60% for each of the mutant clones (single positions). Binding less than 30% observed for both BB4 and Fab 4320 suggest the possibility of structural perturbations not directly relevant or indicative of epitope mapping by this method.

BB4 Binding and epitope mapping are shown for comparative purposes and highlight utility of method to differentiate epitopes. Based on this method and analysis, two regions of higher stringency binding were identified. Select positions within CD138 are highlighted. For example, these include the linear sequence LVEPLR mapping to peptide 2 as described in Example 1. Additional examples include positions D210, G238, and G241 corresponding to peptide 2 and the membrane proximal region of CD138. These highlighted residues are likely major energetic contributors to mAb 4320 binding to CD138.

Of note, additional mutated residues within this region (e.g., D228A, Q232A) also led to reduce antibody but above the threshold cut-off. It is possible that this method may be biased in certain respects that this result is biased by methods, for example due a more dominant, higher affinity biding site or positions represented by these mutants and possible global perturbations to CD138 (syndecan-1 structure) as noted. Nevertheless, this analysis does identify positions likewise identified through the methods provide in Example 1 above and in agreement with the definition of the 4320-CD138 epitope as determined by higher resolution, crystallographic methods as described in Example 3.

### Example 3. Structural Determination of mAb 4320 Paratope-CD138 Epitope by X-Ray Crystallography

A high-resolution structural determination of antibody 4320 in complex with two CD138 related peptides was completed using x-ray crystallography. For this study, the Fab fragment of 4320 was used and complexed with peptides 138-2-5 and 138-6-5 generally corresponding to peptide 2 and peptide 6, respectively and representing two linearly separated regions within CD138 (syndecan-1). The sequence ID of these two peptides is summarized in Table 12. This analysis provided a structure description of the Mab 4320 paratope-CD138 binding interactions as represented by co-crystallographic structure with near atomic resolution. Methods used to generate this molecular complex and the corresponding crystallographic data set use standard methods but are also described herein with modifications and herein for reference.

TABLE 12

Sequences of CD138 peptides used for X-crystallography

| SEQ ID NO | Peptide Designation | Sequence |
|---|---|---|
| 614 | 138-2-5 | H2N-GEAVVLPEVEPGGLTAR-OH |
| 615 | 138-6-5 | H2N-ENTAVVAVEPDRRNQ-OH |

### 4320 Fab Preparation and Purification

The monoclonal antibody 4320 was concentrated to 5 mg/mL in 2 mL and was cleaved using immobilized FabALACTICA (FabALACTICA Fab kit Midispin, Genovis) to produce a Fab fragment. Fab portion was purified from antibody Fc using Protein A chromatography (CaptureSelect resin) pre-equilibrated in PBS. The flow-through that contained the Fab was collected, pooled, and concentrated by ultrafiltration. Further purification of the antibody Fab was achieved by gel filtration chromatography. In brief, 1.5ml and injected on a size exclusion column (Superdex S75 16/60) pre-equilibrated with SEC buffer (10 mM HEPES, 150 mM NaCl, pH 7.5). The Fab eluted as a single peak; corresponding fractions were pooled together and concentrated to approximately 13 mg/mL.

### Crystallization and Freezing

The Fab 4320:peptide complexes were mixed in a 1:50 molar ratio of protein to peptide. The Fab4320:peptide 138-2-5 complex was set up at 11.4 mg/ml in 100+100 nL drops. A large crystal grew from the BCS screen well F7 (0.2 M magnesium chloride hexahydrate, 0.1 M Tris/HCl, 25% (v/v) PEG Smear High and 10 % (v/v) glycerol. The Fab4320:peptide 138-6-5 complex was set up at 11.0 mg/ml in 100+100 nl drops. Smaller crystals grew from the BCS screen well C8 (0.05 M L-arginine, 0.05 M L-glutamic acid monosodium salt hydrate, 28% (v/v) PEG Smear Broad and 5 % (v/v) glycerol.

Freezing conditions were optimized for each of the FAB4320:peptide crystals observed. The Fab 4320:peptide 138-2-5 crystal was transferred to a cryoprotectant solution containing 25 % (v/v) PEG Smear High, 0.1 M Tris/HCl pH 8.0, 0.2 M magnesium chloride and 4 mM peptide 1 and flash-frozen in liquid nitrogen. The Fab 4320: peptide 138-6-5 crystal was transferred to a cryoprotectant solution containing 28% (v/v) PEG Smear Broad, 0.025 M L-arginine, 0.025 M L- glutamic acid, 25% glycerol and 3 mM peptide 2 and flash-frozen in liquid nitrogen.

### Data Collection and Refinement

Data were collected at 100 K at station I04, Diamond Light Source, UK (λ = 0.9795 Å) equipped with an Eiger 16M hybrid-pixel detector. Data were processed using the Xia2 pipeline followed by Aimless for both Fab 4320:peptide co-crystallized complexes. The structures were determined using the Phaser software and a homologous Fab (PDB code: 6NOV) determined to 2.14 Å with the CDR loops removed. Initially only the Fab:peptide 138-2-5complex could be determined with four molecules in the asymmetric unit in space group P22121. Most of the CDR loops were built and correct amino acid side chains were built. This better model of Fab4320 was used in a second attempt to determine the 1.45 Å structure of Fab 4320. In this attempt the variable Ig domains of Fab4320 were found whereas the constant domains needed to be searched for separately since there is a difference in angle between the domains. One Fab molecule in the asymmetric unit was found with peptide 138-2-5 bound in space group P21. The Fab 4320:peptide 138-2-5 complex was refined to 1.45 Å and used as a starting model for final refinement of the Fab 4320:peptide 138-2-5 complex to have the best possible starting model to solve the lower-resolution structure of the Fab4320:peptide 138-2-5 complex (2.05 Å). Refinement was performed using Refmac5). Model building was carried out in Coot. Anisotropic B-factors were refined for the 1.45 Å Fab 4320:peptide 1 complex whereas TLS refinement was used for the 2.05 Å Fab 4320:peptide 138-6-5 complex. The data collection and refinement statistics for both complexes are shown in Table 13.

TABLE 13

Summary of crystallographic data

| Complex | Fab 4320:peptide 138-2-5 | Fab 4320:peptide 138-6-5 |
|---|---|---|
| Resolution (Å) | 62.5 - 1.45 (1.47 - 1.45) | 65.7 - 2.05 (2.09 - 2.05) |
| Wavelength (Å) | 0.9795 | 0.9795 |
| Space group | P21 | P22121 |
| Unit cell (Å) | a = 68.34, b = 49.61 | a = 126.54, b = 129.90 |
| | c = 76.40, β =114.02° | c = 131.19 |
| Completeness (%) | 99.4 (97.5) | 100.0 (100.0) |
| Redundancy | 6.7 (5.8) | 13.1 (12.8) |
| No. of observations / unique reflections | 553 848 / 82 578 | 1 784 910 / 135 743 |
| | (23 286 / 3 988) | (84 619 / 6 601) |
| <I/σ(I)> | 17.9 (2.5) | 11.0 (1.1) |
| CC(½) (%) | 99.9 (87.2) | 99.9 (52.4) |
| R_{merge} (I) (%) | 4.5 (65.1) | 14.0 (315.8) |
| Resolution used for refinement (Å) | 62.5 - 1.45 (1.49 - 1.45) | 65.7 - 2.05 (2.10 - 2.05) |
| R_{model} (F) (%) | 11.3 (17.9) | 19.0 (32.2) |
| R_{free} (F) (%) | 15.5 (25.3) | 22.3 (34.7) |
| No. of non-hydrogen atoms | 4 026 | 14 083 |
| No. of water molecules | 479 | 535 |
| Bond lengths (Å) | 0.016 | 0.014 |
| Bond angles (°) | 1.95 | 1.8 |
| Mean B-factor heavy chain/s A (or A, D, | hc: 23.5 | hc: 25.7, 36.0, 35.0, 27.2 |
| G and K), light chain/s chain B (or | lc: 22.9 | lc: 26.3, 34.9, 33.2, 25.7 |
| chains B, E, H and L) (Å²) | | |
| Mean B-factor solvent (Å²) | 37.2 | 47.6 |
| Mean B-factor peptide chain/s I (or I, F, Jand M) (Å²) | 29.7 | 27.3, 53.9, 40.1, 29.6 |
| Favored regions (%) | 97.9 | 97.8 |
| Allowed regions (%) | 2.1 | 2.2 |
| Outliers (%) | 0.0 | 0.0 |

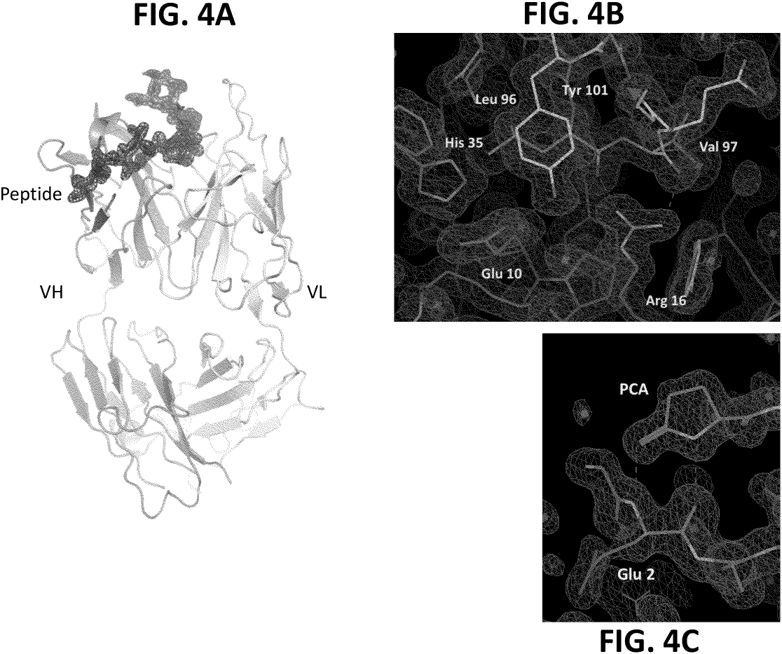

The Fab4320 -peptide-138-2-5 complex is at near atomic resolution and can resolve weak and strong hydrogen bonds (2.5 vs 3.2 Å) In this complex, the heavy chain N-terminal Q1 residue is observed as pyroglutamic acid (PCA) The Fab4320:peptide 1 structure includes a heavy chain with amino acids 1-213 with a loop not traced (aa 127-133), the light chain with amino acids 1-218 and the entire peptide with amino acids 1-16 visible in the electron density map, (FIG. 4). In addition, five glycerol and 476 water molecules have been added to the model.

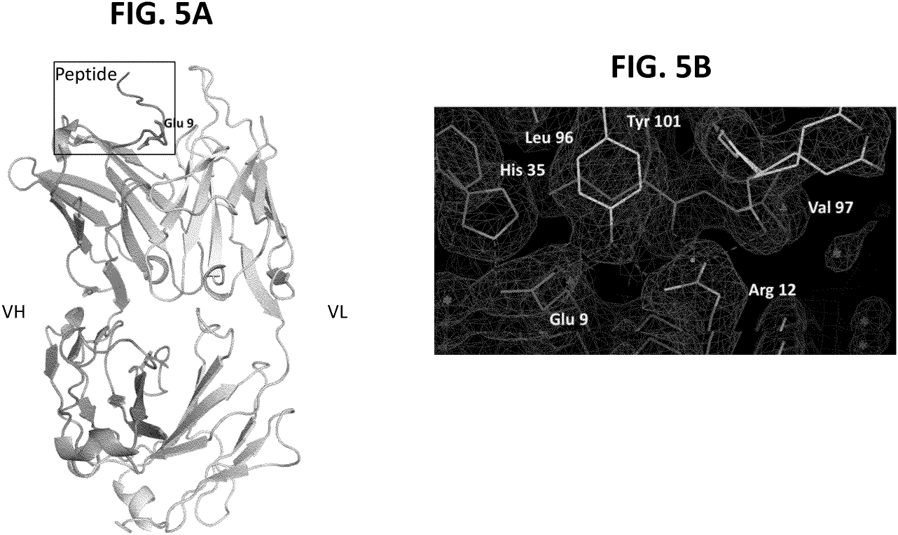

The Fab4320:peptide 138-6-5 structure (FIG. 5) includes 12 polypeptide chains with the peptide in chains I, F, J and M, the Fab heavy chain in chains A, D, G and K and the Fab light chain in chains B, E, H and L. The heavy chains include amino acids 1-212, 2-213, 2-212 and 1-213 for chains A, D, G and K respectively. In all heavy chains, a mobile loop (residues 127-132) is not clearly seen in the electron density maps. The light chains include amino acids 1-219, 1-218, 1-218 and 1-219 for chains B, E, H and L respectively. The peptides include amino acids 5-14, 5-13, 5-13 and 5-13 for chains I, F, J and M respectively. but the interactions are similar in all four complexes. In addition, 13 glycerol and 535 water molecules have been added to the model.

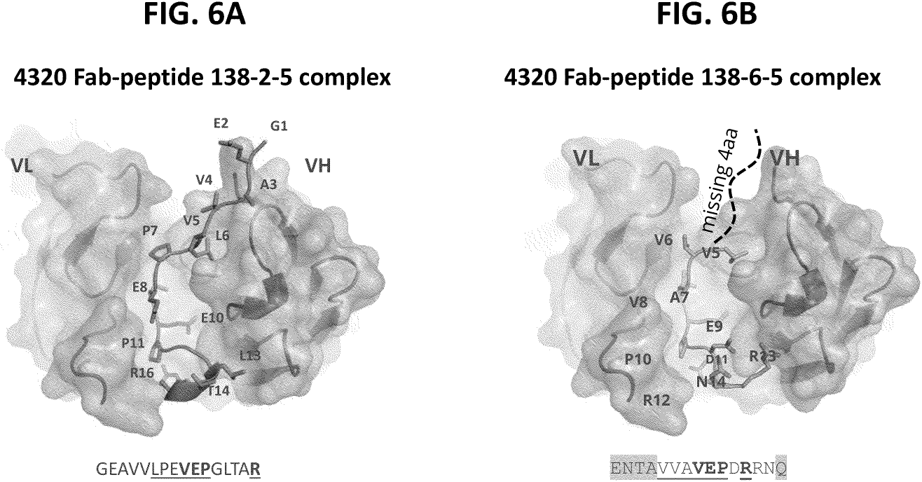

A further refinement and modeling of the two respective structures was made for purpose of describing the 4320 paratope-CD138 binding interfaces in greater detail (FIGS. 6A-6B). In the peptide-138-2-5 complex, the entire 16 aa is ordered and observed in the structure. Only T14 residue (in the peptide) does not interact with antibody. All six CDRs (VH and VL) interact with the peptide. From the perspective of the peptide, 4320 binds to non-contiguously to R16. This observation points to a “core epitope” comprised of the sequence LPEVEPXXXXR (underlined in FIG. 6A). In the peptide 138-6-5 complex, only 10 residues of the 15 residues are observed. The missing N-terminal residues are likely due to the accessibility of the binding site due to crystal contacts; the missing C-terminal residues is proposed to be due to non-contact with the antibody. All six CDRs within the mAB 4320 variable region contact the CD138 peptide region. Analogously to the structure solved for mAb4320 in complex with peptide 138-2-6, 4320 appears to bind non-contiguously to arginine at position 16. This observation points to a “core epitope” comprised of sequence VVAVEPXR (underlined in FIG. 6B).

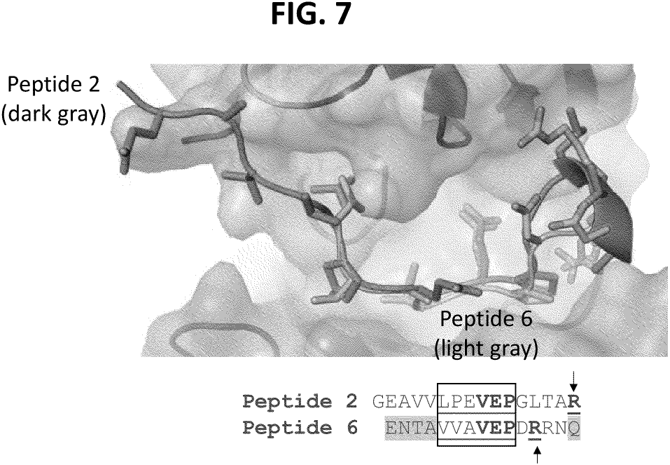

In certain respects, therefore, the two peptides described in the context of 4320 antibody binding and its epitope share a “common core”. This “core” epitope may be described in more general terms as being comprised in total of 13 amino acids in total (peptide 2 and peptide 6), positioned in linearly separated regions of CD138. The core (or minimal) epitope found within each site may be described as hhxVEP (linear) followed by a non-contiguous C-terminus arginine. Additional structural determinants of this epitope appear to include a hydrophobic sequence comprised of 4 contiguous amino acids positioned N terminal to this core epitope. This core epitope is depicted in FIG. 7. Key features of the MAb 4320-CD138 peptide interface based on this structure are summarized in Table 14 below.

TABLE 14

Key Contacts of mAb 4320-CD138 interface

| | | |
|---|---|---|
| | Peptide 2 | Peptide 6 |
| Interface (Å²) | 991 | 694 |
| Hydrogen bonds | 11 | 9 |
| Salt bridges | 2 | 2 |
| Paratope residues | VH: V2, G26, Y27 S28, S31, Y32,Y33,H35,H52,S54,D55,T57,N59,N98, F99,V100,Y101 | VH: V2,Y27,S31,Y32,Y33,H35,H52,S54,D55,T57,N59,N98,F99,V100,Y101 |
| | VL: Y31, K32,D33,K35,Y37 L38,N39,L51, Y54,V55 A60, L96,V97,E98,Y99,Y101 | VL: Y31,K32,D33,K35,Y37 L38,N39,L51, Y54,V55 A60, L96,V97,E98,Y99,Y101 |
| Epitope residues | GEAVVLPEVEPGLTAR All residues except T14 | ENTAVVAVEPDRRNQ All observed residues and possibly Residues 1-4 (ENTA) and Q15 |

### Example 4: Characterization of pH Dependent Binding of Anti-CD138 Antibody Variants by Flow Cytometry

The use of biologics as targeted therapeutics in the treatment of cancer continues to be a very tractable treatment strategy. Many of these antigens, however, while differentially expressed at a higher level on the surface of the tumor to be targeted nevertheless are not exclusively tumor specific but also expressed in normal tissues and cell types. CD138 (syndecan-1) follows this profile; highest expression of CD138 exsits in bone marrow and immune related compartments (plasma cells and plasmablasts) while additional expression of CD138 occurs in select epithelial tissues (e.g., lung, breast), kidney, and the endothelium present under normal physiological conditions. Antibodies and other biologics that preferentially target the tumor within its microenvironment represent a logical and attractive approach to improve target selectivity to tumor sites while reducing undesired “on target” binding/pharmacology in normal tissues. On such strategy involves leveraging the differential pH of the the tumor microenvironment. The pH in the tumor microenvironment is generally acidic (pH 6.0-7.0) due to high metabolic activity (metabolic acidosis), insufficient perfusion, and hypoxia. The design of antibodies with pH selective binding to CD138 at acidic pH (6-6.5) but substantially reduced binding at pH 7.4 embodies such a strategy with the prospect of achieving improved efficacy and therapeutic indices as well as improved antibody PK and safety profiles (e.g., lower drug limited toxicities as is often the case for example of antibody-drug conjugates comprised of highly toxic payloads).

The antibodies described herein and summarized Tables 1-2 are designed to achieve aspects of such selectivity as evidenced through pH dependent target binding in vitro using commonly used methods. In one experiment, select variants of anti-CD138 mAb 4320 were assessed for their capacity to bind to CD138 on the surface of cells under conditions of varying pH. Antibody binding to membrane CD138 was evaluated using the CD138+ human lymphoblastic myeloma cell line (U266) and quantified by flow cytometry. Cells were grown in RPMI1640 with 10% FBS. On the day of the experiment, 0.25 × 10⁶ cells were washed with FACS buffer (PBS + 0.5%BSA) and incubated for 30 minutes at 4° C. with varying concentrations of anti-CD138 antibodies titrated from 10 µg/mL to 0.01 µg/mL in PBS+0.5% BSA, first adjusted to either pH 6.0 or 7.4. Cells were washed (1X) with same pH-matched buffer (and as throughout the experiment) followed by incubation with secondary (detection) goat anti-human Fab( Jackson Immuno Research) conjugated with Alexa Fluor-488 for 30 minutes also at either pH 6.0 or 7.4. Cells were washed once more and resuspended in matching pH buffer and evaluated by flow cytometry. Antibody cell binding was reported as geometric mean fluorescence intensity (MFI). Dose-response curves were generated using GraphPad Prism. Binding data were plotted using nonlinear regression analysis and a 4-parameter curve fit. Parental antibody mAb 4320 was included in most analyses for comparative purposes.

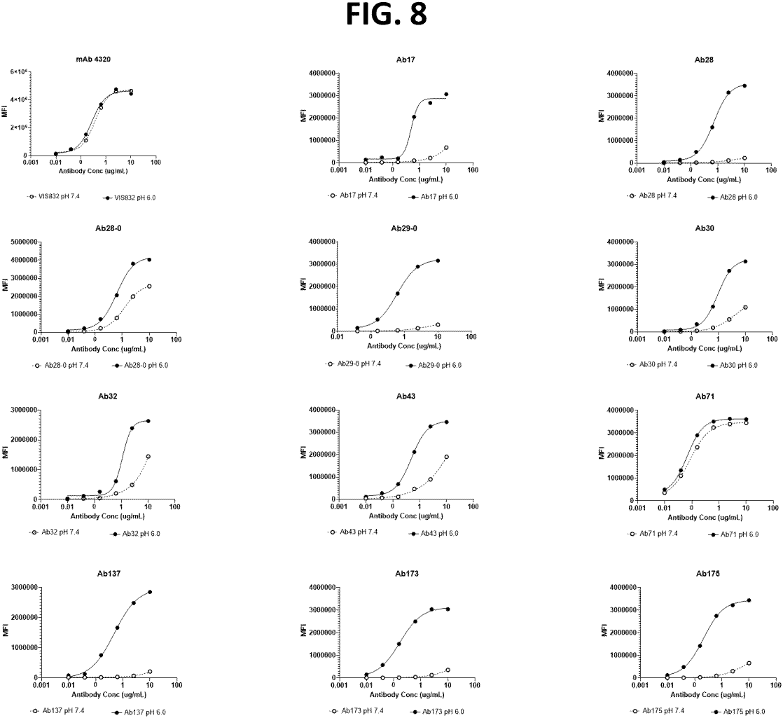

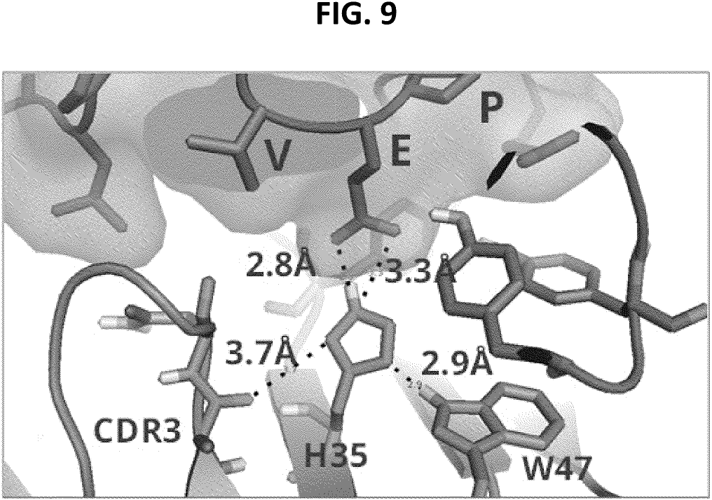

Antibodies 28-0 and Ab137, comprising the corresponding sequences listed in Tables 1 and 2, represent single changes in the mAb 4320 variable region corresponding to VH HCDR3 mutation (N98D) and VL LCDR3 (Y99D), respectively and are incorporated as examples. Additional single mutations include Ab 32 (VH: V100L) and Ab43 (VH: N39L), all of which demonstrated pH selectivity favoring binding to cell surface CD138 at pH 6.0 relative to pH 7.4 in this assay (FIG. 8). Ab137 is highlighted as an example showing highly selective binding to cell surface CD138 at pH 6.0 with negligible binding at pH 7.4. 28-0 represents a single mutation involving HCDR3 and example of a change in the charge of a particular amino acid (N98D) shown to be proximal to H35; the latter residue is located in the HCDR1 of 4320 and corresponds to a key residue within the mAb 4320 paratope that binds to CD138 through engagement of VEP (FIG. 9).

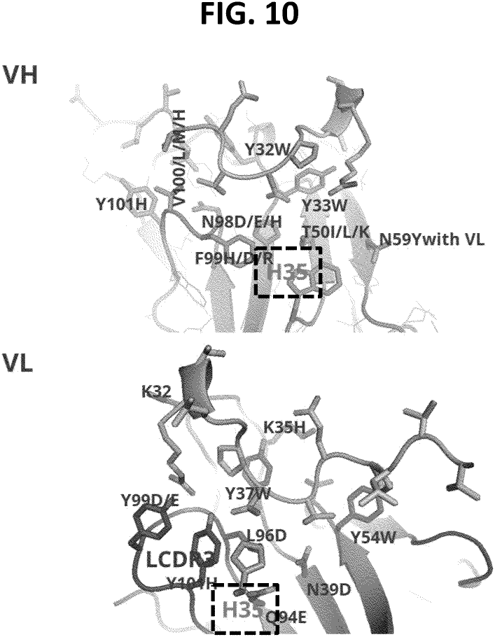

Ab17 is illustrative of one example of an introduction of a histidine into the LCDR1 of the light chain (K35H). This mutation is built upon 28-0 which also involves a charge -based mutation at N98 (N98D) in the minimalized HCDR3 of the heavy chain as described. Several additional mutations (31 in total) using a histidine-scanning approach at select positions within both the VH and VL variable regions of mAb 4320 were evaluated. Ab17 represents the only example in which the addition of a histidine conferred any selectivity (data not shown). While not exhaustive, this example therefore illustrates the apparent limitations of using such a singular approach in which histidine can be used to confer selective binding at pH 6 (approximate to its intrinsic pKa). A structural description summarizing select mutations based on proximity to H35 of the VH of mAb 4320 is visually summarized in FIG. 10.

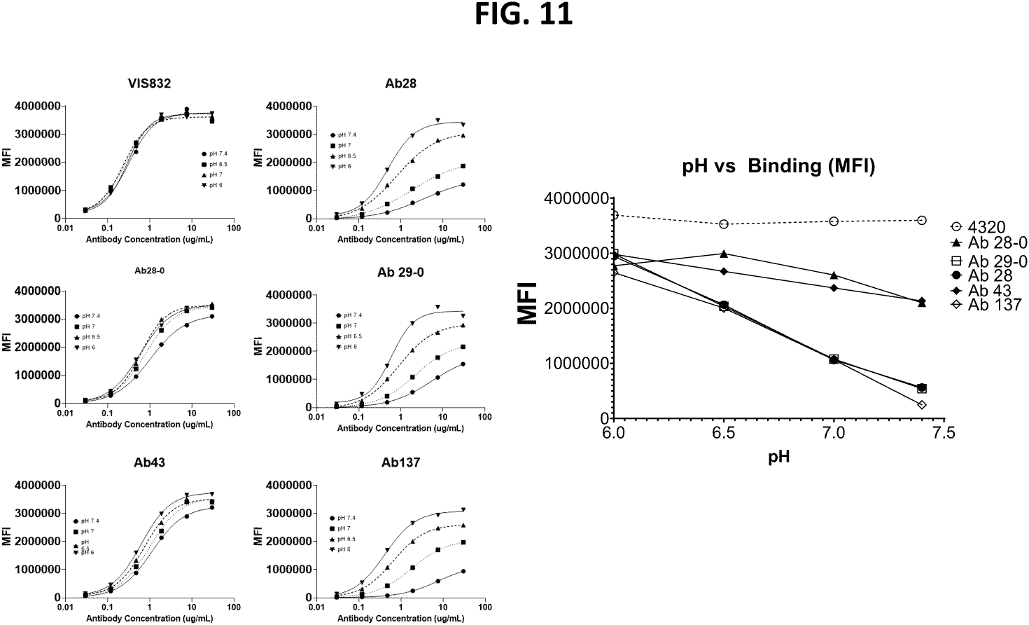

While the anti-CD138 antibody variants described provide key structure-activity examples of achieving pH selective binding of these antibodies to the target (CD138) present on the cell surface of a representative CD138-expressing cell line (U266), the pH within a tumor microenvironment may also exist as a gradient within this wider pH range (e.g., 6.0-7.4). Target binding to cell surface CD138 was therefore further evaluated by pH titration inclusive of pH 6.0, 6.5, 7.0, and 7.4. Binding was evaluated as described. Results are summarized in FIG. 11.

### Example 5. Characterization of pH Dependent Binding and Relative Binding Affinities of Anti-CD138 Antibody Variants to CD138 Extracellular Domain by Biolayer Interferometry.

Example 4 highlights examples of select anti-CD138 antibodies demonstrating pH selective binding to the target present on the cell surface of a CD138+ cell line (U266). The use of this cell line was chosen in part as an example but also illustrates the utility of such engagement in the context of a relevant cellular target representative of multiple myeloma and commonly used for such a purpose in vitro. In furtherance of these data, the relevant binding of select anti-CD138 antibody variants to CD138 extracellular domain was evaluated by biolayer interferometry. The principle of biolayer interferometry (referred herein as “Octet”) are as described in Example 1. Recombinant and soluble version of human CD138 approximately corresponding to amino acid Q18-E251 (Accession number NP-0029833.3) was obtained from a commercial source (Sino Biological, Cat. 11429-H08H).

Recombinant CD138 was chemically conjugated to biotin at reactive primary amino groups of lysines and N-Hydroxysulfo succinimaide (NHS esters.)using standard methods and as described by the manufacturer in accordance with the commercially used kit (EZ-Link, NHS-SS-PEG4 Biotin, ThermoFisher Catalog number 21442). Recombinant CD138 was biotinylated using a ratio of protein: biotin of 1:20. A PEG-4 linker arm of 38 angstroms was used for purposes of achieving appropriate spacing of covalently attached biotin and target protein (CD138) and minimize steric hindrance when binding to strepatavidin. Unlabeled biotin was chromatographically separated from modified CD138 by gel filtration.

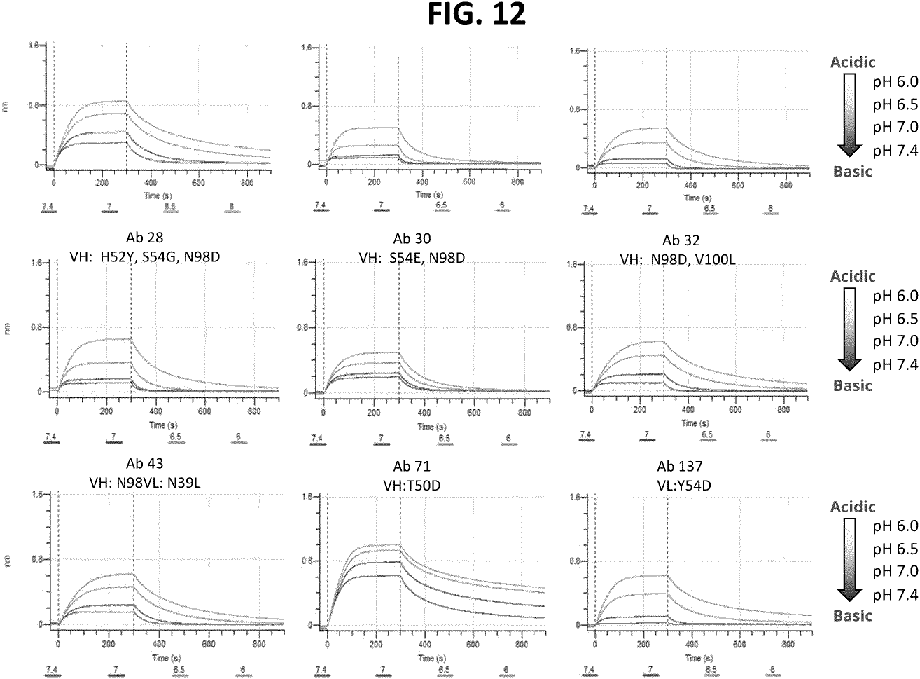

Binding experiments were carried using assay buffer comprised of 100 mM Sodium Phosphate+ 150 mM NaCl + 0.02% Tween-20 and adjusted to target pH of 7.4, 6.0, 6.4, or 6 using appropriate ratios of monobasic and dibasic sodium phosphate buffer. 25 nM biotinylated CD138 was loaded onto Streptavidin capture Octet (SA) tips for 120 seconds (antigen loading step) followed by antibody binding for associated for 300 seconds (association step), and subsequently dissociation for 300 seconds (dissociation step). All steps were carried out at the respective target pH using the corresponding pH adjusted buffers. Results are summarized in FIG. 12.

In general, the pH dependent binding of anti-CD138 variants to recombinant CD138 correlate with results presented in Example 5 in which these antibodies were evaluated for binding to cells expressing the target (CD138) at the cell surface. The examples provided demonstrate a pH titratable binding in which binding is either reduced or largely abrogated as a function of increasing pH (e.g, at pH 7.4). In particular these data point to structure-activity relationships with intended antibody-target binding properties of selective binding wherein differential binding biased toward acid pH relative to more physiological pH (e.g., pH 7.4) is clearly demonstrated. Examples of highly selective binding at acidic pH are also provided. Ab137 and Ab17 include such examples. Such examples also provide evidence of selective target binding at less acidic pH values (e.g., pH 6.5) relative to neutral or pH 7.4 that biologically correlate with more physiological conditions. In one embodiment, such mildly acidic conditions may exist in the context of a non-physiological state. Moreover, this differential binding of these antibodies may also be sufficient to confer selectivity.

As a follow on to these data, the apparent affinity of anti-CD138 antibodies was likewise evaluated using the same Octet-based assay as described but with certain modifications. Modifications to this protocol included lowering the loading concentration of biotinylated CD138 from 25 nM to 10 nM and varying the test antibody concentrations ranging from 25 nM down to 0.8 nM (as 2-fold dilutions). The rationale for doing so was to potentially reduce any target avidity effects while also looking at the analyte concentration dependency. In this experiment binding was measured at either 6.0 or 7.4 Parental antibody mAb 4320 binding was also evaluated as a reference and for comparative purposes. Representative results are summarized in FIG. 13.

In this example, anti-CD138 antibodies mAb 17, Mab 28, and Mab 137 demonstrated no or very low binding to recombinant CD138. The binding signal was essentially equivalent to background. Binding at pH 6.0 was antibody concentration dependent as expected. Parental antibody mAb 4320 demonstrated comparable binding profiles at the two pH conditions tested. These data further demonstrate a lack of selectivity of the parental antibody in contrast to positionally targeted mutations e.g., Mab 17 (VH 98D/VLK35 H), Mab 28 (VH H52Y, S54G, N98D) and mAb 137 (VL: Y99D) highlighted by these examples.

Data were analyzed for purposes of calculating approximate kinetic binding parameters and apparent binding affinities based on these rates. Data was fit assuming a 1:1 binding model. Binding parameters for binding at pH 6.0 are summarized in Table 15.

TABLE 15

CD138 binding parameters for exemplary anti-CD138 antibody variants at pH 6.0

| Sample ID | pH | Antibody Conc. (nM) | ka (1/Ms) | kdis (1/s) | KD (M) |
|---|---|---|---|---|---|
| Ab137 | pH 6.0 | 12.5, 25 | 1.0E+06 | 3.0E-03 | 2.9 E-09 |
| Ab17 | pH 6.0 | 12.5, 25 | 8.6E+05 | 5.1E-03 | 6.2 E-09 |
| Ab28 | pH 6.0 | 12.5, 25 | 1.2E+06 | 4.5E-03 | 4.0E-09 |
| Ab71 | pH 6.0 | 12.5, 25 | 1.2E+06 | 1.1E-03 | 9.8E-10 |
| Ab30 | pH 6.0 | 12.5, 25 | 7.2E+05 | 3.4E-03 | 4.6E-09 |
| Ab32 | pH 6.0 | 12.5, 25 | 5.6E+05 | 3.6E-03 | 6.4E-09 |
| Ab43 | pH 6.0 | 12.5, 25 | 6.3E+05 | 4.1E-03 | 6.5E-09 |

Fit of binding curves depicted in FIG. 13 assumes a 1:1 binding model. Data are presented for binding to soluble CD138 at pH 6.0 only (given the lack of binding for several of the antibodies at pH 7.4) and is based on an average of values derived from antibody concentrations of 25 nM and 12.5 nM. Mab 4320 kinetic parameters are not included in this analysis due to particularly slow off rates (kdis) out of assay range and not amenable given the limitations of this particular method.

### Example 6. Secondary Characterization of pH Dependent Binding and Relative Binding Affinities of Anti-CD138 Antibody Variants to CD138 Extracellular Domain by Surface Plasmon Resonance.

The binding kinetics provided in Example 5, while informative for comparative purposes, present possible technical limitations. This method as described, immobilizes the target (recombinant CD138) through biotinylation-streptavidin capture. Such a method may, in principle, lead to biases in binding due to either chemical modification of the capture ligand and/or avidity effects due to immobilization of this ligand on the biosensor. Moreover, this method depends on diffusion-based principles of dissociation leading to non-equilibrium binding states. Surface plasmon resonance is an orthogonal, commonly used method for determining binding kinetics and deriving affinity values based on these parameters. In this method, antibodies were directly coupled to the sensor chip and probed with several concentrations of recombinant CD138 (the antigen) varying from 100 nM to 41 nM. This evaluation was completed under two different pH conditions, i.e., pH 6.0 and pH 7.5. Parental antibody mAb 4320 was included for comparative purposes.

Antibody-CD138 binding was evaluated on a Carterra LSA high throughput SPR instrument. Binding experiments were carried out using binding buffer comprising 100 mM sodium phosphate, 150 mM NaCl and 0.05% Tween-20 buffer. Buffers were adjusted to target pH of 7.4 and 6.0, by use of sodium hydroxide or HCL, respectively. All steps were also carried out independently at these respective buffer pHs. Antibodies were captured on the SPR (CMDP) chip using an anti-human IgG covalently coupled to the CMDP chip based on standard protocols, as described by the instrument manufacturer, and to create a uniform lawn on the chip surface at a target 25 nM anti-CD138 antibody concentration. Binding to CD138 was achieved by uniformly passing binding analyte with increasing concentrations of CD138 (41 nM-100 nM) for 15 minutes and allowing dissociation for 20 minutes. Analyte was flowed over the chip at a rate of 2 milliliters/minute. Raw binding data were processed using kinetics data analysis software also provided by the manufacturer (Carterra) following as 2X background subtraction that used the first six injections of buffer blanks and the internal machine reference controls. Kinetic data was analysed using a global fit and assuming a 1:1 binding interaction. Further analysis of individual binding curves (sensorgrams) with best fits were used to refine the calculation of kinetic parameters. Limitations in the fit parameters were noted and for this reason affinity calculations based on binding kinetic parameters (Ka and kDis) are considered approximate as reported under these conditions.

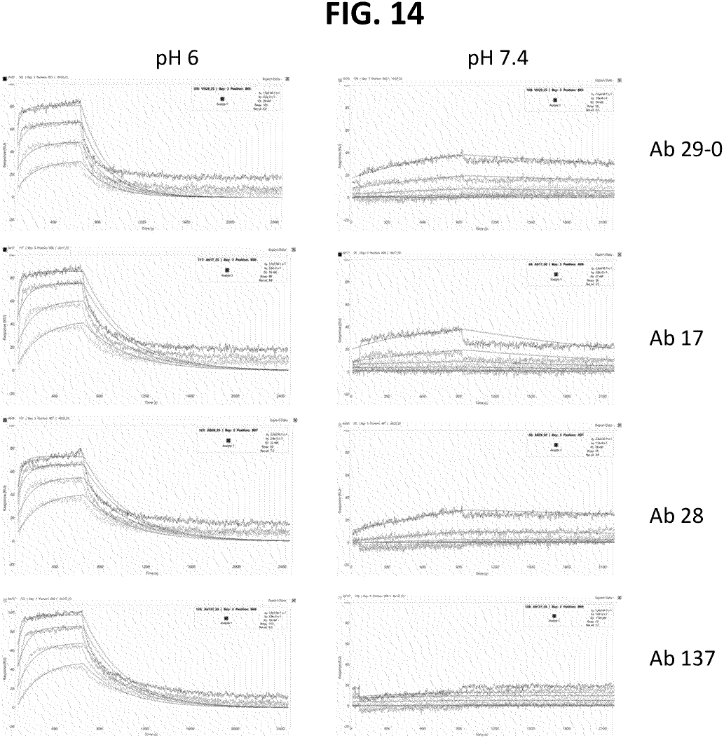

Representative sensorgrams of select antibodies are depicted in FIG. 14. These data visually confirm pH binding selectivity as evidence of greatly reduced binding at pH 7.4 in comparison to pH 6.0. Select antibodies used in this example all exhibited relatively fast off rates (kdis) as shown and as measured under these conditions. These off rates (rates of CD138 dissociation) contribute to the overall affinity values (Kd) of the respective antibodies and intrinsically based on calculation of affinity in terms of the kdis/kon ratios. Binding affinities are summarized in Table 16. The binding parameters of anti-CD138 binding variants generally agree with corresponding values measured by biolayer interferometry and summarized in Example 5.

TABLE 16

CD138-antibody binding parameters measured by SPR*

| | | | | | | |
|---|---|---|---|---|---|---|
| Antibody | pH 6.0 | | | pH 7.4 | | |
| | ka(1/Ms) | kdis (1/s | KD (nM) | ka(1/Ms) | kdis (1/s | KD (nM) |
| 28-0 | 2.0E+5 | 2.0E-3 | 9 nM | 1.7E+4 | 2.8E-4 | 15 nM |
| 29-0 | 1.5E+5 | 4.2E-3 | 28 nM | 1.1E+4 | 1.9E-4 | - |
| Ab17 | 1.7E+5 | 2.6E-3 | 16 nM | 1.3E+4 | 4.8E-4 | -- |
| Ab28 | 2.2E+5 | 2.6E-3 | 12 nM | 3.9E+3 | 1.1E-4 | - |
| Ab71 | 3.0E+5 | 1.8E-3 | 2 nM | 1.0E+4 | 2.8E-4 | 21 nM |
| Ab137 | 1.8E+5 | 2.9E-3 | 16 nM | 1.4E+4 | 1.0E-5 | - |
| MAb 4320 | 3.8E+6 | 7.3E-5 | 0.2 nM | 3.7E+5 | 8.5E-5 | 0.2 nM |

^{∗}KD measurements based on curve fitting parameters; -- (not calculated due to low or negligible binding)

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. An anti-CD138 antibody molecule that selectively binds to CD138 in a tumor microenvironment, wherein the anti-CD138 antibody molecule has a KD value for CD138 at pH 7.4 and a KD value for CD138 at pH of 5.5-7.0, and wherein the ratio of KD for CD138 at pH 7.4 and KD for CD138 at pH of 5.5-7.0 is at least 2.

2-161. (canceled)

162. An anti-CD138 antibody molecule comprising:
(a) a heavy chain variable region (VH), wherein the VH comprises three heavy chain complementarity determining regions (HCDR1, HCDR2, and HCDR3), wherein the VH comprises :
(i) an HCDR1 comprising the amino acid sequence of the HCDR1 of an anti-CD138 antibody as listed in Table 1;
(ii) an HCDR2 comprising the amino acid sequence of the HCDR2 of the anti-CD138 antibody; and
(iii) an HCDR3 comprising the amino acid sequence of the HCDR3 of the anti-CD138 antibody; and
(b) a light chain variable region (VL), wherein the VL comprises three light chain complementarity determining regions (LCDR1, LCDR2, and LCDR3), wherein the VL comprises :
(i) an LCDR1 comprising the amino acid sequence of the LCDR1 of the anti-CD138 antibody;
(ii) an LCDR2 comprising the amino acid sequence of the LCDR2 of the anti-CD138 antibody; and
(iii) an LCDR3 comprising the amino acid sequence of the LCDR3 of the anti-CD138 antibody.

163-167. (canceled)

168. The antibody molecule of claim 162, wherein:
(a) the VH comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody; and/or
(b) the VL comprises an amino acid sequence that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the VH of the anti-CD138 antibody.

169-174. (canceled)

175. The antibody molecule of claim 162, comprising:
(a) a heavy chain (HC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the HC of the anti-CD138 antibody; and/or
(b) a light chain (LC) comprising an amino acid sequence of that differs by no more than 1, 2, 3, 4, 5, 6, 7, 8, 9, 10, 11, 12, 13, 14, or 15 amino acid residues from, or has at least 85, 90, 95, 99 or 100% homology with, the amino acid sequence of the LC of the anti-CD138 antibody.

176-207. (canceled)

208. The antibody molecule of any of embodiments 1-207, which is a synthetic antibody molecule, an isolated antibody molecule, or a humanized antibody molecule; and/or is a monovalent antibody molecule, a multivalent antibody molecule, a monospecific molecule, or a multispecific antibody molecule.

209-212. (canceled)

213. The antibody molecule of any of embodiments 1-212, comprising a heavy chain constant region of IgG chosen from IgG1, IgG2, IgG3, or IgG4, and/or a light chain constant region of kappa or lambda light chain.

214-215. (canceled)

216. The antibody molecule of claim 162, comprising an Fc region comprising one or more mutations to increase the binding affinity to neonatal receptor FcRn and/or the half-life, ADCC, CDC, or ADCP of the antibody molecule.

217-218. (canceled)

219. An antibody-molecule drug conjugate (ADC) comprising the antibody molecule of claim 162 and a non-antibody moiety.

220-221. (canceled)

222. A nucleic acid molecule encoding a heavy chain variable region (VH), a light chain variable region (VL), or both, ofthe antibody molecule of claim 162 .

223. A vector comprising the nucleic acid molecule of claim 222.

224. A cell comprisingthe nucleic acid molecule of claim 222, or a vector comprising the nucleic acid molecule .

225. A kit comprising the antibody molecule of claim 162, an ADC comprising the antibody molecule, or a composition comprising the antibody molecule or the ADC, and instructions to use of the antibody molecule, ADC, or composition.

226. A container comprising the antibody molecule of claim 162, an ADC comprising the antibody molecule, or a composition comprising the antibody molecule or the ADC.

227. A method of producing an anti-CD138 antibody molecule, the method comprising culturingthe cell of claim 224 under conditions that allow production of an antibody molecule, thereby producing the antibody molecule.

228. (canceled)

229. A method of producing an ADC, the method comprising coupling an antibody molecule of claim 162 with a non-antibody moiety, thereby producing the ADC.

230-231. (canceled)

232. A method of treating a cancer, the method comprising administering an effective amount of the antibody molecule of claim 162, an ADC comprising the antibody molecule, or a composition comprising the antibody molecule or the ADC, to a subject in need thereof, thereby treating the cancer.

233. A method of treating an osteolytic disease, the method comprising administering an effective amount of the antibody molecule of claim 162, an ADC comprising the antibody molecule, or a composition comprising the antibody molecule or the ADC, to a subject in need thereof, thereby treating the osteolytic disease.

234-239. (canceled)

240. The method of any of embodiments 231-233, wherein the cancer is a solid tumor or a hematological cancer.

241. The method of claim 232, wherein the cancer is a pancreatic cancer, a breast cancer, renal carcinoma, a lung cancer, a urogenital cancer, a prostate cancer, an ovarian cancer, an endometrial cancer, a uterine cancer, an esophageal cancer, a gastric cancer, a colorectal cancer, a dermal cancer, a squamous cell carcinoma, an adenocarcinoma, a basal cell carcinoma, a mucinous carcinoma, a sarcoma, multiple myeloma, a smoldering myeloma, a plasma cell dyscrasias (light chain amyloidosis), a lymphoma, or a leukemia .

242-246. (canceled)

247. The method of claim 232, further comprising administering to the subject a second therapy or modality for cancer.

248-249. (canceled)

250. A method of targeting an anti-CD138 antibody molecule or an ADC comprising the antibody molecule to a cancer, the method comprising:
providing the anti-CD138 antibody molecule of claim 1 or an ADC comprising the antibody molecule ; and
administering the antibody molecule or the ADC to a subject in need thereof,
thereby targeting the anti-CD138 antibody molecule or the ADC to the cancer.

251-255. (canceled)

256. A method of improving the delivery of an anti-CD138 antibody molecule or an ADC comprising the antibody molecule to a cancer, the method comprising:
providing the anti-CD138 antibody molecule of claim 1 or an ADC comprising the antibody molecule ; and
administering the antibody molecule or the ADC to a subject in need thereof,
thereby improving the delivery of the anti-CD138 antibody molecule or the ADC to the cancer.

257. (canceled)

258. A method of increasing the efficacy of a cancer therapy, the method comprising:
providing a cancer therapy comprising the anti-CD138 antibody molecule of claim 1 or an ADC comprising the antibody molecule ; and
administering the antibody molecule or the ADC to a subject in need thereof,
thereby increasing the efficacy of the cancer therapy.

259. A method of reducing the toxicity of a cancer therapy, the method comprising:
providing a cancer therapy comprising the antibody molecule of claim 1 or an ADC comprising the anti-CD138 antibody molecule ; and
administering the antibody molecule or the ADC to a subject in need thereof,
thereby reducing the toxicity of the cancer therapy.

260-262. (canceled)

263. A method of treating a precancerous condition or preventing a cancer, the method comprising administering to a subject in need thereof an effective amount of the antibody molecule of claim 162, an ADC comprising the antibody molecule, or a composition comprising the antibody molecule or the ADC, thereby treating the precancerous condition or preventing the cancer.

264. (canceled)

265. A method of inhibiting CD138, the method comprising contacting a cell or subject that expresses CD138 the antibody molecule of claim 162, an ADC comprising the antibody molecule, or a composition comprising the antibody molecule or the ADC, thereby inhibiting CD138.

266. (canceled)

267. A method of causing an ADCC activity, the method comprising contacting a cell or subject the antibody molecule of claim 162, an ADC comprising the antibody molecule, or a composition comprising the antibody molecule or the ADC, thereby causing the ADCC activity.

268-269. (canceled)

270. A method of detecting a CD138 molecule, the method comprising contacting a cell or a subject with the antibody molecule of claim 162, thereby detecting the CD138 molecule.

271-272. (canceled)
